SEQUENCE LISTING (260 sequences)

SEQ ID NO: 1 - protein, 120 aa, synthetic construct
  REGION 1..120: note = Description of Artificial Sequence: Synthetic polypeptide
EVQLVESGGGLVQPGGSMRLSCAASGFTFSDYYMAWVRQAPGKGLEWIANINYDGSNTYYADSLKGRFTISRDNSKNTLYLQMNSLRAEDTAVYYCARNWDGYYGYFDVWGQGTTVTVSS

SEQ ID NO: 2 - DNA, 360 bp, synthetic construct
  misc_feature 1..360: note = Description of Artificial Sequence: Synthetic polynucleotide
  source 1..360: mol_type = other DNA
gaggtgcaacttgtggagagcggaggaggtttagtgcaacccggaggcagcatgagactgagctgcgccgccagcggcttcacattctccgactactacatggcttgggtccgacaagctcccggaaaaggactggagtggatcgccaacatcaactacgacggctccaacacctactacgccgactctttaaagggtcgtttcacaatctctcgtgacaacagcaagaacactttatatttacaaatgaactctttaagggccgaggataccgccgtgtactactgcgctcgtaactgggacggctactacggctacttcgacgtgtggggccaaggaaccaccgtgaccgtgagcagc

SEQ ID NO: 3 - protein, 8 aa, synthetic construct
  REGION 1..8: note = Description of Artificial Sequence: Synthetic peptide
GFTFSDYY

SEQ ID NO: 4 - protein, 5 aa, synthetic construct
  REGION 1..5: note = Description of Artificial Sequence: Synthetic peptide
DYYMA

SEQ ID NO: 5 - protein, 6 aa, synthetic construct
  REGION 1..6: note = Description of Artificial Sequence: Synthetic peptide
GFTFSD

SEQ ID NO: 6 - protein, 8 aa, synthetic construct
  REGION 1..8: note = Description of Artificial Sequence: Synthetic peptide
INYDGSNT

SEQ ID NO: 7 - protein, 17 aa, synthetic construct
  REGION 1..17: note = Description of Artificial Sequence: Synthetic peptide
NINYDGSNTYYADSLKG

SEQ ID NO: 8 - protein, 17 aa, synthetic construct
  REGION 1..17: note = Description of Artificial Sequence: Synthetic peptide
NINYDGSNTYYADSLKG

SEQ ID NO: 9 - protein, 13 aa, synthetic construct
  REGION 1..13: note = Description of Artificial Sequence: Synthetic peptide
ARNWDGYYGYFDV

SEQ ID NO: 10 - protein, 11 aa, synthetic construct
  REGION 1..11: note = Description of Artificial Sequence: Synthetic peptide
NWDGYYGYFDV

SEQ ID NO: 11 - protein, 11 aa, synthetic construct
  REGION 1..11: note = Description of Artificial Sequence: Synthetic peptide
NWDGYYGYFDV

SEQ ID NO: 12 - protein, 106 aa, synthetic construct
  REGION 1..106: note = Description of Artificial Sequence: Synthetic polypeptide
DIQLTQSPSSLSASVGDRVTITCRASSSVSHIYWYQQKPGKAPKPWIYRTSNLASGVPSRFSGSGSGTDYTLTISSLQPEDFATYYCQQYHTYPPTFGQGTKLEIK

SEQ ID NO: 13 - DNA, 318 bp, synthetic construct
  misc_feature 1..318: note = Description of Artificial Sequence: Synthetic polynucleotide
  source 1..318: mol_type = other DNA
gatatccagctgacccagtccccttcctctctgtctgcgtctgttggcgatcgtgtcaccatcacttgtcgtgccagcagcagcgtgagccacatttattggtaccaacaaaagcccggcaaagcccctaagccttggatctacagaacctccaatctggccagcggcgtgcccagcagattcagcggaagcggatccggcaccgactacactttaaccatcagctctttacagcccgaggacttcgccacatactactgccagcagtaccacacctatccccccacattcggccaaggaacaaagctggagattaag

SEQ ID NO: 14 - protein, 5 aa, synthetic construct
  REGION 1..5: note = Description of Artificial Sequence: Synthetic peptide
SSVSH

SEQ ID NO: 15 - protein, 10 aa, synthetic construct
  REGION 1..10: note = Description of Artificial Sequence: Synthetic peptide
RASSSVSHIY

SEQ ID NO: 16 - protein, 10 aa, synthetic construct
  REGION 1..10: note = Description of Artificial Sequence: Synthetic peptide
RASSSVSHIY

SEQ ID NO: 17 - skipped

SEQ ID NO: 18 - protein, 7 aa, synthetic construct
  REGION 1..7: note = Description of Artificial Sequence: Synthetic peptide
RTSNLAS

SEQ ID NO: 19 - protein, 7 aa, synthetic construct
  REGION 1..7: note = Description of Artificial Sequence: Synthetic peptide
RTSNLAS

SEQ ID NO: 20 - protein, 9 aa, synthetic construct
  REGION 1..9: note = Description of Artificial Sequence: Synthetic peptide
QQYHTYPPT

SEQ ID NO: 21 - protein, 9 aa, synthetic construct
  REGION 1..9: note = Description of Artificial Sequence: Synthetic peptide
QQYHTYPPT

SEQ ID NO: 22 - protein, 9 aa, synthetic construct
  REGION 1..9: note = Description of Artificial Sequence: Synthetic peptide
QQYHTYPPT

SEQ ID NO: 23 - protein, 244 aa, synthetic construct
  REGION 1..244: note = Description of Artificial Sequence: Synthetic polypeptide
DIQLTQSPSSLSASVGDRVTITCRASSSVSHIYWYQQKPGKAPKPWIYRTSNLASGVPSRFSGSGSGTDYTLTISSLQPEDFATYYCQQYHTYPPTFGQGTKLEIKGSTSGSGKPGSGEGSTKGEVQLVESGGGLVQPGGSMRLSCAASGFTFSDYYMAWVRQAPGKGLEWIANINYDGSNTYYADSLKGRFTISRDNSKNTLYLQMNSLRAEDTAVYYCARNWDGYYGYFDVWGQGTTVTVSS

SEQ ID NO: 24 - DNA, 732 bp, synthetic construct
  misc_feature 1..732: note = Description of Artificial Sequence: Synthetic polynucleotide
  source 1..732: mol_type = other DNA
gatatccagctgacccagtccccttcctctctgtctgcgtctgttggcgatcgtgtcaccatcacttgtcgtgccagcagcagcgtgagccacatttattggtaccaacaaaagcccggcaaagcccctaagccttggatctacagaacctccaatctggccagcggcgtgcccagcagattcagcggaagcggatccggcaccgactacactttaaccatcagctctttacagcccgaggacttcgccacatactactgccagcagtaccacacctatccccccacattcggccaaggaacaaagctggagattaagggctccacctccggaagcggcaaacccggtagcggcgagggctccacaaagggcgaggtgcaacttgtggagagcggaggaggtttagtgcaacccggaggcagcatgagactgagctgcgccgccagcggcttcacattctccgactactacatggcttgggtccgacaagctcccggaaaaggactggagtggatcgccaacatcaactacgacggctccaacacctactacgccgactctttaaagggtcgtttcacaatctctcgtgacaacagcaagaacactttatatttacaaatgaactctttaagggccgaggataccgccgtgtactactgcgctcgtaactgggacggctactacggctacttcgacgtgtggggccaaggaaccaccgtgaccgtgagcagc

SEQ ID NO: 25 - protein, 120 aa, synthetic construct
  REGION 1..120: note = Description of Artificial Sequence: Synthetic polypeptide
QVQLVQSGAEVKKPGASVKLSCKASGYTFTTYWMHWVRQAPGQGLEWIGMIHPNSGSTNYAQKFQGRATLTVDTSTSTAYMELSSLRSEDTAVYYCARDPYDYGEDFDVWGQGTTVTVSS

SEQ ID NO: 26 - DNA, 360 bp, synthetic construct
  misc_feature 1..360: note = Description of Artificial Sequence: Synthetic polynucleotide
  source 1..360: mol_type = other DNA
caagtgcagctggtgcagtccggcgccgaggtgaagaagcccggtgcttccgtgaagctgtcttgcaaagccagcggctacaccttcaccacctattggatgcactgggtccgacaagctcccggtcaaggtctggagtggattggcatgatccaccccaactccggctccaccaactacgcccagaagttccaaggtcgtgccactttaacagtggataccagcaccagcaccgcctacatggagctgagtagtttgaggagcgaggacaccgccgtgtactattgcgctcgtgacccctacgactacggcgaggacttcgacgtgtggggccaaggaacaacagtgaccgtgagcagc

SEQ ID NO: 27 - protein, 8 aa, synthetic construct
  REGION 1..8: note = Description of Artificial Sequence: Synthetic peptide
GYTFTTYW

SEQ ID NO: 28 - protein, 5 aa, synthetic construct
  REGION 1..5: note = Description of Artificial Sequence: Synthetic peptide
TYWMH

SEQ ID NO: 29 - protein, 6 aa, synthetic construct
  REGION 1..6: note = Description of Artificial Sequence: Synthetic peptide
GYTFTT

SEQ ID NO: 30 - protein, 8 aa, synthetic construct
  REGION 1..8: note = Description of Artificial Sequence: Synthetic peptide
IHPNSGST

SEQ ID NO: 31 - protein, 17 aa, synthetic construct
  REGION 1..17: note = Description of Artificial Sequence: Synthetic peptide
MIHPNSGSTNYAQKFQG

SEQ ID NO: 32 - protein, 17 aa, synthetic construct
  REGION 1..17: note = Description of Artificial Sequence: Synthetic peptide
MIHPNSGSTNYAQKFQG

SEQ ID NO: 33 - protein, 13 aa, synthetic construct
  REGION 1..13: note = Description of Artificial Sequence: Synthetic peptide
ARDPYDYGEDFDV

SEQ ID NO: 34 - protein, 11 aa, synthetic construct
  REGION 1..11: note = Description of Artificial Sequence: Synthetic peptide
DPYDYGEDFDV

SEQ ID NO: 35 - protein, 11 aa, synthetic construct
  REGION 1..11: note = Description of Artificial Sequence: Synthetic peptide
DPYDYGEDFDV

SEQ ID NO: 36 - protein, 107 aa, synthetic construct
  REGION 1..107: note = Description of Artificial Sequence: Synthetic polypeptide
DIQMTQSPSSLSASVGDRVTVTCRASQNVNTNVAWYQQKPGKAPKVLIYSASYRNSGVPSRFSGSGSGTDFTLTISSVQPEDFATYYCQQYNSYPFTFGQGTKLEIK

SEQ ID NO: 37 - DNA, 321 bp, synthetic construct
  misc_feature 1..321: note = Description of Artificial Sequence: Synthetic polynucleotide
  source 1..321: mol_type = other DNA
gacatccagatgacccagagccccagctctttaagtgccagcgtgggcgacagagtgacagtgacttgtcgtgccagccagaacgtgaataccaacgtggcttggtaccagcagaagcccggcaaagcccctaaggtgctgatctattccgcgtcttatcgtaactccggcgtgccttcgcgtttttctgggtctggtagcggcaccgacttcactttaacaatcagcagcgttcagcccgaagacttcgccacctactactgccagcagtacaacagctatccctttactttcggtcaagggaccaagctcgagatcaaa

SEQ ID NO: 38 - protein, 6 aa, synthetic construct
  REGION 1..6: note = Description of Artificial Sequence: Synthetic peptide
QNVNTN

SEQ ID NO: 39 - protein, 11 aa, synthetic construct
  REGION 1..11: note = Description of Artificial Sequence: Synthetic peptide
RASQNVNTNVA

SEQ ID NO: 40 - protein, 11 aa, synthetic construct
  REGION 1..11: note = Description of Artificial Sequence: Synthetic peptide
RASQNVNTNVA

SEQ ID NO: 41 - protein, 17 aa, synthetic construct
  REGION 1..17: note = Description of Artificial Sequence: Synthetic peptide
MIHPNSGSTNYAQKFQG

SEQ ID NO: 42 - protein, 7 aa, synthetic construct
  REGION 1..7: note = Description of Artificial Sequence: Synthetic peptide
SASYRNS

SEQ ID NO: 43 - protein, 7 aa, synthetic construct
  REGION 1..7: note = Description of Artificial Sequence: Synthetic peptide
SASYRNS

SEQ ID NO: 44 - protein, 11 aa, synthetic construct
  REGION 1..11: note = Description of Artificial Sequence: Synthetic peptide
DPYDYGEDFDV

SEQ ID NO: 45 - protein, 9 aa, synthetic construct
  REGION 1..9: note = Description of Artificial Sequence: Synthetic peptide
QQYNSYPFT

SEQ ID NO: 46 - protein, 9 aa, synthetic construct
  REGION 1..9: note = Description of Artificial Sequence: Synthetic peptide
QQYNSYPFT

SEQ ID NO: 47 - protein, 245 aa, synthetic construct
  REGION 1..245: note = Description of Artificial Sequence: Synthetic polypeptide
DIQMTQSPSSLSASVGDRVTVTCRASQNVNTNVAWYQQKPGKAPKVLIYSASYRNSGVPSRFSGSGSGTDFTLTISSVQPEDFATYYCQQYNSYPFTFGQGTKLEIKGSTSGSGKPGSGEGSTKGQVQLVQSGAEVKKPGASVKLSCKASGYTFTTYWMHWVRQAPGQGLEWIGMIHPNSGSTNYAQKFQGRATLTVDTSTSTAYMELSSLRSEDTAVYYCARDPYDYGEDFDVWGQGTTVTVSS

SEQ ID NO: 48 - DNA, 735 bp, synthetic construct
  misc_feature 1..735: note = Description of Artificial Sequence: Synthetic polynucleotide
  source 1..735: mol_type = other DNA
gacatccagatgacccagagccccagctctttaagtgccagcgtgggcgacagagtgacagtgacttgtcgtgccagccagaacgtgaataccaacgtggcttggtaccagcagaagcccggcaaagcccctaaggtgctgatctattccgcgtcttatcgtaactccggcgtgccttcgcgtttttctgggtctggtagcggcaccgacttcactttaacaatcagcagcgttcagcccgaagacttcgccacctactactgccagcagtacaacagctatccctttactttcggtcaagggaccaagctcgagatcaaaggctccaccagcggtagcggcaaacccggttccggcgagggctctaccaagggccaagtgcagctggtgcagtccggcgccgaggtgaagaagcccggtgcttccgtgaagctgtcttgcaaagccagcggctacaccttcaccacctattggatgcactgggtccgacaagctcccggtcaaggtctggagtggattggcatgatccaccccaactccggctccaccaactacgcccagaagttccaaggtcgtgccactttaacagtggataccagcaccagcaccgcctacatggagctgagtagtttgaggagcgaggacaccgccgtgtactattgcgctcgtgacccctacgactacggcgaggacttcgacgtgtggggccaaggaacaacagtgaccgtgagcagc

SEQ ID NO: 49 - protein, 120 aa, synthetic construct
  REGION 1..120: note = Description of Artificial Sequence: Synthetic polypeptide
EVQLVESGGGLVQPGGSMRLSCAASGFTFSDYYMAWVRQAPGKGLEWVANINYDGTSTYYADSLKGRFTISRDSSKNTLYLQMNSLRAEDTAVYYCARALDGYYGYLDVWGQGTTVTVSS

SEQ ID NO: 50 - DNA, 360 bp, synthetic construct
  misc_feature 1..360: note = Description of Artificial Sequence: Synthetic polynucleotide
  source 1..360: mol_type = other DNA
gaggtgcagctggtggagtccggaggaggtttagtccaacccggtggcagcatgaggctgtcttgtgctgcctccggcttcactttttctgattactacatggcttgggtccgacaagctcccggaaaaggtttagagtgggtggctaacatcaactacgacggcaccagcacctactatgccgacagcctcaagggcagattcaccatctctcgtgattcgtctaaaaacactttatatttacaaatgaactctttaagagccgaagataccgccgtgtactattgcgctcgtgccctcgacggctactacggatatttagacgtgtggggtcaaggaacaaccgtgaccgtgtccagc

SEQ ID NO: 51 - protein, 8 aa, synthetic construct
  REGION 1..8: note = Description of Artificial Sequence: Synthetic peptide
GFTFSDYY

SEQ ID NO: 52 - protein, 5 aa, synthetic construct
  REGION 1..5: note = Description of Artificial Sequence: Synthetic peptide
DYYMA

SEQ ID NO: 53 - protein, 6 aa, synthetic construct
  REGION 1..6: note = Description of Artificial Sequence: Synthetic peptide
GFTFSD

SEQ ID NO: 54 - protein, 8 aa, synthetic construct
  REGION 1..8: note = Description of Artificial Sequence: Synthetic peptide
INYDGTST

SEQ ID NO: 55 - protein, 17 aa, synthetic construct
  REGION 1..17: note = Description of Artificial Sequence: Synthetic peptide
NINYDGTSTYYADSLKG

SEQ ID NO: 56 - protein, 17 aa, synthetic construct
  REGION 1..17: note = Description of Artificial Sequence: Synthetic peptide
NINYDGTSTYYADSLKG

SEQ ID NO: 57 - protein, 13 aa, synthetic construct
  REGION 1..13: note = Description of Artificial Sequence: Synthetic peptide
ARALDGYYGYLDV

SEQ ID NO: 58 - protein, 11 aa, synthetic construct
  REGION 1..11: note = Description of Artificial Sequence: Synthetic peptide
ALDGYYGYLDV

SEQ ID NO: 59 - protein, 11 aa, synthetic construct
  REGION 1..11: note = Description of Artificial Sequence: Synthetic peptide
ALDGYYGYLDV

SEQ ID NO: 60 - protein, 107 aa, synthetic construct
  REGION 1..107: note = Description of Artificial Sequence: Synthetic polypeptide
DIQLTQSPSSLSASVGDRVTLTCRASQSISNNLHWYQQKPGKAPKLLIKYVSQSISGIPSRFSGSGLGTDFTLTISSVQPEDFATYYCQQSNSWPYTFGQGTKLEIK

SEQ ID NO: 61 - DNA, 321 bp, synthetic construct
  misc_feature 1..321: note = Description of Artificial Sequence: Synthetic polynucleotide
  source 1..321: mol_type = other DNA
gacatccagctgacccagagccctagctctttaagcgctagcgtgggcgatagggtgactctgacttgtcgtgcgtcccaaagcattagcaacaatttacactggtaccagcagaagcccggaaaagcccccaagctgctgatcaaatatgtgagccagagcatctccggcatcccctctcgtttttctggtagcggactgggcaccgactttactttaaccatcagcagcgtccagcccgaggacttcgccacatactactgccagcagagcaacagctggccctatactttcggccaaggaacaaagctggagatcaag

SEQ ID NO: 62 - protein, 6 aa, synthetic construct
  REGION 1..6: note = Description of Artificial Sequence: Synthetic peptide
QSISNN

SEQ ID NO: 63 - protein, 11 aa, synthetic construct
  REGION 1..11: note = Description of Artificial Sequence: Synthetic peptide
RASQSISNNLH

SEQ ID NO: 64 - protein, 11 aa, synthetic construct
  REGION 1..11: note = Description of Artificial Sequence: Synthetic peptide
RASQSISNNLH

SEQ ID NO: 65 - skipped

SEQ ID NO: 66 - protein, 7 aa, synthetic construct
  REGION 1..7: note = Description of Artificial Sequence: Synthetic peptide
YVSQSIS

SEQ ID NO: 67 - protein, 7 aa, synthetic construct
  REGION 1..7: note = Description of Artificial Sequence: Synthetic peptide
YVSQSIS

SEQ ID NO: 68 - protein, 9 aa, synthetic construct
  REGION 1..9: note = Description of Artificial Sequence: Synthetic peptide
QQSNSWPYT

SEQ ID NO: 69 - protein, 9 aa, synthetic construct
  REGION 1..9: note = Description of Artificial Sequence: Synthetic peptide
QQSNSWPYT

SEQ ID NO: 70 - protein, 9 aa, synthetic construct
  REGION 1..9: note = Description of Artificial Sequence: Synthetic peptide
QQSNSWPYT

SEQ ID NO: 71 - protein, 245 aa, synthetic construct
  REGION 1..245: note = Description of Artificial Sequence: Synthetic polypeptide
EVQLVESGGGLVQPGGSMRLSCAASGFTFSDYYMAWVRQAPGKGLEWVANINYDGTSTYYADSLKGRFTISRDSSKNTLYLQMNSLRAEDTAVYYCARALDGYYGYLDVWGQGTTVTVSSGSTSGSGKPGSGEGSTKGDIQLTQSPSSLSASVGDRVTLTCRASQSISNNLHWYQQKPGKAPKLLIKYVSQSISGIPSRFSGSGLGTDFTLTISSVQPEDFATYYCQQSNSWPYTFGQGTKLEIK

SEQ ID NO: 72 - DNA, 735 bp, synthetic construct
  misc_feature 1..735: note = Description of Artificial Sequence: Synthetic polynucleotide
  source 1..735: mol_type = other DNA
gaggtgcagctggtggagtccggaggaggtttagtccaacccggtggcagcatgaggctgtcttgtgctgcctccggcttcactttttctgattactacatggcttgggtccgacaagctcccggaaaaggtttagagtgggtggctaacatcaactacgacggcaccagcacctactatgccgacagcctcaagggcagattcaccatctctcgtgattcgtctaaaaacactttatatttacaaatgaactctttaagagccgaagataccgccgtgtactattgcgctcgtgccctcgacggctactacggatatttagacgtgtggggtcaaggaacaaccgtgaccgtgtccagcggatccacctccggaagcggcaaacccggtagcggcgaaggcagcaccaaaggagacatccagctgacccagagccctagctctttaagcgctagcgtgggcgatagggtgactctgacttgtcgtgcgtcccaaagcattagcaacaatttacactggtaccagcagaagcccggaaaagcccccaagctgctgatcaaatatgtgagccagagcatctccggcatcccctctcgtttttctggtagcggactgggcaccgactttactttaaccatcagcagcgtccagcccgaggacttcgccacatactactgccagcagagcaacagctggccctatactttcggccaaggaacaaagctggagatcaag

SEQ ID NO: 73 - protein, 119 aa, synthetic construct
  REGION 1..119: note = Description of Artificial Sequence: Synthetic polypeptide
EVQLVESGGGLVQPGGSLRLSCAASGFTFSDYYMAWVRQAPGKGLEWVANINYDGSSTFYADSLKGRFTISRDNSKNTLYLQMNSLRAEDTAVYYCGRQVGYYDPMDYWGQGTTVTVSS

SEQ ID NO: 74 - DNA, 357 bp, synthetic construct
  misc_feature 1..357: note = Description of Artificial Sequence: Synthetic polynucleotide
  source 1..357: mol_type = other DNA
gaggtgcagttggtggagagcggaggaggactggtgcagcccggtggctctttaagactcagctgtgccgccagcggatttacattctccgactactacatggcttgggtccgacaagcccccggaaaaggtttagagtgggtggccaacatcaactacgacggctcctccacattctacgccgactctttaaagggtcgtttcaccatctctcgtgacaacagcaaaaatactttatatttacaaatgaactctttaagggccgaggacaccgccgtgtactactgcggtcgtcaagttggctattacgaccccatggactactggggccaaggaactaccgtgaccgtgagcagc

SEQ ID NO: 75 - protein, 8 aa, synthetic construct
  REGION 1..8: note = Description of Artificial Sequence: Synthetic peptide
GFTFSDYY

SEQ ID NO: 76 - protein, 5 aa, synthetic construct
  REGION 1..5: note = Description of Artificial Sequence: Synthetic peptide
DYYMA

SEQ ID NO: 77 - protein, 6 aa, synthetic construct
  REGION 1..6: note = Description of Artificial Sequence: Synthetic peptide
GFTFSD

SEQ ID NO: 78 - protein, 8 aa, synthetic construct
  REGION 1..8: note = Description of Artificial Sequence: Synthetic peptide
INYDGSST

SEQ ID NO: 79 - protein, 17 aa, synthetic construct
  REGION 1..17: note = Description of Artificial Sequence: Synthetic peptide
NINYDGSSTFYADSLKG

SEQ ID NO: 80 - protein, 17 aa, synthetic construct
  REGION 1..17: note = Description of Artificial Sequence: Synthetic peptide
NINYDGSSTFYADSLKG

SEQ ID NO: 81 - protein, 12 aa, synthetic construct
  REGION 1..12: note = Description of Artificial Sequence: Synthetic peptide
GRQVGYYDPMDY

SEQ ID NO: 82 - protein, 10 aa, synthetic construct
  REGION 1..10: note = Description of Artificial Sequence: Synthetic peptide
QVGYYDPMDY

SEQ ID NO: 83 - protein, 10 aa, synthetic construct
  REGION 1..10: note = Description of Artificial Sequence: Synthetic peptide
QVGYYDPMDY

SEQ ID NO: 84 - protein, 106 aa, synthetic construct
  REGION 1..106: note = Description of Artificial Sequence: Synthetic polypeptide
DIQLTQSPSSLSASVGDRVTITCRASSSVSHMYWYQQKPGKAPKPWIYRTSNLASGVPSRFSGSGSGTDYTLTISSMQPEDFATYYCQQYHSYPLTFGQGTKLEIK

SEQ ID NO: 85 - DNA, 318 bp, synthetic construct
  misc_feature 1..318: note = Description of Artificial Sequence: Synthetic polynucleotide
  source 1..318: mol_type = other DNA
gacatccagctgacccagtcccccagctctttatccgctagcgtgggcgatagggtgaccatcacttgtcgtgcgtcttcgtctgtgtctcatatgtactggtaccagcagaagcccggcaaggcccccaagccttggatctatcgtacatccaatcttgcaagcggcgtcccttctcgtttttctggttccgggtctggtaccgactacactttaaccatcagcagcatgcagcccgaggacttcgccacctactactgccagcagtatcactcctatcctttaacttttggccaaggaacaaagttggagatcaag

SEQ ID NO: 86 - protein, 5 aa, synthetic construct
  REGION 1..5: note = Description of Artificial Sequence: Synthetic peptide
SSVSH

SEQ ID NO: 87 - protein, 10 aa, synthetic construct
  REGION 1..10: note = Description of Artificial Sequence: Synthetic peptide
RASSSVSHMY

SEQ ID NO: 88 - protein, 10 aa, synthetic construct
  REGION 1..10: note = Description of Artificial Sequence: Synthetic peptide
RASSSVSHMY

SEQ ID NO: 89 - skipped

SEQ ID NO: 90 - protein, 7 aa, synthetic construct
  REGION 1..7: note = Description of Artificial Sequence: Synthetic peptide
RTSNLAS

SEQ ID NO: 91 - protein, 7 aa, synthetic construct
  REGION 1..7: note = Description of Artificial Sequence: Synthetic peptide
RTSNLAS

SEQ ID NO: 92 - protein, 9 aa, synthetic construct
  REGION 1..9: note = Description of Artificial Sequence: Synthetic peptide
QQYHSYPLT

SEQ ID NO: 93 - protein, 9 aa, synthetic construct
  REGION 1..9: note = Description of Artificial Sequence: Synthetic peptide
QQYHSYPLT

SEQ ID NO: 94 - protein, 9 aa, synthetic construct
  REGION 1..9: note = Description of Artificial Sequence: Synthetic peptide
QQYHSYPLT

SEQ ID NO: 95 - protein, 243 aa, synthetic construct
  REGION 1..243: note = Description of Artificial Sequence: Synthetic polypeptide
DIQLTQSPSSLSASVGDRVTITCRASSSVSHMYWYQQKPGKAPKPWIYRTSNLASGVPSRFSGSGSGTDYTLTISSMQPEDFATYYCQQYHSYPLTFGQGTKLEIKGSTSGSGKPGSGEGSTKGEVQLVESGGGLVQPGGSLRLSCAASGFTFSDYYMAWVRQAPGKGLEWVANINYDGSSTFYADSLKGRFTISRDNSKNTLYLQMNSLRAEDTAVYYCGRQVGYYDPMDYWGQGTTVTVSS

SEQ ID NO: 96 - DNA, 729 bp, synthetic construct
  misc_feature 1..729: note = Description of Artificial Sequence: Synthetic polynucleotide
  source 1..729: mol_type = other DNA
gacatccagctgacccagtcccccagctctttatccgctagcgtgggcgatagggtgaccatcacttgtcgtgcgtcttcgtctgtgtctcatatgtactggtaccagcagaagcccggcaaggcccccaagccttggatctatcgtacatccaatcttgcaagcggcgtcccttctcgtttttctggttccgggtctggtaccgactacactttaaccatcagcagcatgcagcccgaggacttcgccacctactactgccagcagtatcactcctatcctttaacttttggccaaggaacaaagttggagatcaagggcagcacctccggtagcggaaagcccggtagcggcgagggcagcaccaagggagaggtgcagttggtggagagcggaggaggactggtgcagcccggtggctctttaagactcagctgtgccgccagcggatttacattctccgactactacatggcttgggtccgacaagcccccggaaaaggtttagagtgggtggccaacatcaactacgacggctcctccacattctacgccgactctttaaagggtcgtttcaccatctctcgtgacaacagcaaaaatactttatatttacaaatgaactctttaagggccgaggacaccgccgtgtactactgcggtcgtcaagttggctattacgaccccatggactactggggccaaggaactaccgtgaccgtgagcagc

SEQ ID NO: 97 - protein, 115 aa, synthetic construct
  REGION 1..115: note = Description of Artificial Sequence: Synthetic polypeptide
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSS

SEQ ID NO: 98 - DNA, 345 bp, synthetic construct
  misc_feature 1..345: note = Description of Artificial Sequence: Synthetic polynucleotide
  source 1..345: mol_type = other DNA
gaagttcaacttgtgcaaagcggggcagaagtgaaaaaacccggggcgagcgttaaaatatcttgtaaaacaagtggctacaccttcacggagtacaccatccactgggttaaacaagcttctggaaagggacttgaatggatcgggaacataaaccccaacaatgggggcactacttataatcaaaagtttgaggatcgggctaccctcacagtggataagtccacctccacagcttatatggaattgagtagccttaggagcgaggatacagccgtttattattgtgcggcgggctggaactttgactattgggggcaagggacgacggtgacggtgtcctcc

SEQ ID NO: 99 - protein, 8 aa, synthetic construct
  REGION 1..8: note = Description of Artificial Sequence: Synthetic peptide
GYTFTEYT

SEQ ID NO: 100 - protein, 5 aa, synthetic construct
  REGION 1..5: note = Description of Artificial Sequence: Synthetic peptide
EYTIH

SEQ ID NO: 101 - protein, 6 aa, synthetic construct
  REGION 1..6: note = Description of Artificial Sequence: Synthetic peptide
GYTFTE

SEQ ID NO: 102 - protein, 8 aa, synthetic construct
  REGION 1..8: note = Description of Artificial Sequence: Synthetic peptide
INPNNGGT

SEQ ID NO: 103 - protein, 17 aa, synthetic construct
  REGION 1..17: note = Description of Artificial Sequence: Synthetic peptide
NINPNNGGTTYNQKFED

SEQ ID NO: 104 - protein, 17 aa, synthetic construct
  REGION 1..17: note = Description of Artificial Sequence: Synthetic peptide
NINPNNGGTTYNQKFED

SEQ ID NO: 105 - protein, 8 aa, synthetic construct
  REGION 1..8: note = Description of Artificial Sequence: Synthetic peptide
AAGWNFDY

SEQ ID NO: 106 - protein, 6 aa, synthetic construct
  REGION 1..6: note = Description of Artificial Sequence: Synthetic peptide
GWNFDY

SEQ ID NO: 107 - protein, 6 aa, synthetic construct
  REGION 1..6: note = Description of Artificial Sequence: Synthetic peptide
GWNFDY

SEQ ID NO: 108 - protein, 107 aa, synthetic construct
  REGION 1..107: note = Description of Artificial Sequence: Synthetic polypeptide
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 109 - DNA, 321 bp, synthetic construct
  misc_feature 1..321: note = Description of Artificial Sequence: Synthetic polynucleotide
  source 1..321: mol_type = other DNA
gacattgtgatgactcagtctccttcttctctttccgcttccgttggggaccgcgtcactataacttgtaaagcgtcccaagatgtcggcaccgccgttgactggtaccagcaaaaacccgggaaagcgccgaaactgctcatctactgggcttcaacccgccacacgggtgtcccggaccggtttacggggagcggtagtggaaccgatttcactctgaccatttcctcccttcaaccggaagatttcgctgactacttttgtcaacaatataattcatatcccctcactttcggagggggcacgaagttggaaataaag

SEQ ID NO: 110 - protein, 6 aa, synthetic construct
  REGION 1..6: note = Description of Artificial Sequence: Synthetic peptide
QDVGTA

SEQ ID NO: 111 - protein, 11 aa, synthetic construct
  REGION 1..11: note = Description of Artificial Sequence: Synthetic peptide
KASQDVGTAVD

SEQ ID NO: 112 - protein, 11 aa, synthetic construct
  REGION 1..11: note = Description of Artificial Sequence: Synthetic peptide
KASQDVGTAVD

SEQ ID NO: 113 - skipped

SEQ ID NO: 114 - protein, 7 aa, synthetic construct
  REGION 1..7: note = Description of Artificial Sequence: Synthetic peptide
WASTRHT

SEQ ID NO: 115 - protein, 7 aa, synthetic construct
  REGION 1..7: note = Description of Artificial Sequence: Synthetic peptide
WASTRHT

SEQ ID NO: 116 - protein, 10 aa, synthetic construct
  REGION 1..10: note = Description of Artificial Sequence: Synthetic peptide
QQYNSYPLTF

SEQ ID NO: 117 - protein, 9 aa, synthetic construct
  REGION 1..9: note = Description of Artificial Sequence: Synthetic peptide
QQYNSYPLT

SEQ ID NO: 118 - protein, 9 aa, synthetic construct
  REGION 1..9: note = Description of Artificial Sequence: Synthetic peptide
QQYNSYPLT

SEQ ID NO: 119 - protein, 240 aa, synthetic construct
  REGION 1..240: note = Description of Artificial Sequence: Synthetic polypeptide
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKGSTSGSGKPGSGEGSTKGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSS

SEQ ID NO: 120 - DNA, 720 bp, synthetic construct
  misc_feature 1..720: note = Description of Artificial Sequence: Synthetic polynucleotide
  source 1..720: mol_type = other DNA
gacattgtgatgactcagtctccttcttctctttccgcttccgttggggaccgcgtcactataacttgtaaagcgtcccaagatgtcggcaccgccgttgactggtaccagcaaaaacccgggaaagcgccgaaactgctcatctactgggcttcaacccgccacacgggtgtcccggaccggtttacggggagcggtagtggaaccgatttcactctgaccatttcctcccttcaaccggaagatttcgctgactacttttgtcaacaatataattcatatcccctcactttcggagggggcacgaagttggaaataaagggtagcacctctggtagcggcaagcctggctctggcgagggtagtaccaaaggagaagttcaacttgtgcaaagcggggcagaagtgaaaaaacccggggcgagcgttaaaatatcttgtaaaacaagtggctacaccttcacggagtacaccatccactgggttaaacaagcttctggaaagggacttgaatggatcgggaacataaaccccaacaatgggggcactacttataatcaaaagtttgaggatcgggctaccctcacagtggataagtccacctccacagcttatatggaattgagtagccttaggagcgaggatacagccgtttattattgtgcggcgggctggaactttgactattgggggcaagggacgacggtgacggtgtcctcc

SEQ ID NO: 121-123 - skipped (3 entries)

SEQ ID NO: 124 - protein, 8 aa, synthetic construct
  REGION 1..8: note = Description of Artificial Sequence: Synthetic peptide
GGGSGGGS

SEQ ID NO: 125 - DNA, 24 bp, synthetic construct
  misc_feature 1..24: note = Description of Artificial Sequence: Synthetic oligonucleotide
  source 1..24: mol_type = other DNA
ggcggtggaagcggaggaggttcc

SEQ ID NO: 126 - protein, 18 aa, synthetic construct
  REGION 1..18: note = Description of Artificial Sequence: Synthetic peptide
GSTSGSGKPGSGEGSTKG

SEQ ID NO: 127 - DNA, 54 bp, synthetic construct
  misc_feature 1..54: note = Description of Artificial Sequence: Synthetic oligonucleotide
  source 1..54: mol_type = other DNA
ggctccacctccggaagcggcaaacccggtagcggcgagggctccacaaagggc

SEQ ID NO: 128 - protein, 8 aa, synthetic construct
  REGION 1..8: note = Description of Artificial Sequence: Synthetic peptide
KIEAVKSE

SEQ ID NO: 129 - protein, 45 aa, synthetic construct
  REGION 1..45: note = Description of Artificial Sequence: Synthetic polypeptide
TTTPAPRPPTPAPTIASQPLSLRPEACRPAAGGAVHTRGLDFACD

SEQ ID NO: 130 - DNA, 135 bp, synthetic construct
  misc_feature 1..135: note = Description of Artificial Sequence: Synthetic polynucleotide
  source 1..135: mol_type = other DNA
accacgacgccagcgccgcgaccaccaacaccggcgcccaccatcgcgtcgcaacccctgtccctgcgccccgaggcgtgccggccagcggcggggggcgcagtgcacacgagggggctggacttcgcctgtgat

SEQ ID NO: 131 - protein, 24 aa, synthetic construct
  REGION 1..24: note = Description of Artificial Sequence: Synthetic peptide
IYIWAPLAGTCGVLLLSLVITLYC

SEQ ID NO: 132 - DNA, 72 bp, synthetic construct
  misc_feature 1..72: note = Description of Artificial Sequence: Synthetic oligonucleotide
  source 1..72: mol_type = other DNA
atctacatctgggcgcccttggccgggacttgtggggtccttctcctgtcactggttatcaccctttattgc

SEQ ID NO: 133 - protein, 113 aa, synthetic construct
  REGION 1..113: note = Description of Artificial Sequence: Synthetic polypeptide
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - DNA, 339 bp, synthetic construct
  misc_feature 1..339: note = Description of Artificial Sequence: Synthetic polynucleotide
  source 1..339: mol_type = other DNA
ttgagagtgaagttcagcaggagcgcagacgcccccgcctatcagcaaggccagaaccagctctataacgagctcaatttagggcgaagagaggagtacgatgttttggacaagaggcgtggccgggaccccgaaatggggggaaagccgagaaggaagaaccctcaggaaggcttgtacaatgaattgcagaaggataagatggcggaggcatacagtgagattgggatgaaaggcgagcgccggaggggcaaggggcacgatggcctttatcagggtctcagtacagccaccaaggacacctacgacgcccttcacatgcaagccctgccccctcgc

SEQ ID NO: 135 - protein, 41 aa, synthetic construct
  REGION 1..41: note = Description of Artificial Sequence: Synthetic polypeptide
KRGRKKLLYIFKQPFMRPVQTTQEEDGCSCRFPEEEEGGCE

SEQ ID NO: 136 - DNA, 123 bp, synthetic construct
  misc_feature 1..123: note = Description of Artificial Sequence: Synthetic polynucleotide
  source 1..123: mol_type = other DNA
aaacggggcagaaagaaactcctgtatatattcaaacaaccatttatgagaccagtacaaactactcaagaggaagatggctgtagctgccgatttccagaagaagaagaaggaggatgtgaa

SEQ ID NO: 137 - protein, 22 aa, synthetic construct
  REGION 1..22: note = Description of Artificial Sequence: Synthetic peptide
MLLLVTSLLLCELPHPAFLLIP

SEQ ID NO: 138 - protein, 19 aa, synthetic construct
  REGION 1..19: note = Description of Artificial Sequence: Synthetic peptide
MEWTWVFLFLLSVTAGVHS

SEQ ID NO: 139 - protein, 21 aa, synthetic construct
  REGION 1..21: note = Description of Artificial Sequence: Synthetic peptide
MALPVTALLLPLALLLHAARP

SEQ ID NO: 140 - protein, 469 aa, synthetic construct
  REGION 1..469: note = Description of Artificial Sequence: Synthetic polypeptide
DIQLTQSPSSLSASVGDRVTITCRASSSVSHIYWYQQKPGKAPKPWIYRTSNLASGVPSRFSGSGSGTDYTLTISSLQPEDFATYYCQQYHTYPPTFGQGTKLEIKGSTSGSGKPGSGEGSTKGEVQLVESGGGLVQPGGSMRLSCAASGFTFSDYYMAWVRQAPGKGLEWIANINYDGSNTYYADSLKGRFTISRDNSKNTLYLQMNSLRAEDTAVYYCARNWDGYYGYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - DNA, 1407 bp, synthetic construct
  misc_feature 1..1407: note = Description of Artificial Sequence: Synthetic polynucleotide
  source 1..1407: mol_type = other DNA
gatatccagctgacccagtccccttcctctctgtctgcgtctgttggcgatcgtgtcaccatcacttgtcgtgccagcagcagcgtgagccacatttattggtaccaacaaaagcccggcaaagcccctaagccttggatctacagaacctccaatctggccagcggcgtgcccagcagattcagcggaagcggatccggcaccgactacactttaaccatcagctctttacagcccgaggacttcgccacatactactgccagcagtaccacacctatccccccacattcggccaaggaacaaagctggagattaagggctccacctccggaagcggcaaacccggtagcggcgagggctccacaaagggcgaggtgcaacttgtggagagcggaggaggtttagtgcaacccggaggcagcatgagactgagctgcgccgccagcggcttcacattctccgactactacatggcttgggtccgacaagctcccggaaaaggactggagtggatcgccaacatcaactacgacggctccaacacctactacgccgactctttaaagggtcgtttcacaatctctcgtgacaacagcaagaacactttatatttacaaatgaactctttaagggccgaggataccgccgtgtactactgcgctcgtaactgggacggctactacggctacttcgacgtgtggggccaaggaaccaccgtgaccgtgagcagcgggtccaccacgacgccagcgccgcgaccaccaacaccggcgcccaccatcgcgtcgcaacccctgtccctgcgccccgaggcgtgccggccagcggcggggggcgcagtgcacacgagggggctggacttcgcctgtgatatctacatctgggcgcccttggccgggacttgtggggtccttctcctgtcactggttatcaccctttattgcaaacggggcagaaagaaactcctgtatatattcaaacaaccatttatgagaccagtacaaactactcaagaggaagatggctgtagctgccgatttccagaagaagaagaaggaggatgtgaattgagagtgaagttcagcaggagcgcagacgcccccgcctatcagcaaggccagaaccagctctataacgagctcaatttagggcgaagagaggagtacgatgttttggacaagaggcgtggccgggaccccgaaatggggggaaagccgagaaggaagaaccctcaggaaggcttgtacaatgaattgcagaaggataagatggcggaggcatacagtgagattgggatgaaaggcgagcgccggaggggcaaggggcacgatggcctttatcagggtctcagtacagccaccaaggacacctacgacgcccttcacatgcaagccctgccccctcgc

SEQ ID NO: 142 - protein, 470 aa, synthetic construct
  REGION 1..470: note = Description of Artificial Sequence: Synthetic polypeptide
DIQMTQSPSSLSASVGDRVTVTCRASQNVNTNVAWYQQKPGKAPKVLIYSASYRNSGVPSRFSGSGSGTDFTLTISSVQPEDFATYYCQQYNSYPFTFGQGTKLEIKGSTSGSGKPGSGEGSTKGQVQLVQSGAEVKKPGASVKLSCKASGYTFTTYWMHWVRQAPGQGLEWIGMIHPNSGSTNYAQKFQGRATLTVDTSTSTAYMELSSLRSEDTAVYYCARDPYDYGEDFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - DNA, 1410 bp, synthetic construct
  misc_feature 1..1410: note = Description of Artificial Sequence: Synthetic polynucleotide
  source 1..1410: mol_type = other DNA
gacatccagatgacccagagccccagctctttaagtgccagcgtgggcgacagagtgacagtgacttgtcgtgccagccagaacgtgaataccaacgtggcttggtaccagcagaagcccggcaaagcccctaaggtgctgatctattccgcgtcttatcgtaactccggcgtgccttcgcgtttttctgggtctggtagcggcaccgacttcactttaacaatcagcagcgttcagcccgaagacttcgccacctactactgccagcagtacaacagctatccctttactttcggtcaagggaccaagctcgagatcaaaggctccaccagcggtagcggcaaacccggttccggcgagggctctaccaagggccaagtgcagctggtgcagtccggcgccgaggtgaagaagcccggtgcttccgtgaagctgtcttgcaaagccagcggctacaccttcaccacctattggatgcactgggtccgacaagctcccggtcaaggtctggagtggattggcatgatccaccccaactccggctccaccaactacgcccagaagttccaaggtcgtgccactttaacagtggataccagcaccagcaccgcctacatggagctgagtagtttgaggagcgaggacaccgccgtgtactattgcgctcgtgacccctacgactacggcgaggacttcgacgtgtggggccaaggaacaacagtgaccgtgagcagcgggtccaccacgacgccagcgccgcgaccaccaacaccggcgcccaccatcgcgtcgcaacccctgtccctgcgccccgaggcgtgccggccagcggcggggggcgcagtgcacacgagggggctggacttcgcctgtgatatctacatctgggcgcccttggccgggacttgtggggtccttctcctgtcactggttatcaccctttattgcaaacggggcagaaagaaactcctgtatatattcaaacaaccatttatgagaccagtacaaactactcaagaggaagatggctgtagctgccgatttccagaagaagaagaaggaggatgtgaattgagagtgaagttcagcaggagcgcagacgcccccgcctatcagcaaggccagaaccagctctataacgagctcaatttagggcgaagagaggagtacgatgttttggacaagaggcgtggccgggaccccgaaatggggggaaagccgagaaggaagaaccctcaggaaggcttgtacaatgaattgcagaaggataagatggcggaggcatacagtgagattgggatgaaaggcgagcgccggaggggcaaggggcacgatggcctttatcagggtctcagtacagccaccaaggacacctacgacgcccttcacatgcaagccctgccccctcgc

SEQ ID NO: 144 - protein, 470 aa, synthetic construct
  REGION 1..470: note = Description of Artificial Sequence: Synthetic polypeptide
EVQLVESGGGLVQPGGSMRLSCAASGFTFSDYYMAWVRQAPGKGLEWVANINYDGTSTYYADSLKGRFTISRDSSKNTLYLQMNSLRAEDTAVYYCARALDGYYGYLDVWGQGTTVTVSSGSTSGSGKPGSGEGSTKGDIQLTQSPSSLSASVGDRVTLTCRASQSISNNLHWYQQKPGKAPKLLIKYVSQSISGIPSRFSGSGLGTDFTLTISSVQPEDFATYYCQQSNSWPYTFGQGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - DNA, 1410 bp, synthetic construct
  misc_feature 1..1410: note = Description of Artificial Sequence: Synthetic polynucleotide
  source 1..1410: mol_type = other DNA
gaggtgcagctggtggagtccggaggaggtttagtccaacccggtggcagcatgaggctgtcttgtgctgcctccggcttcactttttctgattactacatggcttgggtccgacaagctcccggaaaaggtttagagtgggtggctaacatcaactacgacggcaccagcacctactatgccgacagcctcaagggcagattcaccatctctcgtgattcgtctaaaaacactttatatttacaaatgaactctttaagagccgaagataccgccgtgtactattgcgctcgtgccctcgacggctactacggatatttagacgtgtggggtcaaggaacaaccgtgaccgtgtccagcggatccacctccggaagcggcaaacccggtagcggcgaaggcagcaccaaaggagacatccagctgacccagagccctagctctttaagcgctagcgtgggcgatagggtgactctgacttgtcgtgcgtcccaaagcattagcaacaatttacactggtaccagcagaagcccggaaaagcccccaagctgctgatcaaatatgtgagccagagcatctccggcatcccctctcgtttttctggtagcggactgggcaccgactttactttaaccatcagcagcgtccagcccgaggacttcgccacatactactgccagcagagcaacagctggccctatactttcggccaaggaacaaagctggagatcaaggggtccaccacgacgccagcgccgcgaccaccaacaccggcgcccaccatcgcgtcgcaacccctgtccctgcgccccgaggcgtgccggccagcggcggggggcgcagtgcacacgagggggctggacttcgcctgtgatatctacatctgggcgcccttggccgggacttgtggggtccttctcctgtcactggttatcaccctttattgcaaacggggcagaaagaaactcctgtatatattcaaacaaccatttatgagaccagtacaaactactcaagaggaagatggctgtagctgccgatttccagaagaagaagaaggaggatgtgaattgagagtgaagttcagcaggagcgcagacgcccccgcctatcagcaaggccagaaccagctctataacgagctcaatttagggcgaagagaggagtacgatgttttggacaagaggcgtggccgggaccccgaaatggggggaaagccgagaaggaagaaccctcaggaaggcttgtacaatgaattgcagaaggataagatggcggaggcatacagtgagattgggatgaaaggcgagcgccggaggggcaaggggcacgatggcctttatcagggtctcagtacagccaccaaggacacctacgacgcccttcacatgcaagccctgccccctcgc

SEQ ID NO: 146 - protein, 465 aa, synthetic construct
  REGION 1..465: note = Description of Artificial Sequence: Synthetic polypeptide
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKGSTSGSGKPGSGEGSTKGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - DNA, 1395 bp, synthetic construct
  misc_feature 1..1395: note = Description of Artificial Sequence: Synthetic polynucleotide
  source 1..1395: mol_type = other DNA
gacattgtgatgactcagtctccttcttctctttccgcttccgttggggaccgcgtcactataacttgtaaagcgtcccaagatgtcggcaccgccgttgactggtaccagcaaaaacccgggaaagcgccgaaactgctcatctactgggcttcaacccgccacacgggtgtcccggaccggtttacggggagcggtagtggaaccgatttcactctgaccatttcctcccttcaaccggaagatttcgctgactacttttgtcaacaatataattcatatcccctcactttcggagggggcacgaagttggaaataaagggtagcacctctggtagcggcaagcctggctctggcgagggtagtaccaaaggagaagttcaacttgtgcaaagcggggcagaagtgaaaaaacccggggcgagcgttaaaatatcttgtaaaacaagtggctacaccttcacggagtacaccatccactgggttaaacaagcttctggaaagggacttgaatggatcgggaacataaaccccaacaatgggggcactacttataatcaaaagtttgaggatcgggctaccctcacagtggataagtccacctccacagcttatatggaattgagtagccttaggagcgaggatacagccgtttattattgtgcggcgggctggaactttgactattgggggcaagggacgacggtgacggtgtcctccgggtccaccacgacgccagcgccgcgaccaccaacaccggcgcccaccatcgcgtcgcaacccctgtccctgcgccccgaggcgtgccggccagcggcggggggcgcagtgcacacgagggggctggacttcgcctgtgatatctacatctgggcgcccttggccgggacttgtggggtccttctcctgtcactggttatcaccctttattgcaaacggggcagaaagaaactcctgtatatattcaaacaaccatttatgagaccagtacaaactactcaagaggaagatggctgtagctgccgatttccagaagaagaagaaggaggatgtgaattgagagtgaagttcagcaggagcgcagacgcccccgcctatcagcaaggccagaaccagctctataacgagctcaatttagggcgaagagaggagtacgatgttttggacaagaggcgtggccgggaccccgaaatggggggaaagccgagaaggaagaaccctcaggaaggcttgtacaatgaattgcagaaggataagatggcggaggcatacagtgagattgggatgaaaggcgagcgccggaggggcaaggggcacgatggcctttatcagggtctcagtacagccaccaaggacacctacgacgcccttcacatgcaagccctgccccctcgc

SEQ ID NO: 148 - protein, 468 aa, synthetic construct
  REGION 1..468: note = Description of Artificial Sequence: Synthetic polypeptide
DIQLTQSPSSLSASVGDRVTITCRASSSVSHMYWYQQKPGKAPKPWIYRTSNLASGVPSRFSGSGSGTDYTLTISSMQPEDFATYYCQQYHSYPLTFGQGTKLEIKGSTSGSGKPGSGEGSTKGEVQLVESGGGLVQPGGSLRLSCAASGFTFSDYYMAWVRQAPGKGLEWVANINYDGSSTFYADSLKGRFTISRDNSKNTLYLQMNSLRAEDTAVYYCGRQVGYYDPMDYWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - DNA, 1404 bp, synthetic construct
  misc_feature 1..1404: note = Description of Artificial Sequence: Synthetic polynucleotide
  source 1..1404: mol_type = other DNA
gacatccagctgacccagtcccccagctctttatccgctagcgtgggcgatagggtgaccatcacttgtcgtgcgtcttcgtctgtgtctcatatgtactggtaccagcagaagcccggcaaggcccccaagccttggatctatcgtacatccaatcttgcaagcggcgtcccttctcgtttttctggttccgggtctggtaccgactacactttaaccatcagcagcatgcagcccgaggacttcgccacctactactgccagcagtatcactcctatcctttaacttttggccaaggaacaaagttggagatcaagggcagcacctccggtagcggaaagcccggtagcggcgagggcagcaccaagggagaggtgcagttggtggagagcggaggaggactggtgcagcccggtggctctttaagactcagctgtgccgccagcggatttacattctccgactactacatggcttgggtccgacaagcccccggaaaaggtttagagtgggtggccaacatcaactacgacggctcctccacattctacgccgactctttaaagggtcgtttcaccatctctcgtgacaacagcaaaaatactttatatttacaaatgaactctttaagggccgaggacaccgccgtgtactactgcggtcgtcaagttggctattacgaccccatggactactggggccaaggaactaccgtgaccgtgagcagcgggtccaccacgacgccagcgccgcgaccaccaacaccggcgcccaccatcgcgtcgcaacccctgtccctgcgccccgaggcgtgccggccagcggcggggggcgcagtgcacacgagggggctggacttcgcctgtgatatctacatctgggcgcccttggccgggacttgtggggtccttctcctgtcactggttatcaccctttattgcaaacggggcagaaagaaactcctgtatatattcaaacaaccatttatgagaccagtacaaactactcaagaggaagatggctgtagctgccgatttccagaagaagaagaaggaggatgtgaattgagagtgaagttcagcaggagcgcagacgcccccgcctatcagcaaggccagaaccagctctataacgagctcaatttagggcgaagagaggagtacgatgttttggacaagaggcgtggccgggaccccgaaatggggggaaagccgagaaggaagaaccctcaggaaggcttgtacaatgaattgcagaaggataagatggcggaggcatacagtgagattgggatgaaaggcgagcgccggaggggcaaggggcacgatggcctttatcagggtctcagtacagccaccaaggacacctacgacgcccttcacatgcaagccctgccccctcgc

SEQ ID NO: 150 - protein, 117 aa, synthetic construct
  REGION 1..117: note = Description of Artificial Sequence: Synthetic polypeptide
QVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGGSYNYFDYWGQGTLVTVSS

SEQ ID NO: 151 - DNA, 351 bp, synthetic construct
  misc_feature 1..351: note = Description of Artificial Sequence: Synthetic polynucleotide
  source 1..351: mol_type = other DNA
caagttcagctgcagcagtggggagctggtttactgaagcctagcgagacactgtctttaacatgcgccgtgtacggcggaagcttcagcggcaactattggagctggatcagacagcctcccggtaagggtttagagtggatcggcgagatcaaccactccggctccaccaactataacccctctttaaagtctcgtgtgaccatctccgtggacaccagcaagaaccagttctctttaaagctgagctccgtgacagccgccgacaccgctgtgtattactgtgctcgtggcggcagctacaactacttcgactactggggccaaggtaccctcgtgaccgtgtccagc

SEQ ID NO: 152 - protein, 8 aa, synthetic construct
  REGION 1..8: note = Description of Artificial Sequence: Synthetic peptide
GGSFSGNY

SEQ ID NO: 153 - protein, 5 aa, synthetic construct
  REGION 1..5: note = Description of Artificial Sequence: Synthetic peptide
GNYWS

SEQ ID NO: 154 - protein, 6 aa, synthetic construct
  REGION 1..6: note = Description of Artificial Sequence: Synthetic peptide
GGSFSG

SEQ ID NO: 155 - protein, 7 aa, synthetic construct
  REGION 1..7: note = Description of Artificial Sequence: Synthetic peptide
INHSGST

SEQ ID NO: 156 - protein, 16 aa, synthetic construct
  REGION 1..16: note = Description of Artificial Sequence: Synthetic peptide
EINHSGSTNYNPSLKS

SEQ ID NO: 157 - protein, 16 aa, synthetic construct
  REGION 1..16: note = Description of Artificial Sequence: Synthetic peptide
EINHSGSTNYNPSLKS

SEQ ID NO: 158 - protein, 11 aa, synthetic construct
  REGION 1..11: note = Description of Artificial Sequence: Synthetic peptide
ARGGSYNYFDY

SEQ ID NO: 159 - protein, 9 aa, synthetic construct
  REGION 1..9: note = Description of Artificial Sequence: Synthetic peptide
GGSYNYFDY

SEQ ID NO: 160 - protein, 9 aa, synthetic construct
  REGION 1..9: note = Description of Artificial Sequence: Synthetic peptide
GGSYNYFDY

SEQ ID NO: 161 - protein, 112 aa, synthetic construct
  REGION 1..112: note = Description of Artificial Sequence: Synthetic polypeptide
DIVMTQSPLSLPVTPGEPASISCRSSQSLLHSNGYNYLVWYLQKPGQSPQLLIYLGSIRASGVPDRFSGSGSGTDFTLKISRVEAEDVGVYYCMQPLQTPITFGQGTRLEIK

SEQ ID NO: 162 - DNA, 336 bp, synthetic construct
  misc_feature 1..336: note = Description of Artificial Sequence: Synthetic polynucleotide
  source 1..336: mol_type = other DNA
gacatcgtgatgacacagagccctctgtctttacccgttacccccggtgaacccgctagcatcagctgcagaagctcccagtctttactccacagcaacggctacaactatttagtgtggtatttacagaaacccggccagagcccccagctgctgatttatctgggctccattcgtgctagcggcgtgcccgatagattttccggcagcggaagcggcaccgacttcactttaaagatctctcgtgtggaggccgaggacgtgggcgtctactactgtatgcagcctctgcagacccccattaccttcggccaaggtactcgtctggaaatcaag

SEQ ID NO: 163 - protein, 11 aa, synthetic construct
  REGION 1..11: note = Description of Artificial Sequence: Synthetic peptide
QSLLHSNGYNY

SEQ ID NO: 164 - protein, 16 aa, synthetic construct
  REGION 1..16: note = Description of Artificial Sequence: Synthetic peptide
RSSQSLLHSNGYNYLV

SEQ ID NO: 165 - protein, 16 aa, synthetic construct
  REGION 1..16: note = Description of Artificial Sequence: Synthetic peptide
RSSQSLLHSNGYNYLV

SEQ ID NO: 166 - skipped

SEQ ID NO: 167 - protein, 7 aa, synthetic construct
  REGION 1..7: note = Description of Artificial Sequence: Synthetic peptide
LGSIRAS

SEQ ID NO: 168 - protein, 7 aa, synthetic construct
  REGION 1..7: note = Description of Artificial Sequence: Synthetic peptide
LGSIRAS

SEQ ID NO: 169 - protein, 10 aa, synthetic construct
  REGION 1..10: note = Description of Artificial Sequence: Synthetic peptide
MQPLQTPITF

SEQ ID NO: 170 - protein, 9 aa, synthetic construct
  REGION 1..9: note = Description of Artificial Sequence: Synthetic peptide
MQPLQTPIT

SEQ ID NO: 171 - protein, 9 aa, synthetic construct
  REGION 1..9: note = Description of Artificial Sequence: Synthetic peptide
MQPLQTPIT

SEQ ID NO: 172 - protein, 247 aa, synthetic construct
  REGION 1..247: note = Description of Artificial Sequence: Synthetic polypeptide
DIVMTQSPLSLPVTPGEPASISCRSSQSLLHSNGYNYLVWYLQKPGQSPQLLIYLGSIRASGVPDRFSGSGSGTDFTLKISRVEAEDVGVYYCMQPLQTPITFGQGTRLEIKGSTSGSGKPGSGEGSTKGQVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGGSYNYFDYWGQGTLVTVSS

SEQ ID NO: 173 - DNA, 741 bp, synthetic construct
  misc_feature 1..741: note = Description of Artificial Sequence: Synthetic polynucleotide
  source 1..741: mol_type = other DNA
gacatcgtgatgacacagagccctctgtctttacccgttacccccggtgaacccgctagcatcagctgcagaagctcccagtctttactccacagcaacggctacaactatttagtgtggtatttacagaaacccggccagagcccccagctgctgatttatctgggctccattcgtgctagcggcgtgcccgatagattttccggcagcggaagcggcaccgacttcactttaaagatctctcgtgtggaggccgaggacgtgggcgtctactactgtatgcagcctctgcagacccccattaccttcggccaaggtactcgtctggaaatcaagggcagcaccagcggcagcggaaaacccggaagcggcgagggaagcaccaaaggccaagttcagctgcagcagtggggagctggtttactgaagcctagcgagacactgtctttaacatgcgccgtgtacggcggaagcttcagcggcaactattggagctggatcagacagcctcccggtaagggtttagagtggatcggcgagatcaaccactccggctccaccaactataacccctctttaaagtctcgtgtgaccatctccgtggacaccagcaagaaccagttctctttaaagctgagctccgtgacagccgccgacaccgctgtgtattactgtgctcgtggcggcagctacaactacttcgactactggggccaaggtaccctcgtgaccgtgtccagc

SEQ ID NO: 174 - protein, 54 aa, synthetic construct
  REGION 1..54: note = Description of Artificial Sequence: Synthetic polypeptide
IPYKIEAVKSEPVEPPLPSQLHLMYVAAAAFVLLFFVGCGVLLSRKRRRQHGQL

SEQ ID NO: 175 - protein, 56 aa, synthetic construct
  REGION 1..56: note = Description of Artificial Sequence: Synthetic polypeptide
IPYKIEAVKSEPVEPPLPSQLHLMYVAAAAFVLLFFVGCGVLLSRKRRRQLCIQKL

SEQ ID NO: 176 - protein, 120 aa, synthetic construct
  REGION 1..120: note = Description of Artificial Sequence: Synthetic polypeptide
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDC

SEQ ID NO: 177 - protein, 152 aa, synthetic construct
  REGION 1..152: note = Description of Artificial Sequence: Synthetic polypeptide
AAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLP

SEQ ID NO: 178 - protein, 23 aa, synthetic construct
  REGION 1..23: note = Description of Artificial Sequence: Synthetic peptide
HLMYVAAAAFVLLFFVGCGVLLS

SEQ ID NO: 179 - protein, 306 aa, synthetic construct
  REGION 1..306: note = Description of Artificial Sequence: Synthetic polypeptide
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 180 - protein, 358 aa, synthetic construct
  REGION 1..358: note = Description of Artificial Sequence: Synthetic polypeptide
PCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 181 - protein, 37 aa, synthetic construct
  REGION 1..37: note = Description of Artificial Sequence: Synthetic polypeptide
PCVGSNPCYNQGTCEPTSENPFYRCLCPAKFNGLLCH

SEQ ID NO: 182 - protein, 120 aa, synthetic construct; identical to SEQ ID NO: 176
  REGION 1..120: note = Description of Artificial Sequence: Synthetic polypeptide

SEQ ID NO: 183 - protein, 152 aa, synthetic construct; identical to SEQ ID NO: 177
  REGION 1..152: note = Description of Artificial Sequence: Synthetic polypeptide

SEQ ID NO: 184 - protein, 23 aa, synthetic construct
  REGION 1..23: note = Description of Artificial Sequence: Synthetic peptide
HLMYVAAAAFVLLFFVGCGVLLS

SEQ ID NO: 185 - protein, 37 aa, synthetic construct
  REGION 1..37: note = Description of Artificial Sequence: Synthetic polypeptide
DINECVLSPCRHGASCQNTHGGYRCHCQAGYSGRNCE

SEQ ID NO: 186 - protein, 36 aa, synthetic construct
  REGION 1..36: note = Description of Artificial Sequence: Synthetic polypeptide
DIDDCRPNPCHNGGSCTDGINTAFCDCLPGFRGTFC

SEQ ID NO: 187 - protein, 37 aa, synthetic construct
  REGION 1..37: note = Description of Artificial Sequence: Synthetic polypeptide
DINECASDPCRNGANCTDCVDSYTCTCPAGFSGIHCE

SEQ ID NO: 188 - protein, 32 aa, synthetic construct
  REGION 1..32: note = Description of Artificial Sequence: Synthetic polypeptide
TESSCFNGGTCVDGINSFTCLCPPGFTGSYCQ

SEQ ID NO: 189 - protein, 37 aa, synthetic construct
  REGION 1..37: note = Description of Artificial Sequence: Synthetic polypeptide
DVNECDSQPCLHGGTCQDGCGSYRCTCPQGYTGPNCQ

SEQ ID NO: 190 - protein, 27 aa, synthetic construct
  REGION 1..27: note = Description of Artificial Sequence: Synthetic peptide
DSSPCKNGGKCWQTHTQYRCECPSGWT

SEQ ID NO: 191 - protein, 36 aa, synthetic construct
  REGION 1..36: note = Description of Artificial Sequence: Synthetic polypeptide
LVDECSPSPCQNGATCTDYLGGYSCKCVAGYHGVNC

SEQ ID NO: 192 - protein, 36 aa, synthetic construct
  REGION 1..36: note = Description of Artificial Sequence: Synthetic polypeptide
IDECLSHPCQNGGTCLDLPNTYKCSCPRGTQGVHCE

SEQ ID NO: 193 - protein, 28 aa, synthetic construct
  REGION 1..28: note = Description of Artificial Sequence: Synthetic peptide
CFNNGTCVDQVGGYSCTCPPGFVGERCE

SEQ ID NO: 194 - protein, 39 aa, synthetic construct
  REGION 1..39: note = Description of Artificial Sequence: Synthetic polypeptide
DVNECLSNPCDARGTQNCVQRVNDFHCECRAGHTGRRCE

SEQ ID NO: 195 - protein, 37 aa, synthetic construct; identical to SEQ ID NO: 181
  REGION 1..37: note = Description of Artificial Sequence: Synthetic polypeptide

SEQ ID NO: 196 - protein, 120 aa, synthetic construct; identical to SEQ ID NO: 176
  REGION 1..120: note = Description of Artificial Sequence: Synthetic polypeptide

SEQ ID NO: 197 - protein, 152 aa, synthetic construct; identical to SEQ ID NO: 177
  REGION 1..152: note = Description of Artificial Sequence: Synthetic polypeptide

SEQ ID NO: 198 - protein, 23 aa, synthetic construct
  REGION 1..23: note = Description of Artificial Sequence: Synthetic peptide
HLMYVAAAAFVLLFFVGCGVLLS

SEQ ID NO: 199 - protein, 23 aa, synthetic construct
  REGION 1..23: note = Description of Artificial Sequence: Synthetic peptide
HLMYVAAAAFVLLFFVGCGVLLS

SEQ ID NO: 200 - protein, 10 aa, synthetic construct
  REGION 1..10: note = Description of Artificial Sequence: Synthetic peptide
GRRRRELDPM

SEQ ID NO: 201 - protein, 9 aa, synthetic construct
  REGION 1..9: note = Description of Artificial Sequence: Synthetic peptide
RQRRELDPM

SEQ ID NO: 202 - protein, 8 aa, synthetic construct
  REGION 1..8: note = Description of Artificial Sequence: Synthetic peptide
KIEAVQSE

SEQ ID NO: 203 - protein, 8 aa, synthetic construct
  REGION 1..8: note = Description of Artificial Sequence: Synthetic peptide
VGCGVLLS

SEQ ID NO: 204 - protein, 7 aa, synthetic construct
  REGION 1..7: note = Description of Artificial Sequence: Synthetic peptide
GCGVLLS

SEQ ID NO: 205 - protein, 394 aa, synthetic construct
  REGION 1..394: note = Description of Artificial Sequence: Synthetic polypeptide
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVV

SEQ ID NO: 206 - DNA, 978 bp, synthetic construct
  misc_feature 1..978: note = Description of Artificial Sequence: Synthetic polynucleotide
  source 1..978: mol_type = other DNA
atcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtacaagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcagccgccttcgtgctcctgttctttgtgggctgtggggtgctgctgtcccgcaagcgccggcgg

SEQ ID NO: 207 - protein, 147 aa, synthetic construct
  REGION 1..147: note = Description of Artificial Sequence: Synthetic polypeptide
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 208 - protein, 211 aa, synthetic construct
  REGION 1..211: note = Description of Artificial Sequence: Synthetic polypeptide
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 209 - protein, 205 aa, synthetic construct
  REGION 1..205: note = Description of Artificial Sequence: Synthetic polypeptide
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 210 - protein, 848 aa, synthetic construct
  REGION 1..848: note = Description of Artificial Sequence: Synthetic polypeptide
DIVMTQSPLSLPVTPGEPASISCRSSQSLLHSNGYNYLVWYLQKPGQSPQLLIYLGSIRASGVPDRFSGSGSGTDFTLKISRVEAEDVGVYYCMQPLQTPITFGQGTRLEIKGSTSGSGKPGSGEGSTKGQVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGGSYNYFDYWGQGTLVTVSSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVVGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 211-220 - skipped (10 entries)

SEQ ID NO: 221 - DNA, 2544 bp, synthetic construct
  misc_feature 1..2544: note = Description of Artificial Sequence: Synthetic polynucleotide
  source 1..2544: mol_type = other DNA
gacatcgtgatgacacagagccctctgtctttacccgttacccccggtgaacccgctagcatcagctgcagaagctcccagtctttactccacagcaacggctacaactatttagtgtggtatttacagaaacccggccagagcccccagctgctgatttatctgggctccattcgtgctagcggcgtgcccgatagattttccggcagcggaagcggcaccgacttcactttaaagatctctcgtgtggaggccgaggacgtgggcgtctactactgtatgcagcctctgcagacccccattaccttcggccaaggtactcgtctggaaatcaagggcagcaccagcggcagcggaaaacccggaagcggcgagggaagcaccaaaggccaagttcagctgcagcagtggggagctggtttactgaagcctagcgagacactgtctttaacatgcgccgtgtacggcggaagcttcagcggcaactattggagctggatcagacagcctcccggtaagggtttagagtggatcggcgagatcaaccactccggctccaccaactataacccctctttaaagtctcgtgtgaccatctccgtggacaccagcaagaaccagttctctttaaagctgagctccgtgacagccgccgacaccgctgtgtattactgtgctcgtggcggcagctacaactacttcgactactggggccaaggtaccctcgtgaccgtgtccagcatcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtacaagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagctgcacctcatgtacgtggcagcagccgccttcgtgctcctgttctttgtgggctgtggggtgctgctgtcccgcaagcgccggcggcagcacggtcaactttggttcccagaaggcttcaaggtctccgaagcctccaagaaaaagcgaagggaaccactcggggaagacagtgtagggttgaaacctttgaagaacgccagcgatggagccttgatggatgataaccaaaatgaatggggtgatgaagacctggaaaccaaaaagtttcgctttgaggaacctgtggtaggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggagaccgatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 222 - protein, 10 aa, synthetic construct
  REGION 1..10: note = Description of Artificial Sequence: Synthetic peptide
EQKLISEEDL

SEQ ID NO: 223 - DNA, 30 bp, synthetic construct
  misc_feature 1..30: note = Description of Artificial Sequence: Synthetic oligonucleotide
  source 1..30: mol_type = other DNA
gagcagaagctgattagcgaggaggattta

SEQ ID NO: 224 - protein, 33 aa, synthetic construct
  REGION 1..33: note = Description of Artificial Sequence: Synthetic polypeptide
MEAAVAAPRPRLLLLVLAAAAAAAAALLPGATA

SEQ ID NO: 225 - protein, 126 aa, synthetic construct
  REGION 1..126: note = Description of Artificial Sequence: Synthetic polypeptide
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 226 - protein, 22 aa, synthetic construct
  REGION 1..22: note = Description of Artificial Sequence: Synthetic peptide
MGRGLLRGLWPLHIVLWTRIAS

SEQ ID NO: 227 - protein, 32 aa, synthetic construct
  REGION 1..32: note = Description of Artificial Sequence: Synthetic polypeptide
MLLLVTSLLLCELPHPAFLLIPEQKLISEEDL

SEQ ID NO: 228 - protein, 152 aa, synthetic construct
  REGION 1..152: note = Description of Artificial Sequence: Synthetic polypeptide
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYIRVNRQ

SEQ ID NO: 229 - protein, 194 aa, synthetic construct
  REGION 1..194: note = Description of Artificial Sequence: Synthetic polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 230 - protein, 137 aa, synthetic construct
  REGION 1..137: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 231 - protein, 167 aa, synthetic construct
  REGION 1..167: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCY

SEQ ID NO: 232 - protein, 159 aa, synthetic construct
  REGION 1..159: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGPILLTISILSFFSVALLVIL

SEQ ID NO: 233 - protein, 162 aa, synthetic construct
  REGION 1..162: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGPILLTCPTISILSFFSVALLVIL

SEQ ID NO: 234 - protein, 200 aa, synthetic construct
  REGION 1..200: note = Description of Artificial Sequence: Synthetic polypeptide
ACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 235 - protein, 359 aa, synthetic construct
  REGION 1..359: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 236 - protein, 362 aa, synthetic construct
  REGION 1..362: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 237 - protein, 159 aa, synthetic construct
  REGION 1..159: note = Description of Artificial Sequence: Synthetic polypeptide
MLLLVTSLLLCELPHPAFLLIPTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 238 - protein, 169 aa, synthetic construct
  REGION 1..169: note = Description of Artificial Sequence: Synthetic polypeptide
MLLLVTSLLLCELPHPAFLLIPEQKLISEEDLTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 239 - protein, 381 aa, synthetic construct
  REGION 1..381: note = Description of Artificial Sequence: Synthetic polypeptide
MLLLVTSLLLCELPHPAFLLIPTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 240 - protein, 391 aa, synthetic construct
  REGION 1..391: note = Description of Artificial Sequence: Synthetic polypeptide
MLLLVTSLLLCELPHPAFLLIPEQKLISEEDLTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 241 - protein, 381 aa, synthetic construct; identical to SEQ ID NO: 239
  REGION 1..381: note = Description of Artificial Sequence: Synthetic polypeptide

SEQ ID NO: 242 - protein, 394 aa, synthetic construct
  REGION 1..394: note = Description of Artificial Sequence: Synthetic polypeptide
MLLLVTSLLLCELPHPAFLLIPEQKLISEEDLTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 243 - protein, 384 aa, synthetic construct
  REGION 1..384: note = Description of Artificial Sequence: Synthetic polypeptide
MLLLVTSLLLCELPHPAFLLIPTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 244 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYTCHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKATCTCPSGYTGPACSQDVDECSLGANPCEHAGKCINTLGSFECQCLQGYTGPRCEIDVNECVSNPCQNDATCLDQTGEFQCTCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFTCKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGTGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 245 - protein, 2472 aa, Homo sapiens
SMPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYTCTCPQGYKGADCTEDVDECAMANSNPCEHAGKCVNTDGAFHCECLKGYAGPRCEMDINECHSDPCQNDATCLDKTGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGATCSDQIDECYSSPCLNDGRCIDLVNGYQCNCQPGTSGVNCEINFDDCASNPCIHGTCMDGINRYSCVCSPGFTGQRCNIDIDECASNPCRKGATCINGVNGFRCTCPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFTGGYRCECVPGYQGVNCEYEVDECQNQPCQNGGTCIDLVNHFKCSCPPGTRGLLCEENIDDCARGPHCLNGGQCMDRTGGYSCRCLPGFAGERCEGDINECLSNPCSSEGSLDCIQLTNDYLCVCRSAFTGRHCETFVDVCPQMPCLNGGTCAVASNMPDGFTCRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLIMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLTGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVTCGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 246 - protein, 1235 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWIDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYTCTCPQGYKGADCTEDVDECAMANSNPCEHAGKCVNTDGAFHCECLKGYAGPRCEMDINECHSDPCQNDATCLDKTGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGATCSDQIDECYSSPCLNDGRCIDLVNGYQCNCQPGTSGVNCEINFDDCASNPCIHGTCMDGINRYSCVCSPGFTGQRCNIDIDECASNPCRKGATCINGVNGFRCTCPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFTGGYRCECVPGYQGVNCEYEVDECQNQPCQNGGTCIDLVNHFKCSCPPGTRGMKSSLSIFHPGHCLKL

SEQ ID NO: 247 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDATCDTNPVNGRATCTCPPGFTGGACDQDVDECSTGANPCEHLGRCVNTQGSFLCQCGRGYTGPRCETDVNECLSGPCRNQATCLDRTGQFTCTCMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGTCYDAPGGFRCVCEPGWSGPRCSQSLARDACESQPCRAGGTCSSDGMGFHCTCPPGVQGRQCELLSPCIPNPCEHGGRCESAPGQLPVCSCPQGWQGPRCQQDVDECAGPAPCGPHGTCTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQTGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVTGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLTCQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 248 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFTGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCTCLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQTCKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFTCHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGIRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSTGLKALKPKAEVDEDGVVMCSGPEEGEEVGQAEETGPPSTCQLWSLSGGCGALPQAAMLIPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 249 - protein, 5 aa, Homo sapiens
RKRRR

SEQ ID NO: 250 - protein, 339 aa, Homo sapiens
ILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRNRR

SEQ ID NO: 251 - protein, 292 aa, Homo sapiens
LYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKR

SEQ ID NO: 252 - protein, 295 aa, Homo sapiens
PAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKR

SEQ ID NO: 253 - protein, 7 aa, Homo sapiens
RRRRRRR

SEQ ID NO: 254 - protein, 315 aa, Homo sapiens
PHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRR

SEQ ID NO: 255 - protein, 8 aa, synthetic construct
  REGION 1..8: note = Description of Artificial Sequence: Synthetic peptide
  VARIANT 2: note = Val or Ile
  VARIANT 4: note = Any amino acid
DXEXNPGP

SEQ ID NO: 256 - DNA, 66 bp, synthetic construct
  misc_feature 1..66: note = Description of Artificial Sequence: Synthetic oligonucleotide
  source 1..66: mol_type = other DNA
atgcttctcctggtgacaagccttctgctctgtgaattgccacacccagcattcctcctgattcct

SEQ ID NO: 257 - protein, 50 aa, synthetic construct
  REGION 1..50: note = Description of Artificial Sequence: Synthetic polypeptide
  VARIANT 1..5: note = This region may encompass 1-5 residues
  VARIANT 1..50: note = This sequence may encompass 1-5 (Gly)1-5(Ser)1-5 repeating units
  VARIANT 6..10: note = This region may encompass 1-5 residues
  VARIANT 11..15: note = This region may encompass 1-5 residues
  VARIANT 16..20: note = This region may encompass 1-5 residues
  VARIANT 21..25: note = This region may encompass 1-5 residues
  VARIANT 26..30: note = This region may encompass 1-5 residues
  VARIANT 31..35: note = This region may encompass 1-5 residues
  VARIANT 36..40: note = This region may encompass 1-5 residues
  VARIANT 41..45: note = This region may encompass 1-5 residues
  VARIANT 46..50: note = This region may encompass 1-5 residues
GGGGGSSSSSGGGGGSSSSSGGGGGSSSSSGGGGGSSSSSGGGGGSSSSS

SEQ ID NO: 258 - protein, 4 aa, Homo sapiens
KRKR

SEQ ID NO: 259 - protein, 4 aa, Homo sapiens
RRKR

SEQ ID NO: 260 - protein, 6 aa, synthetic construct
  REGION 1..6: note = Description of Artificial Sequence: Synthetic 6xHis tag
HHHHHH

(19) United States
(12) Patent Application Publication
(10) Pub. No.: US 2025/0066435 A1
(43) Pub. Date: Feb. 27, 2025

(54) PROSTATE CANCER CHIMERIC ANTIGEN RECEPTORS

(71) Applicant: Kite Pharma, Inc., Foster City, CA (US)

(72) Inventors: Matthew Drever, Concord, CA (US); Amy E. Gilbert, San Francisco, CA (US); Samuel T. Haile, Castro Valley, CA (US); Catherine A. Hartzell, Berkeley, CA (US)

(21) Appl. No.: 18/738,550

(22) Filed: Jun. 10, 2024

Related U.S. Application Data
(63) Continuation of application No. 17/553,391, filed on Dec. 16, 2021, now Pat. No. 12,037,605.
(60) Provisional application No. 63/130,529, filed on Dec. 24, 2020.

(51) Int. Cl. C07K 14/47; A61K 39/00; C07K 14/725; C12N 5/0783

(52) U.S. Cl. CPC C07K 14/4702; A61K 39/4611; A61K 39/4631; A61K 39/464493; C07K 14/7051; C12N 5/0636; C12N 5/0637; C12N 5/0638; A61K 2239/31; A61K 2239/48; A61K 2239/58; C07K 2317/51; C07K 2317/515; C07K 2317/53; C07K 2317/565; C07K 2317/622; C07K 2319/03; C07K 2319/715; C12N 2510/00

(57) ABSTRACT

Provided are antibodies, fragments thereof, chimeric antigen receptors (CARs) and T cell receptors (TCRs) comprising one or more of the anti-PMCA antigen binding domains disclosed herein. SynNotch receptors that comprise an anti-PSCA binding domain Provided are polynucleotides encoding antibodies, fragments thereof, CARs, T cell receptors (TCR) and SynNotch receptors. Provided are compositions, cells and cell therapies comprising the same. Further provided are methods of treatment.

### CROSS-REFERENCE TO RELATED APPLICATIONS

This application is a continuation of U.S. application Ser. No. 17/553,391, filed Dec. 16, 2021. U.S. application Ser. No. 17/553,391 claims priority to U.S. Provisional Application No. 63/130,529, filed Dec. 24, 2020. Each of the foregoing documents is hereby incorporated herein by reference in its entirety for all purposes.

### TECHNICAL FIELD

The present disclosure relates to the field of cell therapy, and more specifically, to CARs and/or TCRs that target antigens present on prostate cancer cells.

### REFERENCE TO SEQUENCE LISTING

This application incorporates by reference the Sequence Listing submitted in Computer Readable Form as file K-1096-US-NP_SL.txt, created on Dec. 14, 2021 and containing 296,034 bytes.

### BACKGROUND

Human cancers are by their nature comprised of normal cells that have undergone a genetic or epigenetic conversion to become abnormal cancer cells. In doing so, cancer cells begin to express proteins and other antigens that are distinct from those expressed by normal cells. These aberrant tumor antigens can be used by the body's innate immune system to specifically target and kill cancer cells. However, cancer cells employ various mechanisms to prevent immune cells, such as T and B lymphocytes, from successfully targeting cancer cells.

Current therapies T cell therapies rely on enriched or modified human T cells to target and kill cancer cells in a patient. To increase the ability of T cells to target and kill a particular cancer cell, methods have been developed to engineer T cells to express constructs which direct T cells to a particular target cancer cell. Chimeric antigen receptors (CARs) and engineered T cell receptors (TCRs), which comprise binding domains capable of interacting with a particular tumor antigen, allow T cells to target and kill cancer cells that express the particular tumor antigen. A need exists for CARs and TCRs for targeting and killing cancer cells and, in particular, solid tumor cells, such as prostate cancer cells.

### SUMMARY

Disclosed is an antibody, or antigen binding fragment thereof comprising an anti-PSMA binding domain, wherein the anti-PSMA binding domain comprises sequences of three heavy chain complementarity determining regions (HCDRs) of any one of the heavy chain variable region (HCVR) selected from the group consisting of SEQ ID NOs: 1, 25, 49, and 73, and sequences of three light chain CDRs (LCDRs) of the light chain variable region (LCVR) selected from the group consisting of SEQ ID NOs: 12, 36, 60, and 84.

In certain embodiments, the anti-PSMA binding domain comprises a first domain comprising three heavy chain complementarity determining regions (HCDR1, HCDR2 and HCDR3) and a second domain comprising three light chain complementarity determining regions (LCDR1, LCDR2 and LCDR3), wherein (i) the HCDR1 has a sequence according to any one of SEQ ID NOs: 3-5, 27-29, 51-53, and 75-77; (ii) the HCDR2 has a sequence according to any one of SEQ ID NOs: 6-8, 30-32, 54-56, and 78-80; (iii) the HCDR3 has a sequence according to any one of SEQ ID NOs: 9-11, 33-35, 57-59, and 81-83; (iv) the LCDR1 has a sequence according to any one of SEQ ID NOs: 14-16, 38-40, 62-64, and 86-88; (v) the LCDR2 has a sequence according to any one of SEQ ID NOs: 17-19, 41-43, 65-67, and 89-91; and (vi) the LCDR3 has a sequence according to any one of SEQ ID NOs: 20-22, 44-46, 68-70, and 92-94.

In certain embodiments, the HCDRs comprise: (i) an HCDR1 according to any of SEQ ID NOs: 3-5; an HCDR2 according to any of SEQ ID NOs: 6-8; an HCDR3 according to any one of SEQ ID NOs: 9-11; (ii) an HCDR1 according to any of SEQ ID NOs: 27-29; an HCDR2 according to any of SEQ ID NOs: 30-32; an HCDR3 according to any one of SEQ ID NOs: 33-35; (iii) an HCDR1 according to any of SEQ ID NOs: 51-53; an HCDR2 according to any of SEQ ID NOs: 54-56; an HCDR3 according to any one of SEQ ID NOs: 57-59; or (iv) an HCDR1 according to any of SEQ ID NOs: 75-77; an HCDR2 according to any of SEQ ID NOs: 78-80; an HCDR3 according to any one of SEQ ID NOs: 81-83; and the LCDRs comprise: (i) an LCDR1 according to any of SEQ ID NOs: 14-16; an LCDR2 according to any of SEQ ID NOs: 17-19; an LCDR3 according to any one of SEQ ID NOs: 20-22; (ii) an LCDR1 according to any of SEQ ID NOs: 38-40; an LCDR2 according to any of SEQ ID NOs: 41-43; an LCDR3 according to any one of SEQ ID NOs: 44-46; (iii) an LCDR1 according to any of SEQ ID NOs: 62-64; an LCDR2 according to any of SEQ ID NOs: 65-67; an LCDR3 according to any one of SEQ ID NOs: 68-70; or (iv) an LCDR1 according to any of SEQ ID NOs: 86-88; an LCDR2 according to any of SEQ ID NOs: 89-91; an LCDR3 according to any one of SEQ ID NOs: 92-94.

In certain embodiments, the antigen binding system, antibody, or antigen binding fragment thereof comprises a first domain comprising three heavy chain complementarity determining regions (HCDRs) and a second domain comprising three light chain complementarity determining regions (LCDRs), wherein: the HCDRs and LCDRs comprise: (i) an HCDR1 according to any of SEQ ID NOs: 3-5; an HCDR2 according to any of SEQ ID NOs: 6-8; an HCDR3 according to any one of SEQ ID NOs: 9-11; an LCDR1 according to any of SEQ ID NOs: 14-16; an LCDR2 according to any of SEQ ID NOs: 17-19; an LCDR3 according to any one of SEQ ID NOs: 20-22; (ii) an HCDR1 according to any of SEQ ID NOs: 27-29; an HCDR2 according to any of SEQ ID NOs: 30-32; an HCDR3 according to any one of SEQ ID NOs: 33-35; an LCDR1 according to any of SEQ ID NOs: 38-40; an LCDR2 according to any of SEQ ID NOs: 41-43; an LCDR3 according to any one of SEQ ID NOs: 44-46; (iii) an HCDR1 according to any of SEQ ID NOs: 51-53; an HCDR2 according to any of SEQ ID NOs: 54-56; an HCDR3 according to any one of SEQ ID NOs: 57-59; an LCDR1 according to any of SEQ ID NOs: 62-64; an LCDR2 according to any of SEQ ID NOs: 65-67; an LCDR3 according to any one of SEQ ID NOs: 68-70; or (iv) an HCDR1 according to any of SEQ ID NOs: 75-77; an HCDR2 according to any of SEQ ID NOs: 78-80; an HCDR3 according to any one of SEQ ID NOs: 81-83; an LCDR1 according to any of SEQ ID NOs: 86-88; an LCDR2 according to any of SEQ ID NOs: 89-91; an LCDR3 according to any one of SEQ ID NOs: 92-94.

In certain embodiments, the antibody, or antigen binding fragment thereof comprises a first heavy chain variable domain comprising three HCDRs and a light chain variable domain comprising three LCDRs, wherein: (i) the heavy chain variable domain is at least 80% identical to SEQ ID NO: 1 SEQ ID NO: 25, SEQ ID NO: 49, and SEQ ID NO: 73; and (ii) the light chain variable domain is at least 80% identical to SEQ ID NO: 12, SEQ ID NO: 36, SEQ ID NO: 60, and SEQ ID NO: 84.

In certain embodiments, the antibody, or antigen binding fragment thereof comprises a first heavy chain variable domain comprising three HCDRs and a light chain variable domain comprising three LCDRs, wherein: (i) the heavy chain variable domain is at least 80% identical to SEQ ID NO: 1 and the light chain variable domain is at least 80% identical to SEQ ID NO: 12; (ii) the heavy chain variable domain is at least 80% identical to SEQ ID NO: 25 and the light chain variable domain is at least 80% identical to SEQ ID NO: 36; (iii) the heavy chain variable domain is at least 80% identical to SEQ ID NO: 49 and the light chain variable domain is at least 80% identical to SEQ ID NO: 60; or (iv) the heavy chain variable domain is at least 80% identical to SEQ ID NO: 73 and the light chain variable domain is at least 80% identical to SEQ ID NO: 84.

In certain embodiments, the three HCDRs and the three LCDRs are comprised by a single polypeptide. In certain embodiments, the antigen binding fragment thereof comprises an scFv.

Disclosed is a nucleic acid encoding a disclosed antibody, or antigen binding fragment thereof.

Disclosed is chimeric antigen receptor, comprising the antibody, or antigen binding fragment thereof disclosed herein.

In embodiments the chimeric antigen receptor comprises an antibody, or antigen binding fragment thereof comprising comprises a heavy chain variable domain that is at least 80% identical to SEQ ID NO: 1 and the light chain variable domain that is at least 80% identical to SEQ ID NO: 12; (ii) a heavy chain variable domain that is at least 80% identical to SEQ ID NO: 25 and a light chain variable domain that is at least 80% identical to SEQ ID NO: 36; (iii) a heavy chain variable domain that is at least 80% identical to SEQ ID NO: 49 and a light chain variable domain that is at least 80% identical to SEQ ID NO: 60; (iv) a heavy chain variable domain that is at least 80% identical to SEQ ID NO: 73 and a light chain variable domain that is at least 80% identical to SEQ ID NO: 84; or (v) or a heavy chain variable domain that is at least 80% identical to SEQ ID NO: 97 and a light chain variable domain that is at least 80% identical to SEQ ID NO: 103.

In embodiments the chimeric antigen receptor comprises a transmembrane domain of 4-1BB/CD137, an alpha chain of a T cell receptor, a beta chain of a T cell receptor, CD3 epsilon, CD4, CD5, CD8 alpha, CD9, CD16, CD19, CD22, CD28, CD33, CD37, CD45, CD64, CD80, CD86, CD134, CD137, CD154, or a zeta chain of a T cell receptor, or any combination thereof.

Disclosed is a nucleic acid comprising a chimeric antigen receptor disclosed herein. Disclosed is recombinant vector comprising a nucleic acid disclosed herein. In embodiments the nucleic acid encoding the antibody, antigen binding fragment thereof or chimeric antigen receptor is operatively connected to a constitutively active promotor or a conditionally activated promoter. In embodiments the conditionally activated promoter comprises at least one transcriptional activator binding site. In embodiments the transcriptional activator binding site comprises one or more GAL4 binding sites.

In embodiments a disclosed recombinant vector or nucleic acid further comprises a nucleic acid encoding a dominant negative TGFβ receptor (DN TGFβR), comprising: an extracellular domain (ECD) from a TGF-β receptor and a transmembrane domain (TMD), wherein the recombinant polypeptide lacks amino acid residues responsible for signaling and phosphorylation present in a wild-type TGF-β receptor. In embodiments the ECD is selected from TGF-βRI or TGF-βRII. In embodiments the TMD is selected from TGF-βRI, TGF-βRII, PDGFR, CD4, CD8, CD28, CD127, CD132, CD3ζ, 4-IBB, OX40, ICOS, CTLA-4, PD-1, LAG-3, 2B4, IL-5, IL-7, IL-7Rα, BTLA or mutants of any of the foregoing. In embodiments the DN TGFβR further comprises a heterologous intracellular domain (ICD) which lacks amino acid residues responsible for signaling and phosphorylation present in wild-type TGF-β receptor. In embodiments the DN TGFβR binds TGF-β1.

Disclosed is a s synthetic notch (synNotch) receptor polypeptide comprising from N to C terminus: an extracellular anti-PSCA binding domain, a Notch core domain comprising one or more proteolytic cleavage sites, and an intracellular domain comprising a transcriptional activator comprising a DNA binding domain and a transactivation domain, wherein binding of the binding extracellular anti-PSCA binding domain to PSCA induces cleavage of the Notch core domain at the one or more proteolytic cleavage sites, thereby releasing the intracellular domain and the transcriptional regulator.

In embodiments the anti-PSCA binding domain comprising a first domain comprising three heavy chain complementarity determining regions (HCDR1, HCDR2 and HCDR3) and a second domain comprising three light chain complementarity determining regions (LCDR1, LCDR2 and LCDR3), wherein (i) the HCDR1 has a sequence according to any one of SEQ ID NOs: 152-154, (ii) the HCDR2 has a sequence according to any one of SEQ ID NOs: 155-157; (iii) the HCDR3 has a sequence according to any one of SEQ ID NOs: 158-160; (iv) the LCDR1 has a sequence according to any one of SEQ ID NOs: 163-165; (v) the LCDR2 has a sequence according to any one of SEQ ID NOs: 166-168; and (vi) the LCDR3 has a sequence according to any one of SEQ ID NOs: 169-171. In embodiments the synNotch receptor polypeptide comprises a first heavy chain variable domain comprising the three HCDRs and a light chain variable domain comprising the three LCDRs, wherein: (i) the heavy chain variable domain is at least 80% identical to SEQ ID NO: 150; and (ii) the light chain variable domain is at least 80% identical to SEQ ID NO: 161.

In embodiments the transcriptional regulator is a transcriptional activator. In embodiments the transcriptional activator comprises GAL4, HNF1 alpha or HNF1 beta. In embodiments the transcriptional activator comprises a transactivation domain selected from the group consisting of VP64, RelA (p65), YAP, WWTR1 (TAZ), CREB3 (LZIP), and MyoD.

Disclosed is a nucleic encoding a disclosed synNotch receptor polypeptide.

Disclosed is a recombinant vector comprising a disclosed synNotch receptor polypeptide.

Disclosed is a host cell transformed with a disclosed nucleic acid or disclosed recombinant vector. In embodiments the host cell comprises a T cell or an NK cell.

Disclosed is a pharmaceutical composition comprising a T cell and/or an NK cell transformed with a disclosed nucleic acid or disclosed recombinant vector.

Disclosed is a method of treating disease in a patient in need of thereof, comprising administering a T cell and/or an NK cell transformed with a disclosed nucleic acid or disclosed recombinant vector or the pharmaceutical composition comprising the same to the patient. In embodiments the disease is prostate cancer.

Disclosed is a method of inducing an immune response in a subject or immunizing a subject against a prostate cancer, the method comprising administering to the subject a T cell and/or an NK cell transformed with a disclosed nucleic acid or disclosed recombinant vector or the pharmaceutical composition comprising the same to the patient. In embodiments the T cell and/or the NK cell is allogeneic to the patient. In embodiments the T cell and/or the NK cell is allogeneic to the patient.

### BRIEF DESCRIPTION OF THE DRAWINGS

FIG. 1 is a graph of cytotoxicity vs time for the indicated canonical CARs.

FIG. 2 is a graph of cytotoxicity vs time for the indicated canonical CARs.

FIG. 3 is a graph of cytotoxicity vs time for the indicated canonical CARs.

FIG. 4 is a graph of cytotoxicity vs time for the indicated synNotch activated CARs.

FIG. 5 is a graph of cytotoxicity vs time for the indicated synNotch activated CARs.

FIG. 6 is a graph of cytotoxicity vs time for the indicated synNotch activated CARs.

### DETAILED DESCRIPTION OF THE INVENTION

### Terms

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “e.g.,” as used herein, is used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2^{nd} ed., (2001), CRC Press; “The Dictionary of Cell & Molecular Biology”, 5^{th} ed., (2013), Academic Press; and “The Oxford Dictionary Of Biochemistry And Molecular Biology”, Cammack et al. eds., 2^{nd} ed, (2006), Oxford University Press, provide those of skill in the art with a general dictionary for many of the terms used in this disclosure.

“Administering” refers to the physical introduction of an agent to a subject, such as a modified T cell disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms, “activated” and “activation” refer to the state of a T cell that has been sufficiently stimulated to induce detectable cellular proliferation. In one embodiment, activation may also be associated with induced cytokine production, and detectable effector functions. The term “activated T cells” refers to, among other things, T cells that are proliferating. Signals generated through the TCR alone may be insufficient for full activation of the T cell and one or more secondary or costimulatory signals may also be required. Thus, T cell activation comprises a primary stimulation signal through the TCR/CD3 complex and one or more secondary costimulatory signals. Costimulation may be evidenced by proliferation and/or cytokine production by T cells that have received a primary activation signal, such as stimulation through the TCR/CD3 complex.

The term “agent” may refer to a molecule or entity of any class comprising, or a plurality of molecules or entities, any of which may be, for example, a polypeptide, nucleic acid, saccharide, lipid, small molecule, metal, cell (such as a T cell or NK cell or progenitor of such cells), or organism (for example, a fraction or extract thereof) or component thereof. In some embodiments, an agent may be utilized in isolated or pure form. In some embodiments, an agent may be utilized in a crude or impure form. In some embodiments, an agent may be provided as a population, collection, or library, for example that may be screened to identify or characterize members present therein.

The term “allogeneic” refers to any material derived from one individual which is then introduced to another individual of the same species, e.g., allogeneic T cell transplantation.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

The term “human antibody” is intended to comprise antibodies having variable and constant domain sequences generated, assembled, or derived from human immunoglobulin sequences, or sequences indistinguishable therefrom. In some embodiments, antibodies (or antibody components) may be considered to be “human” even though their amino acid sequences comprise residues or elements not encoded by human germline immunoglobulin sequences (e.g., variations introduced by in vitro random or site-specific mutagenesis or introduced by in vivo somatic mutation). The term “humanized” is intended to comprise antibodies having a variable domain with a sequence derived from a variable domain of a non-human species (e.g., a mouse), modified to be more similar to a human germline encoded sequence. In some embodiments, a “humanized” antibody comprises one or more framework domains having substantially the amino acid sequence of a human framework domain, and one or more complementary determining regions having substantially the amino acid sequence as that of a non-human antibody. In some embodiments, a humanized antibody comprises at least a portion of an immunoglobulin constant region (Fc), generally that of a human immunoglobulin constant domain. In some embodiments, a humanized antibodies may comprise a C_{H}1, hinge, C_{H}2, C_{H}3, and, optionally, a C_{H}4 region of a human heavy chain constant domain.

Antibodies can include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen binding fragments of any of the above. In certain embodiments, antibodies described herein refer to polyclonal antibody populations. Antibodies may also comprise, for example, Fab′ fragments, Fd′ fragments, Fd fragments, isolated CDRs, single chain Fvs, polypeptide-Fc fusions, single domain antibodies (e.g., shark single domain antibodies such as IgNAR or fragments thereof), camelid antibodies, single chain or Tandem diabodies (TandAb®), Anticalins®, Nanobodies® minibodies, BiTE®s, ankyrin repeat proteins or DARPINS®, Avimers®, DARTs, TCR-like antibodies, Adnectins®, Affilins®, Trans-Bodies®, Affibodies®, TrimerX®, MicroProteins, Fynomers®, Centyrins®, and KALBITOR®s.

An immunoglobulin may derive from any of the commonly known isotypes, including but not limited to IgA, secretory IgA, IgG, IgE and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen binding fragment or an antigen-binding portion of any of the aforementioned immunoglobulins, and includes a monovalent and a divalent fragment or portion, and a single chain Ab.

An “antigen binding molecule,” “antigen binding portion,” “antigen binding fragment,” or “antibody fragment” or “antigen binding domain” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR). In certain embodiments, the antigen binding molecule or domain binds to prostate stem cell antigen (PSCA) or prostate-specific membrane antigen (PSMA). In certain embodiments, the antigen binding molecule or domain is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule or domain comprises or consists of avimers.

In some instances, a CDR is substantially identical to one found in a reference antibody (e.g., an antibody of the present disclosure) and/or the sequence of a CDR provided in the present disclosure. In some embodiments, a CDR is substantially identical to a reference CDR (e.g., a CDR provided in the present disclosure) in that it is either identical in sequence or contains between 1, 2, 3, 4, or 5 (e.g., 1-5) amino acid substitutions as compared with the reference CDR. In some embodiments a CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR (e.g., 85-90%, 85-95%, 85-100%, 90-95%, 90-100%, or 95-100%). In some embodiments a CDR is substantially identical to a reference CDR in that it shows at least 96%, 96%, 97%, 98%, 99%, or 100% sequence identity with the reference CDR. In some embodiments a CDR is substantially identical to a reference CDR in that one amino acid within the CDR is deleted, added, or substituted as compared with the reference CDR while the CDR has an amino acid sequence that is otherwise identical with that of the reference CDR. In some embodiments a CDR is substantially identical to a reference CDR in that 2, 3, 4, or 5 (e.g., 2-5) amino acids within the CDR are deleted, added, or substituted as compared with the reference CDR while the CDR has an amino acid sequence that is otherwise identical to the reference CDR. In various embodiments, an antigen binding fragment binds a same antigen as a reference antibody. In various embodiments, an antigen binding fragment cross-competes with the reference antibody, for example, binding to substantially the same or identical epitope as the reference antibody

An antigen binding fragment may be produced by any means. For example, in some embodiments, an antigen binding fragment may be enzymatically or chemically produced by fragmentation of an intact antibody. In some embodiments, an antigen binding fragment may be recombinantly produced (such as by expression of an engineered nucleic acid sequence). In some embodiments, an antigen binding fragment may be wholly or partially synthetically produced. In some embodiments, an antigen binding fragment may have a length of at least about 50, 60, 70, 80, 90, 100, 110, 120, 130, 140, 150, 160, 170, 180, 190 amino acids or more; in some embodiments at least about 200 amino acids (e.g., 50-100, 50-150, 50-200, or 100-200 amino acids).

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

TABLE 1
CDR Numbering

| Loop | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 | H31-H35B | H26-H35B | H26-H32 . . . 34 | H30-H35B |
| | (Kabat Numbering) | | | |
| H1 | H31-H35 | H26-H35 | H26-H32 | H30-H35 |
| | (Chothia Numbering) | | | |
| H2 | H50-H65 | H50-H58 | H52-H56 | H47-H58 |
| H3 | H95-H102 | H95-H102 | H95-H102 | H93-H101 |

The term “Kabat numbering” and like terms are recognized in the art and refer to a system of numbering amino acid residues in the heavy and light chain variable regions of an antibody, or an antigen-binding molecule thereof. In certain aspects, the CDRs of an antibody can be determined according to the Kabat numbering system (see, e.g., Kabat E A & Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

In certain aspects, the CDRs of an antibody can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226). Typically, when using the Kabat numbering convention, the Chothia CDR-HI loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). In a specific embodiment, the CDRs of the antibodies described herein have been determined according to the Chothia numbering scheme.

The terms “constant region” and “constant domain” are interchangeable and have a meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

The term “heavy chain” when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

The term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

An “antigen” refers to a compound, composition, or substance that may stimulate the production of antibodies or a T cell response in a human or animal, including compositions (such as one that includes a tumor-specific protein) that are injected or absorbed into a human or animal. An antigen reacts with the products of specific humoral or cellular immunity, including those induced by heterologous antigens, such as the disclosed antigens. A “target antigen” or “target antigen of interest” is an antigen that is not substantially found on the surface of other normal (desired) cells and to which a binding domain of a TCR or CAR contemplated herein, is designed to bind. A person of skill in the art would readily understand that any macromolecule, including virtually all proteins or peptides, can serve as an antigen. An antigen can be endogenously expressed, i.e. expressed by genomic DNA, or can be recombinantly expressed. An antigen can be specific to a certain tissue, such as a cancer cell, or it can be broadly expressed. In addition, fragments of larger molecules can act as antigens. In one embodiment, antigens are tumor antigens. In one particular embodiment, the antigen is all or a fragment of prostate stem cell antigen (PSCA) or prostate-specific membrane antigen (PSMA). A “target” is any molecule bound by a binding motif, antigen binding system, CAR or antigen binding agent, e.g., an antibody.

“Antigen-specific targeting region” (ASTR) refers to the region of the CAR which targets specific antigens. The targeting regions on the CAR are extracellular. In some embodiments, the antigen-specific targeting regions comprise an antibody or a functional equivalent thereof or a fragment thereof or a derivative thereof and each of the targeting regions target a different antigen. The targeting regions may comprise full length heavy chain, Fab fragments, single chain Fv (scFv) fragments, divalent single chain antibodies or diabodies, each of which are specific to the target antigen. There are, however, numerous alternatives, such as linked cytokines (which leads to recognition of cells bearing the cytokine receptor), affibodies, ligand binding domains from naturally occurring receptors, soluble protein/peptide ligand for a receptor (for example on a tumor cell), peptides, and vaccines to prompt an immune response, which may each be used in various embodiments of this disclosure. In fact, almost any molecule that binds a given antigen with high affinity can be used as an antigen-specific targeting region, as will be appreciated by those of skill in the art.

“Antigen presenting cell” or “APC” refers to cells that process and present antigens to T cells. Exemplary APCs comprise dendritic cells, macrophages, B cells, certain activated epithelial cells, and other cell types capable of TCR stimulation and appropriate T cell costimulation.

An “anti-tumor effect” refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor.

Two events or entities are “associated” with one another if the presence, level, and/or form of one is correlated with that of the other. For example, an entity (e.g., polypeptide, genetic signature, metabolite, microbe, etc.) is considered to be associated with a disease, disorder, or condition, if its presence, level, and/or form correlates with incidence of and/or susceptibility to the disease, disorder, or condition (e.g., across a relevant population). For example, two or more entities are physically “associated” with one another if they interact, directly or indirectly, so that they are and/or remain in physical proximity with one another (e.g., bind). In additional examples, two or more entities that are physically associated with one another are covalently linked or connected to one another, or non-covalently associated, for example by means of hydrogen bonds, van der Waals interaction, hydrophobic interactions, magnetism, and combinations thereof.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Binding affinity” generally refers to the strength of the sum total of non-covalent interactions between a single binding site of a molecule (e.g., an antibody) and its binding partner (e.g., an antigen). Unless indicated otherwise “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIACORE® or KinExA.

The term “KD” (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (sec-1 or 1/s) refers to the dissociation rate constant of a particular antibody-antigen interaction, or the dissociation rate constant of an antibody or antibody-binding fragment. Said value is also referred to as the k₀ᵢr value.

The term “k_{d}” (M-1×sec-1 or 1/M) refers to the association rate constant of a particular antibody-antigen interaction, or the association rate constant of an antibody or antibody-binding fragment.

The term “K_{A}” (M-1 or 1/M) refers to the association equilibrium constant of a particular antibody-antigen interaction, or the association equilibrium constant of an antibody or antibody binding fragment. The association equilibrium constant is obtained by dividing the kₐ by the k_{d}.

The term “binding” generally refers to a non-covalent association between or among two or more entities. Direct binding involves physical contact between entities or moieties. “Indirect” binding involves physical interaction by way of physical contact with one or more intermediate entities. Binding between two or more entities may be assessed in any of a variety of contexts, e.g., where interacting entities or moieties are studied in isolation or in the context of more complex systems (e.g., while covalently or otherwise associated with a carrier entity and/or in a biological system such as a cell).

The terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, BIACORE®, KinExA 3000 instrument (Sapidyne Instruments, Boise, ID), or other assays known in the art. In a specific embodiment, molecules that specifically bind to an antigen bind to the antigen with a K_{A} that is at least 2 logs, 2.5 logs, 3 logs, 4 logs or greater than the K_{A} when the molecules bind to another antigen. Binding may comprise preferential association of a binding motif, antibody, or antigen binding system with a target of the binding motif, antibody, or antigen binding system as compared to association of the binding motif, antibody, or antigen binding system with an entity that is not the target (i.e. non-target). In some embodiments, a binding motif, antibody, or antigen binding system selectively binds a target if binding between the binding motif, antibody, or antigen binding system and the target is greater than 2-fold, greater than 5-fold, greater than 10-fold, 20-fold, 30-fold, 40-fold, 50-fold, 60-fold, 70-fold, 80-fold, 90-fold, or greater than 100-fold as compared with binding of the binding motif, antibody, or antigen binding system and a non-target. In some embodiments, a binding motif, antibody, or antigen binding system selectively binds a target if the binding affinity is less than about 10⁻⁵ M, less than about 10⁻⁶ M, less than about 10⁻⁷ M, less than about 10⁻⁸ M, or less than about 10⁻⁹ M.

In another embodiment, molecules that specifically bind to an antigen bind with a dissociation constant (K_{d}) of about 1×10⁻⁷ M. In some embodiments, the antigen binding molecule specifically binds an antigen with “high affinity” when the K_{d} is about 1×10⁻⁹ M to about 5×10⁻⁹ M. In some embodiments, the antigen binding molecule specifically binds an antigen with “very high affinity” when the K_{d} is 1×10⁻¹⁰ M to about 5×10⁻¹⁰ M. In one embodiment, the antigen binding molecule has a K_{d} of 10⁻⁹ M. In one embodiment, the off-rate is less than about 1×10⁻⁵. In embodiments, the antigen binding molecule binds prostate stem cell antigen (PSCA) or prostate-specific membrane antigen (PSMA) with a K_{d} of about 1×10⁻¹⁰ M to about 5×10⁻¹⁰ M.

In certain embodiments, provided herein is an antibody or an antigen binding molecule thereof that binds to the target human antigen, e.g., In certain embodiments, the antigen binding molecule binds to prostate stem cell antigen (PSCA) or prostate-specific membrane antigen (PSMA) with a 5%, 10%, 15%, 20%, 25%, 30%, 35%, 40%, 45%, 50%, 55%, 60%, 65%, 70% or higher affinity than to another species of the target antigen as measured by, e.g., a radioimmunoassay, surface plasmon resonance, or kinetic exclusion assay. In a specific embodiment, an antibody or an antigen binding molecule thereof described herein, which binds to a target human antigen, will bind to another species of the target antigen with less than 10%, 15%, or 20% of the binding of the antibody or an antigen binding molecule thereof to the human antigen as measured by, e.g., a radioimmunoassay, surface plasmon resonance, or kinetic exclusion assay.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. In some embodiments, the methods of the present disclosure can be used to reduce the tumor size of a tumor derived from, for example, prostate cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In one particular embodiment, the cancer is prostate cancer. The particular cancer can be responsive to chemo- or radiation therapy or the cancer can be refractory. A refractory cancer refers to a cancer that is not amendable to surgical intervention and the cancer is either initially unresponsive to chemo- or radiation therapy or the cancer becomes unresponsive over time.

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

“Chimeric antigen receptor” or “CAR” refers to a molecule engineered to comprise a binding motif and a means of activating immune cells (for example T cells such as naive T cells, central memory T cells, effector memory T cells, NK cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. Similarly an NK cell that has been genetically engineered to express a chimeric antigen receptor may be referred to as a CAR NK cell.

By “decrease” or “lower,” or “lessen,” or “reduce,” or “abate” refers generally to the ability of a composition contemplated herein to produce, elicit, or cause a lesser physiological response (i.e., a downstream effect) compared to the response caused by either the vehicle alone (i.e., an active moiety) or a control molecule/composition. A “decrease” or “reduced” amount is typically a “statistically significant” amount, and may include an decrease that is 1.1, 1.2, 1.5, 2, 2.5, 3, 3.5, 4, 4.5, 5, 5.5, 6, 6.5, 7, 7.5, 8, 8.5, 9, 9.5, 10, 15, 20, 30 or more times (e.g., 500, 1000 times) (including all integers and decimal points in between and above 1, e.g., 1.5, 1.6, 1.7, 1.8, etc.) the response (reference response) produced by vehicle, a control composition.

“Extracellular domain” (or “ECD”) refers to a portion of a polypeptide that, when the polypeptide is present in a cell membrane, is understood to reside outside of the cell membrane, in the extracellular space.

The term “extracellular ligand-binding domain,” as used herein, refers to an oligo- or polypeptide that is capable of binding a ligand, e.g., a cell surface molecule. For example, the extracellular ligand-binding domain may be chosen to recognize a ligand that acts as a cell surface marker on target cells associated with a particular disease state (e.g., cancer). Examples of cell surface markers that may act as ligands include those associated with viral, bacterial and parasitic infections, autoimmune disease and cancer cells.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy, 1999; 6:412-419). The hinge region in a CAR is generally between the transmembrane (TM) and the binding domain. In certain embodiments, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type I membrane proteins such as CD8alpha, CD4, CD28 and CD7, which may be wild-type hinge regions from these molecules or may be altered.

The “transmembrane” region or domain is the portion of the CAR that anchors the extracellular binding portion to the plasma membrane of the immune effector cell, and facilitates binding of the binding domain to the target antigen. The transmembrane domain may be a CD3zeta transmembrane domain, however other transmembrane domains that may be employed include those obtained from CD8alpha, CD4, CD28, CD45, CD9, CD16, CD22, CD33, CD64, CD80, CD86, CD134, CD137, and CD154. In one embodiment, the transmembrane domain is the transmembrane domain of CD137. In certain embodiments, the transmembrane domain is synthetic in which case it would comprise predominantly hydrophobic residues such as leucine and valine.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the terms “intracellular signaling domain” or “signaling domain,” used interchangeably herein, refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the, “signal transduction domain,” and is typically derived from portions of the human CD3 or FcRy chains.

It is known that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular use in the disclosure include those derived from DAP10, DAP12, TCRzeta, FcRgamma, FcRbeta, CD3gamma, CD3delta, CD3epsilon, CD5, CD22, CD79a, CD79b and CD66d.

As used herein, the term, “costimulatory signaling domain.” or “costimulatory domain”, refers to the portion of the CAR comprising the intracellular domain of a costimulatory molecule. Costimulatory molecules are cell surface molecules other than antigen receptors or Fe receptors that provide a second signal required for efficient activation and function of T lymphocytes upon binding to antigen. Examples of such co-stimulatory molecules include CD27, CD28, 4-1 BB (CD137), 0X40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from 4-1 BB, other costimulatory domains are contemplated for use with the CARs described herein. The inclusion of one or more co stimulatory signaling domains may enhance the efficacy and expansion of T cells expressing CAR receptors. The intracellular signaling and costimulatory signaling domains may be linked in any order in tandem to the carboxyl terminus of the transmembrane domain.

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from 4-1BB. CD28, CD137, CD134 and CD278) may more effectively direct antitumor activity as well as increased cytokine secretion, lytic activity, survival and proliferation in CAR expressing T cells in vitro, and in animal models and cancer patients (Milone et al., Molecular Therapy. 2009: 17: 1453-1464; Zhong et al., Molecular Therapy, 2010; 18:413-420; Carpenito et al., PNAS, 2009; 106:3360-3365).

A “costimulatory signal” refers to a signal, which in combination with a primary signal, such as TCR/CD3 ligation, leads to a T cell response, such as, but not limited to, proliferation and/or upregulation or down regulation of key molecules.

A “costimulatory ligand” includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c, CD1-1d, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD1 1a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

A “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody or antigen-binding molecule thereof can be replaced with an amino acid residue with a similar side chain. In general, two sequences are generally considered to be “substantially similar” if they contain a conservative amino acid substitution in corresponding positions. For example, certain amino acids are generally classified as “hydrophobic” or “hydrophilic” amino acids, and/or as having “polar” or “non-polar” side chains. Substitution of one amino acid for another of the same type may be considered a conservative substitution. Exemplary amino acid categorizations are summarized in Tables 2 and 3 below:

TABLE 2

| Amino Acid | 3-Letter | 1-Letter | Property | Property | Hydropathy Index |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

TABLE 3

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

“Combination therapy” refers to those situations in which a subject is simultaneously exposed to two or more therapeutic regimens (e.g., two or more therapeutic moieties). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality (ies) to a subject receiving the other agent(s) or modality (ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

“Corresponding to” may be used to designate the position/identity of a structural element in a molecule or composition through comparison with an appropriate reference molecule or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, for purposes of simplicity, residues in a polypeptide may be designated using a canonical numbering system based on a reference related polypeptide, so that an amino acid “corresponding to” a residue at position 100, for example, need not actually be the 100th amino acid in an amino acid chain provided it corresponds to the residue found at position 100 in the reference polypeptide. Various sequence alignment strategies are available, comprising software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that may be utilized, for example, to identify “corresponding” residues in polypeptides and/or nucleic acids in accordance with the present disclosure.

An antigen binding molecule, such as an antibody, an antigen binding fragment thereof, CAR or TCR, “cross-competes” with a reference binding molecule, such as an antibody or an antigen binding fragment thereof, if the interaction between an antigen and the first antigen binding molecule blocks, limits, inhibits, or otherwise reduces the ability of the reference binding molecule to interact with the antigen. Cross competition can be complete, e.g., binding of the antigen binding molecule to the antigen completely blocks the ability of the reference binding molecule to bind the antigen, or it can be partial, e.g., binding of the antigen binding molecule to the antigen reduces the ability of the reference antigen binding molecule to bind the antigen. In certain embodiments, an antigen binding molecule that cross-competes with a reference antigen binding molecule binds the same or an overlapping epitope as the reference antigen binding molecule. In other embodiments, the antigen binding molecule that cross-competes with a reference antigen binding molecule binds a different epitope than the reference antigen binding molecule. Numerous types of competitive binding assays can be used to determine if one antigen binding molecule competes with another, for example: solid phase direct or indirect radioimmunoassay (RIA); solid phase direct or indirect enzyme immunoassay (EIA); sandwich competition assay (Stahli et al., 1983, Methods in Enzymology 9:242-253); solid phase direct biotin-avidin EIA (Kirkland et al., 1986, J. Immunol. 137:3614-3619); solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82).

A “cytokine,” refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

The term “domain” refers to a portion of an entity. In some embodiments, a “domain” is associated with a structural and/or functional feature of the entity, e.g., so that, when the domain is physically separated from the rest of its parent entity, it substantially or entirely retains the structural and/or functional feature. In some embodiments, a domain may comprise a portion of an entity that, when separated from that (parent) entity and linked or connected with a different (recipient) entity, substantially retains and/or imparts on the recipient entity one or more structural and/or functional features, e.g., that characterized it in the parent entity. In some embodiments, a domain is a portion of a molecule (e.g., a small molecule, carbohydrate, lipid, nucleic acid, or polypeptide). In some embodiments, a domain is a section of a polypeptide; in some such embodiments, a domain is characterized by a structural element (e.g., an amino acid sequence or sequence motif, α-helix character, β-sheet character, coiled-coil character, random coil character, etc.), and/or by a functional feature (e.g., binding activity, enzymatic activity, folding activity, signaling activity, etc.).

The term “dosage form” may be used to refer to a physically discrete unit of an active agent (e.g., an antigen binding system or antibody) for administration to a subject. Generally, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population. The total amount of a therapeutic composition or agent administered to a subject is determined by one or more medical practitioners and may involve administration of more than one dosage forms.

The term “dosing regimen” may be used to refer to a set of one or more unit doses that are administered individually to a subject. In some embodiments, a given therapeutic agent has a recommended dosing regimen, which may involve one or more doses. In some embodiments, a dosing regimen comprises a plurality of doses each of which is separated in time from other doses. In some embodiments, a dosing regimen comprises a plurality of doses and consecutive doses are separated from one another by time periods of equal length; in some embodiments, a dosing regimen comprises a plurality of doses and consecutive doses are separated from one another by time periods of at least two different lengths. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen is periodically adjusted to achieve a desired or beneficial outcome.

“Effector cell” refers to a cell of the immune system that expresses one or more Fc receptors and mediates one or more effector functions. In some embodiments, effector cells may comprise, without limitation, one or more of monocytes, macrophages, neutrophils, dendritic cells, eosinophils, mast cells, platelets, large granular lymphocytes, Langerhans' cells, natural killer (NK) cells, T-lymphocytes, and B-lymphocytes. Effector cells may be of any organism comprising, without limitation, humans, mice, rats, rabbits, and monkeys.

“Effector function” refers to a biological result of interaction of an antibody Fc region with an Fc receptor or ligand. Effector functions comprise, without limitation, antibody-dependent cell-mediated cytotoxicity (ADCC), antibody-dependent cell-mediated phagocytosis (ADCP), and complement-mediated cytotoxicity (CMC). An effector function may be antigen binding dependent, antigen binding independent, or both. ADCC refers to lysis of antibody-bound target cells by immune effector cells. Without wishing to be bound by any theory, ADCC is generally understood to involve Fc receptor (FcR)-bearing effector cells recognizing and subsequently killing antibody-coated target cells (e.g., cells that express on their surface antigens to which an antibody is bound). Effector cells that mediate ADCC may comprise immune cells, comprising yet not limited to, one or more of natural killer (NK) cells, macrophages, neutrophils, eosinophils.

The term “engineered Autologous Cell Therapy,” which can be abbreviated as “eACT™,” also known as adoptive cell transfer, is a process by which a patient's own T cells are collected and subsequently genetically altered to recognize and target one or more antigens expressed on the cell surface of one or more specific tumor cells or malignancies. T cells can be engineered to express, for example, chimeric antigen receptors (CAR) or T cell receptor (TCR). CAR positive (+) T cells are engineered to express an extracellular single chain variable fragment (scFv) with specificity for a particular tumor antigen linked to an intracellular signaling part comprising at least one costimulatory domain and at least one activating domain. The costimulatory domain can be derived from a naturally-occurring costimulatory domain, or a variant thereof, e.g., a variant having a truncated hinge domain (“THD”), and the activating domain can be derived from, e.g., CD3-zeta. In certain embodiments, the CAR is designed to have two, three, four, or more costimulatory domains. The CAR scFv can be designed to target, for example, PSMA, which is a transmembrane protein expressed prostate tissue, including carcinoma.

In some embodiments, the CAR is engineered such that the costimulatory domain is expressed as a separate polypeptide chain. Example CAR T cell therapies and constructs are described in U.S. Patent Publication Nos. 2013/0287748, 2014/0227237, 2014/0099309, and 2014/0050708, which are incorporated by reference in their entirety. “Adoptive cell therapy” or “ACT” involves transfer of immune cells with anti-tumor activity into a subject, e.g., a cancer patient. In some embodiments, ACT is a treatment approach that involves the use of lymphocytes (e.g., engineered lymphocytes) with anti-tumor activity.

An “epitope” refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303). Antibody: antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies may be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques.

“Endogenous” with reference to a gene, protein, and/or nucleic acid refers to the natural presence of that gene, protein, and/or nucleic acid in a cell, such as an immune cell.

“Exogenous” refers to an introduced agent, such as a nucleic acid, gene, or protein, into a cell, for example from an outside source. A nucleic acid introduced into a cell is exogenous even if it encodes a protein which is naturally found in the cell. Such exogenous introduction of a nucleic acid encoding a protein can be used to increase the expression of the protein over the level that would naturally be found in the cell under similar conditions, e.g. without introduction of the exogenous nucleic acid.

The term “excipient” refers to an agent that may be comprised in a composition, for example to provide or contribute to a desired consistency or stabilizing effect. In some embodiments, a suitable excipient may comprise, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

A “fragment” or “portion” of a material or entity as described herein has a structure that comprises a discrete portion of the whole, e.g., of a physical entity or abstract entity. In some embodiments, a fragment lacks one or more moieties found in the whole. In some embodiments, a fragment consists of or comprises a characteristic structural element, domain or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer (e.g., 85-90%, 85-95%, 85-100%, 90-95%, 90-100%, or 95-100%). The whole material or entity may in some embodiments be referred to as the “parent” of the fragment.

The term “fusion polypeptide” or “fusion protein” generally refers to a polypeptide comprising at least two segments. Generally, a polypeptide containing at least two such segments is considered to be a fusion polypeptide if the two segments are moieties that (1) are not comprised in nature in the same peptide, and/or (2) have not previously been linked or connected to one another in a single polypeptide, and/or (3) have been linked or connected to one another through action of the hand of man. In embodiments, a CAR is a fusion protein. In embodiments, a synthetic Notch receptor (synNotch receptor) is a fusion protein.

The term “gene product” or “expression product” generally refers to an RNA transcribed from the gene (pre- and/or post-processing) or a polypeptide (pre- and/or post-modification) encoded by an RNA transcribed from the gene.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell or NK cell, which can either be obtained from a patient or a donor. The cell can be modified to express an exogenous construct, such as, e.g., a chimeric antigen receptor (CAR) or a T cell receptor (TCR), which is incorporated into the cell's genome. Engineering generally comprises manipulation by the hand of man. For example, a polynucleotide is considered to be “engineered” when two or more sequences, that are not linked or connected together in that order in nature, are manipulated by the hand of man to be directly linked or connected to one another in the engineered polynucleotide. In the context of manipulation of cells by techniques of molecular biology, a cell or organism is considered to be “engineered” if it has been manipulated so that its genetic information is altered (e.g., new genetic material not previously present has been introduced, for example by transformation, somatic hybridization, transfection, transduction, or other mechanism, or previously present genetic material is altered or removed, for example by substitution or deletion mutation, or by other protocols). In some embodiments, a binding agent is a modified lymphocyte, e.g., a T cell or NK cell, may be obtained from a patient or a donor. An engineered cell may be modified to express an exogenous construct, such as, e.g., a chimeric antigen receptor (CAR) or a T cell receptor (TCR), which is incorporated into the cell's genome. Progeny of an engineered polynucleotide or binding agent are generally referred to as “engineered” even though the actual manipulation was performed on a prior entity. In some embodiments, “engineered” refers to an entity that has been designed and produced. The term “designed” refers to an agent (i) whose structure is or was selected by the hand of man; (ii) that is produced by a process requiring the hand of man; and/or (iii) that is distinct from natural substances and other known agents.

A “T cell receptor” or “TCR” refers to antigen-recognition molecules present on the surface of T cells. During normal T cell development, each of the four TCR genes, α, β, γ, and δ, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position.

The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

To calculate percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span,” as determined by the algorithm). In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm. Other algorithms are also available for comparison of amino acid or nucleic acid sequences, comprising those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215 (3): 403-410, 1990; Altschul, et al., Methods in Enzymology; Altschul, et al., “Gapped BLAST and PSI-BLAST: a new generation of protein database search programs,” Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying similar sequences, the programs mentioned above generally provide an indication of the degree of similarity. In some embodiments, two sequences are considered to be substantially similar if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are similar and/or identical over a relevant stretch of residues (e.g., 85-90%, 85-95%, 85-100%, 90-95%, 90-100%, or 95-100%). In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues. Sequences with substantial sequence similarity may be homologs of one another.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions.

The terms “improve,” “increase,” “inhibit,” and “reduce” indicate values that are relative to a baseline or other reference measurement. In some embodiments, an appropriate reference measurement may comprise a measurement in certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in comparable system known or expected to respond in a comparable way, in presence of the relevant agent or treatment.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, NK cells and T cell therapies. T cell therapy can include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. No. 5,728,388, and International Publication No. WO 2008/081035.

The T cells or NK cells of the immunotherapy can come from any source known in the art. For example, T cells and NK cells can be differentiated in vitro from a hematopoietic stem cell population or can be obtained from a subject. T cells and NK cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

The term “in vitro” refers to events occurring in an artificial environment, e.g., in a test tube, reaction vessel, cell culture, etc., rather than within a multi-cellular organism. The term “in vitro cell” refers to any cell which is cultured ex vivo. In particular, an in vitro cell can include a T cell or an NK cell. The term “in vivo” refers to events that occur within a multi-cellular organism, such as a human or a non-human animal.

The term “isolated” refers to a substance that (1) has been separated from at least some components with which it was associated at an earlier time or with which the substance would otherwise be associated, and/or (2) is present in a composition that comprises a limited or defined amount or concentration of one or more known or unknown contaminants. An isolated substance, in some embodiments, may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% (e.g., 85-90%, 85-95%, 85-100%, 90-95%, 90-100%, or 95-100%) of other non-substance components with which the substance was associated at an earlier time, e.g., other components or contaminants with which the substance was previously or otherwise would be associated. In certain instances, a substance is isolated if it is present in a composition that comprises a limited or reduced amount or concentration of molecules of a same or similar type. For instance, in certain instances, a nucleic acid, DNA, or RNA substance is isolated if it is present in a composition that comprises a limited or reduced amount or concentration of non-substance nucleic acid, DNA, or RNA molecules. For instance, in certain instances, a polypeptide substance is isolated if it is present in a composition that comprises a limited or reduced amount or concentration of non-substance polypeptide molecules. In certain embodiments, an amount may be, e.g., an amount measured relative to the amount of a desired substance present in a composition. In certain embodiments, a limited amount may be an amount that is no more than 100% of the amount of substance in a composition, e.g., no more than 1%, 5%, 10%, 20%, 30%, 40%, 50%, 60%, 70%, 80%, 90%, or 95% of the amount of substance in a composition (e.g., 85-90%, 85-95%, 85-100%, 90-95%, 90-100%, or 95-100%). In certain instances, a composition is pure or substantially pure with respect to a selected substance. In some embodiments, an isolated substance is about 80%, about 85%, about 90%, about 91%, about 92%, about 93%, about 94%, about 95%, about 96%, about 97%, about 98%, about 99%, or more than about 99% pure (e.g., 85-90%, 85-95%, 85-100%, 90-95%, 90-100%, or 95-100%). A substance is “pure” if it is substantially free of other components or of contaminants. In some embodiments, a substance may still be considered “isolated” or even “pure,” after having been combined with certain other components such as, for example, one or more carriers or excipients (e.g., buffer, solvent, water, etc.); in such embodiments, percent isolation or purity of the substance is calculated without comprising such carriers or excipients.

“Linker” (L) or “linker domain” or “linker region” refers to an oligo- or polypeptide region from about 1 to 100 amino acids in length, for example linking together any of the domains/regions of a CAR, a synNotch receptor, a DN TFGbeta receptor and/or scFv, or ever one of more of those polypeptides together. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linkers include the picornaviral 2A-like linker, CHYSELsequences of porcine teschovirus (P2A), virus (T2A) or combinations, variants and functional equivalents thereof. In other embodiments, the linker sequences may comprise Asp-Val/Ile-Glu-X-Asn-Pro-Gly (2A)-Pro (2B) motif (SEQ ID NO: 255), which results in cleavage between the 2A glycine and the 2B proline. Other linkers will be apparent to those of skill in the art and may be used in connection with this disclosure. A linker may be a portion of a multi-element agent that connects different elements to one another. For example, a polypeptide comprises two or more functional or structural domains may comprise a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. A linker may connect or link together any of the domains/regions of a CAR or a synNotch receptor. In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length (e.g., 1 to 10, 1 to 20, 1 to 30, 1 to 40, 1 to 50, 1 to 60, 1 to 70, 1 to 80, 1 to 90, 1 to 100, 10 to 20, 10 to 30, 10 to 40, 10 to 50, 10 to 60, 10 to 70, 10 to 80, 10 to 90, or 10 to 100 amino acids in length). In some embodiments, a linker is characterized in that it tends not to adopt a rigid three-dimensional structure, and instead provides flexibility to the polypeptide. In another example it may be used to connect to or more polypeptides to be expressed, such as a CAR, a synNotch receptor and/or a TGFβ-DNR as disclosed herein. In some examples, the CAR, the synNotch receptor and/or the DN TGFβ-R are connected by a cleavable linker.

Other linkers include non-cleavable linkers. A number of linkers are employed to realize the subject invention including “flexible linkers.” The latter are rich in glycine. Klein et al., Protein Engineering, Design & Selection Vol. 27, No. 10, pp. 325-330, 2014; Priyanka et al., Protein Sci., 2013 February; 22 (2): 153-167.

In some embodiments, the linker is a synthetic linker. A synthetic linker can have a length of from about 10 amino acids to about 200 amino acids, e.g., from 10 to 25 amino acids, from 25 to 50 amino acids, from 50 to 75 amino acids, from 75 to 100 amino acids, from 100 to 125 amino acids, from 125 to 150 amino acids, from 150 to 175 amino acids, or from 175 to 200 amino acids. A synthetic linker can have a length of from 10 to 30 amino acids, e.g., 10, 11, 12, 13, 14, 15, 16, 17, 18, 19, 20, 21, 22, 23, 24, 25, 26, 27, 28, 29, or 30 amino acids. A synthetic linker can have a length of from 30 to 50 amino acids, e.g., from 30 to 35 amino acids, from 35 to 40 amino acids, from 40 to 45 amino acids, or from 45 to 50 amino acids.

In some embodiments, the linker is a flexible linker. In some embodiments, the linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS), e.g., 1, 2, 3, 4, 5, 6, 7, 8, 9, or 10 or more GS pairs.

The term “lymphocyte” includes natural killer (NK) cells, T cells, or B cells. NK cells are a type of cytotoxic (cell toxic) lymphocyte that represent a component of the inherent immune system. NK cells reject tumors and cells infected by viruses. It works through the process of apoptosis or programmed cell death. They were termed “natural killers” because they do not require activation in order to kill cells. T cells play a role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory T_{SCM} cells, like naive cells, are CD45RO−, CCR7+, CD45RA+, CD62L+ (L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T cells (Tregs, suppressor T cells, or CD4+CD25+ regulatory T cells), Natural Killer T cells (NKT) and Gamma Delta T cells. B-cells, on the other hand, play a role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocking a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

“Nucleic acid” refers to any polymeric chain of nucleotides. A nucleic acid may be DNA, RNA, or a combination thereof. In some embodiments, a nucleic acid comprises one or more natural nucleic acid residues. In some embodiments, a nucleic acid comprises of one or more nucleic acid analogs. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long (e.g., 20 to 100, 20 to 500, 20 to 1000, 20 to 2000, or 20 to 5000 or more residues). In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide.

“Operably linked” refers to a juxtaposition where the components described are in a relationship permitting them to function in their intended manner. For example, a control element “operably linked” to a functional element is associated in such a way that expression and/or activity of the functional element is achieved under conditions compatible with the control element. In embodiments, a promotor is operably linked to nucleic a

A “patient” includes any human who is afflicted with a cancer (e.g., prostate cancer). The terms “subject” and “patient” are used interchangeably herein.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “proliferation” refers to an increase in cell division, either symmetric or asymmetric division of cells. In some embodiments, “proliferation” refers to the symmetric or asymmetric division of T cells. “Increased proliferation” occurs when there is an increase in the number of cells in a treated sample compared to cells in a non-treated sample.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

“Regulatory T cells” (“Treg”, “Treg cells”, or “Tregs”) refer to a lineage of CD4+T lymphocytes that participate in controlling certain immune activities, e.g., autoimmunity, allergy, and response to infection. Regulatory T cells may regulate the activities of T cell populations, and may also influence certain innate immune system cell types. Tregs may be identified by the expression of the biomarkers CD4, CD25 and Foxp3, and low expression of CD127. Naturally occurring Treg cells normally constitute about 5-10% of the peripheral CD4+T lymphocytes. However, Treg cells within a tumor microenvironment (i.e. tumor-infiltrating Treg cells), Treg cells may make up as much as 20-30% of the total CD4+T lymphocyte population.

The term “sample” generally refers to an aliquot of material obtained or derived from a source of interest. In some embodiments, a source of interest is a biological or environmental source. In some embodiments, a source of interest may comprise a cell or an organism, such as a cell population, tissue, or animal (e.g., a human). In some embodiments, a source of interest comprises biological tissue or fluid. In some embodiments, a biological tissue or fluid may comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may comprise an intracellular fluid, an extracellular fluid, an intravascular fluid (blood plasma), an interstitial fluid, a lymphatic fluid, and/or a transcellular fluid. In some embodiments, a biological fluid may comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brochcoalvealar, ductal, nasal, ocular, oral, uterine, vaginal, or other washing or lavage). In some embodiments, a biological sample comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

“Single chain variable fragment”, “single-chain antibody variable fragments” or “scFv” antibodies refer to forms of antibodies comprising the variable regions of only the heavy and light chains, connected by a linker peptide.

The term “stage of cancer” refers to a qualitative or quantitative assessment of the level of advancement of a cancer. In some embodiments, criteria used to determine the stage of a cancer may comprise, without limitation, one or more of where the cancer is located in a body, tumor size, whether the cancer has spread to lymph nodes, whether the cancer has spread to one or more different parts of the body, etc. In some embodiments, cancer may be staged using the so-called TNM System, according to which T refers to the size and extent of the main tumor, usually called the primary tumor; N refers to the number of nearby lymph nodes that have cancer; and M refers to whether the cancer has metastasized. In some embodiments, a cancer may be referred to as Stage 0 (abnormal cells are present without having spread to nearby tissue, also called carcinoma in situ, or CIS; CIS is not cancer, though could become cancer), Stage I-III (cancer is present; the higher the number, the larger the tumor and the more it has spread into nearby tissues), or Stage IV (the cancer has spread to distant parts of the body). In some embodiments, a cancer may be assigned to a stage selected from the group consisting of: in situ; localized (cancer is limited to the place where it started, with no sign that it has spread); regional (cancer has spread to nearby lymph nodes, tissues, or organs): distant (cancer has spread to distant parts of the body); and unknown (there is not enough information to determine the stage).

“Stimulation,” refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex, that specifically binds with a cognate stimulatory ligand present on an antigen present cell. A “stimulatory ligand” is a ligand that when present on an antigen presenting cell (e.g., an APC, a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody (such as OKT3), an MHC Class I molecule loaded with a peptide, a superagonist anti-CD2 antibody, and a superagonist anti-CD28 antibody.

The phrase “therapeutic agent” may refer to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms or human subjects. In some embodiments, an appropriate population may be defined by various criteria, such as a certain age group, gender, genetic background, preexisting clinical conditions, in accordance with presence or absence of a biomarker, etc. In some embodiments, a therapeutic agent is a substance that may be used to alleviate, ameliorate, relieve, inhibit, prevent, delay onset of, reduce severity of, and/or reduce incidence of one or more symptoms or features of a disease, disorder, and/or condition. In some embodiments, a therapeutic agent is an agent that has been or is required to be approved by a government agency before it may be marketed for administration to humans. In some embodiments, a therapeutic agent is an agent for which a medical prescription is required for administration to humans.

A “therapeutically effective amount,” “effective dose,” “effective amount,” or “therapeutically effective dosage” of a therapeutic agent, e.g., engineered CAR T cells or NK cell, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Transformation” refers to any process by which exogenous DNA is introduced into a host cell. Transformation may occur under natural or artificial conditions using various methods. Transformation may be achieved using any known method for the insertion of foreign nucleic acid sequences into a prokaryotic or eukaryotic host cell. In some embodiments, some transformation methodology is selected based on the host cell being transformed and/or the nucleic acid to be inserted. Methods of transformation may comprise, yet are not limited to, viral infection, electroporation, and lipofection. In some embodiments, a “transformed” cell is stably transformed in that the inserted DNA is capable of replication either as an autonomously replicating plasmid or as part of the host chromosome. In some embodiments, a transformed cell may express introduced nucleic acid.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Chimeric Notch receptor” also referred to as “Chimeric Notch receptor,” or “chimeric Notch receptor” or “synthetic Notch receptor” (synNotch receptor) as described in international patent publications WO16138034 and WO18236825, comprises, from N-terminal to C-terminal and in covalent linkage: a) an extracellular ligand binding domain, for example an antigen binding domain, that specifically binds an antigen present on the surface of a cells, such prostate specific membrane antigen (PSMA); b) wherein the synNotch receptor polypeptide has a length of from 50 amino acids to 1000 amino acids, and comprises one or more ligand-binding inducible proteolytic cleavage sites; and c) an intracellular domain, wherein the ligand binding domain is heterologous to the synNotch receptor polypeptide, and wherein binding of ligand to ligand binding domain induces cleavage of the synNotch receptor polypeptide at the one or more ligand binding-inducible proteolytic cleavage sites, thereby releasing the intracellular domain. In some cases, the synNotch receptor polypeptide has a length of from 300 amino acids to 400 amino acids.

In embodiments a synNotch receptor polypeptide comprises a linker interposed between the extracellular ligand binding domain and the Notch receptor polypeptide. In embodiments the intracellular domain is a transcriptional activator, such as a transcription factor. In embodiments, the extracellular ligand binding domain comprises an antibody or antigen binding fragment thereof, that specifically binds to prostate stem cell antigen (PSCA). In some cases, where the first member of the specific binding pair is an antibody, the antibody is a single-chain Fv (scFv), such as an scFv that specifically binds to a prostate stem cell antigen (PSCA). In embodiments, the extracellular ligand binding domain is a nanobody, a single-domain antibody, a diabody, a triabody, or a minibody.

A “transmembrane domain” is a domain of a polypeptide that includes at least one contiguous amino acid sequence that traverses a lipid bilayer when present in the corresponding endogenous polypeptide when expressed in a mammalian cell. For example, a transmembrane domain can include one, two, three, four, five, six, seven, eight, nine, or ten contiguous amino acid sequences that each traverse a lipid bilayer when present in the corresponding endogenous polypeptide when expressed in a mammalian cell. A transmembrane domain can, e.g., include at least one (e.g., two, three, four, five, six, seven, eight, nine, or ten) contiguous amino acid sequence (that traverses a lipid bilayer when present in the corresponding endogenous polypeptide when expressed in a mammalian cell) that has a-helical secondary structure in the lipid bilayer. In some embodiments, a transmembrane domain can include two or more contiguous amino acid sequences (that each traverse a lipid bilayer when present in the corresponding endogenous polypeptide when expressed in a mammalian cell) that form a β-barrel secondary structure in the lipid bilayer. Non-limiting examples of transmembrane domains are described herein. Additional examples of transmembrane domains are known in the art.

The phrase “extracellular side of the plasma membrane” when used to describe the location of a polypeptide means that the polypeptide includes at least one transmembrane domain that traverses the plasma membrane and at least one domain (e.g., at least one antigen-binding domain) that is located in the extracellular space.

The disclosure may employ, unless indicated specifically to the contrary, methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeck, D. H. Margulies, E. M. Shevach and W. Strober, eds., 1991); Annual Review of Immunology; as well as monographs in journals such as Advances in Immunology.

The present disclosure provides antigen binding agents, such as antibodies, chimeric antigen receptors (CARs) and T cell receptors (TCRs) comprising at least an anti-PSMA binding domain. Among other things, the present disclosure provides methods and compositions useful for treatment of cancer and/or for initiating or modulating immune responses. In various embodiments, one or more anti-PSMA binding domain is an scFv. Exemplary anti-PSMA binding domain amino acid sequences, and nucleic acid sequences encoding the same, are provided herein, for example in Tables 4-8. In some embodiments, an antigen binding agent of the present disclosure is a chimeric antigen receptor (CAR). In some embodiments, an antigen binding agent of the present disclosure is an engineered T cell receptor (TCR). In some embodiments, the CARs and/or TCRs are expressed with a dominant negative TFGβ Receptor (DN TFGβ R).

The present disclosure further provides for synthetic Notch receptors (synNotch receptors) comprising at least an anti-PSCA binding domain. In various embodiments, one or more anti-PSCA binding domain is an scFv. An exemplary anti-PSCA binding domain amino acid sequences, and nucleic acid sequence encoding the same, is provided herein, for example in Table 9. In some embodiments, the synNotch receptors CARs and/or TCRs are expressed with a dominant negative TFGβ Receptor.

Various embodiments of the present disclosure provide a vector encoding an anti-PSMA binding domain or antigen anti-PSMA binding agent provided herein, e.g., a vector encoding an anti-PSMA CAR. Various embodiments of the present disclosure provide a vector encoding a DN TFGβ R, e.g., a vector encoding an anti-PSMA CAR and a DN TFGβ R. In some embodiments the DN TFGβ R is encoded in a separate vector from the vector encoding the anti-PSMA CAR. In some embodiments the DN TFGβ R is encoded in the same vector encoding the anti-PSMA CAR. Various embodiments of the present disclosure provide a vector encoding an anti-PSCA synNotch receptor. In some embodiments the DN TFGβ R is encoded in a separate vector from the vector encoding the anti-PSCA synNotch receptor. In some embodiments the DN TFGβ R is encoded in the same vector encoding the anti-PSCA synNotch receptor.

Various embodiments of the present disclosure provide an anti-PSMA antigen binding agent that is a cell encoding or expressing an anti-PSMA antigen binding agent, e.g., a T cell or NK cell engineered to encode or express an anti-PSMA chimeric antigen receptor or TCR. The present disclosure provides immune cells genetically modified with an integrated gene, e.g., a nucleotide sequence of interest (e.g., a constitutive expression construct and/or an inducible expression construct that comprises such nucleotide sequence, for example a synthetic notch (synNotch) receptor inducible expression construct, such as an anti-PSCA synNotch receptor described herein. In embodiments the immune cells are further engineered to express an anti-PSCA synNotch receptor. In embodiments, the immune cells are further engineered to express a DN TFGβ R. In some embodiments, the present disclosure provides methods of treating a subject having a tumor, such as a prostate tumor, comprising administering to the subject an anti-PSMA binding agent therapy described herein and/or a protein therapeutic described herein. In some embodiments, methods further comprise administration of one or more additional therapies (e.g., a second binding agent (e.g., CAR-T cell, CAR-NK cell, TCR-T cell, TIL cell, allogeneic NK cell, and autologous NK cell), an antibody-drug conjugate, an antibody, a bispecific antibody, a T cell-engaging bispecific antibody, an engineered antibody, and/or a polypeptide described herein).

Other features, objects, and advantages of the present disclosure are apparent in the detailed description that follows. It should be understood, however, that the detailed description, while indicating embodiments of the present disclosure, is given by way of illustration only, not limitation.

An anti-PSMA binding domain of the present disclosure may comprise antigen-binding sequences as found in an antibody described herein. In some instances, an anti-PSMA binding domain of the present disclosure comprises an anti-PSMA binding domain described herein, such as an scFv. Unless otherwise indicated, it is to be appreciated the references to PSMA in the present disclosure relate to human PSMA. In various embodiments, an anti-PSMA binding domain of the present disclosure comprises at least one heavy chain CDR (HCDR) provided herein, e.g., at least one HCDR disclosed in any one of Tables 4-8. In various embodiments, an anti-PSMA binding domain of the present disclosure comprises two HCDRs provided herein, e.g., at least two HCDRs disclosed in any one of Tables 4-8. In various embodiments, an anti-PSMA binding domain of the present disclosure comprises three HCDRs provided herein, e.g., three HCDRs disclosed in any one of Tables 4-8. In various embodiments, an anti-PSMA binding domain of the present disclosure comprises at least one light chain CDR (LCDR) provided herein, e.g., at least one LCDR disclosed in any one of Tables 4-8. In various embodiments, an anti-PSMA binding domain of the present disclosure comprises two LCDRs provided herein, e.g., at least two LCDRs disclosed in any one of Tables 4-8. In various embodiments, an anti-PSMA binding domain of the present disclosure comprises three LCDRs provided herein, e.g., three LCDRs disclosed in any one of Tables 4-8.

In various embodiments, an anti-PSMA binding domain of the present disclosure comprises at least one HCDR provided herein, e.g., at least one HCDR disclosed in any one of Tables 4-8, and at least one LCDR provided herein, e.g., at least one LCDR disclosed in any one of Tables 4-8. In various embodiments, an anti-PSMA binding domain of the present disclosure comprises one HCDR provided herein, e.g., at least one HCDR disclosed in any one of Tables 4-8, and one LCDR provided herein, e.g., derived from the same Table of Tables 4-8 as the HCDR(s). In various embodiments, an anti-PSMA binding domain of the present disclosure comprises two HCDRs provided herein, e.g., at least two HCDRs disclosed in any one of Tables 4-8, and two LCDRs provided herein, e.g., at least two LCDRs disclosed in any one of Tables 4-8. In various embodiments, an anti-PSMA binding domain of the present disclosure comprises two HCDRs provided herein, e.g., at least two HCDRs disclosed in any one of Tables 4-8, and two LCDRs provided herein, e.g., derived from the same Table of Tables 4-8 as the HCDR(s). In various embodiments, an anti-PSMA binding domain of the present disclosure comprises three HCDRs provided herein, e.g., three HCDRs disclosed in any one of Tables 4-8, and three LCDRs provided herein, e.g., three LCDRs disclosed in any one of Tables 4-8. In various embodiments, an anti-PSMA binding domain of the present disclosure comprises three HCDRs provided herein, e.g., three HCDRs disclosed in any one of Tables 4-8, and three LCDRs derived from the same Table of Tables 4-8 as the HCDR(s).

In various embodiments, an anti-PSMA binding domain of the present disclosure comprises at least one heavy chain framework region (heavy chain FR) of a heavy chain variable domain disclosed herein, e.g., at least one heavy chain FR of a heavy chain variable domain disclosed in any one of Tables 4-8. In various embodiments, an anti-PSMA binding domain of the present disclosure comprises two heavy chain FRs of a heavy chain variable domain disclosed herein, e.g., at least two heavy chain FRs of a heavy chain variable domain disclosed in any one of Tables 4-8. In various embodiments, an anti-PSMA binding domain of the present disclosure comprises three heavy chain FRs of a heavy chain variable domain disclosed herein, e.g., three heavy chain FRs of a heavy chain variable domain disclosed in any one of Tables 4-8.

In various embodiments, an anti-PSMA binding domain of the present disclosure comprises at least one light chain FR of a light chain variable domain disclosed herein, e.g., at least one light chain FR of a light chain variable domain disclosed in any one of Tables 4-8. In various embodiments, an anti-PSMA binding domain of the present disclosure comprises two light chain FRs of a light chain variable domain disclosed herein, e.g., at least two light chain FRs of a light chain variable domain disclosed in any one of Tables 4-8. In various embodiments, an anti-PSMA binding domain of the present disclosure comprises three light chain FRs of a light chain variable domain disclosed herein, e.g., three light chain FRs of a light chain variable domain disclosed in any one of Tables 4-8.

In various embodiments, an anti-PSMA binding domain of the present disclosure comprises at least one heavy chain FR of a heavy chain variable domain disclosed herein, e.g., at least one heavy chain FR of a heavy chain variable domain disclosed in any one of Tables 4-8, and at least one light chain FR of a light chain variable domain disclosed herein, e.g., at least one light chain FR of a light chain variable domain disclosed in any one of Tables 4-8. In various embodiments, an anti-PSMA binding domain of the present disclosure comprises one heavy chain FR of a heavy chain variable domain disclosed herein, e.g., at least one heavy chain FR of a heavy chain variable domain disclosed in any one of Tables 4-8, and one light chain FR of a light chain variable domain disclosed herein, e.g., derived from the same Table of Tables 4-8 as the heavy chain FR(s). In various embodiments, an anti-PSMA binding domain of the present disclosure comprises two heavy chain FRs of a heavy chain variable domain disclosed herein, e.g., at least two heavy chain FRs of a heavy chain variable domain disclosed in any one of Tables 4-8, and two light chain FRs of a light chain variable domain disclosed herein, e.g., at least two light chain FRs of a light chain variable domain disclosed in any one of Tables 4-8. In various embodiments, an anti-PSMA binding domain of the present disclosure comprises two heavy chain FRs of a heavy chain variable domain disclosed herein, e.g., at least two heavy chain FRs of a heavy chain variable domain disclosed in any one of Tables 4-8, and two light chain FRs of a light chain variable domain disclosed herein, e.g., derived from the same Table of Tables 4-8 as the heavy chain FR(s). In various embodiments, an anti-PSMA binding domain of the present disclosure comprises three heavy chain FRs of a heavy chain variable domain disclosed herein, e.g., three heavy chain FRs of a heavy chain variable domain disclosed in any one of Tables 4-8, and three light chain FRs of a light chain variable domain disclosed herein, e.g., three light chain FRs of a light chain variable domain disclosed in any one of Tables 4-8. In various embodiments, an anti-PSMA binding domain of the present disclosure comprises three heavy chain FRs of a heavy chain variable domain disclosed herein, e.g., three light chain FRs of a light chain variable domain disclosed in any one of Tables 4-8, and three light chain FRs derived from the same Table of Tables 4-8 as the heavy chain FR(s).

Exemplary antibody sequences provided in Tables 4-8 are suitable for use in any antibody format, comprising, e.g., a tetrameric antibody, a monospecific antibody, a bispecific antibody, an antigen binding fragment, or a binding motif. Heavy chain variable domains and light chain variable domains and portions thereof provided in Tables 4-8 may be comprised in anti-PSMA binding domain.

In various embodiments, an anti-PSMA binding domain of the present disclosure comprises one, two, or three FRs that together or each individually have at least 75% identity (e.g., at least 75%, at least 80%, at least 90%, at least 95%, or 100%, e.g., 85-90%, 85-95%, 85-100%, 90-95%, 90-100%, or 95-100%) to corresponding FR(s) of a heavy chain variable domain of a heavy chain variable domain disclosed in in any one of Tables 4-8. In various embodiments, an anti-PSMA binding domain of the present disclosure comprises one, two, or three FRs that together or each individually have at least 75% identity (e.g., at least 75%, at least 80%, at least 90%, at least 95%, or 100%) to corresponding FR(s) of a light chain variable domain of a light chain variable domain disclosed in any one of Tables 4-8.

In various embodiments, an anti-PSMA binding domain of the present disclosure comprises at least one heavy chain variable domain having at least 75% sequence identity to a heavy chain variable domain disclosed in any one of Tables 4-8 (e.g., at least 75%, at least 80%, at least 90%, at least 95%, or 100% identity; e.g., 85-90%, 85-95%, 85-100%, 90-95%, 90-100%, or 95-100%). In various embodiments, an anti-PSMA binding domain of the present disclosure comprises two heavy chain variable domains each having at least 75% sequence identity to a heavy chain variable domain disclosed in Tables 4-8 (e.g., at least 75%, at least 80%, at least 90%, at least 95%, or 100% identity; e.g., 85-90%, 85-95%, 85-100%, 90-95%, 90-100%, or 95-100%), which heavy chain variable domains may be same or different.

In various embodiments, an anti-PSMA binding domain of the present disclosure comprises at least one light chain variable domain having at least 75% sequence identity to a light chain variable domain disclosed in any one of Tables 4-8 (e.g., at least 75%, at least 80%, at least 90%, at least 95%, or 100% identity; e.g., 85-90%, 85-95%, 85-100%, 90-95%, 90-100%, or 95-100%). In various embodiments, an anti-PSMA binding domain of the present disclosure comprises two light chain variable domains each having at least 75% sequence identity to a light chain variable domain disclosed in any one of Tables 4-8 (e.g., at least 75%, at least 80%, at least 90%, at least 95%, or 100% identity; e.g., 85-90%, 85-95%, 85-100%, 90-95%, 90-100%, or 95-100%), which light chain variable domains may be same or different.

In various embodiments, an anti-PSMA binding domain of the present disclosure comprises at least one heavy chain variable domain having at least 75% sequence identity to a heavy chain variable domain disclosed in any one of Tables 4-8 (e.g., at least 75%, at least 80%, at least 90%, at least 95%, or 100% identity; e.g., 85-90%, 85-95%, 85-100%, 90-95%, 90-100%, or 95-100%) and at least one light chain variable domain having at least 75% sequence identity to a light chain variable domain disclosed in any one of Tables 4-8 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-PSMA binding domain of the present disclosure comprises one heavy chain variable domain having at least 75% sequence identity to a heavy chain variable domain disclosed in any one of Tables 4-8 (e.g., at least 75%, at least 80%, at least 90%, at least 95%, or 100% identity; e.g., 85-90%, 85-95%, 85-100%, 90-95%, 90-100%, or 95-100%) and one light chain variable domain having at least 75% sequence identity to a light chain variable domain disclosed in any one of Tables 4-8 (e.g., at least 75%, at least 80%, at least 90%, at least 95%, or 100% identity; e.g., 85-90%, 85-95%, 85-100%, 90-95%, 90-100%, or 95-100%), where the heavy chain variable domain and light chain variable domain are optionally derived from the same Table of Tables 4-8.

In various embodiments, an anti-PSMA binding domain of the present disclosure comprises two heavy chain variable domains each having at least 75% sequence identity to a heavy chain variable domain disclosed in Tables 4-8 (e.g., at least 75%, at least 80%, at least 90%, at least 95%, or 100% identity; e.g., 85-90%, 85-95%, 85-100%, 90-95%, 90-100%, or 95-100%) and two light chain variable domains each having at least 75% sequence identity to a light chain variable domain disclosed in Tables 4-8 (e.g., at least 75%, at least 80%, at least 90%, at least 95%, or 100% identity; e.g., 85-90%, 85-95%, 85-100%, 90-95%, 90-100%, or 95-100%), where, in various embodiments, (i) each of the heavy chain variable domains may be same or different; (ii) each of the light chain variable domains may be same or different; (iii) at least one heavy chain variable domain and at least one light chain variable domain may be derived from the same Table of Tables 4-8; or (iv) the two heavy chain variable domains and the two light chain variable domains are all derived from the same Table of Tables 4-8. Each of Tables 4-8 represents the heavy chain variable domain and light chain variable domain sequences of an exemplary antibody, comprising (i) the heavy chain variable domain of the exemplary antibody; (ii) a DNA sequence encoding the heavy chain variable domain (iii) three heavy chain variable domain CDRs of the heavy chain variable domain, according to IMGT, Kabat, and Chothia numbering; (iv) the light chain variable domain of the exemplary antibody; (v) a DNA sequence encoding the light chain variable domain; and (vi) three light chain variable domain CDRs of the light chain variable domain, according to IMGT, Kabat, and Chothia numbering. Information provided in each table provides framework amino acid sequences, as well as nucleotide sequences encoding each CDR amino acid sequence and nucleotide sequences encoding corresponding FR amino acid sequence.

In various embodiments an anti-PSMA binding domain may comprise a heavy chain variable domain of the present disclosure (e.g., having at least 75% sequence identity to a heavy chain variable domain of any one of Tables 4-8, e.g., at least 80%, 85%, 90%, 95%, or 100% identity; e.g., 85-90%, 85-95%, 85-100%, 90-95%, 90-100%, or 95-100%), a light chain variable domain of the present disclosure (e.g., having at least 75% sequence identity to a light chain variable domain of any one of Tables 4-8, e.g., at least 80%, 85%, 90%, 95%, or 100% identity; e.g., 85-90%, 85-95%, 85-100%, 90-95%, 90-100%, or 95-100%), and a linker (e.g., a linker according to SEQ ID NO: 126. In various embodiments an anti-PSMA binding domain may comprise a leader sequence, a heavy chain variable domain of the present disclosure (e.g., having at least 75% sequence identity to a heavy chain variable domain of any one of Tables 4-8, e.g., at least 80%, 85%, 90%, 95%, or 100% identity; e.g., 85-90%, 85-95%, 85-100%, 90-95%, 90-100%, or 95-100%), a light chain variable domain of the present disclosure (e.g., having at least 75% sequence identity to a light chain variable domain of any one of Tables 4-8, e.g., at least 80%, 85%, 90%, 95%, or 100% identity; e.g., 85-90%, 85-95%, 85-100%, 90-95%, 90-100%, or 95-100%), and a linker. If provided with an amino acid or nucleotide sequence of an anti-PSMA binding domain comprising a heavy chain variable domain of the present disclosure and a light chain variable domain of the present disclosure, the linker joining the two variable domains will be apparent from the sequence in view of the present disclosure. If provided with an amino acid or nucleotide sequence of an anti-PSMA biding domain comprising a heavy chain variable domain of the present disclosure and a light chain variable domain of the present disclosure, the leader sequence will be apparent in view of the present disclosure. For the avoidance of doubt, a heavy chain variable domain and a light chain variable domain of the present disclosure may be present in any orientation, e.g., an orientation in which the heavy chain variable domain is C terminal of the light chain variable domain or in which the heavy chain variable domain is N terminal of the light chain variable domain. In various embodiments an anti-PSMA biding domain may comprise a linker according to SEQ ID NO: 126.

In certain embodiments, an anti-PSMA binding domain of the present disclosure comprises an anti-PSMA biding domain that comprises a heavy chain variable domain of the present disclosure, a light chain variable domain of the present disclosure, and a linker having at least 75% sequence identity to SEQ ID NO: 8 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-PSMA binding domain of the present disclosure comprises an anti-PSMA biding domain that comprises a linker according to SEQ ID NO: 126. In certain embodiments, an anti-PSMA binding domain of the present disclosure comprises an anti-PSMA biding domain that comprises a heavy chain variable domain of the present disclosure, a light chain variable domain of the present disclosure, and a leader sequence having at least 75% sequence identity to SEQ ID NO: 48 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-PSMA binding domain of the present disclosure comprises an anti-PSMA biding domain that comprises a CSF2RA leader sequence according to SEQ ID NO: 137 (MLLLVTSLLLCELPHPAFLLIP; SEQ ID NO: 137). In embodiments a leader sequence may be encoded by nucleic acid sequence at least 75% sequence identity to atgcttctcctggtgacaagccttctgctctgtgaattgccacacccagcattcctcctgattcct (SEQ ID NO: 256) (e.g., at least 75%, at least 80%, at least 90%, at least 95%, or 100% identity; e.g., 85-90%, 85-95%, 85-100%, 90-95%, 90-100%, or 95-100%). In certain embodiments, an anti-PSMA binding domain of the present disclosure comprises an anti-PSMA biding domain that comprises a heavy chain variable domain of the present disclosure, a light chain variable domain of the present disclosure, a linker of the present disclosure, and a leader sequence of the present disclosure.

A binding agent of the present disclosure that is based on an exemplary antibody provided herein, such as for example Abs 1-5, may be provided in any fragment or format, comprising a heavy chain variable domain according to the indicated exemplary antibody and a light chain variable domain according to the indicated exemplary antibody.

TABLE 4
Exemplary Antibody Sequences 1 (Ab1)

| SEQ ID | | |
|---|---|---|
| NO: | Description | Sequence |
| 1 | Heavy Chain | EVQLVESGGGLVQPGGSMRLSCAASGFTFSDYYMAWVRQAP |
| | Variable | GKGLEWIANINYDGSNTYYADSLKGRFTISRDNSKNTLYLQM |
| | Domain | NSLRAEDTAVYYCARNWDGYYGYFDVWGQGTTVTVSS |
| 2 | VH (DNA) | Gaggtgcaacttgtggagagcggaggaggtttagtgcaacccggaggcagcatgagactgag |
| | | ctgcgccgccagcggcttcacattctccgactactacatggcttgggtccgacaagctcccggaa |
| | | aaggactggagtggatcgccaacatcaactacgacggctccaacacctactacgccgactcttta |
| | | aagggtcgtttcacaatctctcgtgacaacagcaagaacactttatatttacaaatgaactctttaagg |
| | | gccgaggataccgccgtgtactactgcgctcgtaactgggacggctactacggctacttcgacgt |
| | | gtggggccaaggaaccaccgtgaccgtgagcagc |
| 3 | CDRH1 | GFTFSDYY |
| | IMGT (Prot) | |
| 4 | CDRH1 | DYYMA |
| | Kabat (Prot) | |
| 5 | CDRH1 | GFTFSD |
| | Chothia | |
| | (Prot) | |
| 6 | CDRH2 | INYDGSNT |
| | IMGT (Prot) | |
| 7 | CDRH2 | NINYDGSNTYYADSLKG |
| | Kabat (Prot) | |
| 8 | CDRH2 | NINYDGSNTYYADSLKG |
| | Chothia | |
| | (Prot) | |
| 9 | CDRH3 | ARNWDGYYGYFDV |
| | IMGT (Prot) | |
| 10 | CDRH3 | NWDGYYGYFDV |
| | Kabat (Prot) | |
| 11 | CDRH3 | NWDGYYGYFDV |
| | Chothia | |
| | (Prot) | |
| 12 | Light Chain | DIQLTQSPSSLSASVGDRVTITCRASSSVSHIYWYQQKPGKAP |
| | Variable | KPWIYRTSNLASGVPSRFSGSGSGTDYTLTISSLQPEDFATYYC |
| | Domain | QQYHTYPPTFGQGTKLEIK |
| 13 | VL (DNA) | Gatatccagctgacccagtccccttcctctctgtctgcgtctgttggcgatcgtgtcaccatcacttg |
| | | tcgtgccagcagcagcgtgagccacatttattggtaccaacaaaagcccggcaaagcccctaag |
| | | ccttggatctacagaacctccaatctggccagcggcgtgcccagcagattcagcggaagcggat |
| | | ccggcaccgactacactttaaccatcagctctttacagcccgaggacttcgccacatactactgcc |
| | | agcagtaccacacctatccccccacattcggccaaggaacaaagctggagattaag |
| 14 | CDRL1 | SSVSH |
| | IMGT (Prot) | |
| 15 | CDRL1 | RASSSVSHIY |
| | Kabat (Prot) | |
| 16 | CDRL1 | RASSSVSHIY |
| | Chothia | |
| | (Prot) | |
| 17 | CDRL2 | RTS |
| | IMGT (Prot) | |
| 18 | CDRL2 | RTSNLAS |
| | Kabat (Prot) | |
| 19 | CDRL2 | RTSNLAS |
| | Chothia | |
| | (Prot) | |
| 20 | CDRL3 | QQYHTYPPT |
| | IMGT (Prot) | |
| 21 | CDRL3 | QQYHTYPPT |
| | Kabat (Prot) | |
| 22 | CDRL3 | QQYHTYPPT |
| | Chothia | |
| | (Prot) | |
| 23 | ScFv | DIQLTQSPSSLSASVGDRVTITCRASSSVSHIYWYQQKPGKAP |
| | | KPWIYRTSNLASGVPSRFSGSGSGTDYTLTISSLQPEDFATYYC |
| | | QQYHTYPPTFGQGTKLEIKGSTSGSGKPGSGEGSTKGEVQLVE |
| | | SGGGLVQPGGSMRLSCAASGFTFSDYYMAWVRQAPGKGLE |
| | | WIANINYDGSNTYYADSLKGRFTISRDNSKNTLYLQMNSLRA |
| | | EDTAVYYCARNWDGYYGYFDVWGQGTTVTVSS |
| 24 | ScFv | Gatatccagctgacccagtccccttcctctctgtctgcgtctgttggcgatcgtgtcaccatcacttg |
| | | tcgtgccagcagcagcgtgagccacatttattggtaccaacaaaagcccggcaaagcccctaag |
| | | ccttggatctacagaacctccaatctggccagcggcgtgcccagcagattcagcggaagcggat |
| | | ccggcaccgactacactttaaccatcagctctttacagcccgaggacttcgccacatactactgcc |
| | | agcagtaccacacctatccccccacattcggccaaggaacaaagctggagattaagggctccac |
| | | ctccggaagcggcaaacccggtagcggcgagggctccacaaagggcgaggtgcaacttgtgg |
| | | agagcggaggaggtttagtgcaacccggaggcagcatgagactgagctgcgccgccagcggc |
| | | ttcacattctccgactactacatggcttgggtccgacaagctcccggaaaaggactggagtggatc |
| | | gccaacatcaactacgacggctccaacacctactacgccgactctttaaagggtcgtttcacaatct |
| | | ctcgtgacaacagcaagaacactttatatttacaaatgaactctttaagggccgaggataccgccgt |
| | | gtactactgcgctcgtaactgggacggctactacggctacttcgacgtgtggggccaaggaacca |
| | | ccgtgaccgtgagcagc |

TABLE 5
Exemplary Antibody Sequences 2 (Ab2)

| SEQ | | |
|---|---|---|
| ID | | |
| NO: | Description | Sequence |
| 25 | Heavy Chain | QVQLVQSGAEVKKPGASVKLSCKASGYTFTTYWMHWVRQA |
| | Variable | PGQGLEWIGMIHPNSGSTNYAQKFQGRATLTVDTSTSTAYME |
| | Domain | LSSLRSEDTAVYYCARDPYDYGEDFDVWGQGTTVTVSS |
| 26 | VH (DNA) | Caagtgcagctggtgcagtccggcgccgaggtgaagaagcccggtgcttccgtgaagctgtctt |
| | | gcaaagccagcggctacaccttcaccacctattggatgcactgggtccgacaagctcccggtcaa |
| | | ggtctggagtggattggcatgatccaccccaactccggctccaccaactacgcccagaagttcca |
| | | aggtcgtgccactttaacagtggataccagcaccagcaccgcctacatggagctgagtagtttga |
| | | ggagcgaggacaccgccgtgtactattgcgctcgtgacccctacgactacggcgaggacttcga |
| | | cgtgtggggccaaggaacaacagtgaccgtgagcagc |
| 27 | CDRH1 | GYTFTTYW |
| | IMGT (Prot) | |
| 28 | CDRH1 | TYWMH |
| | Kabat (Prot) | |
| 29 | CDRH1 | GYTFTT |
| | Chothia | |
| | (Prot) | |
| 30 | CDRH2 | IHPNSGST |
| | IMGT (Prot) | |
| 31 | CDRH2 | MIHPNSGSTNYAQKFQG |
| | Kabat (Prot) | |
| 32 | CDRH2 | MIHPNSGSTNYAQKFQG |
| | Chothia | |
| | (Prot) | |
| 33 | CDRH3 | ARDPYDYGEDFDV |
| | IMGT (Prot) | |
| 34 | CDRH3 | DPYDYGEDFDV |
| | Kabat (Prot) | |
| 35 | CDRH3 | DPYDYGEDFDV |
| | Chothia | |
| | (Prot) | |
| 36 | Light Chain | DIQMTQSPSSLSASVGDRVTVTCRASQNVNTNVAWYQQKPG |
| | Variable | KAPKVLIYSASYRNSGVPSRFSGSGSGTDFTLTISSVQPEDFAT |
| | Domain | YYCQQYNSYPFTFGQGTKLEIK |
| 37 | VL (DNA) | Gacatccagatgacccagagccccagctctttaagtgccagcgtgggcgacagagtgacagtg |
| | | acttgtcgtgccagccagaacgtgaataccaacgtggcttggtaccagcagaagcccggcaaag |
| | | cccctaaggtgctgatctattccgcgtcttatcgtaactccggcgtgccttcgcgtttttctgggtctg |
| | | gtagcggcaccgacttcactttaacaatcagcagcgttcagcccgaagacttcgccacctactact |
| | | gccagcagtacaacagctatccctttactttcggtcaagggaccaagctcgagatcaaa |
| 38 | CDRL1 | QNVNTN |
| | IMGT (Prot) | |
| 39 | CDRL1 | RASQNVNTNVA |
| | Kabat (Prot) | |
| 40 | CDRL1 | RASQNVNTNVA |
| | Chothia | |
| | (Prot) | |
| 41 | CDRL2 | MIHPNSGSTNYAQKFQG |
| | IMGT (Prot) | |
| 42 | CDRL2 | SASYRNS |
| | Kabat (Prot) | |
| 43 | CDRL2 | SASYRNS |
| | Chothia | |
| | (Prot) | |
| 44 | CDRL3 | DPYDYGEDFDV |
| | IMGT (Prot) | |
| 45 | CDRL3 | QQYNSYPFT |
| | Kabat (Prot) | |
| 46 | CDRL3 | QQYNSYPFT |
| | Chothia | |
| | (Prot) | |
| 47 | ScFv | DIQMTQSPSSLSASVGDRVTVTCRASQNVNTNVAWYQQKPG |
| | | KAPKVLIYSASYRNSGVPSRFSGSGSGTDFTLTISSVQPEDFAT |
| | | YYCQQYNSYPFTFGQGTKLEIKGSTSGSGKPGSGEGSTKGQV |
| | | QLVQSGAEVKKPGASVKLSCKASGYTFTTYWMHWVRQAPG |
| | | QGLEWIGMIHPNSGSTNYAQKFQGRATLTVDTSTSTAYMELS |
| | | SLRSEDTAVYYCARDPYDYGEDFDVWGQGTTVTVSS |
| 48 | ScFv | Gacatccagatgacccagagccccagctctttaagtgccagcgtgggcgacagagtgacagtg |
| | | acttgtcgtgccagccagaacgtgaataccaacgtggcttggtaccagcagaagcccggcaaag |
| | | cccctaaggtgctgatctattccgcgtcttatcgtaactccggcgtgccttcgcgtttttctgggtctg |
| | | gtagcggcaccgacttcactttaacaatcagcagcgttcagcccgaagacttcgccacctactact |
| | | gccagcagtacaacagctatccctttactttcggtcaagggaccaagctcgagatcaaaggctcca |
| | | ccagcggtagcggcaaacccggttccggcgagggctctaccaagggccaagtgcagctggtgc |
| | | agtccggcgccgaggtgaagaagcccggtgcttccgtgaagctgtcttgcaaagccagcggcta |
| | | caccttcaccacctattggatgcactgggtccgacaagctcccggtcaaggtctggagtggattgg |
| | | catgatccaccccaactccggctccaccaactacgcccagaagttccaaggtcgtgccactttaac |
| | | agtggataccagcaccagcaccgcctacatggagctgagtagtttgaggagcgaggacaccgc |
| | | cgtgtactattgcgctcgtgacccctacgactacggcgaggacttcgacgtgtggggccaaggaa |
| | | caacagtgaccgtgagcagc |

TABLE 6
Exemplary Antibody Sequences 3 (Ab3)

| SEQ | | |
|---|---|---|
| ID | | |
| NO: | Description | Sequence |
| 49 | Heavy Chain | EVQLVESGGGLVQPGGSMRLSCAASGFTFSDYYMAWVRQAP |
| | Variable | GKGLEWVANINYDGTSTYYADSLKGRFTISRDSSKNTLYLQM |
| | Domain | NSLRAEDTAVYYCARALDGYYGYLDVWGQGTTVTVSS |
| 50 | VH (DNA) | Gaggtgcagctggtggagtccggaggaggtttagtccaacccggtggcagcatgaggctgtctt |
| | | gtgctgcctccggcttcactttttctgattactacatggcttgggtccgacaagctcccggaaaaggt |
| | | ttagagtgggtggctaacatcaactacgacggcaccagcacctactatgccgacagcctcaagg |
| | | gcagattcaccatctctcgtgattcgtctaaaaacactttatatttacaaatgaactctttaagagccga |
| | | agataccgccgtgtactattgcgctcgtgccctcgacggctactacggatatttagacgtgtggggt |
| | | caaggaacaaccgtgaccgtgtccagc |
| 51 | CDRH1 | GFTFSDYY |
| | IMGT (Prot) | |
| 52 | CDRH1 | DYYMA |
| | Kabat (Prot) | |
| 53 | CDRH1 | GFTFSD |
| | Chothia | |
| | (Prot) | |
| 54 | CDRH2 | INYDGTST |
| | IMGT (Prot) | |
| 55 | CDRH2 | NINYDGTSTYYADSLKG |
| | Kabat (Prot) | |
| 56 | CDRH2 | NINYDGTSTYYADSLKG |
| | Chothia | |
| | (Prot) | |
| 57 | CDRH3 | ARALDGYYGYLDV |
| | IMGT (Prot) | |
| 58 | CDRH3 | ALDGYYGYLDV |
| | Kabat (Prot) | |
| 59 | CDRH3 | ALDGYYGYLDV |
| | Chothia | |
| | (Prot) | |
| 60 | Light Chain | DIQLTQSPSSLSASVGDRVTLTCRASQSISNNLHWYQQKPGKA |
| | Variable | PKLLIKYVSQSISGIPSRFSGSGLGTDFTLTISSVQPEDFATYYC |
| | Domain | QQSNSWPYTFGQGTKLEIK |
| 61 | VL (DNA) | Gacatccagctgacccagagccctagctctttaagcgctagcgtgggcgatagggtgactctga |
| | | cttgtcgtgcgtcccaaagcattagcaacaatttacactggtaccagcagaagcccggaaaagcc |
| | | cccaagctgctgatcaaatatgtgagccagagcatctccggcatcccctctcgtttttctggtagcg |
| | | gactgggcaccgactttactttaaccatcagcagcgtccagcccgaggacttcgccacatactact |
| | | gccagcagagcaacagctggccctatactttcggccaaggaacaaagctggagatcaag |
| 62 | CDRL1 | QSISNN |
| | IMGT (Prot) | |
| 63 | CDRL1 | RASQSISNNLH |
| | Kabat (Prot) | |
| 64 | CDRL1 | RASQSISNNLH |
| | Chothia | |
| | (Prot) | |
| 65 | CDRL2 | YVS |
| | IMGT (Prot) | |
| 66 | CDRL2 | YVSQSIS |
| | Kabat (Prot) | |
| 67 | CDRL2 | YVSQSIS |
| | Chothia | |
| | (Prot) | |
| 68 | CDRL3 | QQSNSWPYT |
| | IMGT (Prot) | |
| 69 | CDRL3 | QQSNSWPYT |
| | Kabat (Prot) | |
| 70 | CDRL3 | QQSNSWPYT |
| | Chothia | |
| | (Prot) | |
| 71 | ScFv | EVQLVESGGGLVQPGGSMRLSCAASGFTFSDYYMAWVRQAP |
| | | GKGLEWVANINYDGTSTYYADSLKGRFTISRDSSKNTLYLQM |
| | | NSLRAEDTAVYYCARALDGYYGYLDVWGQGTTVTVSSGSTS |
| | | GSGKPGSGEGSTKGDIQLTQSPSSLSASVGDRVTLTCRASQSIS |
| | | NNLHWYQQKPGKAPKLLIKYVSQSISGIPSRFSGSGLGTDFTL |
| | | TISSVQPEDFATYYCQQSNSWPYTFGQGTKLEIK |
| 72 | ScFv | gaggtgcagctggtggagtccggaggaggtttagtccaacccggtggcagcatgaggctgtctt |
| | | gtgctgcctccggcttcactttttctgattactacatggcttgggtccgacaagctcccggaaaaggt |
| | | ttagagtgggtggctaacatcaactacgacggcaccagcacctactatgccgacagcctcaagg |
| | | gcagattcaccatctctcgtgattcgtctaaaaacactttatatttacaaatgaactctttaagagccga |
| | | agataccgccgtgtactattgcgctcgtgccctcgacggctactacggatatttagacgtgtggggt |
| | | caaggaacaaccgtgaccgtgtccagcggatccacctccggaagcggcaaacccggtagcgg |
| | | cgaaggcagcaccaaaggagacatccagctgacccagagccctagctctttaagcgctagcgtg |
| | | ggcgatagggtgactctgacttgtcgtgcgtcccaaagcattagcaacaatttacactggtaccag |
| | | cagaagcccggaaaagcccccaagctgctgatcaaatatgtgagccagagcatctccggcatcc |
| | | cctctcgtttttctggtagcggactgggcaccgactttactttaaccatcagcagcgtccagcccga |
| | | ggacttcgccacatactactgccagcagagcaacagctggccctatactttcggccaaggaacaa |
| | | agctggagatcaag |

TABLE 7
Exemplary Antibody Sequences 4 (Ab4)

| SEQ | | |
|---|---|---|
| ID | | |
| NO: | Description | Sequence |
| 73 | Heavy Chain | EVQLVESGGGLVQPGGSLRLSCAASGFTFSDYYMAWVRQAP |
| | Variable | GKGLEWVANINYDGSSTFYADSLKGRFTISRDNSKNTLYLQM |
| | Domain | NSLRAEDTAVYYCGRQVGYYDPMDYWGQGTTVTVSS |
| 74 | VH (DNA) | Gaggtgcagttggtggagagcggaggaggactggtgcagcccggtggctctttaagactcagc |
| | | tgtgccgccagcggatttacattctccgactactacatggcttgggtccgacaagcccccggaaaa |
| | | ggtttagagtgggtggccaacatcaactacgacggctcctccacattctacgccgactctttaaag |
| | | ggtcgtttcaccatctctcgtgacaacagcaaaaatactttatatttacaaatgaactctttaagggcc |
| | | gaggacaccgccgtgtactactgcggtcgtcaagttggctattacgaccccatggactactgggg |
| | | ccaaggaactaccgtgaccgtgagcagc |
| 75 | CDRH1 | GFTFSDYY |
| | IMGT (Prot) | |
| 76 | CDRH1 | DYYMA |
| | Kabat (Prot) | |
| 77 | CDRH1 | GFTFSD |
| | Chothia | |
| | (Prot) | |
| 78 | CDRH2 | INYDGSST |
| | IMGT (Prot) | |
| 79 | CDRH2 | NINYDGSSTFYADSLKG |
| | Kabat (Prot) | |
| 80 | CDRH2 | NINYDGSSTFYADSLKG |
| | Chothia | |
| | (Prot) | |
| 81 | CDRH3 | GRQVGYYDPMDY |
| | IMGT (Prot) | |
| 82 | CDRH3 | QVGYYDPMDY |
| | Kabat (Prot) | |
| 83 | CDRH3 | QVGYYDPMDY |
| | Chothia | |
| | (Prot) | |
| 84 | Light Chain | DIQLTQSPSSLSASVGDRVTITCRASSSVSHMYWYQQKPGKAP |
| | Variable | KPWIYRTSNLASGVPSRFSGSGSGTDYTLTISSMQPEDFATYY |
| | Domain | CQQYHSYPLTFGQGTKLEIK |
| 85 | VL (DNA) | Gacatccagctgacccagtcccccagctctttatccgctagcgtgggcgatagggtgaccatcac |
| | | ttgtcgtgcgtcttcgtctgtgtctcatatgtactggtaccagcagaagcccggcaaggcccccaag |
| | | ccttggatctatcgtacatccaatcttgcaagcggcgtcccttctcgtttttctggttccgggtctggta |
| | | ccgactacactttaaccatcagcagcatgcagcccgaggacttcgccacctactactgccagcag |
| | | tatcactcctatcctttaacttttggccaaggaacaaagttggagatcaag |
| 86 | CDRL1 | SSVSH |
| | IMGT (Prot) | |
| 87 | CDRL1 | RASSSVSHMY |
| | Kabat (Prot) | |
| 88 | CDRL1 | RASSSVSHMY |
| | Chothia | |
| | (Prot) | |
| 89 | CDRL2 | RTS |
| | IMGT (Prot) | |
| 90 | CDRL2 | RTSNLAS |
| | Kabat (Prot) | |
| 91 | CDRL2 | RTSNLAS |
| | Chothia | |
| | (Prot) | |
| 92 | CDRL3 | QQYHSYPLT |
| | IMGT (Prot) | |
| 93 | CDRL3 | QQYHSYPLT |
| | Kabat (Prot) | |
| 94 | CDRL3 | QQYHSYPLT |
| | Chothia | |
| | (Prot) | |
| 95 | ScFv | DIQLTQSPSSLSASVGDRVTITCRASSSVSHMYWYQQKPGKAP |
| | | KPWIYRTSNLASGVPSRFSGSGSGTDYTLTISSMQPEDFATYY |
| | | CQQYHSYPLTFGQGTKLEIKGSTSGSGKPGSGEGSTKGEVQL |
| | | VESGGGLVQPGGSLRLSCAASGFTFSDYYMAWVRQAPGKGL |
| | | EWVANINYDGSSTFYADSLKGRFTISRDNSKNTLYLQMNSLR |
| | | AEDTAVYYCGRQVGYYDPMDYWGQGTTVTVSS |
| 96 | ScFv | Gacatccagctgacccagtcccccagctctttatccgctagcgtgggcgatagggtgaccatcac |
| | | ttgtcgtgcgtcttcgtctgtgtctcatatgtactggtaccagcagaagcccggcaaggcccccaag |
| | | ccttggatctatcgtacatccaatcttgcaagcggcgtcccttctcgtttttctggttccgggtctggta |
| | | ccgactacactttaaccatcagcagcatgcagcccgaggacttcgccacctactactgccagcag |
| | | tatcactcctatcctttaacttttggccaaggaacaaagttggagatcaagggcagcacctccggta |
| | | gcggaaagcccggtagcggcgagggcagcaccaagggagaggtgcagttggtggagagcgg |
| | | aggaggactggtgcagcccggtggctctttaagactcagctgtgccgccagcggatttacattctc |
| | | cgactactacatggcttgggtccgacaagcccccggaaaaggtttagagtgggtggccaacatca |
| | | actacgacggctcctccacattctacgccgactctttaaagggtcgtttcaccatctctcgtgacaac |
| | | agcaaaaatactttatatttacaaatgaactctttaagggccgaggacaccgccgtgtactactgcg |
| | | gtcgtcaagttggctattacgaccccatggactactggggccaaggaactaccgtgaccgtgagc |
| | | agc |

TABLE 8
Exemplary Antibody Sequences 5 (Ab5)

| SEQ | | |
|---|---|---|
| ID | | |
| NO: | Description | Sequence |
| 97 | Heavy Chain | EVQLVQSGAEVKKPGASVKISCKTSGYTFTEYTIHWVKQASG |
| | Variable | KGLEWIGNINPNNGGTTYNQKFEDRATLTVDKSTSTAYMELS |
| | Domain | SLRSEDTAVYYCAAGWNFDYWGQGTTVTVSS |
| 98 | VH (DNA) | Gaagttcaacttgtgcaaagcggggcagaagtgaaaaaacccggggcgagcgttaaaatatctt |
| | | gtaaaacaagtggctacaccttcacggagtacaccatccactgggttaaacaagcttctggaaag |
| | | ggacttgaatggatcgggaacataaaccccaacaatgggggcactacttataatcaaaagtttgag |
| | | gatcgggctaccctcacagtggataagtccacctccacagcttatatggaattgagtagccttagg |
| | | agcgaggatacagccgtttattattgtgcggcgggctggaactttgactattgggggcaagggac |
| | | gacggtgacggtgtcctcc |
| 99 | CDRH1 | GYTFTEYT |
| | IMGT (Prot) | |
| 100 | CDRH1 | EYTIH |
| | Kabat (Prot) | |
| 101 | CDRH1 | GYTFTE |
| | Chothia | |
| | (Prot) | |
| 102 | CDRH2 | INPNNGGT |
| | IMGT (Prot) | |
| 103 | CDRH2 | NINPNNGGTTYNQKFED |
| | Kabat (Prot) | |
| 104 | CDRH2 | NINPNNGGTTYNQKFED |
| | Chothia | |
| | (Prot) | |
| 105 | CDRH3 | AAGWNFDY |
| | IMGT (Prot) | |
| 106 | CDRH3 | GWNFDY |
| | Kabat (Prot) | |
| 107 | CDRH3 | GWNFDY |
| | Chothia | |
| | (Prot) | |
| 108 | Light Chain | DIVMTQSPSSLSASVGDRVTITCKASQDVGTAVDWYQQKPGK |
| | Variable | APKLLIYWASTRHTGVPDRFTGSGSGTDFTLTISSLQPEDFAD |
| | Domain | YFCQQYNSYPLTFGGGTKLEIK |
| 109 | VL (DNA) | Gacattgtgatgactcagtctccttcttctctttccgcttccgttggggaccgcgtcactataacttgta |
| | | aagcgtcccaagatgtcggcaccgccgttgactggtaccagcaaaaacccgggaaagcgccga |
| | | aactgctcatctactgggcttcaacccgccacacgggtgtcccggaccggtttacggggagcggt |
| | | agtggaaccgatttcactctgaccatttcctcccttcaaccggaagatttcgctgactacttttgtcaa |
| | | caatataattcatatcccctcactttcggagggggcacgaagttggaaataaag |
| 110 | CDRL1 | QDVGTA |
| | IMGT (Prot) | |
| 111 | CDRL1 | KASQDVGTAVD |
| | Kabat (Prot) | |
| 112 | CDRL1 | KASQDVGTAVD |
| | Chothia | |
| | (Prot) | |
| 113 | CDRL2 | WAS |
| | IMGT (Prot) | |
| 114 | CDRL2 | WASTRHT |
| | Kabat (Prot) | |
| 115 | CDRL2 | WASTRHT |
| | Chothia | |
| | (Prot) | |
| 116 | CDRL3 | QQYNSYPLTF |
| | IMGT (Prot) | |
| 117 | CDRL3 | QQYNSYPLT |
| | Kabat (Prot) | |
| 118 | CDRL3 | QQYNSYPLT |
| | Chothia | |
| | (Prot) | |
| 119 | ScFv | DIVMTQSPSSLSASVGDRVTITCKASQDVGTAVDWYQQKPGK |
| | | APKLLIYWASTRHTGVPDRFTGSGSGTDFTLTISSLQPEDFAD |
| | | YFCQQYNSYPLTFGGGTKLEIKGSTSGSGKPGSGEGSTKGEVQ |
| | | LVQSGAEVKKPGASVKISCKTSGYTFTEYTIHWVKQASGKGL |
| | | EWIGNINPNNGGTTYNQKFEDRATLTVDKSTSTAYMELSSLR |
| | | SEDTAVYYCAAGWNFDYWGQGTTVTVSS |
| 120 | ScFv | Gacattgtgatgactcagtctccttcttctctttccgcttccgttggggaccgcgtcactataacttgta |
| | | aagcgtcccaagatgtcggcaccgccgttgactggtaccagcaaaaacccgggaaagcgccga |
| | | aactgctcatctactgggcttcaacccgccacacgggtgtcccggaccggtttacggggagcggt |
| | | agtggaaccgatttcactctgaccatttcctcccttcaaccggaagatttcgctgactacttttgtcaa |
| | | caatataattcatatcccctcactttcggagggggcacgaagttggaaataaagggtagcacctct |
| | | ggtagcggcaagcctggctctggcgagggtagtaccaaaggagaagttcaacttgtgcaaagcg |
| | | gggcagaagtgaaaaaacccggggcgagcgttaaaatatcttgtaaaacaagtggctacaccttc |
| | | acggagtacaccatccactgggttaaacaagcttctggaaagggacttgaatggatcgggaacat |
| | | aaaccccaacaatgggggcactacttataatcaaaagtttgaggatcgggctaccctcacagtgg |
| | | ataagtccacctccacagcttatatggaattgagtagccttaggagcgaggatacagccgtttattat |
| | | tgtgcggcgggctggaactttgactattgggggcaagggacgacggtgacggtgtcctcc |

Chimeric antigen receptors (CARs) are engineered receptors that may direct or redirect T cells or NK cells (e.g., patient or donor T or NK cells) to target a selected antigen. A CAR may be engineered to recognize an antigen and, when bound to that antigen, activate the immune cell to attack and destroy the cell bearing that antigen. When these antigens exist on tumor cells, an immune cell that expresses the CAR may target and kill the tumor cell. CARs generally comprise an extracellular binding motif that mediates antigen binding (e.g., an anti-PSMA binding domain), a transmembrane domain that spans, or is understood to span, the cell membrane when the CAR is present at a cell surface or cell membrane, and an intracellular (or cytoplasmic) signaling domain.

According to at least one non-limiting view, there have been at least three “generations” of CAR compositions. In a first generation of CARs, a binding motif (e.g., a single chain fragment variable, binding motif) is linked or connected to a signaling domain (e.g., CD32) via a transmembrane domain, optionally comprising a hinge domain and one or more spacers. In a second generation of CARs, a costimulatory domain (CM1, such as CD28, 4-1BB, or OX-40) is introduced with the signaling domain (e.g., CD3ζ). In a third generation of CARs, a second costimulatory domain (CM2) is comprised.

TCRs are heterodimers composed of an α-chain and a β-chain. TCR signaling requires recruitment of signaling proteins that generate an immune synapse. In addition, TCR localization at the plasma membrane depends on CD3 complex, which is expressed in T cells. Engineered single chain TCRs may be generated, e.g., using transmembrane and signaling domains of CAR constructs, methods and constructs for which are known (e.g., sTCR and TCR-CAR molecules, e.g., fusion of a TCRβ chain with CD28 TM and CD28 and CD3ζ signaling modules). An anti-PSMA binding system of the present disclosure may comprise one or more antigen binding motifs that bind PSMA. In some embodiments, an antigen binding system further comprises a costimulatory domain, and/or an extracellular domain (e.g., a “hinge” or “spacer” region), and/or a transmembrane domain, and/or an intracellular (signaling) domain, and/or a CD3-zeta or CD3-epsilon activation domain. In some embodiments, an anti-PSMA binding system of the present disclosure comprises at least a binding motif that binds human PSMA, a costimulatory domain, an extracellular domain, a transmembrane domain, and a CD3-zeta or CD3-epsilon activating domain.

In some embodiments, an anti-PSMA CAR of the present disclosure may comprise an antigen binding system that comprises one or more, or all, of a leader peptide (P), an anti-PSMA binding domain (B), a costimulatory protein's extracellular domain (E), a transmembrane domain (T), a costimulatory domain (C), a second costimulatory domain (C′), and an activation domain (A). In some instances, an anti-PSMA CAR is configured according to the following: B E T A. In some instances, an anti-PSMA CAR is configured according to the following: P B E T A. In some instances, an anti-PSMA CAR is configured according to the following: B E T C A. In some instances, an anti-PSMA CAR is configured according to the following: P B E T C A. In some instances, an anti-PSMA CAR is configured according to the following: B E T C C′ A. In some instances, an anti-PSMA CAR is configured according to the following: P B E T C C′ A. In some embodiments, the an anti-PSMA CAR comprises a VH and a VL, optionally wherein the CAR is configured according to the following: P-VH-VL-E-T-C-A or P-VL-VH-E-T-C-A. In some embodiments, the VH and the VL are connected by a linker (L), optionally wherein the CAR is configured according to the following, from N-terminus to C-terminus: P-VH-L-VL-E-T-C-A or P-VH-L-VL-E-T-C-A.

One or more antigen binding motifs determine the target(s) of an antigen binding system. A binding motif of an antigen binding system may comprise any an anti-PSMA binding domain, e.g., an antibody provided by the present disclosure, e.g., a binding motif of the present disclosure. Binding domain are used in chimeric antigen receptors at least in part because they may be engineered to be expressed as part of a single chain along with the other CAR components. See, for example, U.S. Pat. Nos. 7,741,465, and 6,319,494 as well as Eshhar et al., Cancer Immunol Immunotherapy (1997) 45:131-136, Krause et al., J. Exp. Med., Volume 188, No. 4, 1998 (619-626); Finney et al., Journal of Immunology, 1998, 161:2791-2797, each of which is incorporated herein by reference with respect to binding domains in CARs. A binding domain or scFv, is a single chain antigen binding fragment comprising a heavy chain variable domain and a light chain variable domain, which heavy chain variable domain and light chain variable domain are linked or connected together. See, for example, U.S. Pat. Nos. 7,741,465, and 6,319,494 as well as Eshhar et al., Cancer Immunol Immunotherapy (1997) 45:131-136, each of which is incorporated herein by reference with respect to binding motif domains. When derived from a parent antibody, a binding motif may retain some of, retain all of, or essentially retain the parent antibody's binding of a target antigen. In some embodiments, a CAR contemplated herein comprises antigen-specific binding domain that may be a scFv (a murine, human or humanized scFv) that binds an antigen expressed on a cancer cell. In a certain embodiment, the scFv binds PSMA.

In certain embodiments, the CARs contemplated herein may comprise linker residues between the various domains, e.g., between VH and VL domains, added for appropriate spacing conformation of the molecule. CARs contemplated herein, may comprise one, two, three, four, or five or more linkers. In some embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G) n; glycine-serine polymers (G1 5S1 5) n (SEQ ID NO: 257), where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs described herein. Glycine accesses more phi-psi space than even alanine, and is much less restricted than residues with longer side chains (see Scheraga, Rev. Computational Chem. 11173-142 (1992)). Other linkers contemplated herein include Whitlow linkers (see Whitlow, Protein Eng. 6 (8): 989-95 (1993)). The ordinarily skilled artisan will recognize that design of a CAR in some embodiments may include linkers that are all or partially flexible, such that the linker may include a flexible linker as well as one or more portions that confer less flexible structure to provide for a desired CAR structure. In one embodiment, any of the constructs described herein may comprise a “GS” linker. In another embodiment, any of the constructs described herein comprise a “GSG” linker. In an example a glycine-serine linker comprises or consists of the amino acid sequence GS (SEQ ID NO: 121), which may be encoded by the nucleic acid sequence according to ggatcc (SEQ ID NO: 122) or gggtcc (SEQ ID NO: 123). In an example a glycine-serine linker comprises or consists of the amino acid sequence GGGSGGGS (SEQ ID NO: 124), which may be encoded by the nucleic acid sequence according to ggeggtggaagcggaggaggttcc (SEQ ID NO: 125). In another embodiment, the CARs described herein comprise the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) of SEQ ID NO: 126 (GSTSGSGKPGSGEGSTKG (SEQ ID NO: 126). In an embodiment, a linker is encoded by a nucleic acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid sequence according ggctccacctccggaagcggcaaacccggtagcggcgagggctccacaaagggc (SEQ ID NO: 127)

In embodiments, a CAR comprises a scFv that further comprises a variable region linking sequence. A “variable region linking sequence,” is an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In one embodiment, the variable region linking sequence is 1, 2, 3, 4, 5, 6, 7, 8, 9, 10, 11, 12, 13, 14, 15, 16, 17, 18, 19, 20, 21, 22, 23, 24, 25, or more amino acids long.

In embodiments, the binding domain of the CAR is followed by one or more “spacer domains,” which refers to the region that moves the antigen binding domain away from the effector cell surface to enable proper cell/cell contact, antigen binding and activation (Patel et al., Gene Therapy, 1999; 6:412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region.

The binding domain of the CAR may generally be followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain. The hinge domain may be derived either from a natural, synthetic, semi-synthetic, or recombinant source. The hinge domain may include the amino acid sequence of a naturally occurring immunoglobulin hinge region or an altered immunoglobulin hinge region.

In some embodiments, an Antigen binding system of the present disclosure may comprise a hinge that is, is from, or is derived from (e.g., comprises all or a fragment of) an immunoglobulin-like hinge domain. In some embodiments, a hinge domain is from or derived from an immunoglobulin. In some embodiments, a hinge domain is selected from the hinge of IgG1, IgG2, IgG3, IgG4, IgA, IgD, IgE, or IgM, or a fragment thereof.

A hinge may be derived from a natural source or from a synthetic source. Hinge domains suitable for use in the CARs described herein include the hinge region derived from the extracellular regions of type 1 membrane proteins such as CD8α, CD4, CD28 and CD7, which may be wild-type hinge regions from these molecules or may be altered, for example a truncated CD28 hinge domain. A hinge may be derived from a natural source or from a synthetic source. In some embodiments, an Antigen binding system of the present disclosure may comprise a hinge that is, is from, or is derived from (e.g., comprises all or a fragment of) CD2, CD3 delta, CD3 epsilon, CD3 gamma, CD4, CD7, CD8α, CD8β, CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3 DPI), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA1-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof. In certain embodiments, a CAR does not comprise a CD28 hinge. In embodiments, the hinge domain comprises a CD8α hinge region. In embodiments the CARs described herein comprise a hinge domain from CD8α having the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) of SEQ ID NO: 129 (TTTPAPRPPTPAPTIASQPLSLRPEACRPAAGGAVHTRGLDFACD (SEQ ID NO: 129)). In embodiments, hinge domain from CD8α is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to accacgacgccagegccgegaccaccaacaccggegcccaccategegtcgcaacccctgtccctgcgccccgaggcgtgccggcc agcggcggggggcgcagtgcacacgagggggctggacttcgcctgtgat (SEQ ID NO: 130).

Polynucleotide and polypeptide sequences of these hinge domains are known. In some embodiments, the polynucleotide encoding a hinge domain comprises a nucleotide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% (e.g., 85-90%, 85-95%, 85-100%, 90-95%, 90-100%, or 95-100%) identical to a nucleotide sequence known. In some embodiments, the polypeptide sequence of a hinge domain comprises a polypeptide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% (e.g., 85-90%, 85-95%, 85-100%, 90-95%, 90-100%, or 95-100%) identical to a known polypeptide sequence.

In general, a “transmembrane domain” (e.g., of an antigen binding system) refers to a domain having an attribute of being present in the membrane when present in a molecule at a cell surface or cell membrane (e.g., spanning a portion or all of a cellular membrane). A costimulatory domain for an antigen binding system of the present disclosure may further comprise a transmembrane domain and/or an intracellular signaling domain. It is not required that every amino acid in a transmembrane domain be present in the membrane. For example, in some embodiments, a transmembrane domain is characterized in that a designated stretch or portion of a protein is substantially located in the membrane. Amino acid or nucleic acid sequences may be analyzed using a variety of algorithms to predict protein subcellular localization (e.g., transmembrane localization). The programs psort (PSORT.org) and Prosite (prosite.expasy.org) are exemplary of such programs.

The type of transmembrane domain comprised in an antigen binding system described herein is not limited to any type. In some embodiments, a transmembrane domain is selected that is naturally associated with a binding motif and/or intracellular domain. In some instances, a transmembrane domain comprises a modification of one or more amino acids (e.g., deletion, insertion, and/or substitution), e.g., to avoid binding of such domains to a transmembrane domain of the same or different surface membrane proteins to minimize interactions with other members of the receptor complex.

A transmembrane domain may be derived either from a natural or from a synthetic source. Where the source is natural, a domain may be derived from any membrane-bound or transmembrane protein. Exemplary transmembrane domains may be derived from (e.g., may comprise at least a transmembrane domain of) an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof. In some embodiments, a transmembrane domain may be synthetic (and can, e.g., comprise predominantly hydrophobic residues such as leucine and valine). In some embodiments, a triplet of phenylalanine, tryptophan and valine are comprised at each end of a synthetic transmembrane domain. In some embodiments, a transmembrane domain is directly linked or connected to a cytoplasmic domain. In some embodiments, a short oligo- or polypeptide linker (e.g., between 2 and 10 amino acids in length) may form a linkage between a transmembrane domain and an intracellular domain. In some embodiments, a linker is a glycine-serine doublet. In embodiments the CARs described herein comprise a TM domain from CD8α having the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) of SEQ ID NO: 131 (IYIWAPLAGTCGVLLLSLVITLYC (SEQ ID NO: 131)). In embodiments, TM domain from CD8α is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to atctacatctgggcgcccttggccgggacttgtggggtccttctcctgtcactggttatcaccctttattgc (SEQ ID NO: 132).

Polynucleotide and polypeptide sequences of transmembrane domains provided herein are known. In some embodiments, the polynucleotide encoding a transmembrane domain comprises a nucleotide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% (e.g., 85-90%, 85-95%, 85-100%, 90-95%, 90-100%, or 95-100%) identical to a nucleotide sequence known. In some embodiments, the polypeptide sequence of a transmembrane domain comprises a polypeptide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% (e.g., 85-90%, 85-95%, 85-100%, 90-95%, 90-100%, or 95-100%) identical to a polypeptide sequence known.

Optionally, short spacers may form linkages between any or some of the extracellular, transmembrane, and intracellular domains of the CAR.

Intracellular signaling domains that may transduce a signal upon binding of an antigen to an immune cell are known, any of which may be comprised in an antigen binding system of the present disclosure. For example, cytoplasmic sequences of a T cell receptor (TCR) are known to initiate signal transduction following TCR binding to an antigen (see, e.g., Brownlie et al., Nature Rev. Immunol. 13:257-269 (2013)).

In some embodiments, CARs contemplated herein comprise an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. In some embodiments, a signaling domain and/or activation domain comprises an immunoreceptor tyrosine-based activation motif (ITAM). Examples of ITAM containing cytoplasmic signaling sequences comprise those derived from TCR zeta, FcR gamma, FcR beta, CD3 zeta, CD3 gamma, CD3 delta, CD3 epsilon, CD5, CD22, CD79a, CD79b, and CD66d (see, e.g., Love et al., Cold Spring Harb. Perspect. Biol. 2:a002485 (2010); Smith-Garvin et al., Annu. Rev. Immunol. 27:591-619 (2009)). In certain embodiments, suitable signaling domains comprise, without limitation, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

The term “effector function” refers to a specialized function of the cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain may be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

It is known that signals generated through the TCR alone are insufficient for full activation of the T cell and that a secondary or costimulatory signal may also be required. Thus, T cell activation may be said to be mediated by two distinct classes of intracellular signaling domains: primary signaling domains that initiate antigen-dependent primary activation through the TCR (e.g., a TCR/CD3 complex) and costimulatory signaling domains that act in an antigen independent manner to provide a secondary or costimulatory signal. In some embodiments, a CAR contemplated herein comprises an intracellular signaling domain that comprises one or more “costimulatory signaling domain” and a “primary signaling domain.”

Illustrative examples of ITAM containing primary signaling domains that are useful in the present disclosure include those derived from TCRζ, FcRγ, FcRβ, DAP12, CD3γ, CD38, CD38, CD3ζ, CD22, CD79a, CD79b, and CD66d. In some embodiments, a CAR comprises a CD3ζ primary signaling domain and one or more costimulatory signaling domains. The intracellular primary signaling and costimulatory signaling domains may be linked in any order in tandem to the carboxyl terminus of the transmembrane domain. In one embodiment, the CARs have a CD3ζ domain having the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) of SEQ ID NO: 133. LRVKFSRSADAPAYQQGQNQLYNELNLGRREEYDVLDKRRGRDPEMGGKPRRKNPQE GLYNELQKDKMAEAYSEIGMKGERRRGKGHDGLYQGLSTATKDTYDALHMQALPPR (SEQ ID NO: 133). In embodiments, a CD33 domain is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to

(SEQ ID NO: 134)
ttgagagtgaagttcagcaggagcgcagacgcccccgcctatcagcaaggc
cagaaccagctctataacgagctcaatttagggcgaagagaggagtacgat
gttttggacaagaggcgtggccgggaccccgaaatggggggaaagccgaga
aggaagaaccctcaggaaggcttgtacaatgaattgcagaaggataagatg
gcggaggcatacagtgagattgggatgaaaggcgagcgccggaggggcaag
gggcacgatggcctttatcagggtctcagtacagccaccaaggacacctac
gacgcccttcacatgcaagccctgccccctcgc.

CARs contemplated herein comprise one or more costimulatory signaling domains to enhance the efficacy and expansion of T cells expressing CAR receptors. As used herein, the term, “costimulatory signaling domain,” or “costimulatory domain”, refers to an intracellular signaling domain of a costimulatory molecule. In some embodiments, costimulatory molecules may include CD27, CD28, CD137 (4-IBB), OX40 (CD134), CD30, CD40, PD-I, ICOS (CD278), CTLA4, LFA-1, CD2, CD7, LIGHT, TRIM, LCK3, SLAM, DAPIO, LAG3, HVEM, and NKD2C, and CD83. In embodiments, the CARs described herein comprise a 4-IBB costimulatory domain having the amino acid sequence of having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 135. KRGRKKLLYIFKQPFMRPVQTTQEEDGCSCRFPEEEEGGCE (SEQ ID NO: 135). In embodiments, a 4-IBB costimulatory domain is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to, which may be encoded by the nucleic acid sequence according to

(SEQ ID NO: 136)
aaacggggcagaaagaaactcctgtatatattcaaacaaccatttatgaga
ccagtacaaactactcaagaggaagatggctgtagctgccgatttccagaa
gaagaagaaggaggatgtgaa.

The engineered CARs described herein may also comprise an N-terminal signal peptide or tag at the N-terminus of the scFv or antigen binding domain. In one embodiment, a heterologous signal peptide may be used. The antigen binding domain or scFV may be fused to a leader or a signal peptide that directs the nascent protein into the endoplasmic reticulum and subsequent translocation to the cell surface. It is understood that, once a polypeptide containing a signal peptide is expressed at the cell surface, the signal peptide is generally proteolytically removed during processing of the polypeptide in the endoplasmic reticulum and translocation to the cell surface. Thus, a polypeptide such as the CAR constructs described herein, are generally expressed at the cell surface as a mature protein lacking the signal peptide, whereas the precursor form of the polypeptide includes the signal peptide. Any suitable signal sequence known in the art may be used. Similarly any known tag sequence known in the art may also be used. In one embodiment a signal sequence is a CSF2RA signal sequence. In embodiments, the CARs described herein comprise a CSF2RA signal sequence having the amino acid sequence of having at least 75% sequence identity to (such as, at least 75%, at least 80%, at least 90%, at least 95%, or 100% identity; e.g., 85-90%, 85-95%, 85-100%, 90-95%, 90-100%, or 95-100%) to SEQ ID NO: 137;

(SEQ ID NO: 137)
MLLLVTSLLLCELPHPAFLLIP
SEQ ID
(SEQ ID NO: 138)
MEWTWVFLFLLSVTAGVHS,
(SEQ ID NO: 139)
MALPVTALLLPLALLLHAARP.

Components of a CAR may be exchanged or “swapped” using routine techniques of biotechnology for equivalent components. To provide just a few non-limiting and partial examples, a CAR of the present disclosure may comprise a binding motif as provided herein in combination with a hinge provided herein and a costimulatory domain provided herein. In certain examples, a CAR of the present disclosure may comprise a leader sequence as provided herein together with a binding motif as provided herein in combination with a hinge provided herein and s costimulatory domain provided herein.

The present disclosure comprises conjugates in which an antibody of the present disclosure is associated with a therapeutic agent or a detectable moiety. In various embodiments, the therapeutic agent is an anti-cancer agent as provided herein. In certain embodiments, provided conjugate comprises one or more detectable moieties, i.e., is “labeled” with one or more such moieties. In some such embodiments, a conjugate of the present disclosure is useful in diagnostic or imaging applications, e.g., diagnosing or imaging cancer. Any of a wide variety of detectable moieties may be used in labeled antibody conjugates described herein. Suitable detectable moieties comprise, without limitation: various ligands, radionuclides; fluorescent dyes; chemiluminescent agents (such as, for example, acridinum esters, stabilized dioxetanes, and the like); bioluminescent agents; spectrally resolvable inorganic fluorescent semiconductors nanocrystals (i.e., quantum dots); microparticles; metal nanoparticles (e.g., gold, silver, copper, platinum, etc.); nanoclusters; paramagnetic metal ions; enzymes; colorimetric labels (such as, for example, dyes, colloidal gold, and the like); biotin; dioxigenin; haptens; and proteins for which antisera or monoclonal antibodies are available.

The present disclosure comprises nucleic acids encoding anti-PSMA binding domains provided herein. The present disclosure comprises nucleic acids encoding antibodies of the provided herein, comprising, without limitation, nucleic acids encoding anti-PSMA binding domains. The present disclosure comprises nucleic acids encoding antigen binding systems provided herein, comprising without limitation nucleic acids encoding anti-PSMA chimeric antigen receptors. The nucleic acid sequence of SEQ ID NO: 2 comprises and provides exemplary nucleic acid sequences corresponding to and encoding each of SEQ ID NOs: 1 and 3-11. The nucleic acid sequence of SEQ ID NO: 13 comprises and provides exemplary nucleic acid sequences corresponding to and encoding each of SEQ ID NOs: 12 and 14-22. The nucleic acid sequence of SEQ ID NO: 24 comprises and provides exemplary nucleic acid sequence corresponding to and encoding SEQ ID NOs: 23.

The nucleic acid sequence of SEQ ID NO: 26 comprises and provides exemplary nucleic acid sequences corresponding to and encoding each of SEQ ID NOs: 25 and 27-35. The nucleic acid sequence of SEQ ID NO: 37 comprises and provides exemplary nucleic acid sequences corresponding to and encoding each of SEQ ID NOs: 36 and 38-46. The nucleic acid sequence of SEQ ID NO: 48 comprises and provides an exemplary nucleic acid sequence corresponding to and encoding SEQ ID NOs: 47.

The nucleic acid sequence of SEQ ID NO: 50 comprises and provides exemplary nucleic acid sequences corresponding to and encoding each of SEQ ID NOs: 49 and 51-59. The nucleic acid sequence of SEQ ID NO: 61 comprises and provides exemplary nucleic acid sequences corresponding to and encoding each of SEQ ID NOs: 60 and 62-70. The nucleic acid sequence of SEQ ID NO: 72 comprises and provides an exemplary nucleic acid sequence corresponding to and encoding SEQ ID NOs: 71.

The nucleic acid sequence of SEQ ID NO: 74 comprises and provides exemplary nucleic acid sequences corresponding to and encoding each of SEQ ID NOs: 73 and 75-83. The nucleic acid sequence of SEQ ID NO: 85 comprises and provides exemplary nucleic acid sequences corresponding to and encoding each of SEQ ID NOs: 84 and 86-94. The nucleic acid sequence of SEQ ID NO: 76 comprises and provides an exemplary nucleic acid sequence corresponding to and encoding SEQ ID NOs: 95.

The nucleic acid sequence of SEQ ID NO: 98 comprises and provides exemplary nucleic acid sequences corresponding to and encoding each of SEQ ID NOs: 97 and 99-101. The nucleic acid sequence of SEQ ID NO: 109 comprises and provides exemplary nucleic acid sequences corresponding to and encoding each of SEQ ID NOs: 108 and 110-118. The nucleic acid sequence of SEQ ID NO: 108 comprises and provides an exemplary nucleic acid sequence corresponding to and encoding SEQ ID NOs: 107.

In one embodiment described herein, an anti-PSMA CAR construct has an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 140. DIQLTQSPSSLSASVGDRVTITCRASSSVSHIYWYQQKPGKAPKPWIYRTSNLASGVPSR FSGSGSGTDYTLTISSLQPEDFATYYCQQYHTYPPTFGQGTKLEIKGSTSGSGKPGSGEG STKGEVQLVESGGGLVQPGGSMRLSCAASGFTFSDYYMAWVRQAPGKGLEWIANINY DGSNTYYADSLKGRFTISRDNSKNTLYLQMNSLRAEDTAVYYCARNWDGYYGYFDV WGQGTTVTVSSGSTTTPAPRPPTPAPTIASQPLSLRPEACRPAAGGAVHTRGLDFACDIY IWAPLAGTCGVLLLSLVITLYCKRGRKKLLYIFKQPFMRPVQTTQEEDGCSCRFPEEEEG GCELRVKFSRSADAPAYQQGQNQLYNELNLGRREEYDVLDKRRGRDPEMGGKPRRKN PQEGLYNELQKDKMAEAYSEIGMKGERRRGKGHDGLYQGLSTATKDTYDALHMQAL PPR (SEQ ID NO: 140). In embodiments an anti-PSMA CAR is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence to according

(SEQ ID NO: 141)
gatatccagctgacccagtccccttcctctctgtctgcgtctgttggcgat
cgtgtcaccatcacttgtcgtgccagcagcagcgtgagccacatttattgg
taccaacaaaagcccggcaaagcccctaagccttggatctacagaacctcc
aatctggccagcggcgtgcccagcagattcagcggaagcggatccggcacc
gactacactttaaccatcagctctttacagcccgaggacttcgccacatac
tactgccagcagtaccacacctatccccccacattcggccaaggaacaaag
ctggagattaagggctccacctccggaagcggcaaacccggtagcggcgag
ggctccacaaagggcgaggtgcaacttgtggagagcggaggaggtttagtg
caacccggaggcagcatgagactgagctgcgccgccagcggcttcacattc
tccgactactacatggcttgggtccgacaagctcccggaaaaggactggag
tggatcgccaacatcaactacgacggctccaacacctactacgccgactct
ttaaagggtcgtttcacaatctctcgtgacaacagcaagaacactttatat
ttacaaatgaactctttaagggccgaggataccgccgtgtactactgcgct
cgtaactgggacggctactacggctacttcgacgtgtggggccaaggaacc
accgtgaccgtgagcagcgggtccaccacgacgccagcgccgcgaccacca
acaccggcgcccaccatcgcgtcgcaacccctgtccctgcgccccgaggcg
tgccggccagcggcggggggcgcagtgcacacgagggggctggacttcgcc
tgtgatatctacatctgggcgcccttggccgggacttgtggggtccttctc
ctgtcactggttatcaccctttattgcaaacggggcagaaagaaactcctg
tatatattcaaacaaccatttatgagaccagtacaaactactcaagaggaa
gatggctgtagctgccgatttccagaagaagaagaaggaggatgtgaattg
agagtgaagttcagcaggagcgcagacgcccccgcctatcagcaaggccag
aaccagctctataacgagctcaatttagggcgaagagaggagtacgatgtt
ttggacaagaggcgtggccgggaccccgaaatggggggaaagccgagaagg
aagaaccctcaggaaggcttgtacaatgaattgcagaaggataagatggcg
gaggcatacagtgagattgggatgaaaggcgagcgccggaggggcaagggg
cacgatggcctttatcagggtctcagtacagccaccaaggacacctacgac
gcccttcacatgcaagccctgccccctcgc.

In one embodiment described herein, an anti-PSMA CAR construct has an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 142. DIQMTQSPSSLSASVGDRVTVTCRASQNVNTNVAWYQQKPGKAPKVLIYSASYRNSGV PSRFSGSGSGTDFTLTISSVQPEDFATYYCQQYNSYPFTFGQGTKLEIKGSTSGSGKPGSG EGSTKGQVQLVQSGAEVKKPGASVKLSCKASGYTFTTYWMHWVRQAPGQGLEWIGM IHPNSGSTNYAQKFQGRATLTVDTSTSTAYMELSSLRSEDTAVYYCARDPYDYGEDFD VWGQGTTVTVSSGSTTTPAPRPPTPAPTIASQPLSLRPEACRPAAGGAVHTRGLDFACDI YIWAPLAGTCGVLLLSLVITLYCKRGRKKLLYIFKQPFMRPVQTTQEEDGCSCRFPEEEE GGCELRVKFSRSADAPAYQQGQNQLYNELNLGRREEYDVLDKRRGRDPEMGGKPRRK NPQEGLYNELQKDKMAEAYSEIGMKGERRRGKGHDGLYQGLSTATKDTYDALHMQA LPPR (SEQ ID NO: 142). In embodiments an anti-PSMA CAR is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to

(SEQ ID NO: 143)
gacatccagatgacccagagccccagctctttaagtgccagcgtgggcgac
agagtgacagtgacttgtcgtgccagccagaacgtgaataccaacgtggct
tggtaccagcagaagcccggcaaagcccctaaggtgctgatctattccgcg
tcttatcgtaactccggcgtgccttcgcgtttttctgggtctggtagcggc
accgacttcactttaacaatcagcagcgttcagcccgaagacttcgccacc
tactactgccagcagtacaacagctatccctttactttcggtcaagggacc
aagctcgagatcaaaggctccaccagcggtagcggcaaacccggttccggc
gagggctctaccaagggccaagtgcagctggtgcagtccggcgccgaggtg
aagaagcccggtgcttccgtgaagctgtcttgcaaagccagcggctacacc
ttcaccacctattggatgcactgggtccgacaagctcccggtcaaggtctg
gagtggattggcatgatccaccccaactccggctccaccaactacgcccag
aagttccaaggtcgtgccactttaacagtggataccagcaccagcaccgcc
tacatggagctgagtagtttgaggagcgaggacaccgccgtgtactattgc
gctcgtgacccctacgactacggcgaggacttcgacgtgtggggccaagga
acaacagtgaccgtgagcagcgggtccaccacgacgccagcgccgcgacca
ccaacaccggcgcccaccatcgcgtcgcaacccctgtccctgcgccccgag
gcgtgccggccagcggcggggggcgcagtgcacacgagggggctggacttc
gcctgtgatatctacatctgggcgcccttggccgggacttgtggggtcctt
ctcctgtcactggttatcaccctttattgcaaacggggcagaaagaaactc
ctgtatatattcaaacaaccatttatgagaccagtacaaactactcaagag
gaagatggctgtagctgccgatttccagaagaagaagaaggaggatgtgaa
ttgagagtgaagttcagcaggagcgcagacgcccccgcctatcagcaaggc
cagaaccagctctataacgagctcaatttagggcgaagagaggagtacgat
gttttggacaagaggcgtggccgggaccccgaaatggggggaaagccgaga
aggaagaaccctcaggaaggcttgtacaatgaattgcagaaggataagatg
gcggaggcatacagtgagattgggatgaaaggcgagcgccggaggggcaag
gggcacgatggcctttatcagggtctcagtacagccaccaaggacacctac
gacgcccttcacatgcaagccctgccccctcgc.

In one embodiment described herein, an anti-PSMA CAR construct has an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 144 EVOLVESGGGLVQPGGSMRLSCAASGFTFSDYYMAWVRQAPGKGLEWVANINYDGTS TYYADSLKGRFTISRDSSKNTLYLQMNSLRAEDTAVYYCARALDGYYGYLDVWGQGT TVTVSSGSTSGSGKPGSGEGSTKGDIQLTQSPSSLSASVGDRVTLTCRASQSISNNLHWY QQKPGKAPKLLIKYVSQSISGIPSRFSGSGLGTDFTLTISSVQPEDFATYYCQQSNSWPYT FGQGTKLEIKGSTTTPAPRPPTPAPTIASQPLSLRPEACRPAAGGAVHTRGLDFACDIYIW APLAGTCGVLLLSLVITLYCKRGRKKLLYIFKQPFMRPVQTTQEEDGCSCRFPEEEEGGC ELRVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQALPP R (SEQ ID NO: 144). In embodiments an anti-PSMA CAR is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to

(SEQ ID NO: 145)
gaggtgcagctggtggagtccggaggaggtttagtccaacccggtggcagc
atgaggctgtcttgtgctgcctccggcttcactttttctgattactacatg
gcttgggtccgacaagctcccggaaaaggtttagagtgggtggctaacatc
aactacgacggcaccagcacctactatgccgacagcctcaagggcagattc
accatctctcgtgattcgtctaaaaacactttatatttacaaatgaactct
ttaagagccgaagataccgccgtgtactattgcgctcgtgccctcgacggc
tactacggatatttagacgtgtggggtcaaggaacaaccgtgaccgtgtcc
agcggatccacctccggaagcggcaaacccggtagcggcgaaggcagcacc
aaaggagacatccagctgacccagagccctagctctttaagcgctagcgtg
ggcgatagggtgactctgacttgtcgtgcgtcccaaagcattagcaacaat
ttacactggtaccagcagaagcccggaaaagcccccaagctgctgatcaaa
tatgtgagccagagcatctccggcatcccctctcgtttttctggtagcgga
ctgggcaccgactttactttaaccatcagcagcgtccagcccgaggacttc
gccacatactactgccagcagagcaacagctggccctatactttcggccaa
ggaacaaagctggagatcaaggggtccaccacgacgccagcgccgcgacca
ccaacaccggcgcccaccatcgcgtcgcaacccctgtccctgcgccccgag
gcgtgccggccagcggcggggggcgcagtgcacacgagggggctggacttc
gcctgtgatatctacatctgggcgcccttggccgggacttgtggggtcctt
ctcctgtcactggttatcaccctttattgcaaacggggcagaaagaaactc
ctgtatatattcaaacaaccatttatgagaccagtacaaactactcaagag
gaagatggctgtagctgccgatttccagaagaagaagaaggaggatgtgaa
ttgagagtgaagttcagcaggagcgcagacgcccccgcctatcagcaaggc
cagaaccagctctataacgagctcaatttagggcgaagagaggagtacgat
gttttggacaagaggcgtggccgggaccccgaaatggggggaaagccgaga
aggaagaaccctcaggaaggcttgtacaatgaattgcagaaggataagatg
gcggaggcatacagtgagattgggatgaaaggcgagcgccggaggggcaag
gggcacgatggcctttatcagggtctcagtacagccaccaaggacacctac
gacgcccttcacatgcaagccctgccccctcgc.

In one embodiment described herein, an anti-PSMA CAR construct has an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 146. DIVMTQSPSSLSASVGDRVTITCKASQDVGTAVDWYQQKPGKAPKLLIYWASTRHTGV PDRFTGSGSGTDFTLTISSLQPEDFADYFCQQYNSYPLTFGGGTKLEIKGSTSGSGKPGSG EGSTKGEVQLVQSGAEVKKPGASVKISCKTSGYTFTEYTIHWVKQASGKGLEWIGNINP NNGGTTYNQKFEDRATLTVDKSTSTAYMELSSLRSEDTAVYYCAAGWNFDYWGQGTT VTVSSGSTTTPAPRPPTPAPTIASQPLSLRPEACRPAAGGAVHTRGLDFACDIYIWAPLAG TCGVLLLSLVITLYCKRGRKKLLYIFKQPFMRPVQTTQEEDGCSCRFPEEEEGGCELRVK FSRSADAPAYQQGQNQLYNELNLGRREEYDVLDKRRGRDPEMGGKPRRKNPQEGLYN ELQKDKMAEAYSEIGMKGERRRGKGHDGLYQGLSTATKDTYDALHMQALPPR (SEQ ID NO: 146). In embodiments an anti-PSMA CAR is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to

(SEQ ID NO: 147)
gacattgtgatgactcagtctccttcttctctttccgcttccgttggggac
cgcgtcactataacttgtaaagcgtcccaagatgtcggcaccgccgttgac
tggtaccagcaaaaacccgggaaagcgccgaaactgctcatctactgggct
tcaacccgccacacgggtgtcccggaccggtttacggggagcggtagtgga
accgatttcactctgaccatttcctcccttcaaccggaagatttcgctgac
tacttttgtcaacaatataattcatatcccctcactttcggagggggcacg
aagttggaaataaagggtagcacctctggtagcggcaagcctggctctggc
gagggtagtaccaaaggagaagttcaacttgtgcaaagcggggcagaagtg
aaaaaacccggggcgagcgttaaaatatcttgtaaaacaagtggctacacc
ttcacggagtacaccatccactgggttaaacaagcttctggaaagggactt
gaatggatcgggaacataaaccccaacaatgggggcactacttataatcaa
aagtttgaggatcgggctaccctcacagtggataagtccacctccacagct
tatatggaattgagtagccttaggagcgaggatacagccgtttattattgt
gcggcgggctggaactttgactattgggggcaagggacgacggtgacggtg
tcctccgggtccaccacgacgccagcgccgcgaccaccaacaccggcgccc
accatcgcgtcgcaacccctgtccctgcgccccgaggcgtgccggccagcg
gcggggggcgcagtgcacacgagggggctggacttcgcctgtgatatctac
atctgggcgcccttggccgggacttgtggggtccttctcctgtcactggtt
atcaccctttattgcaaacggggcagaaagaaactcctgtatatattcaaa
caaccatttatgagaccagtacaaactactcaagaggaagatggctgtagc
tgccgatttccagaagaagaagaaggaggatgtgaattgagagtgaagttc
agcaggagcgcagacgcccccgcctatcagcaaggccagaaccagctctat
aacgagctcaatttagggcgaagagaggagtacgatgttttggacaagagg
cgtggccgggaccccgaaatggggggaaagccgagaaggaagaaccctcag
gaaggcttgtacaatgaattgcagaaggataagatggcggaggcatacagt
gagattgggatgaaaggcgagcgccggaggggcaaggggcacgatggcctt
tatcagggtctcagtacagccaccaaggacacctacgacgcccttcacatg
caagccctgccccctcgc.

In one embodiment described herein, an anti-PSMA CAR construct has an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 148. DIQLTQSPSSLSASVGDRVTITCRASSSVSHMYWYQQKPGKAPKPWIYRTSNLASGVPS RFSGSGSGTDYTLTISSMQPEDFATYYCQQYHSYPLTFGQGTKLEIKGSTSGSGKPGSGE GSTKGEVQLVESGGGLVQPGGSLRLSCAASGFTFSDYYMAWVRQAPGKGLEWVANIN YDGSSTFYADSLKGRFTISRDNSKNTLYLQMNSLRAEDTAVYYCGRQVGYYDPMDYW GQGTTVTVSSGSTTTPAPRPPTPAPTIASQPLSLRPEACRPAAGGAVHTRGLDFACDIYI WAPLAGTCGVLLLSLVITLYCKRGRKKLLYIFKQPFMRPVQTTQEEDGCSCRFPEEEEG GCELRVKFSRSADAPAYQQGQNQLYNELNLGRREEYDVLDKRRGRDPEMGGKPRRKN PQEGLYNELQKDKMAEAYSEIGMKGERRRGKGHDGLYQGLSTATKDTYDALHMQAL PPR (SEQ ID NO: 148). In embodiments an anti-PSMA CAR is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to

(SEQ ID NO: 149)
gacatccagctgacccagtcccccagctctttatccgctagcgtgggcgat
agggtgaccatcacttgtcgtgcgtcttcgtctgtgtctcatatgtactgg
taccagcagaagcccggcaaggcccccaagccttggatctatcgtacatcc
aatcttgcaagcggcgtcccttctcgtttttctggttccgggtctggtacc
gactacactttaaccatcagcagcatgcagcccgaggacttcgccacctac
tactgccagcagtatcactcctatcctttaacttttggccaaggaacaaag
ttggagatcaagggcagcacctccggtagcggaaagcccggtagcggcgag
ggcagcaccaagggagaggtgcagttggtggagagcggaggaggactggtg
cagcccggtggctctttaagactcagctgtgccgccagcggatttacattc
tccgactactacatggcttgggtccgacaagcccccggaaaaggtttagag
tgggtggccaacatcaactacgacggctcctccacattctacgccgactct
ttaaagggtcgtttcaccatctctcgtgacaacagcaaaaatactttatat
ttacaaatgaactctttaagggccgaggacaccgccgtgtactactgcggt
cgtcaagttggctattacgaccccatggactactggggccaaggaactacc
gtgaccgtgagcagcgggtccaccacgacgccagcgccgcgaccaccaaca
ccggcgcccaccatcgcgtcgcaacccctgtccctgcgccccgaggcgtgc
cggccagcggcggggggcgcagtgcacacgagggggctggacttcgcctgt
gatatctacatctgggcgcccttggccgggacttgtggggtccttctcctg
tcactggttatcaccctttattgcaaacggggcagaaagaaactcctgtat
atattcaaacaaccatttatgagaccagtacaaactactcaagaggaagat
ggctgtagctgccgatttccagaagaagaagaaggaggatgtgaattgaga
gtgaagttcagcaggagcgcagacgcccccgcctatcagcaaggccagaac
cagctctataacgagctcaatttagggcgaagagaggagtacgatgttttg
gacaagaggcgtggccgggaccccgaaatggggggaaagccgagaaggaag
aaccctcaggaaggcttgtacaatgaattgcagaaggataagatggcggag
gcatacagtgagattgggatgaaaggcgagcgccggaggggcaaggggcac
gatggcctttatcagggtctcagtacagccaccaaggacacctacgacgcc
cttcacatgcaagccctgccccctcgc.

Notch receptors are single pass transmembrane proteins that mediate cell-cell contact signaling and play a central role in development and other aspects of cell-to-cell communication between two contacting cells, in which one contacting cell has the Notch receptor, and the other contacting cell is a cell that exhibits a ligand on its surface which binds to the corresponding Notch receptor. The engagement of native Notch and Delta, it's native ligand, leads to two-step proteolysis of the Notch receptor that ultimately causes the release of the intracellular portion of the receptor from the membrane into the cytoplasm, where it moves to the nucleus. There the released domain alters cell behavior by functioning as a transcriptional regulator. Notch receptors are involved in and are required for a variety of cellular functions during development and are critical for the function of numerous cell-types across species.

Synthetic Notch receptors (synNotch receptors) disclosed herein can display one or more different binding moieties on the cell surface, for example, scFVs, nanobodies, single chain T-cell receptors, to name a few, that recognize a prostate specific cancer antigen (PSCA) causing the release of the intracellular, transcriptional regulatory portion of the receptor from the membrane into the cytoplasm resulting in transcriptional regulation. Engineered cells bearing synNotch receptors that encounter their specific target antigen (PSCA) will then be cleaved such that their cytosolic or intercellular fragment is free to translocate into the cell nucleus to regulate the transcription of any open reading frame (ORF) under the control of a synthetic promoter, for example a GAL4 promoter. As disclosed herein the ORF expressed is an anti-PSMA chimeric antigen T-cell receptor (CAR-T) that targets a separate, distinct target antigen (PSMA) for target cell killing, only after the priming target antigen (prostate specific cancer antigen (PSCA) in the synNotch receptors disclosed herein) detected by the synNotch receptor has been detected. This enables highly-specific combinatorial antigen pattern recognition to allow greater discrimination between diseased or cancerous cells and healthy cells. This could greatly enable the application of engineered CAR-T cells to safely target tumors with less side-effects on healthy tissue.

SynNotch receptor polypeptides of the present disclosure comprise: a) an extracellular anti-PSCA binding domain; b) a Notch receptor core polypeptide, where the Notch receptor polypeptide has a length of from 50 amino acids to 1000 amino acids, and comprises one or more ligand-inducible (PSCA binding) proteolytic cleavage sites; and c) an intracellular domain. Binding of the anti-PSCA binding domain to PSCA, for example on the surface of a prostate cancer cell, induces cleavage of the Notch receptor core polypeptide at the one or more ligand-inducible proteolytic cleavage sites, thereby releasing the intracellular domain. Release of the intracellular domain induces the expression of an anti-PSMA CAR in a cell that produces the anti-PSCA synNotch receptor polypeptide. The extracellular domain comprises an amino acid sequence that is heterologous to the Notch core receptor polypeptide. The intracellular domain comprises an amino acid sequence that is heterologous to the Notch core receptor polypeptide.

A synNotch receptor polypeptide of the present disclosure comprises an extracellular domain that includes an antigen binding domain that specifically binds prostate specific cancer antigen (PSCA), i.e. an anti-PSCA binding domain. In embodiments the anti-PSCA binding may comprise antigen-binding sequences as found in an antibody described herein. In some instances, an anti-PSCA binding domain comprises an antigen binding fragment described herein, such as an scFv. Unless otherwise indicated, it is to be appreciated the references to PSCA in the present disclosure relate to human PSCA. In various embodiments, an anti-PSCA binding motif comprises at least one heavy chain CDR (HCDR) provided herein, e.g., at least one HCDR disclosed in Table 9. In various embodiments, an anti-PSCA binding domain comprises two HCDRs provided herein, e.g., at least two HCDRs disclosed in Table 9. In various embodiments, an anti-PSCA binding domain comprises three HCDRs provided herein, e.g., three HCDRs disclosed in Table 9. In various embodiments, an anti-PSCA binding domain comprises at least one light chain CDR (LCDR) provided herein, e.g., at least one LCDR disclosed in Table 9. In various embodiments, an anti-PSCA binding domain comprises two LCDRs provided herein, e.g., at least two LCDRs disclosed in Table 9. In various embodiments, an anti-PSCA binding domain comprises three LCDRs provided herein, e.g., three LCDRs disclosed in Table 9.

In various embodiments, an anti-PSCA binding domain comprises at least one HCDR provided herein, e.g., at least one HCDR disclosed in Table 9, and at least one LCDR provided herein, e.g., at least one LCDR disclosed in Table 9. In various embodiments, an anti-PSCA binding domain comprises one HCDR provided herein, e.g., at least one HCDR disclosed in Table 9, and one LCDR provided herein, e.g., derived from the same Table of Table 9 as the HCDR(s). In various embodiments, an anti-PSCA binding domain comprises two HCDRs provided herein, e.g., at least two HCDRs disclosed in Table 9, and two LCDRs provided herein, e.g., at least two LCDRs disclosed in Table 9. In various embodiments, an anti-PSCA binding domain comprises two HCDRs provided herein, e.g., at least two HCDRs disclosed in Table 9, and two LCDRs provided herein, e.g., derived from the same Table of Table 9 as the HCDR(s). In various embodiments, an anti-PSCA binding domain comprises three HCDRs provided herein, e.g., three HCDRs disclosed in Table 9, and three LCDRs provided herein, e.g., three LCDRs disclosed in Table 9. In various embodiments, an anti-PSCA binding domain comprises three HCDRs provided herein, e.g., three HCDRs disclosed in Table 9, and three LCDRs derived from the same Table of Table 9 as the HCDR(s).

In various embodiments, an anti-PSCA binding domain comprises at least one heavy chain framework region (heavy chain FR) of a heavy chain variable domain disclosed herein, e.g., at least one heavy chain FR of a heavy chain variable domain disclosed in Table 9. In various embodiments, an anti-PSCA binding domain comprises two heavy chain FRs of a heavy chain variable domain disclosed herein, e.g., at least two heavy chain FRs of a heavy chain variable domain disclosed in Table 9. In various embodiments, an anti-PSCA binding domain comprises three heavy chain FRs of a heavy chain variable domain disclosed herein, e.g., three heavy chain FRs of a heavy chain variable domain disclosed in Table 9.

In various embodiments, an anti-PSCA binding domain comprises at least one light chain FR of a light chain variable domain disclosed herein, e.g., at least one light chain FR of a light chain variable domain disclosed in Table 9. In various embodiments, an anti-PSCA binding domain comprises two light chain FRs of a light chain variable domain disclosed herein, e.g., at least two light chain FRs of a light chain variable domain disclosed in Table 9. In various embodiments, an anti-PSCA binding domain comprises three light chain FRs of a light chain variable domain disclosed herein, e.g., three light chain FRs of a light chain variable domain disclosed in Table 9.

In various embodiments, an anti-PSCA binding domain comprises at least one heavy chain FR of a heavy chain variable domain disclosed herein, e.g., at least one heavy chain FR of a heavy chain variable domain disclosed in Table 9, and at least one light chain FR of a light chain variable domain disclosed herein, e.g., at least one light chain FR of a light chain variable domain disclosed in Table 9. In various embodiments, an anti-PSCA binding domain comprises one heavy chain FR of a heavy chain variable domain disclosed herein, e.g., at least one heavy chain FR of a heavy chain variable domain disclosed in Table 9, and one light chain FR of a light chain variable domain disclosed herein, e.g., derived from the same Table of Table 9 as the heavy chain FR(s). In various embodiments, an anti-PSCA binding domain comprises two heavy chain FRs of a heavy chain variable domain disclosed herein, e.g., at least two heavy chain FRs of a heavy chain variable domain disclosed in Table 9, and two light chain FRs of a light chain variable domain disclosed herein, e.g., at least two light chain FRs of a light chain variable domain disclosed in Table 9. In various embodiments, an anti-PSCA binding domain comprises two heavy chain FRs of a heavy chain variable domain disclosed herein, e.g., at least two heavy chain FRs of a heavy chain variable domain disclosed in Table 9, and two light chain FRs of a light chain variable domain disclosed herein, e.g., derived from the same Table of Table 9 as the heavy chain FR(s). In various embodiments, an anti-PSCA binding domain comprises three heavy chain FRs of a heavy chain variable domain disclosed herein, e.g., three heavy chain FRs of a heavy chain variable domain disclosed in Table 9, and three light chain FRs of a light chain variable domain disclosed herein, e.g., three light chain FRs of a light chain variable domain disclosed in Table 9. In various embodiments, an anti-PSCA binding domain comprises three heavy chain FRs of a heavy chain variable domain disclosed herein, e.g., three light chain FRs of a light chain variable domain disclosed in Table 9, and three light chain FRs derived from the same Table of Table 9 as the heavy chain FR(s).

Exemplary antibody sequences provided in Table 9 are suitable for use in any antibody format, comprising, e.g., a tetrameric antibody, a monospecific antibody, a bispecific antibody, an antigen binding fragment, or a binding motif. Heavy chain variable domains and light chain variable domains and portions thereof provided in Table 9 may be comprised in a binding motif.

In various embodiments, an anti-PSCA binding domain comprises one, two, or three FRs that together or each individually have at least 75% identity (e.g., at least 75%, at least 80%, at least 90%, at least 95%, or 100%, e.g., 85-90%, 85-95%, 85-100%, 90-95%, 90-100%, or 95-100%) to corresponding FR(s) of a heavy chain variable domain of a heavy chain variable domain disclosed in in Table 9. In various embodiments, an anti-PSCA binding domain comprises one, two, or three FRs that together or each individually have at least 75% identity (e.g., at least 75%, at least 80%, at least 90%, at least 95%, or 100%) to corresponding FR(s) of a light chain variable domain of a light chain variable domain disclosed in Table 9.

In various embodiments, an anti-PSCA binding domain comprises at least one heavy chain variable domain having at least 75% sequence identity to a heavy chain variable domain disclosed in Table 9 (e.g., at least 75%, at least 80%, at least 90%, at least 95%, or 100% identity; e.g., 85-90%, 85-95%, 85-100%, 90-95%, 90-100%, or 95-100%).

In various embodiments, an anti-PSCA binding domain comprises at least one light chain variable domain having at least 75% sequence identity to a light chain variable domain disclosed in Table 9 (e.g., at least 75%, at least 80%, at least 90%, at least 95%, or 100% identity; e.g., 85-90%, 85-95%, 85-100%, 90-95%, 90-100%, or 95-100%).

In various embodiments, an anti-PSCA binding domain comprises at least one heavy chain variable domain having at least 75% sequence identity to a heavy chain variable domain disclosed in Table 9 (e.g., at least 75%, at least 80%, at least 90%, at least 95%, or 100% identity; e.g., 85-90%, 85-95%, 85-100%, 90-95%, 90-100%, or 95-100%) and at least one light chain variable domain having at least 75% sequence identity to a light chain variable domain disclosed in Table 9 (e.g., at least 75%, at least 80%, at least 90%, at least 95%, or 100% identity; e.g., 85-90%, 85-95%, 85-100%, 90-95%, 90-100%, or 95-100%).

Table 9 represents the heavy chain variable domain and light chain variable domain sequences of an exemplary antibody, comprising (i) the heavy chain variable domain of the exemplary antibody; (ii) a DNA sequence encoding the heavy chain variable domain (iii) three heavy chain variable domain CDRs of the heavy chain variable domain, according to IMGT, Kabat, and Chothia numbering; (iv) the light chain variable domain of the exemplary antibody; (v) a DNA sequence encoding the light chain variable domain; and (vi) three light chain variable domain CDRs of the light chain variable domain, according to IMGT, Kabat, and Chothia numbering. Information provided in each table provides framework amino acid sequences, as well as nucleotide sequences encoding each CDR amino acid sequence and nucleotide sequences encoding corresponding FR amino acid sequence.

In various embodiments an anti-PSCA binding domain may comprise a heavy chain variable domain (e.g., having at least 75% sequence identity to a heavy chain variable domain in Table 9, e.g., at least 80%, 85%, 90%, 95%, or 100% identity; e.g., 85-90%, 85-95%, 85-100%, 90-95%, 90-100%, or 95-100%), a light chain variable domain (e.g., having at least 75% sequence identity to a light chain variable domain in Table 9, e.g., at least 80%, 85%, 90%, 95%, or 100% identity; e.g., 85-90%, 85-95%, 85-100%, 90-95%, 90-100%, or 95-100%), and a linker (e.g., a linker according to SEQ ID NO: 126). In various embodiments a binding motif may comprise a leader sequence, a heavy chain variable domain (e.g., having at least 75% sequence identity to a heavy chain variable domain of any one of Table 9, e.g., at least 80%, 85%, 90%, 95%, or 100% identity; e.g., 85-90%, 85-95%, 85-100%, 90-95%, 90-100%, or 95-100%), a light chain variable domain (e.g., having at least 75% sequence identity to a light chain variable domain of any one of Table 9, e.g., at least 80%, 85%, 90%, 95%, or 100% identity; e.g., 85-90%, 85-95%, 85-100%, 90-95%, 90-100%, or 95-100%), and a linker. The linker joining the two variable domains will be apparent from the sequence in view of the present disclosure. For the avoidance of doubt, a heavy chain variable domain and a light chain variable domain may be present in any orientation, e.g., an orientation in which the heavy chain variable domain is C terminal of the light chain variable domain or in which the heavy chain variable domain is N terminal of the light chain variable domain. In various embodiments an anti-PSCA binding domain may comprise a linker according to SEQ ID NO: 126.

In certain embodiments, an anti-PSCA binding domain comprises a binding domain that comprises a heavy chain variable domain of the present disclosure, a light chain variable domain of the present disclosure, and a linker having at least 75% sequence identity to SEQ ID NO: 126 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-PSCA binding domain comprises a binding motif that comprises a linker according to SEQ ID NO: 126. In certain embodiments, an anti-PSCA binding domain comprises a binding motif that comprises a heavy chain variable domain of the present disclosure, a light chain variable domain of the present disclosure, and a leader sequence having at least 75% sequence identity to SEQ ID NO: 137, SEQ ID NO: 138, or SEQ ID NO: 139 (e.g., at least 75%, at least 80%, at least 90%, at least 95%, or 100% identity; e.g., 85-90%, 85-95%, 85-100%, 90-95%, 90-100%, or 95-100%). In certain embodiments, an anti-PSCA binding domain comprises a binding motif that comprises a CSF2RA leader sequence according to SEQ ID NO: 137.

In certain embodiments, an anti-PSCA binding domain comprises a binding motif that comprises a heavy chain variable domain of the present disclosure, a light chain variable domain of the present disclosure, a linker of the present disclosure, and a leader sequence of the present disclosure.

A binding agent that is based on an exemplary antibody provided herein, such as for example Ab6, may be provided in any fragment or format, comprising a heavy chain variable domain according to the indicated exemplary antibody and a light chain variable domain according to the indicated exemplary antibody.

TABLE 9
Exemplary Antibody Sequences 6 (Ab6)

| SEQ | | |
|---|---|---|
| ID | | |
| NO: | Description | Sequence |
| 150 | Heavy Chain | QVQLQQWGAGLLKPSETLSLTCAVYGGSFSGNYWSWIRQPP |
| | Variable | GKGLEWIGEINHSGSTNYNPSLKSRVTISVDTSKNQFSLKLSS |
| | Domain | VTAADTAVYYCARGGSYNYFDYWGQGTLVTVSS |
| 151 | VH (DNA) | Caagttcagctgcagcagtggggagctggtttactgaagcctagcgagacactgtctttaacatg |
| | | cgccgtgtacggcggaagcttcagcggcaactattggagctggatcagacagcctcccggtaa |
| | | gggtttagagtggatcggcgagatcaaccactccggctccaccaactataacccctctttaaagtc |
| | | tcgtgtgaccatctccgtggacaccagcaagaaccagttctctttaaagctgagctccgtgacag |
| | | ccgccgacaccgctgtgtattactgtgctcgtggcggcagctacaactacttcgactactggggc |
| | | caaggtaccctcgtgaccgtgtccagc |
| 152 | CDRH1 | GGSFSGNY |
| | IMGT (Prot) | |
| 153 | CDRH1 | GNYWS |
| | Kabat (Prot) | |
| 154 | CDRH1 | GGSFSG |
| | Chothia | |
| | (Prot) | |
| 155 | CDRH2 | INHSGST |
| | IMGT (Prot) | |
| 156 | CDRH2 | EINHSGSTNYNPSLKS |
| | Kabat (Prot) | |
| 157 | CDRH2 | EINHSGSTNYNPSLKS |
| | Chothia | |
| | (Prot) | |
| 158 | CDRH3 | ARGGSYNYFDY |
| | IMGT (Prot) | |
| 159 | CDRH3 | GGSYNYFDY |
| | Kabat (Prot) | |
| 160 | CDRH3 | GGSYNYFDY |
| | Chothia | |
| | (Prot) | |
| 161 | Light Chain | DIVMTQSPLSLPVTPGEPASISCRSSQSLLHSNGYNYLVWYLQ |
| | Variable | KPGQSPQLLIYLGSIRASGVPDRFSGSGSGTDFTLKISRVEAED |
| | Domain | VGVYYCMQPLQTPITFGQGTRLEIK |
| 162 | VL (DNA) | Gacatcgtgatgacacagagccctctgtctttacccgttacccccggtgaacccgctagcatcag |
| | | ctgcagaagctcccagtctttactccacagcaacggctacaactatttagtgtggtatttacagaaa |
| | | cccggccagagcccccagctgctgatttatctgggctccattcgtgctagcggcgtgcccgatag |
| | | attttccggcagcggaagcggcaccgacttcactttaaagatctctcgtgtggaggccgaggac |
| | | gtgggcgtctactactgtatgcagcctctgcagacccccattaccttcggccaaggtactcgtctg |
| | | gaaatcaag |
| 163 | CDRL1 | QSLLHSNGYNY |
| | IMGT (Prot) | |
| 164 | CDRL1 | RSSQSLLHSNGYNYLV |
| | Kabat (Prot) | |
| 165 | CDRL1 | RSSQSLLHSNGYNYLV |
| | Chothia | |
| | (Prot) | |
| 166 | CDRL2 | LGS |
| | IMGT (Prot) | |
| 167 | CDRL2 | LGSIRAS |
| | Kabat (Prot) | |
| 168 | CDRL2 | LGSIRAS |
| | Chothia | |
| | (Prot) | |
| 169 | CDRL3 | MQPLQTPITF |
| | IMGT (Prot) | |
| 170 | CDRL3 | MQPLQTPIT |
| | Kabat (Prot) | |
| 171 | CDRL3 | MQPLQTPIT |
| | Chothia | |
| | (Prot) | |
| 172 | ScFv | DIVMTQSPLSLPVTPGEPASISCRSSQSLLHSNGYNYLVWYLQ |
| | | KPGQSPQLLIYLGSIRASGVPDRFSGSGSGTDFTLKISRVEAED |
| | | VGVYYCMQPLQTPITFGQGTRLEIKGSTSGSGKPGSGEGSTK |
| | | GQVQLQQWGAGLLKPSETLSLTCAVYGGSFSGNYWSWIRQP |
| | | PGKGLEWIGEINHSGSTNYNPSLKSRVTISVDTSKNQFSLKLS |
| | | SVTAADTAVYYCARGGSYNYFDYWGQGTLVTVSS |
| 173 | ScFv | gacatcgtgatgacacagagccctctgtctttacccgttacccccggtgaacccgctagcatcag |
| | | ctgcagaagctcccagtctttactccacagcaacggctacaactatttagtgtggtatttacagaaa |
| | | cccggccagagcccccagctgctgatttatctgggctccattcgtgctagcggcgtgcccgatag |
| | | attttccggcagcggaagcggcaccgacttcactttaaagatctctcgtgtggaggccgaggac |
| | | gtgggcgtctactactgtatgcagcctctgcagacccccattaccttcggccaaggtactcgtctg |
| | | gaaatcaagggcagcaccagcggcagcggaaaacccggaagcggcgagggaagcaccaa |
| | | aggccaagttcagctgcagcagtggggagctggtttactgaagcctagcgagacactgtctttaa |
| | | catgcgccgtgtacggcggaagcttcagcggcaactattggagctggatcagacagcctcccg |
| | | gtaagggtttagagtggatcggcgagatcaaccactccggctccaccaactataacccctctttaa |
| | | agtctcgtgtgaccatctccgtggacaccagcaagaaccagttctctttaaagctgagctccgtga |
| | | cagccgccgacaccgctgtgtattactgtgctcgtggcggcagctacaactacttcgactactgg |
| | | ggccaaggtaccctcgtgaccgtgtccagc |

As noted above, a synNotch receptor polypeptide comprises a Notch core receptor polypeptide having a length of from 50 amino acids to 1000 amino acids and comprising one or more ligand-inducible proteolytic cleavage sites. In embodiments, the Notch core receptor polypeptide present in a synNotch receptor polypeptide of the present disclosure has a length of from 50 amino acids to 1000 amino acids, e.g., from 50 amino acids to 75 amino acids, from 75 amino acids to 100 amino acids, from 100 amino acids to 150 amino acids, from 150 amino acids to 200 amino acids, from 200 amino acids to 250 amino acids, from 250 a to 300 amino acids, from 300 amino acids to 350 amino acids, from 350 amino acids to 400 amino acids, from 400 amino acids to 450 amino acids, from 450 amino acids to 500 amino acids, from 500 amino acids to 550 amino acids, from 550 amino acids to 600 amino acids, from 600 amino acids to 650 amino acids, from 650 amino acids to 700 amino acids, from 700 amino acids to 750 amino acids, from 750 amino acids to 800 amino acids, from 800 amino acids to 850 amino acids, from 850 amino acids to 900 amino acids, from 900 amino acids to 950 amino acids, or from 950 amino acids to 1000 amino acids. In some embodiments, the Notch core receptor polypeptide present in a synNotch receptor polypeptide of the present disclosure has a length of from 300 amino acids to 400 amino acids. In some embodiments, the Notch core receptor polypeptide present in a synNotch receptor polypeptide of the present disclosure has a length of from 300 amino acids to 350 amino acids. In some embodiments, the Notch core receptor polypeptide present in a synNotch receptor polypeptide of the present disclosure has a length of from 300 amino acids to 325 amino acids. In some embodiments, the Notch core receptor polypeptide present in a synNotch receptor polypeptide of the present disclosure has a length of from 350 amino acids to 400 amino acids. In some embodiments, the Notch core receptor polypeptide present in a synNotch receptor polypeptide of the present disclosure has a length of from 750 amino acids to 850 amino acids. In some embodiments, the Notch core receptor polypeptide present in a synNotch receptor polypeptide of the present disclosure has a length of from 50 amino acids to 75 amino acids. In some embodiments, the Notch core receptor polypeptide present in a synNotch receptor polypeptide of the present disclosure has a length of from 310 amino acids to 320 amino acids, e.g., 310 amino acids, 311 amino acids, 312 amino acids, 313 amino acids, 314 amino acids. 315 amino acids, 316 amino acids, 317 amino acids, 318 amino acids, 319 amino acids, or 320 amino acids. In some embodiments, the Notch core receptor polypeptide present in a synNotch receptor polypeptide of the present disclosure has a length of 315 amino acids. In some embodiments, the Notch core receptor polypeptide present in a synNotch receptor polypeptide of the present disclosure has a length of from 360 amino acids to 370 amino acids, e.g., 360 amino acids, 361 amino acids, 362 amino acids, 363 amino acids 364 amino acids, 365 amino acids, 366 amino acids. 367 amino acids, 368 amino acids, 369 amino acids, or 370 amino acids. In some embodiments, the Notch core receptor polypeptide present in a synNotch receptor polypeptide of the present disclosure has a length of 367 amino acids.

In some embodiments, the Notch core receptor polypeptide present in a synNotch receptor polypeptide of the present disclosure comprises an amino acid sequence having at least 75%, at least 80%, at least 85%, at least 90%, at least 95%, at least 98%, at least 99%, or 100%, amino acid sequence identity to the following amino acid sequence: IPYKIEAVKSEPVEPPLPSQLHLMYVAAAAFVLLFFVGCGVLLSRKRRRQHGQL (SEQ ID NO: 174); where the Notch core receptor polypeptide comprises an S2 proteolytic cleavage site and an S3 proteolytic cleavage site; where the Notch receptor polypeptide has a length of from 50 amino acids (aa) to 65 aa, e.g., 50, 51, 52, 53, 54, 55, 56, 57, 58, 59, 60, 61, 62, 63, 64, or 65 aa.

In some embodiments, the Notch core receptor polypeptide present in a syn Notch receptor polypeptide of the present disclosure comprises an amino acid sequence having at least 75%, at least 80%, at least 85%, at least 90%, at least 95%, at least 98%, at least 99%, or 100%, amino acid sequence identity to the following amino acid sequence: IPYKIEAVKSEPVEPPLPSQLHLMYVAAAAFVLLFFVGCGVLLSRKRRRQLCIQKL (SEQ ID NO: 175); where the TM domain is underlined; where the Notch core receptor polypeptide comprises an S2 proteolytic cleavage site and an S3 proteolytic cleavage site; where the Notch receptor polypeptide has a length of from 50 amino acids (aa) to 65 aa, e.g., 50, 51, 52, 53, 54, 55, 56, 57, 58, 59, 60, 61, 62, 63, 64, or 65 aa.

In some embodiments, the Notch core receptor polypeptide present in a syn Notch receptor polypeptide of the present disclosure comprises, in order from N-terminus to C-terminus: i) a LNR-A segment; ii) a LNR-B segment; iii) a LNR-C segment; iv) an HD-N segment, v) an HD-C segment; and vi) a TM domain. A LNR-A segment, LNR-B segment, and LNR-C segment can collectively be referred to as an “LNR segment.”

An LNR segment can comprise an amino acid sequence having at least 75%, at least 80%, at least 85%, at least 90%, at least 95%, at least 98%, at least 99%, or 100%, amino acid sequence identity to the amino acid sequence: following PPQIEEACELPECQVDAGNKVCNLQCNNHACGWDGGDCSLNFNDPWKNCTQSLQCW KYFSDGHCDSQCNSAGCLFDGFDCQLTEGQCNPLYDQYCKDHFSDGHCDQGCNSAEC EWDGLDC (SEQ ID NO: 176); and can have a length of from 118 to 122 amino acids (e.g., 118, 119, 120, 121, or 122 amino acids).

An HD segment (HD-N plus HD-C) can comprise an amino acid sequence having at least 75%, at least 80%, at least 85%, at least 90%, at least 95%, at least 98%, at least 99%, or 100%, amino acid identity the sequence to following amino acid sequence: AAGTLVLVVLLPPDQLRNNSFHFLRELSHVLHTNVVFKRDAQGQQMIFPYYGHEEELR KHPIKRSTVGWATSSLLPGTSGGRQRRELDPMDIRGSIVYLEIDNRQCVQSSSQCFQSAT DVAAFLGALASLGSLNIPYKIEAVKSEPVEPPLP (SEQ ID NO:177); and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCGVLLS (SEQ ID NO:178); and can have a length of 21, 22, 23, 24, or 25 amino acids.

In some embodiments, a Notch receptor polypeptide comprises an amino acid sequence having at least 75%, at least 80%, at least 85%, at least 90%, at least 95%, at least 98%, at least 99%, or 100%, amino acid sequence identity to the following amino acid sequence: PPQIEEACELPECQVDAGNKVCNLQCNNHACGWDGGDCSLNFNDPWKNCTQSLQCW KYFSDGHCDSQCNSAGCLFDGFDCQLTEGQCNPLYDQYCKDHFSDGHCDQGCNSAEC EWDGLDCAEHVPERLAAGTLVLVVLLPPDQLRNNSFHFLRELSHVLHTNVVFKRDAQG QQMIFPYYGHEEELRKHPIKRSTVGWATSSLLPGTSGGRQRRELDPMDIRGSIVYLEIDN RQCVQSSSQCFQSATDVAAFLGALASLGSLNIPYKIEAVKSEPVEPPLPSQLHLMYVAA AAFVLLFFVGCGVLLS (SEQ ID NO: 179); and has a length of from 300 amino acids to 310 amino acids (e.g., 300, 301, 302, 303, 304, 305, 306, 307, 308, 309, or 310 amino acids).

In some embodiments, a Notch core receptor polypeptide comprises an amino acid sequence having at least 75%, at least 809%, at least 85%, at least 90%, at least 95%, at least 98%, at least 99%, or 100%, amino acid sequence identity to the following amino acid sequence: PCVGSNPCYNQGTCEPTSENPFYRCLCPAKFNGLLCHILDYSFTGGAGRDIPPPQI EEACELPECQVDAGNKVCNLQCNNHACGWDGGDCSLNFNDPWKNCTQSLQCWKYFS DGHCDSQCNSAGCLFDGFDCQLTEGQCNPLYDQYCKDHFSDGHCDQGCNSAECEWDG LDCAEHVPERLAAGTLVLVVLLPPDQLRNNSFHFLRELSHVLHTNVVFKRDAQGQQMI FPYYGHEEELRKHPIKRSTVGWATSSLLPGTSGGRQRRELDPMDIRGSIVYLEIDNRQCV QSSSQCFQSATDVAAFLGALASLGSLNIPYKIEAVKSEPVEPPLPSQLHLMYVAAAAFVL LFFVGCGVLLS (SEQ ID NO:180); and has a length of from 350 amino acids to 370 amino acids (e.g., 350 351, 352, 353, 354, 355, 356, 357, 358, 359, 360, 361, 362, 363, 364, 365, 366, 367, 368, 369, or 370 amino acids).

In some embodiments, the Notch core receptor polypeptide present in a synNotch receptor polypeptide of the present disclosure comprises, in order from N-terminus to C-terminus: i) a single EGF repeat; ii) an LNR segment: iii) an HD-N segment, iv) an HD-C segment; and v) a TM domain.

An EGF repeat can comprise an amino acid sequence having at least 75%, at least 80%, at least 85%, at least 90%, at least 95%, at least 98%, at least 99%, or 100%, amino acid sequence identity to the following sequence: PCVGSNPCYNQGTCEPTSENPFYRCLCPAKFNGLLCH (SEQ ID NO: 181); and can have a length of 35 amino acids to 40 amino acids (e.g., 35, 36, 37, 38, 39, or 40 amino acids.

An LNR segment can comprise an amino acid sequence having at least 75%, at least 80%, at least 85%, at least 90%, at least 95%, at least 98%, at least 99%, or 100%, amino acid sequence identity 10 the following amino acid sequence: PPQIEEACELPECQVDAGNKVCNLQCNNHACGWDGGDCSLNFNDPWKNCTQSLQCW KYFSDGHCDSQCNSAGCLFDGFDCQLTEGQCNPLYDQYCKDHFSDGHCDQGCNSAEC EWDGLDC (SEQ ID NO:182); and can have a length of from 118 to 122 amino acids (e.g., 118, 119, 120, 121, or 122 amino acids).

An HD segment (HD-N plus HD-C) can comprise an amino acid sequence having at least 75%, at least 80%, at least 85%, at least 90%, at least 95%, at least 98%, at least 99%, or 100%. amino acid sequence the following identity to amino acid sequence: AAGTLVLVVLLPPDQLRNNSFHFLRELSHVLHTNVVFKRDAQGQQMIFPYYGHEEELR KHPIKRSTVGWATSSLLPGTSGGRQRRELDPMDIRGSIVYLEIDNRQCVQSSSQCFQSAT DVAAFLGALASLGSLNIPYKIEAVKSEPVEPPLP (SEQ ID NO: 183); and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCGVLLS (SEQ ID NO:184); and can have a length of 21, 22, 23, 24, or 25 amino acids.

In some embodiments, a Notch core receptor polypeptide comprises a synthetic linker. For example, In some embodiments, a Notch core receptor polypeptide comprises, in order from N-terminus to C-terminus: i) a synthetic linker; ii) an EGF repeat; iii) an LNR segment; iv) an HD-N segment, v) an HD-C segment; and vi) a TM domain.

A synthetic linker can have a length of from about 10 amino acids (aa) to about 200 aa, e.g., from 10 aa to 25 aa, from 25 aa to 50 aa, from 50 aa to 75 aa, from 75 aa to 100 aa. from 100 aa to 125 aa, from 125 aa to 150 aa, from 150 aa to 175 aa, or from 175 aa to 200 aa. A synthetic linker can have a length of from 10 aa to 30 aa, e.g., 10, 11, 12, 13, 14, 15, 16, 17, 18, 19, 20, 21, 22, 23, 24, 25, 26, 27, 28, 29, or 30 aa. A synthetic linker can have a length of from 30 aa to 50 aa, e.g., from 30 aa to 35 aa, from 35 aa to 40 aa, from 40 aa to 45 aa, or from 45 aa to 50 aa.

In some instances, a synthetic linker, as described herein, may include an extracellular protein structural domain or a portion thereof. Extracellular protein structural domains suitable for use as a synthetic linker include but are not limited to e.g., Ig-like extracellular structural domains. Fc extracellular structural domains, fibronectin extracellular structural domains and the like. In some instances, a synthetic linker may include a plurality of extracellular protein structural domains where the plurality may include a plurality of the same domain or a plurality of different domains.

In some embodiments, the Notch core receptor polypeptide present in a synNotch receptor polypeptide of the present disclosure comprises, in order from N-terminus to C-terminus: i) from two to eleven EGF repeats; ii) an LNR segment; iii) an HD-N segment, iv) an HD-C segment; and v) a TM domain.

In some embodiments, the Notch core receptor polypeptide present in a syn Notch receptor polypeptide of the present disclosure comprises, in order from N-terminus to C-terminus: i) two EGF repeats; ii) an LNR segment; iii) an HD-N segment, iv) an HD-C segment; and v) a TM domain. In some embodiments, the Notch core receptor polypeptide present in a synNotch receptor polypeptide of the present disclosure comprises, in order from N-terminus to C-terminus: i) three EGF repeats; ii) an LNR segment; iii) an HD-N segment, iv) an HD-C segment; and v) a TM domain. In some embodiments, the Notch core receptor polypeptide present in a synNotch receptor polypeptide of the present disclosure comprises, in order from N-terminus to C-terminus: i) four EGF repeats; ii) an LNR segment; iii) an HD-N segment, iv) an HD-C segment; and v) a TM domain. In some embodiments, the Notch core receptor polypeptide present in a synNotch receptor polypeptide of the present disclosure comprises, in order from N-terminus to C-terminus: i) five EGF repeats; ii) an LNR segment; iii) an HD-N segment, iv) an HD-C segment; and v) a TM domain. In some embodiments, the Notch core receptor polypeptide present in a synNotch receptor polypeptide of the present disclosure comprises, in order from N-terminus to C-terminus: i) six EGF repeats; ii) an LNR segment; iii) an HD-N segment, iv) an HD-C segment; and v) a TM domain. In some embodiments, the Notch core receptor polypeptide present in a synNotch receptor polypeptide of the present disclosure comprises, in order from N-terminus to C-terminus: i) seven EGF repeats; ii) an LNR segment; iii) an HD-N segment, iv) an HD-C segment; and v) a TM domain. In some embodiments, the Notch core receptor polypeptide present in a synNotch receptor polypeptide of the present disclosure comprises, in order from N-terminus to C-terminus: i) eight EGF repeats; ii) an LNR segment; iii) an HD-N segment, iv) an HD-C segment; and v) a TM domain. In some embodiments, the Notch core receptor polypeptide present in a synNotch receptor polypeptide of the present disclosure comprises, in order from N-terminus to C-terminus: i) nine EGF repeats: ii) an LNR segment; iii) an HD-N segment, iv) an HD-C segment; and v) a TM domain. In some embodiments, the Notch core receptor polypeptide present in a synNotch receptor polypeptide of the present disclosure comprises, in order from N-terminus to C-terminus: i) ten EGF repeats; ii) an LNR segment; iii) an HD-N segment, iv) an HD-C segment; and v) a TM domain. In some embodiments, the Notch core receptor polypeptide present in a synNotch receptor polypeptide of the present disclosure comprises, in order from N-terminus to C-terminus: i) eleven EGF repeats; ii) an LNR segment; iii) an HD-N segment, iv) an HD-C segment; and v) a TM domain.

An EGF repeat can comprise an amino acid sequence having at least 75%, at least 80%, at least 85%, at least 90%, at least 95%, at least 98%, at least 99%, or 100%, amino acid sequence identity to DINECVLSPCRHGASCONTHGGYRCHCQAGYSGRNCE; SEQ ID NO:185 and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to DIDDCRPNPCHNGGSCTDGINTAFCDCLPGFRGTFC; SEQ ID NO: 186 and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to DINECASDPCRNGANCTDCVDSYTCTCPAGFSGIHCE; (SEQ ID NO: 187 and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to TESSCFNGGTCVDGINSFTCLCPPGFTGSYCQ; SEQ ID NO: 188 and can have a length of from 30 amino acids (aa) to 35 aa (e.g., 30, 31, 32, 33, 34, or 35 aa).

An EGF repeat can comprise an amino acid sequence having at least 75%, at least 80%, at least 85%, at least 90%, at least 95%, at least 98%, at least 99%, or 100%, amino acid sequence identity to DVNECDSQPCLHGGTCQDGCGSYRCTCPQGYTGPNCQ; SEQ ID NO: 189 and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to DSSPCKNGGKCWQTHTQYRCECPSGWT; SEQ ID NO: 190 and can have a length of from 25 amino acids (aa) to 30 aa, e.g., 25, 26, 27, 28, 29, or 30 aa.

An EGF repeat can comprise an amino acid sequence having at least 75%, at least 80%, at least 85%, at least 90%, at least 95%, at least 98%, at least 99%, or 100%, amino acid sequence identity to LVDECSPSPCQNGATCTDYLGGYSCKCVAGYHGVNC; SEQ ID NO: 191 and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to IDECLSHPCQNGGTCLDLPNTYKCSCPRGTQGVHCE; SEQ ID NO: 192 and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to CFNNGTCVDQVGGYSCTCPPGFVGERCE; SEQ ID NO: 193 and can have a length of from 25 amino acids (aa) to 30 aa, e.g., 25, 26, 27, 28, 29, or 30 aa.

An EGF repeat can comprise an amino acid sequence having at least 75%, at least 80%, at least 85%, at least 90%, at least 95%, at least 98%, at least 99%, or 100%, amino acid sequence identity to DVNECLSNPCDARGTQNCVQRVNDFHCECRAGHTGRRCE; (SEQ ID NO: 194) and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to PCVGSNPCYNQGTCEPTSENPFYRCLCPAKFNGLLCH (SEQ ID NO: 195); and can have a length of 35 amino acids to 40 amino acids (e.g., 35, 36, 37, 38, 39, or 40 amino acids.

An LNR segment can comprise an amino acid sequence having at least 75%, at least 80%, at least 85%, at least 90%, at least 95%, at least 98%, at least 99%, or 100%, amino acid sequence identity 10 the following amino acid sequence: PPQIEEACELPECQVDAGNKVCNLQCNNHACGWDGGDCSLNFNDPWKNCTQSLQCW KYFSDGHCDSQCNSAGCLFDGFDCQLTEGQCNPLYDQYCKDHFSDGHCDQGCNSAEC EWDGLDC (SEQ ID NO: 196); and can have a length of from 118 to 122 amino acids (e.g., 118, 119, 120, 121, or 122 amino acids).

An HD segment (HD-N plus HD-C) can comprise an amino acid sequence having at least 75%, at least 80%, at least 85%, at least 90%, at least 95%, at least 98%, at least 99%, or 100%. amino acid sequence identity the to following amino acid sequence: AAGTLVLVVLLPPDQLRNNSFHFLRELSHVLHTNVVFKRDAQGQQMIFPYYGHEEELR KHPIKRSTVGWATSSLLPGTSGGRQRRELDPMDIRGSIVYLEIDNRQCVQSSSQCFQSAT DVAAFLGALASLGSLNIPYKIEAVKSEPVEPPLP (SEQ ID NO: 197); and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCGVLLS (SEQ ID NO: 198; and can have a length of 21, 22, 23, 24, or 25 amino acids.

In some embodiments, a Notch core receptor polypeptide comprises a synthetic linker. For example, In some embodiments, a Notch core receptor polypeptide comprises, in order from N-terminus to C-terminus: i) two to eleven EGF repeats; ii) a synthetic linker; iii) an LNR segment; iv) an HD-N segment, v) an HD-C segment; and vi) a TM domain.

A synthetic linker can have a length of from about 10 amino acids (aa) to about 200 aa, e.g., from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A synthetic linker can have a length of from 10 aa to 30 aa. e.g., 10, 11, 12, 13, 14, 15, 16, 17, 18, 19, 20, 21, 22, 23, 24, 25, 26, 27, 28, 29, or 30 aa. A synthetic linker can have a length of from 30 aa to 50 aa, e.g., from 30 aa to 35 aa, from 35 aa to 40 aa, from 40 aa to 45 aa, or from 45 aa to 50 aa.

Notch receptor polypeptide comprising an HD-C segment and a TM domain

In some embodiments, a synNotch receptor polypeptide comprises, in order from N-terminus to C-terminus: i) an HD-C segment; and ii) a TM domain, where the synNotch receptor polypeptide does not include an LNR segment. In some embodiments, the LNR segment is replaced with a heterologous polypeptide.

A transmembrane segment can comprise an amino acid sequence having at least 75%, at least 80%, at least 85%, at least 90%, at least 95%, at least 98%, at least 99%, or 100%, amino acid sequence identity to the following amino acid sequence:
HLMYVAAAAFVLLFFVGCGVLLS (SEQ ID NO: 199); and can have a length of 21, 22, 23, 24, or 25 amino acids.

As discussed above, a synNotch receptor polypeptide of the present disclosure comprises: a) an extracellular anti-PSCA binding domain; b) a Notch core receptor polypeptide having a length of from 50 amino acids to 1000 amino acids, and comprising one or more ligand-inducible proteolytic cleavage sites; and c) an intracellular domain, where binding of the of extracellular anti-PSCA binding domain to PSCA induces cleavage of the Notch receptor polypeptide at the one or more ligand-inducible proteolytic cleavage sites, thereby releasing the intracellular domain.

In some embodiments, the synNotch receptor polypeptide includes only one ligand-inducible proteolytic cleavage site. In some embodiments, the synNotch receptor polypeptide includes two ligand-inducible proteolytic cleavage sites. In some embodiments, the synNotch receptor polypeptide includes three ligand-inducible proteolytic cleavage sites. For simplicity, ligand-inducible cleavage sites will be referred to herein as “SI,” “S2,” and “S3” ligand-inducible proteolytic cleavage sites.

In some embodiments, the synNotch receptor polypeptide includes an SI ligand-inducible proteolytic cleavage site. An SI ligand-inducible proteolytic cleavage site can be located between the HD-N segment and the HD-C segment. In some embodiments, the SI ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. In some embodiments, an amino acid sequence comprising an SI ligand-inducible proteolytic cleavage site can have the amino acid sequence GRRRRELDPM (SEQ ID NO: 200), where cleavage occurs between the “RE” sequence. As another example, an amino acid sequence comprising an SI ligand-inducible proteolytic cleavage site can have the amino acid sequence RQRRELDPM (SEQ ID NO: 201), where cleavage occurs between the “RE” sequence.

In some embodiments, the synNotch receptor polypeptide includes an S2 ligand-inducible proteolytic cleavage site. An S2 ligand-inducible proteolytic cleavage site can be located within the HD-C segment. In some embodiments, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. In some embodiments, amino acid sequence comprising an S2 ligand-inducible proteolytic cleavage site can have the amino acid sequence KIEAVKSE (SEQ ID NO: 128), where cleavage occurs between the “AV” sequence. As another example, an amino acid sequence comprising an S2 ligand-inducible proteolytic cleavage site can have the amino acid sequence KIEAVQSE (SEQ ID NO: 202), where cleavage occurs between the “AV” sequence.

In some embodiments, the synNotch receptor polypeptide includes an S3 ligand-inducible proteolytic cleavage site. An S3 ligand-inducible proteolytic cleavage site can be located within the TM domain. In some embodiments, the S3 ligand-inducible proteolytic cleavage site is a gamma-secretase (γ-secretase) cleavage site. A γ-secretase cleavage site can comprise a Gly-Val dipeptide sequence, where the enzyme cleaves between the Gly and the Val. In some embodiments, an S3 ligand-inducible proteolytic cleavage site has the amino acid sequence VGCGVLLS (SEQ ID NO: 203), where cleavage occurs between the “GV” sequence. In some embodiments, an S3 ligand-inducible proteolytic cleavage site comprises the amino acid sequence GCGVLLS (SEQ ID NO. 204).

In some embodiments, the synNotch receptor polypeptide lacks an SI ligand-inducible proteolytic cleavage site. In some embodiments, the synNotch receptor polypeptide lacks an S2 ligand-inducible proteolytic cleavage site. In some embodiments, the synNotch receptor polypeptide lacks an S3 ligand-inducible proteolytic cleavage site. In some embodiments, the synNotch receptor polypeptide lacks both an SI ligand-inducible proteolytic cleavage site and an S2 ligand-inducible proteolytic cleavage site. In some embodiments, the synNotch receptor polypeptide includes an S3 ligand-inducible proteolytic cleavage site; and lacks both an SI ligand-inducible proteolytic cleavage site and an S2 ligand-inducible proteolytic cleavage site.

In some embodiments, the synNotch receptor polypeptide comprises a Notch core that comprises an amino acid sequence having at least 75%, at least 80%, at least 85%, at least 90%, at least 95%, at least 98%, at least 99%, or 100%, amino acid sequence identity to the following amino acid sequence: ILDYSFTGGAGRDIPPPQIEEACELPECQVDAGNKVCNLQCNNHACGWDGGDCSLNFN DPWKNCTQSLQCWKYFSDGHCDSQCNSAGCLFDGFDCQLTEGQCNPLYDQYCKDHFS DGHCDQGCNSAECEWDGLDCAEHVPERLAAGTLVLVVLLPPDQLRNNSFHFLRELSHV LHTNVVFKRDAQGQQMIFPYYGHEEELRKHPIKRSTVGWATSSLLPGTSGGRQRRELDP MDIRGSIVYLEIDNRQCVQSSSQCFQSATDVAAFLGALASLGSLNIPYKIEAVKSEPVEPP LPSQLHLMYVAAAAFVLLFFVGCGVLLSRKRRRQHGQLWFPEGFKVSEASKKKRREPL GEDSVGLKPLKNASDGALMDDNQNEWGDEDLETKKFRFEEPVV (SEQ ID NO: 205). In embodiments a notch core is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to:

(SEQ ID NO: 206)
atcctggactacagcttcacaggtggcgctgggcgcgacattcccccacc
gcagattgaggaggcctgtgagctgcctgagtgccaggtggatgcaggca
ataaggtctgcaacctgcagtgtaataatcacgcatgtggctgggatggt
ggcgactgctccctcaacttcaatgacccctggaagaactgcacgcagtc
tctacagtgctggaagtattttagcgacggccactgtgacagccagtgca
actcggccggctgcctctttgatggcttcgactgccagctcaccgaggga
cagtgcaaccccctgtatgaccagtactgcaaggaccacttcagtgatgg
ccactgcgaccagggctgtaacagtgccgaatgtgagtgggatggcctag
actgtgctgagcatgtacccgagcggctggcagccggcaccctggtgctg
gtggtgctgcttccacccgaccagctacggaacaactccttccactttct
gcgggagctcagccacgtgctgcacaccaacgtggtcttcaagcgtgatg
cgcaaggccagcagatgatcttcccgtactatggccacgaggaagagctg
cgcaagcacccaatcaagcgctctacagtgggttgggccacctcttcact
gcttcctggtacaagtggtgggcgccagcgcagggagctggaccccatgg
acatccgtggctccattgtctacctggagatcgacaaccggcaatgtgtg
cagtcatcctcgcagtgcttccagagtgccaccgatgtggctgccttcct
aggtgctcttgcgtcacttggcagcctcaatattccttacaagattgagg
ccgtgaagagtgagccggtggagcctccgctgccctcgcagctgcacctc
atgtacgtggcagcagccgccttcgtgctcctgttctttgtgggctgtgg
ggtgctgctgtcccgcaagcgccggcgg.

In some embodiments, the chimeric Notch receptor polypeptide contains all or a portion of human Notch1, Notch2, Notch3, or Notch4. In some embodiments, the chimeric Notch receptor polypeptide contains all or a portion of SEQ ID NO: 244 SEQ ID NO: 245, SEQ ID NO: 246. SEQ ID NO: 247, or SEQ ID NO: 248. In some embodiments, a “portion” of Notch comprises the three NLR domains, the transmembrane domain, and a short cytosolic fragment including the native Nuclear Localization Sequence (NLS) of Notch.

Human neurogenic locus notch homolog protein 1 preprotein NP_060087.3:

(SEQ ID NO: 244)
MPPLLAPLLCLALLPALAARGPRCSQPGETCLNGGKCEAANGTEACVCGG
AFVGPRCQDPNPCLSTPCKNAGTCHVVDRRGVADYACSCALGFSGPLCLT
PLDNACLTNPCRNGGTCDLLTLTEYKCRCPPGWSGKSCQQADPCASNPCA
NGGQCLPFEASYTCHCPPSFHGPTCRQDVNECGQKPGLCRHGGTCHNEVG
SYRCVCRATHTGPNCERPYVPCSPSPCQNGGTCRPTGDVTHECACLPGFT
GQNCEENIDDCPGNNCKNGGACVDGVNTYNCRCPPEWTGQYCTEDVDECQ
LMPNACQNGGTCHNTHGGYNCVCVNGWTGEDCSENIDDCASAACFHGATC
HDRVASFYCECPHGRTGLLCHLNDACISNPCNEGSNCDTNPVNGKATCTC
PSGYTGPACSQDVDECSLGANPCEHAGKCINTLGSFECQCLQGYTGPRCE
IDVNECVSNPCQNDATCLDQTGEFQCTCMPGYEGVHCEVNTDECASSPCL
HNGRCLDKINEFQCECPTGFTGHLCQYDVDECASTPCKNGAKCLDGPNTY
TCVCTEGYTGTHCEVDIDECDPDPCHYGSCKDGVATFTCLCRPGYTGHHC
ETNINECSSQPCRHGGTCQDRDNAYLCFCLKGTTGPNCEINLDDQASSPC
DSGTCLDKIDGYECACEPGYTGSMCNINIDECAGNPCHNGGTCEDGINGF
TCRCPEGHDPTCLSEVNECNSNPCVHGACRDSLNGYKCDCDPGWSGTNCD
INNNECESNPCVNGGTCKDMTSGYVCTCREGFSGPNCQTNINECASNPCL
NQGTCIDDVAGYKCNCLLPYTGATCEVVLAPCAPSPCRNGGECRQSEDYE
SFSCVCPTGWQGQTCEVDINECVLSPCRHGASCQNTHGGYRCHCQAGYSG
RNCETDIDDCRPNPCHNGGSCTDGINTAFCDCLPGFRGTFCEEDINECAS
DPCRNGANCTDCVDSYTCTCPAGFSGIHCENNTPDCTESSCFNGGTCVDG
INSFTCLCPPGFTGSYCQHDVNECDSQPCLHGGTCQDGCGSYRCTCPQGY
TGPNCQNLVHWCDSSPCKNGGKCWQTHTQYRCECPSGWTGLYCDVPSVSC
EVAAQRQGVDVARLCQHGGLCVDAGNTHHCRCQAGYTGSYCEDLVDECSP
SPCQNGATCTDYLGGYSCKCVAGYHGVNCSEEIDECLSHPCQNGGTCLDL
PNTYKCSCPRGTQGVHCEINVDDCNPPVDPVSRSPKCFNNGTCVDQVGGY
SCTCPPGFVGERCEGDVNECLSNPCDARGTQNCVQRVNDFHCECRAGHTG
RRCESVINGCKGKPCKNGGTCAVASNTARGFTCKCPAGFEGATCENDART
CGSLRCLNGGTCISGPRSPTCLCLGPFTGPECQFPASSPCLGGNPCYNQG
TCEPTSESPFYRCLCPAKFNGLLCHILDYSFGGGAGRDIPPPLIEEACEL
PECQEDAGNKVCSLQCNNHACGWDGGDCSLNFNDPWKNCTQSLQCWKYFS
DGHCDSQCNSAGCLFDGFDCQRAEGQCNPLYDQYCKDHFSDGHCDQGCNS
AECEWDGLDCAEHVPERLAAGTLVVVVLMPPEQLRNSSFHFLRELSRVLH
TNVVFKRDAHGQQMIFPYYGREEELRKHPIKRAAEGWAAPDALLGQVKAS
LLPGGSEGGRRRRELDPMDVRGSIVYLEIDNRQCVQASSQCFQSATDVAA
FLGALASLGSLNIPYKIEAVQSETVEPPPPAQLHFMYVAAAAFVLLFFVG
CGVLLSRKRRRQHGQLWFPEGFKVSEASKKKRREPLGEDSVGLKPLKNAS
DGALMDDNQNEWGDEDLETKKFRFEEPVVLPDLDDQTDHRQWTQQHLDAA
DLRMSAMAPTPPQGEVDADCMDVNVRGPDGFTPLMIASCSGGGLETGNSE
EEEDAPAVISDFIYQGASLHNQTDRTGETALHLAARYSRSDAAKRLLEAS
ADANIQDNMGRTPLHAAVSADAQGVFQILIRNRATDLDARMHDGTTPLIL
AARLAVEGMLEDLINSHADVNAVDDLGKSALHWAAAVNNVDAAVVLLKNG
ANKDMQNNREETPLFLAAREGSYETAKVLLDHFANRDITDHMDRLPRDIA
QERMHHDIVRLLDEYNLVRSPQLHGAPLGGTPTLSPPLCSPNGYLGSLKP
QGVGKKVRKPSSKGLACGSKEAKDIKARRKKSQDGKGCLLDSSGMLSPVD
SLESPHGYLSDVASPPLLPSPFQQSPSVPLNHLPGMPDTHLGTGHLNVAA
KPEMAALGGGGRLAFETGPPRLSHLPVASGTSTVLGSSSGGALNFTVGGS
TSLNGQCEWLSRLQSGMVPNQYNPLRGSVAPGPLSTQAPSLQHGMVGPLH
SSLAASALSQMMSYQGLPSTRLATQPHLVQTQQVQPQNLQMQQQNLQPAN
IQQQQSIQPPPPPPQPHLGVSSAASGHLGRSFLSGEPSQADVQPLGPSSL
AVHTILPQESPALPTSLPSSLVPPVTAAQFLTPPSQHSYSSPVDNTPSHQ
LQVPEHPFLTPSPESPDQWSSSSPHSNVSDWSEGVSSPPTSMQSQIARIP
EAFK.

Haman neurogenic locus notch homolog protein 2 isoform I preprotein NP_077719.2:

(SEQ ID NO: 245)
SMPALRPALLWALLALWLCCAAPAHALQCRDGYEPCVNEGMCVTYHNGTG
YCKCPEGFLEYCQHRDPCEKNRCQNGGTCVAQAMLGKATCRCASGFTGED
CQYSTSHPCFVSRPCLNGGTCHMLSRDTVECTCQVGFTGKECQWTDACLS
HPCANGSTCTTVANQFSCKCLTGFTGQKCETDVNECDIPGHCQHGGTCLN
LPGSYQCQCPQGFTGQYCDSLYVPCAPSPCVNGGTCRQTGDFTFECNCLP
GFEGSTCERNIDDCPNHRCQNGGVCVDGVNTYNCRCPPQWTGQFCTEDVD
ECLLQPNACQNGGTCANRNGGYGCVCVNGWSGDDCSENIDDCAFASCTPG
STCIDRVASFSCMCPEGKAGLLCHLDDACISNPCHKGALCDTNPLNGQYT
CTCPQGYKGADCTEDVDECAMANSNPCEHAGKCVNTDGAFHCECLKGYAG
PRCEMDINECHSDPCQNDATCLDKTGGFTCLCMPGFKGVHCELEINECQS
NPCVNNGQCVDKVNRFQCLCPPGFTGPVCQIDIDDCSSTPCLNGAKCIDH
PNGVECQCATGFTGVLCEENIDNCDPDPCHHGQCQDGIDSYTCICNPGYM
GATCSDQIDECYSSPCLNDGRCIDLVNGYQCNCQPGTSGVNCEINFDDCA
SNPCIHGTCMDGINRYSCVCSPGFTGQRCNIDIDECASNPCRKGATCING
VNGFRCTCPEGPHHPSCYSQVNECLSNPCIHGNCTGGLSGYKCLCDAGWV
GINCEVDKNECLSNPQONGGTCDNLVNGYRCTCKKGFKGYNCQVNIDECA
SNPCLNQGTCFDDISGYTCHCVLPYTGKNCQTVLAPCSPNPCENAAVCKE
SPNFESYTCLCAPGWQGQRCTIDIDECISKPCMNHGLCHNTQGSYMCECP
PGFSGMDCEEDIDDCLANPCQNGGSCMDGVNTFSCLCLPGFTGDKCQTDM
NECLSEPCKNGGTCSDYVNSYTCKCQAGFDGVHCENNINECTESSCFNGG
TCVDGINSFSCLCPVGFTGSFCLHEINECSSHPCLNEGTCVDGLGTYRCS
CPLGYTGKNCQTLVNLCSRSPCKNKGTCVQKKAESQCLCPSGWAGAYCDV
PNVSCDIAASRRGVLVEHLCQHSGVCINAGNTHYCQCFLGYTGSYCEEQL
DECASNPCQHGATCSDFTGGYRCECVPGYOGVNCEYEVDECQNQPCQNGG
TCIDLVNHFKCSCPPGTRGLLCEENIDDCARGPHCLNGGQCMDRTGGYSC
RCLPGFAGERCEGDINECLSNPCSSEGSLDCIQLTNDYLCVCRSAFTGRH
CETFVDVCPQMPCLNGGTCAVASNMPDGFTCRCPPGFSGARCQSSCGQVK
CRKGEQCVHTASGPRCFCPSPRDCESGCASSPCQHGGSCHPQRQPPYYSC
QCAPPFSGSRCELYTAPPSTPPATCLSQYCADKARDGVCDEACNSHACQW
DGGDCSLIMENPWANCSSPLPCWDYINNQCDELCNTVECLFDNFECQGNS
KTCKYDKYCADHFKDNHGDQGCNSEECGWDGLDCAADQPENLAEGTLVIV
VLMPPEQLLQDARSFLRALGTLLHTNLRIKRDSQGELMVYPYYGEKSAAM
KKQRMTRRSLPGEQEQEVAGSKVFLEIDNRQCVQDSDHCFKNTDAAAALL
ASHAIQGTLSYPLVSVVSESLTPERTQLLYLLAVAVVIILFILLGVIMAK
RKRKHGSLWLPEGFTLRRDASNHKRREPVGQDAVGLKNLSVQVSEANLTG
TGTSEHWVDDEGPQPKKVKAEDEALLSEEDDPIDRRPWTQQHLEAADIRR
TPSLALTPPQAEQEVDVLDVNVRGPDGCTPLMLASLRGGSSDLSDEDEDA
EDSSANIITDLVYQGASLQAQTDRTGEMALHLAARYSRADAAKRLLDAGA
DANAQDNMGRCPLHAAVAADAQGVFQILIRNRVTDLDARMNDGTTPLILA
ARLAVEGMVAELINCQADVNAVDDHGKSALHWAAAVNNVEATLLLLKNGA
NRDMQDNKEETPLFLAAREGSYEAAKILLDHFANRDITDHMDRLPRDVAR
DRMHHDIVRLLDEYNVTPSPPGTVLTSALSPVTCGPNRSFLSLKHTPMGK
KSRRPSAKSTMPTSLPNLAKEAKDAKGSRRKKSLSEKVQLSESSVTLSPV
DSLESPHTYVSDTTSSPMITSPGILQASPNPMLATAAPPAPVHAQHALSF
SNLHEMQPLAHGASTVLPSVSQLLSHHHTVSPGSGSAGSLSRLHPVPVPA
DWMNRMEVNETQYNEMFGMVLAPAEGTHPGIAPQSRPPEGKHITTPREPL
PPTVTFQLIPKGSIAQPAGAPQPQSTCPPAVAGPLPTMYQIPEMARLPSV
AFPTAMMPQQDGQVAQTILPAYHPFPASVGKYPTPPSQHSYASSNAAERT
PSHSGHLOGEHPYLTPSPESPDQWSSSSPHSASDWSDVTTSPTPGGAGGG
QRGPGTHMSEPPHNNMQVYA.

Human neurogenic locus notch homolog protein 2 isoform 2 precursor NP_001186930.1:

(SEQ ID NO: 246)
MPALRPALLWALLALWLCCAAPAHALQCRDGYEPCVNEGMCVTYHNGTGY
CKCPEGFLGEYCQHRDPCEKNRCQNGGTCVAQAMLGKATCRCASGFTGED
CQYSTSHPCFVSRPCLNGGTCHMLSRDTYECTCQVGFTGKECQWIDACLS
HPCANGSTCTTVANQFSCKCLTGFTGQKCETDVNECDIPGHCQHGGTCLN
LPGSYQCQCPQGFTGQYCDSLYVPCAPSPCVNGGTCRQTGDFTFECNCLP
GFEGSTCERNIDDCPNHRCQNGGVCVDGVNTYNCRCPPQWTGQFCTEDVD
ECLLQPNACQNGGTCANRNGGYGCVCVNGWSGDDCSENIDDCAFASCTPG
STCIDRVASFSCMCPEGKAGLLCHLDDACISNPCHKGALCDTNPLNGQYT
CTCPQGYKGADCTEDVDECAMANSNPCEHAGKCVNTDGAFHCECLKGYAG
PRCEMDINECHSDPCQNDATCLDKTGGFTCLCMPGFKGVHCELEINECQS
NPCVNNGQCVDKVNRFQCLCPPGFTGPVCQIDIDDCSSTPCLNGAKCIDH
PNGYECQCATGFTGVLCEENIDNCDPDPCHHGQCQDGIDSYTCICNPGYM
GATCSDQIDECYSSPCLNDGRCIDLVNGYQCNCQPGTSGVNCEINFDDAS
NPCIHGTCMDGINRYSVVCSPGFTGQRCNIDIDECASNPCRKGATCINGV
NGFRCTCPEGPHHPSCYSQVNECLSNPCIHGNCTGGLSGYKCLCDAGWVG
INCEVDKNECLSNPCQNGGTCDNLVNGYRCTCKKGFKGYNCQVNIDECAS
NPCLNQGTCFDDISGYTCHCVLPYTGKNCQTVLAPCSPNPCENAAVCKES
PNFESYTCLCAPGWQGQRCTIDIDECISKPCMNHGLCHNTQGSYMCECPP
GFSGMDCEEDIDDCLANPCQNGGSCMDGVNTFSCLCLPGFTGDKCQTDMN
ECLSEPCKNGGTCSDYVNSYTCKCQAGFDGVHCENNINECTESSCFNGGT
CVDGINSFSCLCPVGFTGSFCLHEINECSSHPCLNEGTCVDGLGTYRCSC
PLGYTGKNCQTLVNLCSRSPCKNKGTVQKKAESQCLCPSGWAGAYCDVPN
VSCDIAASRRGVLVEHLCQHSGVCINAGNTHYCQCPLGYTGSYCEEQLDE
CASNPCQHGATCSDFTGGYRCECVPGYQGVNCEYEVDECQNQPCQNGGTC
IDLVNHFKCSCPPGTRGMKSSLSIFHPGHCLKL.

Human neurogenic locus notch homolog protein 3 precursor NP_000426.2:

(SEQ ID NO: 247)
MGPGARGRRRRRRPMSPPPPPPPVRALPLLLLLAGPGAAAPPCLDGSPCA
NGGRCTQLPSREAACLCPPGWVGERCQLEDPCHSGPCAGRGVCQSSVVAG
TARFSCRCPRGFRGPDCSLPDPCLSSPCAHGARCSVGPDGRFLCSCPPGY
QGRSCRSDVDECRVGEPCRHGGTCLNTPGSFRCQCPAGYTGPLCENPAVP
CAPSPCRNGGTCRQSGDLTYDCACLPGFEQNCEVNVDDCPGHRCLNGGTC
VDGVNTYNCQCPPEWTGQFCTEDVDECQLQPNACHNGGTCFNTLGGHSCV
CVNGWTGESCSQNIDDCATAVCFHGATCHDRVASFYCACPMGKTGLLCHL
DDACVSNPCHEDATCDTNPVNGRATCTCPPGFTGGACDQDVDECSTGANP
CEHLGRCVNTQGSFLCQCGRGYTGPRCETDVNECLSGPCRNQATCLDRTG
QFTCTCMAGFTGTYCEVDIDECQSSPCVNGGVCKDRVNGFSCTCPSGFSG
STCQLDVDECASTPCRNGAKCVDQPDGYECRCAEGFEGTLCDRNVDDCSP
DPCHHGRCVDGIASFSCACAPGYTGTRCESQVDECRSQPCRHGGKCLDLV
DKYLCRCPSGTTGVNCEVNIDDCASNPCTFGVCRDGINRYDCVCQPGFTG
PLCNVEINECASSPCGEGGSCVDGENGFRCLCPPGSLPPLCLPPSHPCAH
EPCSHGTCYDAPGGFRCVCEPGWSGPRCSQSLARDACESQPCRAGGTCSS
DGMGFHCTCPPGVQQRQCELLSPCIPNPCEHGGRCESAPGQLPVCSCPQG
WQGPRCQQDVDECAGPAPCGPHGTCTNLAGSFSCTCHGGYTGPSCDQDIN
DCDPNPCLNGGSCQDGVGSFSCSCLPGFAGPRCARDVDECLSNPCGPGTC
TDHVASFTCTCPPGYGGFHCEODLPDCSPSSCFNGGTCVDGVNSFSCLCR
PGYTGAHCQHEADPCLSRPCLHGGVCSAAHPGFRCTCLESFTGPQCQTLV
DWCSRQPCOQGGRCVQTGAYCLCPPGWSGRLCDIRSLPCREAAAQTGVRL
EQLCQAGGQCVDEDSSHYCVCPEGRTGSHCEQEVDPCLAQPCQHGGTCRG
YMGGYMCECLPGYNGDNCEDDVDECASQPCQHGGSCIDLVARYLCSCPPG
TLGVLCEINEDDCGPGPPLDSGPRCLHNGTCVDLVGGFRCTCPPGYTGLR
CEADINECRSGACHAAHTRDCLQDPGGGFRCLCHAGFSGPRCQTVLSPCE
SQPCQHGGQCRPSPGPGGGLTFTCHCAQPFWGPRCERVARSCRELQCPVG
VPCQQTPRGPRCACPPGLSGPSCRSFPGSPPGASNASCAAAPCLHGGSCR
PAPLAPFFRCACAQGWTGPRCEAPAAAPEVSEEPRCPRAACQAKRGDQRC
DRECNSPGCGWDGGDCSLSVGDPWRQCEALQCWRLFNNSRCDPACSSPAC
LYDNFDCHAGGRERTCNPVYEKYCADHFADGRCDQGCNTEECGWDGLDCA
SEVPALLARGVLVLTVLLPPEELLRSSADFLQRLSAILRTSLRFRLDAHG
QAMVFPYHRPSPGSEPRARRELAPEVTGSVVMLEIDNRLCLQSPENDHCF
PDAQSAADYLGALSAVERLDFPYPLRDVRGEPLEPPEPSVPLLPLLVAGA
VLLLVILVLGVMVARRKREHSTLWFPEGFSLHKDVASGHKGRREPVGQDA
LGMKNMAKGESLMGEVATDWMDTECPEAKRLKVEEPGMGAEEAVDCRQWT
QHHLVAADIRVAPAMALTPPQGDADADGMDVNVRGPDGFTPLMLASFCGG
ALEPMPTEEDEADDTSASIISDLTCQGAQLGARTDRTGETALHLAARYAR
ADAAKRLLDAGADTNAQDHSGRTPLHTAVTADAQGVFQILIRNRSTDLDA
RMADGSTALILAARLAVEGMVEELIASHADVNAVDELGKSALHWAAAVNN
VEATLALLKNGANKDMQDSKEETPLFLAAREGSYEAAKLLLDHFANREIT
DHLDRLPRDVAQERLHQDIVRLLDQPSGPRSPPGPHGLGPLLCPPGAFLP
GLKAAQSGSKKSRRPPGKAGLGPQGPRGRGKKLTIACPGPLADSSVTLSP
VDSLDSPRPFGGPPASPGGFPLEGPYAAATATAVSLAQLGGPGRAGLGRQ
PPGGCVLSLGLLNPVAVPLDWARLPPPAPPGPSFLLPLAPGPQLLNPGTP
VSPQERPPPYLAVPGHGEEYPAAGAHSSPPKARFLRVPSEHPYLTPSPES
PEHWASPSPPSLSDWSESTPSPATATGAMATTTGALPAQPLPLSVPSSLA
QAQTQLGPQPEVTPKRQVLA.

Human neurogenic locus notch homolog protein 4 preprotein NP_004548.3:

(SEQ ID NO: 248)
MQPPSLLLLLLLLLLLCVSVVRPRGLLCGSFPEPCANGGTCLSLSLGQGT
CQCAPGFLGETCQFPDPCQNAQLCQNGGSCQALLPAPLGLPSSPSPLTPS
FLCTCLPGFTGERCQAKLEDPCPPSFCSKRGRCHIQASGRPQCSCMPGWT
GEQCQLRDFCSANPCVNGGVCLATYPQIQCHCPPGFEGHACERDVNECFQ
DPGPCPKGTSCHNTLGSFQCLCPVGQEGPRCELRAGPCPPRGCSNGGTCQ
LMPEKDSTFHLCLCPPGFTGPDCEVNPDNCVSHQCQNGGTCQDGLDTYTC
LCPETWTGWDCSEDVDECETQGPPHCRNGGTCQNSAGSFHCVCVSGWGGT
SCEENLDDCIAATCAPGSTCIDRVGSFSCLCPPGRTGLLCHLEDMCLSQP
CHGDAQCSTNPLTGSTLCLCQPGYSGPTCHQDLDECLMAQQGPSPCEHGG
SCLNTPGSFNCLCPPGYTGSRCEADHNECLSQPCHPGSTCLDLLATFHCL
CPPGLEGQLCEVETNECASAPCLNHADCHDLLNGFQCTCLPGFSGTRCEE
DIDECRSSPCANGGQCQDQPGAFHCKCLPGFEGPRCQTEVDECLSDPCPV
GASCLDLPGAFFCLCPSGFTGQLCEVPLCAPNLCQPKQTCKDQKDKANCL
CPDGSPGCAPPEDNCTCHHGHCQRSSCVCDVGWTGPECEAELGGCISAPC
AHGGTCYPQPSGYNCTCPTGYTGPTCSEEMTACHSGPCLNGGSCNPSPGG
YYCTCPPSHTGPQCQTSTDYCVSAPCFNGGTCVNRPGTFSCLCAMGFQGP
RCEGKLRPSCADSPCRNRATCQDSPQGPRCLCPTGYTGGSCQTLMDLCAQ
KPCPRNSHCLQTGPSFHCLCLQGWTGPLCNLPLSSCQKAALSQGIDVSSL
CHNGGLCVDSGPSYFCHCPPGFQGSLCQDHVNPCESRPCQNGATCMAQPS
GYLCQCAPGYDGONCSKELDACQSQPCHNHGTCTPKPGGFHCACPPGFVG
LRCEGDVDECLDQPCHPTGTAACHSLANAFYCQCLPGHTGQWCEVEIDPC
HSQPCFHGGTCEATAGSPLGFTCHCPKGFEGPTCSHRAPSCGFHHCHHGG
LCLPSPKPGFPPRCACLSGYGGPDCLTPPAPKGCGPPSPCLYNGSCSETT
GLGGPGFRCSCPHSSPGPRCQKPGAKGCEGRSGDGACDAGOSGPGGNWDG
GDCSLGVPDPWKGCPSHSRCWLLFRDGQCHPQCDSEECLFDGTDCETPPA
CTPAYDQYCHDHFHNGHCEKGCNTAECGWDGGDCRPEDGDPEWGPSLALL
VVLSPPALDQQLFALARVLSLTLRVGLWVRKDRDGRDMVYPYPGARAEEK
LGGIRDPTYQERAAPQTQPLGKETDSLSAGFVVVMGVDLSRCGPDHPASR
CPWDPGLLLRFLAAMAAVGALEPLLPGPLLAVHPHAGTAPPANQLPWPVL
CSPVAGVILLALGALLVLQLIRRRRREHGALWLPPGFTRRPRTQSAPHRR
RPPLGEDSTGLKALKPKAEVDEDGVVMCSGPEEGEEVGQAEETGPPSTCQ
LWSLSGGCGALPQAAMLIPPQESEMEAPDLDTRGPDGVTPLMSAVCCGEV
QSGTFQGAWLGCPEPWEPLLDGGACPQAHTVGTGETPLHLAARFSRPTAA
RRLLEAGANPNQPDRAGRTPLHAAVAADAREVCQLLLRSRQTAVDARTED
GTTPLMLAARLAVEDLVEELIAAQADVGARDKWGKTALHWAAAVNNARAA
RSLLQAGADKDAQDNREQTPLFLAAREGAVEVAQLLLGLGAARELRDQAG
LAPADVAHQRNHWDLLTLLEGAGPPEARHKATPGREAGPFPRARTVSVSV
PPHGGGALPRCRTLSAGAGPRGGGACLQARTWSVDLAARGGGAYSHCRSL
SGVGAGGGPTPRGRRFSAGMRGPRPNPAIMRGRYGVAAGRGGRVSTDDWP
CDWVALGACGSASNIPIPPPCLTPSPERGSPQLDCGPPALQHMPINQGGE
GKK.

In some embodiments, the Notch core of the chimeric Notch receptor polypeptide contains a portion of SEQ ID NO: 244 SEQ ID NO: 245, SEQ ID NO: 246. SEQ ID NO: 247, or SEQ ID NO: 248. In some embodiments, the chimeric Notch receptor polypeptide contains 50 to 1000 amino acids of SEQ ID NO: 244 SEQ ID NO: 245, SEQ ID NO: 246, SEQ ID NO: 247, or SEQ ID NO: 248. In some embodiments, the chimeric Notch receptor polypeptide contains 50 to 900 amino acids. 100 to 800 amino acids, 200 to 700 amino acids. 300 to 600 amino acids, 400 to 500 amino acids of SEQ ID NO: 244 SEQ ID NO: 245, SEQ ID NO: 246, SEQ ID NO: 247, or SEQ ID NO: 248. In some embodiments, the amino acid sequence of Notch, as described herein, is at least 80% identical to a corresponding amino acid sequence in SEQ ID NO: 244 SEQ ID NO: 245, SEQ ID NO: 246, SEQ ID NO: 247, or SEQ ID NO: 248. In some embodiments, the amino acid sequence of Notch is 80%, 81%, 82%, 83%, 84%, 85%, 86%, 87%, 88%, 89%, 90%, 91%, 92%, 93%, 94%, 95%, 96%, 97%, 98%, or 99% identical to a corresponding amino acid sequence in SEQ ID NO: 244 SEQ ID NO: 245, SEQ ID NO: 246, SEQ ID NO: 247, or SEQ ID NO: 248. In some embodiments, the amino acid sequence of Notch, as described herein, can vary from the amino acid sequence of SEQ ID NO: 244 SEQ ID NO: 245, SEQ ID NO: 246, SEQ ID NO: 247, or SEQ ID NO: 248 by 1 to 50 amino acids, e.g., 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95. or 100 amino acids. Human Notch 1 core regulatory domain (human Notch 1 core regulatory region, hN1 c): ile1427 after the last EGF repeat on NECD of human Notch I (NP_060087) was used as the N-terminus of hN1 c. The last Arg 1762 of the S consecutive basic amino acids (RKRRR (SEQ ID NO: 249)) at the C. terminus of the transmembrane domain serves as the C-terminus of hN 1C. The sequences of hN1c and mN1c were highly similar.

(SEQ ID NO: 250)
ILDYSFGGGAGRDIPPPLIEEACELPECQEDAGNKVCSLQCNNHACGWDGG
DCSLNFNDPWKNCTQSLQCWKVFSDGHCDSQCNSAGCLFDGFDCQRAEGQC
NPLYDQYCKDHFSDGHCDQGCNSAECEWDGLDCAEHVPERLAAGTLVVVVL
MPPEQLRNSSFHFLRELSRVLHTNVVFKRDAHGQQMIFPYYGREEELRKHP
IKRAAEGWAAPDALLGQVKASLLPGGSEGGRRRRELDPMDVRGSIVYLEID
NRQCVQASSQCFQSATDVAAFLGALASLGSLNIPYKIEAVQSETVEPPPPA
QLHFMYVAAAAFVLLFFVGCGVLLSRKRRRNRR

with hN1c at positions 1-294, 295-336 is TMD. Human Notch2 core regulatory domain (human Notch2 core regulatory region, hN2 c): leu 1413 after the last EGF repeat on NECD of human Notch2 (NP_077719) was used as the N-terminus of hN2 c. The last Arg1704 of the 4 consecutive basic amino acids (KRKR (SEQ ID NO: 258)) at the C-terminus of the transmembrane domain serves as the C-terminus of hN 2C. LYTAPPSTPPATCLSQYCADKARDGVCDEACNSHACQWDGGDCSLTMENPWANCSSP LPCWDYINNQCDELCNTVECLFDNFECQGNSKTCKYDKYCADHFKDNHCDQGCNSEE CGWDGLDCAADQPENLAEGTLVIVVLMPPEQLLQDARSFLRALGTLLHTNLRIKRDSQ GELMVYPYYGEKSAAMKKQRMTRRSLPGEQEQEVAGSKVFLEIDNRQCVQDSDHCFK NTDAAAALLASHAIQGTLSYPLVSVVSESLTPERTQLLYLLAVAVVIILFIILLGVIMAKR KR (SEQ ID NO: 251). Human Notch3 core regulatory domain (human Notch3 core regulatory region, hN3 c): pro1375 after the last EGF repeat on NECD of human Notch3 (NP-000426) was used as the N-terminus of hN3 c. The last Arg 1669 of the 4 consecutive basic amino acids (RRKR SEQ ID NO: 259)) at the C-terminus of the transmembrane domain served as the C-terminus of hN 3C. PAAAPEVSEEPRCPRAACQAKRGDQRCDRECNSPGCGWDGGDCSLSVGDPWRQCEAL QCWRLENNSRCDPACSSPACLYDNEDCHAGGRERTCNPVYEKYCADHFADGRCDQGC NTEECGWDGLDCASEVPALLARGVLVLTVLLPPEELLRSSADFLQRLSAILRTSLRFRLD AHGQAMVFPYHRPSPGSEPRARRELAPEVIGSVVMLEIDNRLCLQSPENDHCFPDAQSA ADYLGALSAVERLDFPYPLRDVRGEPLEPPEPSVPLLPLLVAGAVLLLVILVLGVMVAR RKR (SEQ ID NO: 252). Human Notch4 core regulatory domain (human Notch4 core regulatory region. hN3 c): pro1162 after the last EGF repeat on NECD of human Notch4 (NP-004548) was used as the N-terminus of hN4 c. The last Arg1476 of the 5 consecutive basic amino acids (RRRRRRR (SEQ ID NO: 253)) at the C-terminus of the transmembrane domain serves as the C-terminus of hN 4C.

(SEQ ID NO: 254)
PHSSPGPRCQKPGAKGCEGRSGDGACDAGCSGPGGNWDGGDCSLGVPDPWK
GCPSHSRCWLLFRDGQCHPQCDSEECLFDGYDCETPPACTPAYDQYCHDHF
HNGHCEKGCNTAECGWDGGDCRPEDGDPEWGPSLALLVVLSPPALDQQLFA
LARVLSLTLRVGLWVRKDRDGRDMVYPYPGARAEEKLGGTRDPTYQERAAP
QTQPLGKETDSLSAGFVVVMGVDLSRCGPDHPASRCPWDPGLLLRFLAAMA
AVGALEPLLPGPLLAVHPHAGTAPPANQLPWPVLCSPVAGVILLALGALLV
LQLIRRRRR.

As noted above, a synNotch receptor polypeptide of the present disclosure comprises an intracellular domain that is released following binding of the anti-PSCA binding domain to PSCA, where binding of the synNotch receptor polypeptide to PSCA induces cleavage of an above-mentioned proteolytic cleavage site. The intracellular domain comprises an amino acid sequence that is heterologous to the synNotch receptor polypeptide. In other words, the intracellular domain comprises an amino acid sequence that is not naturally present in a synNotch receptor polypeptide.

The intracellular domain, when released from the synNotch receptor polypeptide, provides an effector function, where the effector functions change in transcription of a target gene, which in this disclosure is the expression of an anti-PSMA CAR. In some embodiments, the intracellular domain, when released from the synNotch receptor polypeptide, provides for an increase in transcription of an anti-PSMA CAR.

In some cases, a transcription factor may be an artificial transcription factor (ATF) including but not limited to e.g., Zinc-finger-based artificial transcription factors (including e.g., those described in Sera T. Adv Drug Deliv Rev. 2009 61 (7-8): 513-26; Collins et al. Curr Opin Biotechnol. 2003 14 (4): 371-8; Onori et al. BMC Mol Biol. 2013 14:3 the disclosures of which are incorporated herein by reference in their entirety). In some embodiments, the intracellular domain is a transcriptional activator.

In some cases, the transcriptional activator is GALA-VP16. In some cases, the transcriptional activator is GAL4-VP64. In some cases, the transcriptional activator is Tbx21. In some cases the transcriptional activator is an engineered protein, such as a zinc finger or TALE based DNA binding domain fused to an effector domain such as VP64 (transcriptional activation) or KRAB (transcriptional repression). A variety of other transcriptional transactivators are known in the art is suitable for use.

In some cases, the intracellular domain comprises an amino acid sequence having at least 75%, at least 80%, at least 85%, at least 90%, at least 95%, at least 98%, at least 99%, or 100%, amino acid sequence identity to the following GAL4-VP64 sequence:

(SEQ ID NO: 207)
MKLLSSIEQACDICRLKKLKCSKEKPKCAKCLKNNWECRYSPKTKRSPLTR
AHLTEVESRLERLEQLFLLIFPREDLDMILKMDSLQDIKALLTGLFVQDNV
NKDAVTDRLASVETDMPLTLRQHRISATSSSEESSNKGQRQLTVS

In some cases, the intracellular domain comprises an amino acid sequence having at least 75%, at least 80%, at least 85%, at least 90%, at least 95%, at least 98%, at least 99%, or 100%. amino acid sequence identity to the following GAL4-VP64 sequence: MKLLSSIEQACDICRLKKLKCSKEKPKCAKCLKNNWECRYSPKTKRSPLTRAHLTEVES RLERLEQLFLLIFPREDLDMILKMDSLQDIKALLTGLFVQDNVNKDAVTDRLASVETDM PLTLRQHRISATSSSEESSNKGQRQLTVSAAAGGSGGSGGSDALDDFDLDMLGSDALDD FDLDMLGSDALDDFDLDMLGSDALDDEDLDMLGS (SEQ ID NO: 208); and has a length of from 208 to 214 amino acids (e.g., 208, 209, 210, 211, 212, 213. or 214 amino acids).

In some cases, the intracellular domain comprises an amino acid sequence having at least 75%, at least 80%, at least 85%, at least 90%, at least 95%, at least 98%, at least 99%, or 100%. amino acid sequence identity to the following GAL4-VP64 sequence: MKLLSSIEQACDICRLKKLKCSKEKPKCAKCLKNNWECRYSPKTKRSPLTRAHLTEVES RLERLEQLFLLIFPREDLDMILKMDSLQDIKALLTGLFVQDNVNKDAVTDRLASVETDM PLTLRQHRISATSSSEESSNKGQRQLTVSGGGSGGGSDALDDFDLDMLGSDALDDFDLD MLGSDALDDFDLDMLGSDALDDFDLDML (SEQ ID NO: 209); and has a length of from 208 to 214 amino acids (e.g., 203, 203, 204, 205, 206, 207, or 208 amino acids).

The present disclosure comprises nucleic acids encoding antigen binding systems provided herein, comprising without limitation nucleic acids encoding anti-PSCA synNotch antigen receptors. The nucleic acid sequence of SEQ ID NO: 151 comprises and provides exemplary nucleic acid sequences corresponding to and encoding each of SEQ ID NOs: 150 and 152-160. The nucleic acid sequence of SEQ ID NO: 162 comprises and provides exemplary nucleic acid sequences corresponding to and encoding each of SEQ ID NOs: 161 and 163-191. The nucleic acid sequence of SEQ ID NO: 173 comprises and provides exemplary nucleic acid sequence corresponding to and encoding SEQ ID NOs: 172.

In an embodiment an anti-PSCA synNotch receptor peptide comprises or consist of an amino acid sequence having at least 75%, at least 80%, at least 85%, at least 90%, at least 95%.

at least 98%, at least 99%, or 100%, amino acid sequence identity to DIVMTQSPLSLPVTPGEPASISCRSSQSLLHSNGYNYLVWYLQKPGQSPQLLIYLGSIRAS GVPDRFSGSGSGTDFTLKISRVEAEDVGVYYCMQPLQTPITFGQGTRLEIKGSTSGSGKP GSGEGSTKGQVQLQQWGAGLLKPSETLSLTCAVYGGSFSGNYWSWIRQPPGKGLEWIG EINHSGSTNYNPSLKSRVTISVDTSKNQFSLKLSSVTAADTAVYYCARGGSYNYFDYWG QGTLVTVSSILDYSFTGGAGRDIPPPQIEEACELPECQVDAGNKVCNLQCNNHACGWDG GDCSLNFNDPWKNCTQSLQCWKYFSDGHCDSQCNSAGCLFDGFDCQLTEGQCNPLYD QYCKDHFSDGHCDQGCNSAECEWDGLDCAEHVPERLAAGTLVLVVLLPPDQLRNNSF HFLRELSHVLHTNVVFKRDAQGQQMIFPYYGHEEELRKHPIKRSTVGWATSSLLPGTSG GRQRRELDPMDIRGSIVYLEIDNRQCVQSSSQCFQSATDVAAFLGALASLGSLNIPYKIE AVKSEPVEPPLPSQLHLMYVAAAAFVLLFFVGCGVLLSRKRRRQHGQLWFPEGFKVSE ASKKKRREPLGEDSVGLKPLKNASDGALMDDNQNEWGDEDLETKKFRFEEPVVGSMK LLSSIEQACDICRLKKLKCSKEKPKCAKCLKNNWECRYSPKTKRSPLTRAHLTEVESRLE RLEQLFLLIFPREDLDMILKMDSLQDIKALLTGLFVQDNVNKDAVTDRLASVETDMPLT LRQHRISATSSSEESSNKGQRQLTVSGGGSGGGSDALDDFDLDMLGSDALDDFDLDML GSDALDDFDLDMLGSDALDDFDLDML (SEQ ID NO: 210). In embodiments an anti-PSCA synNotch receptor is encoded by a nucleic acid having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the nucleic acid having the sequence according to

(SEQ ID NO: 221)
gacatcgtgatgacacagagccctctgtctttacccgttacccccggtgaa
cccgctagcatcagctgcagaagctcccagtctttactccacagcaacggc
tacaactatttagtgtggtatttacagaaacccggccagagcccccagctg
ctgatttatctgggctccattcgtgctagcggcgtgcccgatagattttcc
ggcagcggaagcggcaccgacttcactttaaagatctctcgtgtggaggcc
gaggacgtgggcgtctactactgtatgcagcctctgcagacccccattacc
ttcggccaaggtactcgtctggaaatcaagggcagcaccagcggcagcgga
aaacccggaagcggcgagggaagcaccaaaggccaagttcagctgcagcag
tggggagctggtttactgaagcctagcgagacactgtctttaacatgcgcc
gtgtacggcggaagcttcagcggcaactattggagctggatcagacagcct
cccggtaagggtttagagtggatcggcgagatcaaccactccggctccacc
aactataacccctctttaaagtctcgtgtgaccatctccgtggacaccagc
aagaaccagttctctttaaagctgagctccgtgacagccgccgacaccgct
gtgtattactgtgctcgtggcggcagctacaactacttcgactactggggc
caaggtaccctcgtgaccgtgtccagcatcctggactacagcttcacaggt
ggcgctgggcgcgacattcccccaccgcagattgaggaggcctgtgagctg
cctgagtgccaggtggatgcaggcaataaggtctgcaacctgcagtgtaat
aatcacgcatgtggctgggatggtggcgactgctccctcaacttcaatgac
ccctggaagaactgcacgcagtctctacagtgctggaagtattttagcgac
ggccactgtgacagccagtgcaactcggccggctgcctctttgatggcttc
gactgccagctcaccgagggacagtgcaaccccctgtatgaccagtactgc
aaggaccacttcagtgatggccactgcgaccagggctgtaacagtgccgaa
tgtgagtgggatggcctagactgtgctgagcatgtacccgagcggctggca
gccggcaccctggtgctggtggtgctgcttccacccgaccagctacggaac
aactccttccactttctgcgggagctcagccacgtgctgcacaccaacgtg
gtcttcaagcgtgatgcgcaaggccagcagatgatcttcccgtactatggc
cacgaggaagagctgcgcaagcacccaatcaagcgctctacagtgggttgg
gccacctcttcactgcttcctggtacaagtggtgggcgccagcgcagggag
ctggaccccatggacatccgtggctccattgtctacctggagatcgacaac
cggcaatgtgtgcagtcatcctcgcagtgcttccagagtgccaccgatgtg
gctgccttcctaggtgctcttgcgtcacttggcagcctcaatattccttac
aagattgaggccgtgaagagtgagccggtggagcctccgctgccctcgcag
ctgcacctcatgtacgtggcagcagccgccttcgtgctcctgttctttgtg
ggctgtggggtgctgctgtcccgcaagcgccggcggcagcacggtcaactt
tggttcccagaaggcttcaaggtctccgaagcctccaagaaaaagcgaagg
gaaccactcggggaagacagtgtagggttgaaacctttgaagaacgccagc
gatggagccttgatggatgataaccaaaatgaatggggtgatgaagacctg
gaaaccaaaaagtttcgctttgaggaacctgtggtaggatccatgaaactc
cttagcagcatcgaacaggcttgcgacatctgcaggttgaaaaaactcaag
tgctcaaaagaaaagcctaagtgcgcaaagtgccttaaaaacaattgggaa
tgtcgctatagccccaagacaaagcggagccctctcacgagagcacacctg
actgaggtagaatctcgcttggagaggctggaacagcttttcctgcttatc
tttccacgcgaggatctcgatatgatcctcaaaatggactccctccaggac
atcaaagctctgctgactggactgtttgtacaggataatgtgaacaaggac
gctgtgacagacagattggcaagcgtggagaccgatatgcccctgaccctt
agacagcaccggatcagtgccacctcttctagcgaggaaagttcaaataaa
ggacagcgccagctgacggtgagtggcggtggaagcggaggaggttccgac
gctcttgatgatttcgatctcgacatgctgggatcagacgctctcgacgac
ttcgatttggacatgcttggatccgacgctctcgatgatttcgacctcgac
atgctcggatccgatgctctggatgactttgatcttgatatgctg

As disclosed herein the intracellular domain is a polypeptide that, when released upon binding of the anti-PSCA binding domain to PSCA, induces production, in a cell that expresses the synNotch polypeptide, of an anti-PSMA CAR.

As the intracellular domain of a synNotch receptor polypeptide of the present disclosure, when released upon binding of the first member of the specific binding pair to a second member of the specific binding pair, may induce the expression of various polypeptides as described herein, in some instances, induced expression of two or more polypeptides may generate a logic gated circuit. Such logic gated circuits may include but are not limited to e.g., “AND gates”, “OR gates”, “NOT gates” and combinations thereof including e.g., higher order gates including e.g., higher order AND gates, higher order OR gates, higher order NOT gates, higher order combined gates (i.e., gates using some combination of AND, OR and/or NOT gates).

In some embodiments an anti-PSCA synNotch receptor comprises a Myc tag, such as having at least 75% sequence identity to (such as, at least 75%, at least 80%, at least 90%, at least 95%, or 100% identity; e.g., 85-90%, 85-95%, 85-100%, 90-95%, 90-100%, or 95-100%) to the amino acid sequence set forth as SEQ ID NO. 75 (EQKLISEEDL: SEQ ID NO: 222). In an embodiment, a Myc tag is encoded by the nucleic acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) to gagcagaagctgattagcgaggaggattta (SEQ ID NO: 223).

Both engineered T cell receptors (TCR) and chimeric antigen receptor (CAR) therapies harness the specificity and immunotherapeutic effect of T cells for the treatment of a wide variety of malignancies. Some studies suggest that these therapies may be susceptible to the suppressive factors in the TME that result from T cell suppression by TGF-β (Bendle et al., J Immunol, 191:3232-3239 (2013) and Vong et al., Blood, 130:1791 (2017)). The present disclosure contemplates the use of the DN TGF-β Receptors described herein in combination with either TCR or CAR therapies as a way to maintain, or in some cases, restore TCR and/or CAR expansion in the presence of TGF-β suppression.

Chimeric antigen receptor (CAR) T cell therapy provides another therapeutic approach against tumor progression. Clinically, investigators have demonstrated that CAR expansion and persistence is correlated with therapeutic efficacy. Without being bound by any theory, it is believed that TGF-β repressed T cell populations found in the TME may be limiting CAR T cell expansion and persistence in patients who do not respond to CAR therapy. The resulting inhibitory cytokines in the TME are believed to limit CAR cell function and expansion. Thus, TGF-β could limit the efficacy of therapeutic engineered T cells.

Combining any CAR constructs or TCRs as described herein with a DN TGF-β receptors may restore, maintain or enhance the therapeutic effect of CAR T therapy challenged by TGF-β suppression. Thus, in one embodiment described herein, the DN TGF-β receptors, for example DN TGF-βRI or RII, are co-expressed in a T cell or an NK cell with an anti-PSMA CAR, either a constitutively or conditionally expressed as described herein. In some embodiment, the DN TGF-β receptors, for example DN TGF-βRI or RII, are co-expressed in a T cell or NK cell with an anti-PSMA CAR, such as described herein. In some embodiments the he DN TGF-β receptors, for example DN TGF-βRI or RII, are co-expressed in a T cell or NK cell with an anti-PSMA CAR and an anti-PSCA synNotch receptor, such as described herein. DN TGF-β receptors are described in International Patent Publication No. PCT/US2020/070157, which is hereby incorporated herein by reference in its entirety.

Dominant negative TGF-β Receptors are designed to inhibit the immunosuppressive effects of TGF-β in the TME. These constructs may also stimulate cytokine signaling to enhance T cell or NK function in the TME. The constructs described herein may be used alone or in combination with each other, and/or in combination with other immunotherapies, in order to inhibit TGF-β induced immunosuppression.

The engineered TGF-β receptors may comprise an N-terminal signal peptide at the N-terminus, for example at the N-terminus of the extracellular ligand binding domain of DN TGF-βRI. In one embodiment, a heterologous signal peptide may be used. The extracellular domain of a DN TGF-βRI may be fused to a leader or a signal peptide that directs the nascent protein into the endoplasmic reticulum and subsequent translocation to the cell surface. It is understood that, once a polypeptide containing a signal peptide is expressed at the cell surface, the signal peptide is generally proteolytically removed during processing of the polypeptide in the endoplasmic reticulum and translocation to the cell surface. Thus, a polypeptide such as a DN TGF-βRI is generally expressed at the cell surface as a mature protein lacking the signal peptide, whereas the precursor form of the polypeptide includes the signal peptide. Any suitable signal sequence may be used. In one embodiment described herein, the DN TGF-βRI comprises the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) of SEQ ID NO: 224 or a portion thereof.

(SEQ ID NO: 224)
MEAAVAAPRPRLLLLVLAAAAAAAAALLPGATA.

A signal peptide or leader may facilitate the glycosylation of DN TGF-βRI. The signal sequence or leader is a peptide sequence generally present at either the N-terminus or C-terminus of newly synthesized proteins that directs their entry into the secretory pathway. In the present disclosure, the signal peptide is joined to the N-terminus of the extracellular antigen-binding domain of the DN TGF-βRI as a fusion protein. In one embodiment, the DN TGF βRI comprises an extracellular ligand binding domain having at least 75% sequence identity to (such as, at least 75%, at least 80%, at least 90%, at least 95%, or 100% identity; e.g., 85-90%, 85-95%, 85-100%, 90-95%, 90-100%, or 95-100%) the wild-type TGF-βRI and a signal peptide at the N-terminus of the extracellular domain TGF-βRI, having at least 75% sequence identity to (such as, at least 75%, at least 80%, at least 90%, at least 95%, or 100% identity; e.g., 85-90%, 85-95%, 85-100%, 90-95%, 90-100%, or 95-100%) the amino acid sequence of SEQ ID NO: 225.

(SEQ ID NO: 225)
MEAAVAAPRPRLLLLVLAAAAAAAAALLPGATALQCFCHLCTKDNFTCVTD
GLCFVSVTETTDKVIHNSMCIAEIDLIPRDRPFVCAPSSKTGSVTTTYCCN
QDHCNKIELPTTVKSSPGLGPVEL.

The engineered DN TGF-βRII constructs may also comprise an N-terminal signal peptide at the N-terminus of the extracellular ligand binding domain of TGF-βRII. In one embodiment, a heterologous signal peptide may be used. The extracellular domain of a DN TGF-βRII may be fused to a leader or a signal peptide that directs the nascent protein into the endoplasmic reticulum and subsequent translocation to the cell surface. It is understood that, once a polypeptide containing a signal peptide is expressed at the cell surface, the signal peptide is generally proteolytically removed during processing of the polypeptide in the endoplasmic reticulum and translocation to the cell surface. Thus, a polypeptide such as a DN TGF-βRII is generally found at the cell surface as a mature protein lacking the signal peptide, whereas the precursor form of the polypeptide includes the signal peptide. Any suitable signal sequence may be used. In one embodiment described herein, the DN TGF-βRII constructs described herein comprise a signal sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) the amino acid sequence of SEQ ID NO: 226 or a portion thereof. MGRGLLRGLWPLHIVLWTRIAS (SEQ ID NO: 226). In another embodiment, the signal sequence is derived from Colony Stimulating Factor 2 Receptor Alpha subunit (CSF2Rα) comprising the amino acid sequence at least 75% sequence identity to (such as, at least 75%, at least 80%, at least 90%, at least 95%, or 100% identity; e.g., 85-90%, 85-95%, 85-100%, 90 95%, 90-100%, or 95-100%) of SEQ ID NO: 137 or a portion thereof. MLLLVTSLLLCELPHPAFLLIP (SEQ ID NO: 137). The signal sequences described herein may also be optionally used with any suitable protein tag, including but not limited to: V5-tag, myc-tag, HA-tag, Spot-tag, NE-tag. In one embodiment described herein, the signal sequence and tag comprise the amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 227. MLLLVTSLLLCELPHPAFLLIPEQKLISEEDL (SEQ ID NO: 227).

It is understood that use of this signal peptide is exemplary. Any suitable signal peptide, as are well known in the art, may be applied to the DN TGF-βRI or RII to provide cell surface expression in an immune cell. Useful signal peptides may be derived from cell surface proteins naturally expressed in the T cell NK cell or precursor cell thereof, including any of the signal peptides of the polypeptides disclosed herein. Thus, any suitable signal peptide may be utilized to direct the DN TGF-βRI RII to be expressed at the cell surface of a T cell or NK cell.

In embodiments, a DN TGF-βRI comprises an amino acid sequence at least 75% sequence identity to (such as, at least 75%, at least 80%, at least 90%, at least 95%, or 100% identity; e.g., 85-90%, 85-95%, 85-100%, 90-95%, 90-100%, or 95-100%) the amino acid sequence of SEQ ID NO: 228.

(SEQ ID NO: 228)
MEAAVAAPRPRLLLLVLAAAAAAAAALLPGATALQCFCHLCTKDNFTCVTD
GLCFVSVTETTDKVIHNSMCIAEIDLIPRDRPFVCAPSSKTGSVTTTYCCN
QDHCNKIELPTTVKSSPGLGPVELAAVIAGPVCFVCISLMLMVYIRVNRQ.

In one embodiment a DN TGF-βRII comprises an amino acid sequence at least 75% sequence identity to (such as, at least 75%, at least 80%, at least 90%, at least 95%, or 100% identity; e.g., 85-90%, 85-95%, 85-100%, 90-95%, 90-100%, or 95-100%) the amino acid sequence of SEQ ID NO: 229:

(SEQ ID NO: 229)
MGRGLLRGLWPLHIVLWTRIASTIPPHVQKSVNNDMIVTDNNGAVKFPQLC
KFCDVRFSTCDNQKSCMSNCSITSICEKPQEVCVAVWRKNDENITLETVCH
DPKLPYHDFILEDAASPKCIMKEKKKPGETFFMCSCSSDECNDNIIFSEEY
NTSNPDLLLVIFQVTGISLLPPLGVAISVIIIFYCYRVNRQ.

In an embodiment a DN TGF-βRII comprises an amino acid sequence at least 75% sequence identity to (such as, at least 75%, at least 80%, at least 90%, at least 95%, or 100% identity; e.g., 85-90%, 85-95%, 85-100%, 90-95%, 90-100%, or 95-100%) the amino acid sequence of SEQ ID NO: 230.

(SEQ ID NO: 230)
TIPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIMK
EKKKPGETFFMCSCSSDECNDNIIFSEEYNTSNPD.

In one embodiment described herein, the DN TGF-βRII comprises an amino acid sequence at least 75% sequence identity to (such as, at least 75%, at least 80%, at least 90%, at least 95%, or 100% identity; e.g., 85-90%, 85-95%, 85-100%, 90-95%, 90-100%, or 95-100%) the wild-type TGF-βRII as shown in the amino acid sequence of SEQ ID NO: 231.

(SEQ ID NO: 231)
TIPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIMK
EKKKPGETFFMCSCSSDECNDNIIFSEEYNTSNPDLLLVIFQVTGISLLPP
LGVAISVIIIFYCY.

In one embodiment described herein, the DN TGF-βRII comprises an amino acid sequence at least 75% sequence identity to (such as, at least 75%, at least 80%, at least 90%, at least 95%, or 100% identity; e.g., 85-90%, 85-95%, 85-100%, 90-95%, 90-100%, or 95-100%) the amino acid sequence of SEQ ID NO: 232.

(SEQ ID NO: 232)
TIPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIMK
EKKKPGETFFMCSCSSDECNDNIIFSEEYNTSNPDSGPILLTISILSFFSV
ALLVIL.

In one embodiment described herein, the DN TGF βRII comprises an amino acid sequence at least 75% sequence identity to (such as, at least 75%, at least 80%, at least 90%, at least 95%, or 100% identity; e.g., 85-90%, 85-95%, 85-100%, 90-95%, 90-100%, or 95-100%) as shown in the amino acid sequence of SEQ ID NO: 233.

(SEQ ID NO: 233)
TIPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIMK
EKKKPGETFFMCSCSSDECNDNIIFSEEYNTSNPDSGPILLTCPTISILSF
FSVALLVIL.

In one embodiment described herein, the DN TGF-βRII comprises an amino acid sequence at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 234.

(SEQ ID NO: 234)
ACVLWKKRIKPIVWPSLPDHKKTLEHLCKKPRKNLNVSFNPESFLDCQIHR
VDDIQARDEVEGFLQDTFPQQLEESEKQRLGGDVQSPNCPSEDVVITPESF
GRDSSLTCLAGNVSACDAPILSSSRSLDCRESGKNGPHVYQDLLLSLGTTN
STLPPPFSLQSGILTLNPVAQGQPILTSLGSNQEEAYVTMSSFYQNQ.

In one embodiment described herein, the DN TGF-βRII comprises an amino acid sequence at least 75% sequence identity to (such as, at least 75%, at least 80%, at least 90%, at least 95%, or 100% identity e.g., 85-90%, 85-95%, 85-100%, 90-95%, 90-100%, or 95-100%) the amino acid sequence of SEQ ID NO: 235.

(SEQ ID NO: 235)
TIPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIMK
EKKKPGETFFMCSCSSDECNDNIIFSEEYNTSNPDSGPILLTISILSFFSV
ALLVILACVLWKKRIKPIVWPSLPDHKKTLEHLCKKPRKNLNVSFNPESFL
DCQIHRVDDIQARDEVEGFLQDTFPQQLEESEKQRLGGDVQSPNCPSEDVV
ITPESFGRDSSLTCLAGNVSACDAPILSSSRSLDCRESGKNGPHVYQDLLL
SLGTTNSTLPPPFSLQSGILTLNPVAQGQPILTSLGSNQEEAYVTMSSFYQ
NQ.

In one embodiment described herein, the DN TGF-βRII comprises an amino acid sequence at least 75% sequence identity to (such as, at least 75%, at least 80%, at least 90%, at least 95%, or 100% identity; e.g., 85-90%, 85-95%, 85-100%, 90-95%, 90-100%, or 95-100%) to the amino acid sequence of SEQ ID NO: 236.

(SEQ ID NO: 236)
TIPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIMK
EKKKPGETFFMCSCSSDECNDNIIFSEEYNTSNPDSGPILLTCPTISILSF
FSVALLVILACVLWKKRIKPIVWPSLPDHKKTLEHLCKKPRKNLNVSFNPE
SFLDCQIHRVDDIQARDEVEGFLQDTFPQQLEESEKQRLGGDVQSPNCPSE
DVVITPESFGRDSSLTCLAGNVSACDAPILSSSRSLDCRESGKNGPHVYQD
LLLSLGTTNSTLPPPFSLQSGILTLNPVAQGQPILTSLGSNQEEAYVTMSS
FYQNQ.

In an embodiment an engineered DN TGF-βRII comprises an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) the amino acid sequence of SEQ ID NO: 237.

(SEQ ID NO: 237)
MLLLVTSLLLCELPHPAFLLIPTIPPHVQKSVNNDMIVTDNNGAVKFPQLC
KFCDVRFSTCDNQKSCMSNCSITSICEKPQEVCVAVWRKNDENITLETVCH
DPKLPYHDFILEDAASPKCIMKEKKKPGETFFMCSCSSDECNDNIIFSEEY
NTSNPD.

In an embodiment an engineered DN TGF-βRII comprises an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID NO: 238.

(SEQ ID NO: 238)
MLLLVTSLLLCELPHPAFLLIPEQKLISEEDLTIPPHVQKSVNNDMIVTDN
NGAVKFPQLCKFCDVRFSTCDNQKSCMSNCSITSICEKPQEVCVAVWRKND
ENITLETVCHDPKLPYHDFILEDAASPKCIMKEKKKPGETFFMCSCSSDEC
NDNIIFSEEYNTSNPD.

In an embodiment an engineered DN TGF-βRII comprises an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID: 239.

(SEQ ID NO: 239)
MLLLVTSLLLCELPHPAFLLIPTIPPHVQKSVNNDMIVTDNNGAVKFPQLC
KFCDVRFSTCDNQKSCMSNCSITSICEKPQEVCVAVWRKNDENITLETVCH
DPKLPYHDFILEDAASPKCIMKEKKKPGETFFMCSCSSDECNDNIIFSEEY
NTSNPDSGPILLTISILSFFSVALLVILACVLWKKRIKPIVWPSLPDHKKT
LEHLCKKPRKNLNVSFNPESFLDCQIHRVDDIQARDEVEGFLQDTFPQQLE
ESEKQRLGGDVQSPNCPSEDVVITPESFGRDSSLTCLAGNVSACDAPILSS
SRSLDCRESGKNGPHVYQDLLLSLGTTNSTLPPPFSLQSGILTLNPVAQGQ
PILTSLGSNQEEAYVTMSSFYQNQ.

In an embodiment an engineered DN TGF-βRII comprises an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID: 240.

(SEQ ID NO: 240)
MLLLVTSLLLCELPHPAFLLIPEQKLISEEDLTIPPHVQKSVNNDMIVTDN
NGAVKFPQLCKFCDVRFSTCDNQKSCMSNCSITSICEKPQEVCVAVWRKND
ENITLETVCHDPKLPYHDFILEDAASPKCIMKEKKKPGETFFMCSCSSDEC
NDNIIFSEEYNTSNPDSGPILLTISILSFFSVALLVILACVLWKKRIKPIV
WPSLPDHKKTLEHLCKKPRKNLNVSFNPESFLDCQIHRVDDIQARDEVEGF
LQDTFPQQLEESEKQRLGGDVQSPNCPSEDVVITPESFGRDSSLTCLAGNV
SACDAPILSSSRSLDCRESGKNGPHVYQDLLLSLGTTNSTLPPPFSLQSGI
LTLNPVAQGQPILTSLGSNQEEAYVTMSSFYQNQ.

In an embodiment an engineered DN TGF-βRII comprises an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID: 241.

(SEQ ID NO: 241)
MLLLVTSLLLCELPHPAFLLIPTIPPHVQKSVNNDMIVTDNNGAVKFPQLC
KFCDVRFSTCDNQKSCMSNCSITSICEKPQEVCVAVWRKNDENITLETVCH
DPKLPYHDFILEDAASPKCIMKEKKKPGETFFMCSCSSDECNDNIIFSEEY
NTSNPDSGPILLTISILSFFSVALLVILACVLWKKRIKPIVWPSLPDHKKT
LEHLCKKPRKNLNVSFNPESFLDCQIHRVDDIQARDEVEGFLQDTFPQQLE
ESEKQRLGGDVQSPNCPSEDVVITPESFGRDSSLTCLAGNVSACDAPILSS
SRSLDCRESGKNGPHVYQDLLLSLGTTNSTLPPPFSLQSGILTLNPVAQGQ
PILTSLGSNQEEAYVTMSSFYQNQ.

In an embodiment an engineered DN TGF-βRII comprises an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID: 242.

(SEQ ID NO: 242)
MLLLVTSLLLCELPHPAFLLIPEQKLISEEDLTIPPHVQKSVNNDMIVTDN
NGAVKFPQLCKFCDVRFSTCDNQKSCMSNCSITSICEKPQEVCVAVWRKND
ENITLETVCHDPKLPYHDFILEDAASPKCIMKEKKKPGETFFMCSCSSDEC
NDNIIFSEEYNTSNPDSGPILLTCPTISILSFFSVALLVILACVLWKKRIK
PIVWPSLPDHKKTLEHLCKKPRKNLNVSFNPESFLDCQIHRVDDIQARDEV
EGFLQDTFPQQLEESEKQRLGGDVQSPNCPSEDVVITPESFGRDSSLTCLA
GNVSACDAPILSSSRSLDCRESGKNGPHVYQDLLLSLGTTNSTLPPPFSLQ
SGILTLNPVAQGQPILTSLGSNQEEAYVTMSSFYQNQ.

In an embodiment an engineered DN TGF-βRII comprises an amino acid sequence having at least 75% sequence identity to (such as, at least 75%, at least 80%, at least 90%, at least 95%, or 100% identity; e.g., 85-90%, 85-95%, 85-100%, 90-95%, 90-100%, or 95-100%) SEQ ID: 243.

(SEQ ID NO: 243)
MLLLVTSLLLCELPHPAFLLIPTIPPHVQKSVNNDMIVTDNNGAVKFPQLC
KFCDVRFSTCDNQKSCMSNCSITSICEKPQEVCVAVWRKNDENITLETVCH
DPKLPYHDFILEDAASPKCIMKEKKKPGETFFMCSCSSDECNDNIIFSEEY
NTSNPDSGPILLTCPTISILSFFSVALLVILACVLWKKRIKPIVWPSLPDH
KKTLEHLCKKPRKNLNVSFNPESFLDCQIHRVDDIQARDEVEGFLQDTFPQ
QLEESEKQRLGGDVQSPNCPSEDVVITPESFGRDSSLTCLAGNVSACDAPI
LSSSRSLDCRESGKNGPHVYQDLLLSLGTTNSTLPPPFSLQSGILTLNPVA
QGQPILTSLGSNQEEAYVTMSSFYQNQ.

The present disclosure contemplates, the expression of polynucleotides encoding the anti-PSMA binding domains disclosed herein and the anti-PSMA CARs disclosed herein, co-expression of polynucleotides comprising the engineered DN TGF-β Receptors with constitutively expressed anti-PSMA CARs, conditionally expressed anti-PSMA CARs and anti-PSCA synNotch receptor described herein, fragments thereof, cells and compositions comprising the same, and vectors that express polypeptides. “Polypeptide,” “polypeptide fragment,” “peptide” and “protein” are, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the polypeptides contemplated herein comprise a signal (or leader) sequence at the N-terminal end of the protein, which co-translationally or post-translationally directs transfer of the protein.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in some embodiments, it may be desirable to improve the binding affinity and/or other biological properties of the engineered DN TGF-β Receptors, engineered anti-PSMA CARs and engineered anti-PSCA synNotch Receptors by introducing one or more substitutions, deletions, additions and/or insertions. Preferably, polypeptides of the disclosure include polypeptides having at least about 50%, 60%, 65%, 70%, 75%, 85%, 90%, 95%, 98%, or 99% amino acid identity thereto. Polypeptides of the disclosure include variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. Polypeptides include “polypeptide fragments.” Polypeptide fragments refer to a polypeptide, which may be monomeric or multi-meric that has an amino-terminal deletion, a carboxyl-terminal deletion, and/or an internal deletion or substitution of a naturally occurring or recombinantly-produced polypeptide. In certain embodiments, a polypeptide fragment may comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long.

The polypeptide may also be fused in-frame or conjugated to a linker or other sequence for case of synthesis, purification or identification of the polypeptide (e.g., poly-His), or to enhance binding of the polypeptide to a solid support.

As noted above, polypeptides of the present disclosure may be altered in various ways including amino acid substitutions, deletions, truncations, and insertions. Methods for such manipulations are generally known in the art. For example, amino acid sequence variants of a reference polypeptide may be prepared by mutations in the DNA. Methods for mutagenesis and nucleotide sequence alterations are well known in the art. See, for example, Kunkel (1985, Proc. Natl. Acad. Sci. USA. 82:488-492), Kunkel et al., (1987, Methods in Enzymol, 154:367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present disclosure and still obtain a functional molecule that encodes a variant or derivative polypeptide with desirable characteristics.

Polypeptide variants further include glycosylated forms, aggregative conjugates with other molecules, and covalent conjugates with unrelated chemical moieties (e.g., pegylated molecules). Covalent variants may be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

Where expression of two or more polypeptides is desired, the polynucleotide sequences encoding them may be separated by an IRES sequence as discussed elsewhere herein. In another embodiment, two or more polypeptides may be expressed as a fusion protein that comprises one or more self-cleaving polypeptide sequences. In other embodiments, they are expressed from different promotors and can be in two or more (such as three) vectors. In some embodiments, an anti-PSMA CARs encoded in the same vector as an engineered DN TGF-β Receptor and is operably linked to the same promotor as the engineered DN TGF-β Receptor where the sequences are separated by an IRES sequence. In some embodiments, an anti-PSMA CARs encoded in the same vector as an engineered DN TGF-β Receptor is operably linked to a different promotor than the promotor the engineered DN TGF-β Receptor. In some embodiments, an anti-PSMA CARS encoded in the same vector as an engineered DN TGF-β Receptor is operably linked to a conditionally active promotor and the engineered DN TGF-β Receptor is operably linked to a constitutively active promotor. In some embodiments, an anti-PSCA synNotch receptor is encoded in a different vector than either an anti-PSMA CARs or an engineered DN TGF-β Receptor, which could be on the same or different vectors. In some embodiments, a nucleic acid encoding an anti-PSCA synNotch receptor is operably connected to a constitutively active promotor. In some embodiments, a nucleic acid encoding an anti-PSMA CAR is operably connected to a constitutively active promotor. In some embodiments, a nucleic acid encoding an anti-PSMA CAR is operably connected to a conditionally active promotor, for example active upon binding of the transcriptional activator domain of an anti-PSCA synNotch receptor. In some embodiments, a nucleic acid encoding an DN TGF-β Receptor is operably connected to a constitutively active promotor. In embodiments the transcriptional activator domain of an anti-PSCA synNotch receptor comprises GAL4-VP64 and the conditionally active promoter comprises one or more GAL4 binding sites, such as 1, 2, 3, 4, 5, 6, or 7 GAL4 binding sites. In an embodiment a GAL4 binding site has the nucleic acid sequence according to.

Polypeptides of the present disclosure include fusion polypeptides. In some embodiments, fusion polypeptides and polynucleotides encoding fusion polypeptides are provided. Fusion polypeptides and fusion proteins refer to a polypeptide having at least two, three, four, five, six, seven, eight, nine, or ten or more polypeptide segments. Fusion polypeptides are typically linked C-terminus to N-terminus, although they may also be linked C-terminus to C-terminus, N-terminus to N-terminus, or N-terminus to C-terminus. The polypeptides of the fusion protein may be in any order or a specified order. Fusion polypeptides or fusion proteins may also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other common techniques. Ligated DNA sequences comprising the fusion polypeptide are operably linked to suitable transcriptional or translational control elements as discussed elsewhere herein.

In one embodiment, a fusion partner comprises a sequence that assists in expressing the protein (an expression enhancer) at higher yields than the native recombinant protein. Other fusion partners may be selected so as to increase the solubility of the protein or to enable the protein to be targeted to desired intracellular compartments or to facilitate transport of the fusion protein through the cell membrane.

Fusion polypeptides may further comprise a polypeptide cleavage signal between each of the polypeptide domains described herein. In addition, polypeptide site may be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5 (8); 616-26).

Suitable protease cleavages sites and self-cleaving peptides are known to the skilled person (see, e.g., in Ryan et al., 1997. J Gener. Viral. 78, 699-722; Scymczak et al. (2004) Nature Biotech. 5, 589-594). Exemplary protease cleavage sites include, but are not limited to the cleavage sites of potyvirus Nia proteases (e.g., tobacco etch virus protease), potyvirus HC proteases, potyvirus Pl (P35) proteases, byovirus Nia proteases, byovirus RNA-2-encoded proteases, aphthovirus L proteases, enterovirus 2A proteases, rhinovirus 2A proteases, picoma 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites may be used. In other embodiments, self cleaving peptides may include those polypeptide sequences obtained from potyvirus and cardiovirus 2A peptides, FMDV (foot-and-mouth disease virus), equine rhinitis A virus, Thosea asigna virus and porcine teschovirus. In other embodiments, the self-cleaving polypeptide site comprises a 2A or 2A-like site, sequence or domain (Donnelly et al., 2001. J Gen. Viral. 82:1027-1041).

Generally, it is understood that any appropriate viral vector or vectors may be used for transduction of the engineered constructs described herein. In one embodiment described herein, a cell (e.g., T cell or NK cell) is transduced with a retroviral vector, e.g., a lentiviral vector, encoding an engineered DN TGF-β Receptor construct and an engineered anti-PSMA CARS and/or engineered anti-PSCA synNotch receptors as described herein. The transduced T cells elicits a stable, long-term, and persistent response.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in some embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis encephalitis virus (CAEV); equine infectious anemia virus (EIAV); feline immunodeficiency virus (FIV); bovine immune deficiency virus (BIV); and simian immunodeficiency virus (SIV).

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

As will be evident to one of skill in the art, the term “viral vector” is widely used to refer cither to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-retroviral viral sequences. In one embodiment, a hybrid vector refers to a vector or transfer plasmid comprising retroviral e.g., lentiviral, sequences for reverse transcription, replication, integration and/or packaging.

In some embodiments, the terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the disclosure and are present in DNA form in the DNA plasmids of the disclosure. In one embodiment described herein, the expression vector is a lentivirus expression vector.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, Rand U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R, and U5. The U3 region contains the enhancer and promoter elements. The U5 region is the sequence between the primer binding site and the R region and contains the polyadenylation sequence. The R (repeat) region is flanked by the U3 and U5 regions. The LTR is composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., 1995. J of Virology, Vol. 69, No. 4; pp. 2101-2109. Several retroviral vectors use the minimal packaging signal (also referred to as the psi [′P] sequence) needed for encapsidation of the viral genome. Thus, as used herein, the terms “packaging sequence,” “packaging signal,” “psi” and the symbol “P,” are used in reference to the non-coding sequence required for encapsidation of retroviral RNA strands during viral particle formation.

In various embodiments, vectors comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3 ‘) LTR U3 region is used as a template for the left (5’) LTR U3 region during viral replication and, thus, the viral transcript cannot be made without the U3 enhancer-promoter. In a further embodiment of the disclosure, the 3′LTR is modified such that the U5 region is replaced, for example, with an ideal poly(A) sequence. It should be noted that modifications to the LTRs such as modifications to the 3′LTR, the 5′LTR, or both 3′ and 5′LTRs, are also contemplated herein.

An additional safety enhancement is provided by replacing the U3 region of the 5′LTR with a heterologous promoter to drive transcription of the viral genome during production of viral particles. Examples of heterologous promoters which may be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter may be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication.

The “R region” refers to the region within retroviral LTRs beginning at the start of the capping group (i.e., the start of transcription) and ending immediately prior to the start of the poly A tract. The R region is also defined as being flanked by the U3 and U5 regions. The R region plays a role during reverse transcription in permitting the transfer of nascent DNA from one end of the genome to the other.

As used herein, the term “FLAP element” refers to a nucleic acid whose sequence includes the central polypurine tract and central termination sequences (cPPT and CTS) of a includes the central polypurine tract and central termination sequences (cPPT and CTS) of a retrovirus, e.g., HIV-I or HIV-2. Suitable FLAP elements are described in U.S. Pat. No. 6,682,907 and in Zennou, et al., 2000, Cell, 101:173. During HIV-I reverse transcription, central initiation of the plus-strand DNA at the central polypurine tract (cPPT) and central termination at the central termination sequence (CTS) lead to the formation of a three-stranded DNA structure: the HIV-I central DNA flap. While not wishing to be bound by any theory, the DNA flap may act as a cis-active determinant of lentiviral genome nuclear import and/or may increase the titer of the virus.

In one embodiment, retroviral or lentiviral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J Virol. 65:1053; and Cullen et al., 1991. Cell 58:423), and the hepatitis B virus post-transcriptional regulatory element (HPRE). Generally, the RNA export element is placed within the 3′ UTR of a gene, and may be inserted as one or multiple copies.

In other embodiments, expression of heterologous sequences in viral vectors is increased by incorporating post-transcriptional regulatory elements, efficient polyadenylation sites, and optionally, transcription termination signals into the vectors. A variety of posttranscriptional regulatory elements may increase expression of a heterologous nucleic acid at the protein, e.g., woodchuck hepatitis virus post-transcriptional regulatory element (WPRE; Zufferey et al., 1999, J Virol., 73:2886); the post-transcriptional regulatory element present in hepatitis B virus (HPRE) (Huang et al., Mol. Cell. Biol., 5:3864); and the like (Liu et al., 1995, Genes Dev., 9:1766).

In some embodiments, vectors may include regulatory oligonucleotides having transcriptional or translational regulatory activity. Such an oligonucleotide can be used in a variety of gene expression configurations for regulating control of expression. A transcriptional regulatory oligonucleotide, can increase (enhance) or decrease (silence) the level of expression of a recombinant expression construct. Regulatory oligonucleotides may selectively regulate expression in a context specific manner, including, for example, for conferring tissue specific, developmental stage specific, or the like expression of the polynucleotide, including constitutive or inducible expression. A regulatory oligonucleotide of the disclosure also can be a component of an expression vector or of a recombinant nucleic acid molecule comprising the regulatory oligonucleotide operatively linked to an expressible polynucleotide. A regulatory element can be of various lengths from a few nucleotides to several hundred nucleotides.

Elements directing the efficient termination and polyadenylation of the heterologous nucleic acid transcripts increases heterologous gene expression. Transcription termination signals are generally found downstream of the polyadenylation signal. In some embodiments, vectors comprise a polyadenylation sequence 3′ of a polynucleotide encoding a polypeptide to be expressed. The term “poly A site” or “poly A sequence” as used herein denotes a DNA sequence which directs both the termination and polyadenylation of the nascent RNA transcript by RNA polymerase II. Polyadenylation sequences may promote mRNA stability by addition of a poly A tail to the 3′ end of the coding sequence and thus, contribute to increased translational efficiency. Efficient polyadenylation of the recombinant transcript is desirable as transcripts lacking a poly A tail are unstable and are rapidly degraded. Illustrative examples of poly A signals that may be used in a vector of the disclosure, includes an ideal poly A sequence (e.g., AATAAA, ATTAAA, AGTAAA), a bovine growth hormone poly A sequence (BGHpA), a rabbit β-globin poly A sequence (rβgpA), or another suitable heterologous or endogenous poly A sequence known in the art.

Also described herein are “codon-optimized” nucleic acids. A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells) by replacing at least one, more than one, or a significant number, of codons of the native sequence with codons that are more frequently or most frequently used in the genes of that species. Codon optimization does not alter the amino acid sequence of the encoded protein.

The codon-optimized nucleotide sequences presented in the instant disclosure can present improved properties related to expression efficacy. In some embodiments, the DNA sequence to be transcribed may be optimized to facilitate more efficient transcription and/or translation. In some embodiments, the DNA sequence may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA sequence may be optimized regarding cryptic splice sites, mRNA secondary structure, stable free energy of mRNA, repetitive sequences, RNA instability motif, and/or other elements relevant to mRNA processing and stability; the DNA sequence may be optimized regarding codon usage bias, codon adaptability, internal chi sites, ribosomal binding sites (e.g., IRES), premature polyA sites, Shine-Dalgarno (SD) sequences, and/or other elements relevant to translation; and/or the DNA sequence may be optimized regarding codon context, codon-anticodon interaction, translational pause sites, and/or other elements relevant to protein folding.

The vectors may have one or more LTRs, wherein any LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase therapeutic gene expression (e.g., poly (A) sequences), and may optionally comprise a WPRE or HPRE. The skilled artisan would appreciate that many other different embodiments may be fashioned from the existing embodiments of the disclosure.

A “host cell” includes cells transfected, infected, or transduced in vivo, ex vivo, or in vitro with a recombinant vector or a polynucleotide of the disclosure. Host cells may include packaging cells, producer cells, and cells infected with viral vectors. In some embodiments, host cells infected with viral vector of the disclosure are administered to a subject in need of therapy. In certain embodiments, the term “target cell” is used interchangeably with host cell and refers to transfected, infected, or transduced cells of a desired cell type. In some embodiments, the target cell is a T cell.

Large scale viral particle production is often necessary to achieve a reasonable viral titer. Viral particles are produced by transfecting a transfer vector into a packaging cell line that comprises viral structural and/or accessory genes, e.g., gag, pol, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. A retroviral/lentiviral transfer vector of the present disclosure may be introduced into a packaging cell line, via transfection, transduction or infection, to generate a producer cell or cell line. The packaging vectors of the present disclosure may be introduced into human cells or cell lines by common methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene may be linked physically to genes encoding by the packaging vector, e.g., by IRES or self-cleaving viral peptides.

Viral envelope proteins (env) determine the range of host cells which may ultimately be infected and transformed by recombinant retroviruses generated from the cell lines. In the case of lentiviruses, such as HIV-1, HIV-2, SIV, FIV and EIV, the env proteins include gp41 and gp120. In some embodiments, the viral env proteins expressed by packaging cells of the disclosure are encoded on a separate vector from the viral gag and pol genes, as has been previously described.

Illustrative examples of retroviral-derived env genes which may be employed in the embodiments described herein include, but are not limited to: MLV envelopes, IOAI envelope, BAEV, FcLV-B, RDI 14, SSAV, Ebola, Sendai, FPV (Fowl plague virus), and influenza virus envelopes. Similarly, genes encoding envelopes from RNA viruses (e.g., RNA virus families of Picomaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Rcoviridae, Bimaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples include, FcLV, VEE, HFVW, WDSV, SFV, Rabies, ALV, BIV, BL V, EBV, CAEV, SNV, ChTL V, STLV, MPMV SMRV, RAV, FuSV, MH2, AEV, AMV, CTIO, and EIAV.

In other embodiments, envelope proteins for pseudotyping a virus of present disclosure include, but are not limited to any of the following virus: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpes viruses such as Herpes simplex virus types 1 and 2, varicella zoster, cytomegalovirus, Epstein-Barr virus (EBV), human herpesviruses (HHV), human herpesvirus type 6 and 8, Human immunodeficiency virus (HIV), papilloma virus, murine gamma herpes virus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-Co V), West Nile virus, or any encephaliltis causing virus.

The terms “pseudotype” or “pseudotyping” as used herein, refer to a virus whose viral envelope proteins have been substituted with those of another virus possessing other characteristics. For example, HIV may be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells.

As used herein, the term “packaging cell lines” is used in reference to cell lines that do not contain a packaging signal, but do stably or transiently express viral structural proteins and replication enzymes (e.g., gag, pol and env) which are necessary for the correct packaging of viral particles. Any suitable cell line may be employed to prepare packaging cells of the disclosure. Generally, the cells are mammalian cells. In another embodiment, the cells used to produce the packaging cell line are human cells. Suitable cell lines which may be used to produce the packaging cell line include, for example, CHO cells, BHK cells, MDCK cells, C3H 10TI/2 cells, FLY cells, Psi-2 cells, BOSC 23 cells, P A317 cells, WEHI cells, COS cells, BSC 1 cells, BSC 40 cells, BMT 10 cells, VERO cells, W138 cells, MRC5 cells, A549 cells, HTI080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Methods of preparing viral stock solutions are known in the art and are illustrated by, e.g., Y. Soncoka et al. (1995) Nucl. Acids Res. 23:628-633, and N. R. Landau et al. (1992) J Virol. 66:5110-5113. Infectious virus particles may be collected from the packaging cells using conventional techniques. For example, the infectious particles may be collected by cell lysis, or collection of the supernatant of the cell culture, as is known in the art. Optionally, the collected virus particles may be purified if desired. Suitable purification techniques are well known to those skilled in the art.

The delivery of a gene(s) or other polynucleotide sequence using a retroviral or lentiviral vector by means of viral infection rather than by transfection is referred to as “transduction.” In one embodiment, retroviral vectors are transduced into a cell through infection and provirus integration. In certain embodiments, a target cell, e.g., a T cell or NK cell, is “transduced” if it comprises a gene or other polynucleotide sequence delivered to the cell by infection using a viral or retroviral vector. In some embodiments, a transduced cell comprises one or more genes or other polynucleotide sequences delivered by a retroviral or lentiviral vector in its cellular genome.

In some embodiments, host cells expressing one ore more of the constructs of the disclosure (anti-PSMA CAR, anti-PSCA synNotch receptor, and/or). The host cells may be transduced with one or more viral vectors comprising nucleic acid sequences encoding one or more polypeptides expressing a DN TGF-β receptor construct of the disclosure and an engineered TCR and/or a CAR. The host cells may be administered to a subject to treat and/or prevent T cell malignancies. Other methods relating to the use of viral vectors in gene therapy, which may be utilized according to certain embodiments of the present disclosure, may be found in, e.g., Kay, M. A. (1997) Chest 111 (6 Supp.): 138S-142S; Ferry, N. and Heard, J. M. (1998) Hum. Gene Ther. 9:1975-81; Shiratory, Y. et al., (1999) Liver 19:265-74; Oka, K. et al., (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J Hepatol. 23:746-58; Brody, S. L. and Crystal, R. G. (1994) Ann. NY Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lec, H. C. et al., (2000) Nature 408:483-8.

The compositions described herein may comprise one or more polynucleotides, polypeptides, vectors comprising same, and T cell compositions, as contemplated herein. One embodiment described herein is a composition comprising a modified T cell that co-expresses one or more engineered DN TGF-β Receptors described herein with an engineered TCR and/or CAR. Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions of the present disclosure may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl

cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In one embodiment described herein, compositions of the present disclosure comprise an amount of modified T cells contemplated herein. It may generally be stated that a pharmaceutical composition comprising the T cells contemplated herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of cells will depend upon the ultimate use for which the composition is intended as will the type of cells included therein. T cells modified to express an engineered TCR or CAR may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-7, IL-15, IL-12, TNF-alpha, IL-18, and TNF-beta, GM-CSF, IL-4, IL-13, Flt3-L, RANTES, MIP1α, etc.) as described herein to enhance engraftment and function of infused T cells.

Generally, compositions comprising the cells activated and expanded as described herein may be utilized in the treatment and prevention of diseases that arise in individuals who are immunocompromised or immunosuppressed. In some, compositions comprising the modified T cells contemplated herein are used in the treatment of cancers. The modified T cells described herein may be administered either alone, or as a pharmaceutical composition in combination with carriers, diluents, excipients, and/or with other components such as IL-2, IL-7, and/or IL-15 or other cytokines or cell populations. In some embodiments, pharmaceutical compositions contemplated herein comprise an amount of genetically modified T cells, in combination with one or more pharmaceutically or physiologically acceptable carriers, diluents or excipients.

Pharmaceutical compositions comprising modified T cells contemplated herein may further comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for parenteral administration, e.g., intravascular (intravenous or intra-arterial), intraperitoneal or intramuscular administration.

The liquid pharmaceutical compositions, whether they be solutions, suspensions or other like form, may include one or more of the following: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation may be enclosed in ampoules, disposable syringes or multiple dose vials made of glass or plastic. Sterile injectable pharmaceutical composition are also included.

In some embodiments, compositions contemplated herein comprise an effective amount of an expanded modified T cell composition, alone or in combination with one or more therapeutic agents. Thus, the T cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics and anti-viral agents. Such therapeutic agents may be accepted in the art as a treatment for a disease state as described herein, such as a cancer. In one embodiment the compositions contemplated herein may also be administered with inhibitors of TGF-β, for example the small molecule inhibitor LY55299. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

In certain embodiments, compositions comprising T cells contemplated herein may be administered in conjunction with any number of chemotherapeutic agents. Illustrative examples of chemotherapeutic agents include but are not limited to alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trictylenephosphoramide, triethylenethiophosphaoramide and trimethylolomelamine resume; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, didcoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RPS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of other therapeutic agents may be used in conjunction with the compositions described herein. In one embodiment, the composition comprising T cells is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

In some embodiments, NSAIDs are chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol or proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary disease-modifying anti-rheumatic drugs (DMARDs) include azathioprine, cyclophosphamide, cyclosporine, methotrexate, penicillamine, leflunomide, sulfasalazine, hydroxychloroquine, Gold (oral (auranofin) and intramuscular) and minocycline.

In other embodiments, the therapeutic antibodies suitable for combination with the CAR modified T cells contemplated herein, include but are not limited to, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ccromeximab, clotuzumab, ensituximab, ertumaxomab, etaracizumab, farictuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxctumomab, namatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8.

In some embodiments, the compositions described herein are administered in conjunction with a cytokine. By “cytokine” as used herein is meant a generic term for proteins released by one cell population that act on another cell as intercellular mediators. Examples of such cytokines are lymphokines, monokines, chemokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; IL-15, a tumor necrosis factor such as TNF-α or TNF-β; and other polypeptide factors including LIF and kit ligand (KL). As used herein, the term cytokine includes proteins from natural sources or from recombinant cell culture, and biologically active equivalents of the native sequence cytokines.

The present disclosure contemplates, in part, genetically modified T cell redirected to a target cell, e.g., a tumor or cancer cell, and that comprises the engineered DN TGF-β Receptors described herein and an engineered TCR or CAR having a binding domain that binds to target antigens on the cells, including the CAR-DN TGF-β Receptor constructs described herein. Cancer cells may also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In one embodiment, the target cell expresses an antigen, e.g., target antigen. In one embodiment, the target cell is a pancreatic parenchymal cell, pancreatic duct cell, hepatic cell, cardiac muscle cell, skeletal muscle cell, osteoblast, skeletal myoblast, neuron, vascular endothelial cell, pigment cell, smooth muscle cell, glial cell, fat cell, bone cell, chondrocyte, pancreatic islet cell, CNS cell, PNS cell, liver cell, adipose cell, hepatic cell, renal cell, lung cell, skin cell, ovary cell, follicular cell, epithelial cell, immune cell, or an endothelial cell.

In certain embodiments, the target cell is part of a pancreatic tissue, neural tissue, cardiac tissue, bone marrow, muscle tissue, bone tissue, skin tissue, liver tissue, hair follicles, vascular tissue, adipose tissue, lung tissue, and kidney tissue.

In a one embodiment, the target cell is a tumor cell. In another embodiment, the target cell is a cancer cell, such as a cell in a patient with cancer. Exemplary cells that may be killed with the disclosed methods include cells of prostate tumors. In one embodiment, the target cell is a malignant cell of the prostate.

The modified T cells and NK cells contemplated herein provide improved adoptive immunotherapy for use in the treatment of various conditions including, without limitation, cancer, infectious disease, autoimmune disease, inflammatory disease, and immunodeficiency. In some embodiments, the specificity of a primary T cell is redirected to tumor or cancer cells by genetically modifying the primary T cell engineered to co-express a TCR or CAR contemplated herein with an engineered DN TGF-β Receptor. For example, with the CAR-DN TGF-β Receptor constructs described herein, the antigen binding domain of the scFv of the CAR directs the T cell to the cells expressing the tumor antigen, and the TGF-β Receptor constructs described herein protect the population of such T cells by inhibiting the suppressive effect of TGF-β.

In one embodiment of the present disclosure includes a type of cellular therapy where T cells are modified to co-express an engineered DN TGF-β Receptor with an engineered TCR or CAR, including the CAR-DN TGF-Receptor constructs described herein, that target cancer cells expressing a target antigen, and the modified T cell is infused to a recipient in need thereof. The infused cell is thus able to kill tumor cells in the recipient with minimal impact from TGF-β suppression.

Any cell may be used as a host cell for the polynucleotides, the vectors, or the polypeptides of the present disclosure. In some embodiments, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. acruginosa; and Streptomyces. In some embodiments, the cell is a human cell. In some embodiments, the cell is an immune cell. In some embodiments, the immune cell is selected from the group consisting of a T cell, a B cell, a tumor infiltrating lymphocyte (TIL), a TCR expressing cell, a natural killer (NK) cell, a dendritic cell, a granulocyte, an innate lymphoid cell, a megakaryocyte, a monocyte, a macrophage, a platelet, a thymocyte, and a myeloid cell. In one embodiment, the immune cell is a T cell. In another embodiment, the immune cell is an NK cell. In certain embodiments, the T cell is a tumor-infiltrating lymphocyte (TIL), autologous T cell, engineered autologous T cell (eACT™), an allogeneic T cell, a heterologous T cell, or any combination thereof. Unlike antibody therapies or standalone TCR or CAR modified T cells, T cells (or any cells as described above) modified to co-express the engineered DN TGF-Receptors described herein with an engineered TCR or CAR are able to not only replicate in vivo, and thus contribute to long-term persistence that may lead to sustained cancer therapy, but have the added advantage of avoiding the suppressive impact of TGF-β. Thus, in one embodiment described herein is a method of inhibiting the activity of TGF-β comprising administering to a subject in need thereof a therapeutically effective amount of the modified T cell co-expressing the DN TGF-β Receptors described herein and a TCR or CAR as described herein.

In another embodiment, T cells co-expressing the DN TGF-β Receptors with an engineered TCR or CAR as described herein may undergo T cell expansion such that a population of therapeutic T cells may remain or persist for an extended period. Thus, another embodiment described herein is a method of expanding a population of T cells comprising administering to a subject in need thereof a therapeutically effective amount of the T cells described herein.

In one embodiment described herein, the population of T cells remains between at between about 50% to about 100% after 7 days, at between about 60% to about 90% after 7 days, or at between about 70% to about 80% after 7 days. In another embodiment described herein, the population of T cells remains at about 50% after 7 days, at about 60% after 7 days, at about 70% after 7 days, at about 80% after 7 days, at about 90% after 7 days or at about 100% after 7 days.

In another embodiment described herein, administration of the modified T cells described herein results in an expansion of transduced T cells in the presence of TGF-1 by about 10% to about 100%, about 20% to about 90%, or about 30% to about 80%. In another embodiment described herein, administration of the modified T cells described herein results in an expansion of transduced T cells in the presence of TGF-β1 by about 1%, about 5%, about 10%, about 20%, about 30%, about 40%, about 50%, 50%, about 55%, about 60%, about 65%, about 70%, about 75%, about 80%, about 85%, about 90%, about 95%, or about 100%.

In one embodiment described herein, administration of a modified T cell from a codon-optimized sequence as described herein results in an increase in expression efficiency by about 50%, about 55%, about 60%, about 65%, about 70%, about 75%, about 80%, about 85%, about 90%, about 95%, or about 100%. In another embodiment described herein, administration of a modified T cell from a codon-optimized sequence as described herein results in an increase in transduction efficiency by about 50%, about 55%, about 60%, about 65%, about 70%, about 75%, about 80%, about 85%, about 90%, about 95%, or about 100%.

Without being bound by any theory, it is believed that despite the promising use of CAR T cell therapy, constitutive tonic signaling in the absence of tumor antigen can result in reduced efficacy, poor CAR T cell survival and toxicity (Ajina et al., Mol Cancer Ther., 17 (9): 1795-1815 (2018)). Thus, a CAR T cell therapy with reduced tonic signaling results superior performance. In one embodiment described herein, administration of a modified T cell from a codon-optimized sequence as described herein in the absence of tumor antigen results in a reduction of cytokine release by about 10%, about 20%, about 30%, about 40%, about 50%, 50%, about 55%, about 60%, about 65%, about 70%, about 75%, about 80%, about 85%, about 90%, about 95%, or about 100%. In one embodiment described herein, administration of a modified T cell from a codon-optimized sequence in the absence of tumor antigen results in a reduction of cytokine release by about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In another embodiment, administration of the modified T cell from a codon-optimized sequence as described herein results in a reduction in tumor volume of between about 50% to about 10% to about 100%, about 20% to about 90%, or about 30% to about 80%. In another embodiment the reduction in tumor volume is about 10%, about 20%, about 30%, about 40%, about 50%, 50%, about 55%, about 60%, about 65%, about 70%, about 75%, about 80%, about 85%, about 90%, about 95%, or about 100%.

Another embodiment described herein is a method of treating a cancer in a subject in need thereof comprising administering an effective amount, e.g., therapeutically effective amount of a composition comprising T cells co-expressing the DN TGF-β Receptors and a TCR or CAR as described herein. The quantity and frequency of administration will be determined by such factors as the condition of the patient, and the type and severity of the patient's disease, although appropriate dosages may be determined by clinical trials.

Another embodiment described herein is a method of treating a hepatic cancer in a subject in need thereof comprising administering an effective amount, e.g., therapeutically effective amount of a composition comprising T cells co-expressing the DN TGF-β Receptors and a TCR or CAR as described herein, including the CAR-DN TGF-β Receptor constructs described herein. The quantity and frequency of administration will be determined by such factors as the condition of the patient, and the type and severity of the patient's disease, although appropriate dosages may be determined by clinical trials.

In other embodiments, compositions comprising T cell genetically modified with a vector comprising a promoter operably linked to a polynucleotide encoding a DN TGF-β Receptor and a polynucleotide encoding a TCR or CAR, including the CAR-DN TGF-Receptor constructs described herein, are used in the treatment of solid tumors or cancers including, without limitation, liver cancer, pancreatic cancer, lung cancer, breast cancer, bladder cancer, brain cancer, bone cancer, thyroid cancer, kidney cancer, skin cancer or virus induced cancers.

In some embodiments, compositions comprising T cell genetically modified with a vector comprising a promoter operably linked to a polynucleotide encoding a DN TGF-Receptor and a polynucleotide encoding a CAR and/or a synNotch molecule, including the CAR-DN TGF-β Receptor constructs described herein, comprises an antigen-specific binding domain that binds an epitope of PSCA or PSMA are used in the treatment of various cancers.

In other embodiments, methods comprising administering a therapeutically effective amount of modified T cells contemplated herein or a composition comprising the same, to a patient in need thereof, alone or in combination with one or more therapeutic agents, are provided. In certain embodiments, the cells of the disclosure are used in the treatment of patients at risk for developing a cancer. Thus, the present disclosure provides methods for the treatment or prevention of a cancer comprising administering to a subject in need thereof, a therapeutically effective amount of the modified T cells of the disclosure.

One of ordinary skill in the art would recognize that multiple administrations of the compositions of the disclosure may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In certain embodiments, it may be desirable to administer activated T cells to a subject and then subsequently redraw blood (or have an apheresis performed), activate T cells therefrom according to the present disclosure, and reinfuse the patient with these activated and expanded T cells. This process may be carried out multiple times every few weeks. In certain embodiments, T cells may be activated from blood draws of from 10 cc to 400 cc. Not to be bound by theory, using this multiple blood draw/multiple reinfusion protocol may serve to select out certain populations of T cells.

The administration of the compositions contemplated herein may be carried out in any convenient manner, including by aerosol inhalation, injection, ingestion, transfusion, implantation or transplantation. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a subject in need thereof is administered an effective amount of a composition to increase a cellular immune response to a cancer in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, which are well described in the art; e.g., Current Protocols in Immunology, Edited by: John E. Coligan, Ada M. Kruisbeck, David H. Margulies, Ethan M. Shevach, Warren Strober (2001) John Wiley & Sons, NY, N.Y.

In the case of T cell-mediated killing, CAR-ligand binding initiates CAR signaling to the T cell, resulting in activation of a variety of T cell signaling pathways that induce the T cell to produce or release proteins capable of inducing target cell apoptosis by various mechanisms. These T cell-mediated mechanisms include (but are not limited to) the transfer of intracellular cytotoxic granules from the T cell into the target cell, T cell secretion of proinflammatory cytokines that may induce target cell killing directly (or indirectly via recruitment of other killer effector cells), and up regulation of death receptor ligands (e.g. FasL) on the T cell surface that induce target cell apoptosis following binding to their cognate death receptor (e.g. Fas) on the target cell.

In embodiments described herein is a method of treating a subject diagnosed with a cancer, comprising removing T cells from the subject, genetically modifying said T cells with a vector comprising a nucleic acid encoding an engineered DN TGF-β Receptor and an anti-PSMA CAR as contemplated herein, including the anti-PSMA CAR-DN TGF-β Receptor constructs described herein, thereby producing a population of modified T cells, and administering the population of modified T cells to the same subject. In embodiments described herein is a method of treating a subject diagnosed with a cancer, comprising removing T cells from the subject, genetically modifying said T cells with a vector comprising a nucleic acid encoding an engineered DN TGF-β Receptor and an anti-PSMA CAR as contemplated herein, including the anti-PSMA CAR-DN TGF-β Receptor constructs described herein, thereby producing a population of modified T cells, and administering the population of modified T cells to the same subject nucleic acid construct encoding an anti-PSCA synNotch molecule.

In certain embodiments, the present disclosure also provides methods for stimulating an effector cell mediated immune modulator response to a target cell population in a subject comprising the steps of administering to the subject an immune effector cell population expressing a nucleic acid construct encoding an engineered DN TGF-β Receptor and an anti-PSMA CAR molecule, including the CAR-DN TGF-β Receptor constructs described herein. In some example the immune effector cell population further express a nucleic acid construct encoding an anti-PSCA synNotch molecule.

The methods for administering the cell compositions described herein includes any method which is effective to result in reintroduction of ex vivo genetically modified immune effector cells that either directly express an engineered CAR in the subject or on reintroduction of the genetically modified progenitors of immune effector cells that on introduction into a subject differentiate into mature immune effector cells that express the engineered CAR, engineered DN TGF-Receptor and an anti-PSMA CAR molecule, including the CAR-DN TGF-β Receptor constructs described herein. In some example the immune effector cell population further express a nucleic acid construct encoding an anti-PSCA synNotch molecule. One method comprises transducing peripheral blood T cells ex vivo with a nucleic acid construct in accordance with the present disclosure and returning the transduced cells into the subject.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

### EXAMPLES

### Example 1

CD3⁺ cells obtained from ALLCells® (Alameda, California) were isolated from peripheral blood mononuclear cells obtained from healthy donors and frozen down in CryoStor® cell cryopreservation media (Sigma Aldrich®). Pan CD3+ T cells were isolated from leukopaks containing peripheral blood mononuclear cells (PBMCs) by negative selection using a commercially available kit from STEMCELL Technologies™ (Vancouver, Canada) according to manufacturer's directions and then frozen in liquid nitrogen. Chimeric antigen receptor (CAR) T cells were generated from frozen human Pan CD3+ T cells. Before lentivirus transduction, the CD3+ pan T cells were thawed, and activated ex vivo using anti-CD3/CD28 Dynabeads®. (ThermoFisher Scientific) and 100 IU/ml exogenous interleukin-2 (IL-2) according to manufacturer recommendations. The activated cells were rested overnight. One day after anti-CD3/CD28 bead activation, T cells were plated and transduced with a lentiviral vector (with anti-PSMA CAR). Canonical CARs (CARs without synNotch receptor transcriptional activation) were transduced with a multiplicity of infection (MOI) of 6-9. Synthetic notch (synNotch) constructs were dually transduced with a synNotch lentivirus vector MOI of 6-9 and a CAR payload lentivirus vector MOI of 6-9. T cells were de-beaded 3 days after transduction and evaluated for expression and cytotoxicity assays between days 8 and 11 following transduction.

Canonicals CAR vectors included an EF1a promoter to drive constitutive expression of the anti-PSMA CAR. synNotch vectors included an EF1a promoter to drive constitutive expression of an anti-PSCA synNotch receptor. The CAR payload vectors (for synNotch receptor activated conditional expression) included 5× gal4 binding sites-minimal CMV promoter to drive conditional expression of the anti-PSMA CAR. synNotch constructs were transduced with synNotch and CAR payload vectors simultaneously as described above.

The anti-PSMA CARs used in the following examples included a CSF2RA signal sequence, an anti-PSMA scFv, CD8α hinge domain, an CD8α transmembrane domain, a 4-1BB co-stimulatory domain, and a CD3zeta signaling domain. The synNotch receptor included an anti-PSCA scFv, a murine Notch core, a gal4 DNA binding domain, and a vp64 transactivation domain. A myc tag was included to simulate ligand induced activation of the synNotch receptors.

Human prostate cell line 22RV1 and human leukemia cell line K562 were obtained from the American Type Culture Collection (ATCC). Cells were cultured at 37° C., 5% CO₂, in either Roswell Park Memorial Institute (RPMI; 22RV1) or Iscove's Modified Dulbecco's Medium (IMDM; K562), supplemented with 1% penicillin/streptomycin and 10% fetal bovine serum (FBS). 22RV1 and K562 cells were engineered to knockout (KO) or overexpress PSCA and PSMA as described in Table 10.

TABLE 10

| Cell Line | PSCA | PSMA |
|---|---|---|
| 22RV1 | − | + |
| 22RV1-PSMA KO | − | − |
| 22RV1-PSMA KO-PSCA | + | − |
| 22RV1-PSCA | + | + |
| K562 | − | − |
| K562-PSCA | + | − |
| K562-PSMA | − | + |
| K562-PSCA-PSMA | + | + |

### Example 2

Seven days after transduction, T cells transduced as described in example 1 were harvested to determine transduction efficiencies. 1.5×10⁵ T cells were plated in 96 well plates in duplicate. For canonical CAR T cells, T cells were cultured overnight in hTCM (human T cell media), that included of x-vivo 15 (Lonza (Basel, Switzerland)), 5% human serum (Valley Biomedical (Winchester, Virginia)), and 1% Glutamax (Gibco). For synNotch T receptor expressing cells, the T cells were cultured in the presence and absence of myc beads overnight to stimulate the synNotch receptor and thereby induce CAR expression. After overnight culture, CAR expression was measured using recombinant PSMA (rPSMA) containing a 6×His tag (SEQ ID NO: 260). The cells were stained with an anti-His antibody and the stained cells were detected via flow cytometry. Only cells bound by the rPSMA (and anti-His) are detected to measure expression. Table 11 shows canonical CAR expression. Table 12 shows synNotch and CAR payload expression.

TABLE 11

| Donor ID | Construct | scFv | % CAR+ | |
|---|---|---|---|---|
| #1 | 104 | Ab4 | 91.5% | 90.0% |
| | 101 | Ab1 | 95.4% | 95.4% |
| | 103 | Ab3 | 90.0% | 91.0% |
| | 105 | Ab5 | 84.4% | 83.9% |
| | UTD | | 0.9% | 1.1% |
| #2 | 104 | Ab4 | 90.7% | 92.0% |
| | 101 | Ab1 | 95.6% | 96.3% |
| | 103 | Ab3 | 93.2% | 92.7% |
| | 105 | Ab5 | 88.4% | 88.1% |
| | UTD | | 0.9% | 1.0% |
| #3 | 104 | Ab4 | 91.0% | 92.1% |
| | 101 | Ab1 | 95.6% | 96.5% |
| | 103 | Ab3 | 88.4% | 87.9% |
| | 105 | Ab5 | 83.2% | 83.6% |
| | UTD | | 0.7% | 0.7% |

TABLE 12

| Donor | | | | | % CAR+ | | % CAR+ | |
|---|---|---|---|---|---|---|---|---|
| ID | Construct | scFv | % synNotch | | (Unstimulated) | | (Stimulated) | |
| #1 | Ab6 | Ab1 | 61.9% | 61.6% | 3.7% | 3.7% | 41.5% | 41.9% |
| | SynNotch/201 | | | | | | | |
| | Ab6 | Ab2 | 71.1% | 71.2% | 1.2% | 1.5% | 26.1% | 24.2% |
| | SynNotch/202 | | | | | | | |
| | Ab6 | Ab3 | 64.7% | 65.0% | 3.2% | 3.2% | 50.5% | 50.3% |
| | SynNotch/203 | | | | | | | |
| | Ab6 | Ab5 | 59.8% | 59.6% | 2.5% | 2.8% | 52.4% | 52.6% |
| | SynNotch/205 | | | | | | | |
| | UTD | | 1.0% | 1.0% | 0.9% | 1.1% | 0.6% | 0.8% |
| #2 | Ab6 | Ab1 | 59.3% | 58.8% | 6.5% | 6.1% | 49.1% | 51.8% |
| | SynNotch/201 | | | | | | | |
| | Ab6 | Ab2 | 67.5% | 67.4% | 1.4% | 1.6% | 24.5% | 24.6% |
| | SynNotch/202 | | | | | | | |
| | Ab6 | Ab3 | 62.8% | 62.5% | 5.6% | 5.5% | 57.5% | 57.4% |
| | SynNotch/203 | | | | | | | |
| | Ab6 | Ab5 | 58.7% | 56.8% | 4.8% | 4.2% | 58.7% | 60.5% |
| | SynNotch/205 | | | | | | | |
| | UTD | | 0.8% | 0.9% | 0.9% | 1.0% | 0.9% | 1.3% |
| #3 | Ab6 | Ab1 | 66.7% | 66.5% | 6.2% | 6.2% | 44.0% | 43.3% |
| | SynNotch/201 | | | | | | | |
| | Ab6 | Ab2 | 77.7% | 77.8% | 1.4% | 1.4% | 42.5% | 40.9% |
| | SynNotch/202 | | | | | | | |
| | Ab6 | Ab3 | 70.0% | 70.4% | 4.6% | 4.6% | 52.3% | 51.5% |
| | SynNotch/203 | | | | | | | |
| | Ab6 | Ab5 | 66.5% | 66.3% | 5.5% | 5.3% | 57.1% | 56.9% |
| | SynNotch/205 | | | | | | | |
| | UTD | | 1.1% | 1.0% | 0.7% | 0.7% | 1.1% | 1.1% |

### Example 3

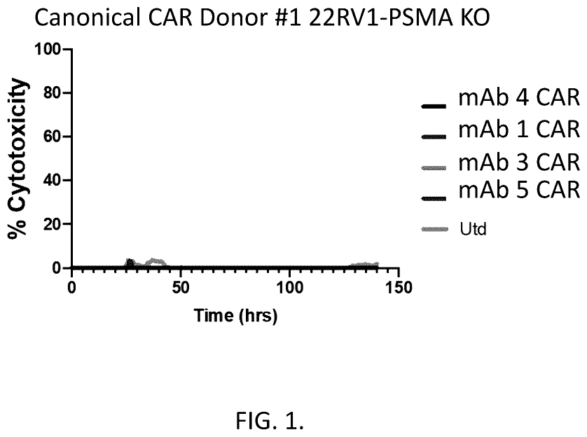

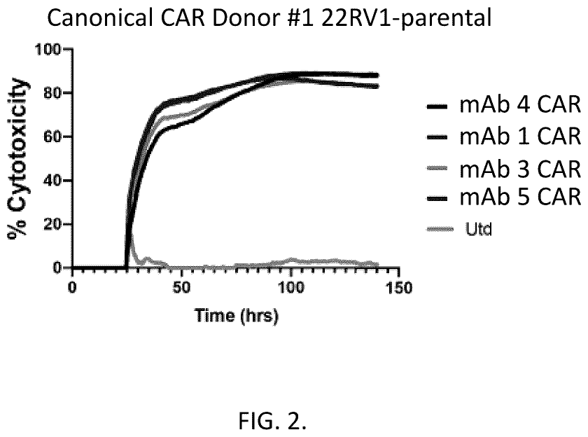

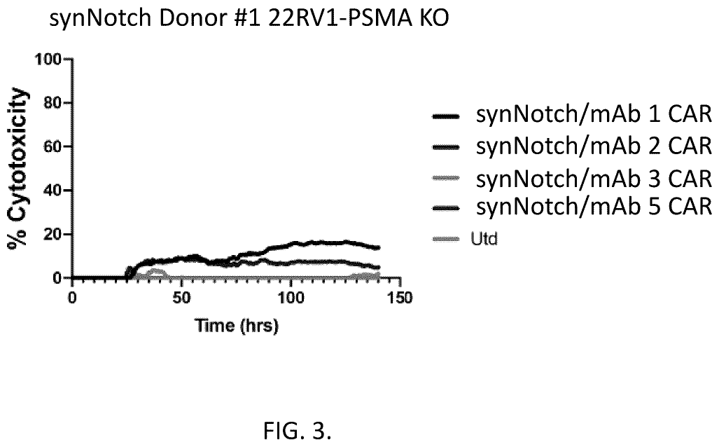

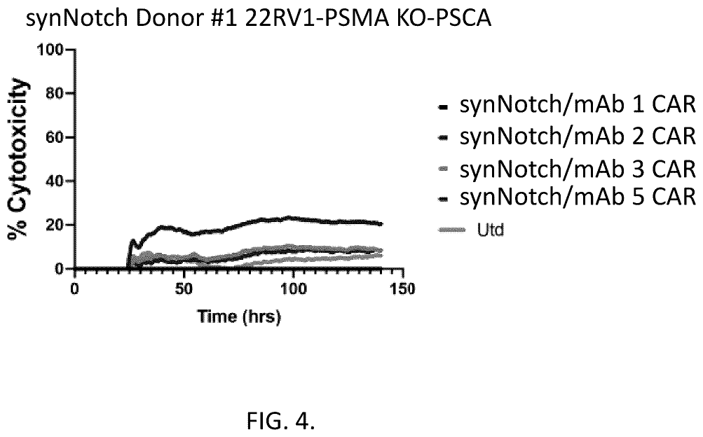

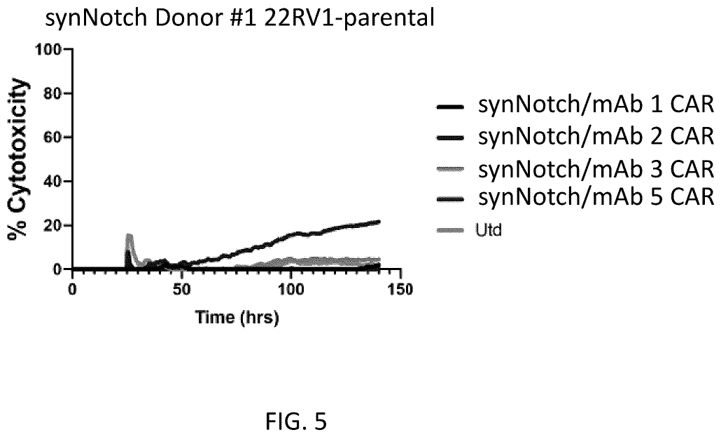

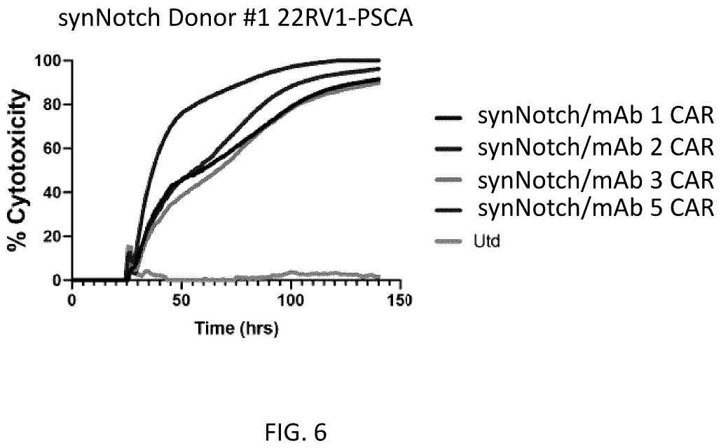

For T cell-dependent cytotoxicity assays, T cells cytotoxicity of T cells transduce as described in example 1 was measured in real time at an effector (T cell)-to-target cell (as described in Table 10) ratio (E: T) of 1:1. Target cells were plated on xCELLigence plates overnight with impedance measured every 15 min. Briefly, 50 μl of hTCM is added to the xCELLigence plate and a measurement is taken to create a baseline for the plate. Then 3×10⁴ of each of the 4 different 22RV1 cell lines were plated on xCELLigence plates in 50 μl and impedance was measured every 15 min overnight (16-18 hrs). Lastly, 3×10⁴ CAR+ effectors are added in 100 μl and impedance is measured every 15 min for an additional 72-96 hrs. The instrument takes each of the readings, called “Cell Index” and calculates a “% Cytotoxicity” based on how the Tumor cells alone have grown on the same plate. Target cells for canonical CAR T cells were 22RV1-PSMA knockout (KO) (FIG. 1) and 22RV1 (FIG. 2). Target cells for synNotch T cells were 22RV1-PSMA KO (FIG. 3), 22RV1-PSMA KO-PSCA (FIG. 4), 22RV1 (FIG. 5), and 22RV1-PSCA (FIG. 6).

### Example 4

As described in Table 10 5×10⁴ target cells were co-cultured with 5×10⁴ CAR+ T cells in a 96 well plate in hTCM media in a total volume of 200 μl for 72 hrs. Supernatants were then harvested and cytokine release (IFNγ and IL-2) was determined via Ella with IFNy or IL-2 cartridges per manufactures recommendations (Protein Simple, San Jose CA).

TABLE 13
IFNγ release from canonical CARs co-cultured with 22RV1 cells.

| DONER #1: IFNγ | 4211 | 4212 | 4215 | 1550 | UTD |
|---|---|---|---|---|---|
| 22RV1-PSMA KO | 11.1 | 12.4 | 5.89 | 3.62 | 5.69 |
| | 7.31 | 20.5 | 6.06 | 118 | 6.48 |
| 22RV1 | 3919 | 2053 | 1132 | 899 | 6.28 |
| | 3678 | 1963 | 1364 | 820 | 5.65 |
| 22RV1-PSCA | 4699 | 2548 | 1673 | 1160 | 7.28 |
| | 3857 | 1973 | 1597 | 1103 | 6.6 |
| 22RV1-PSMA KO-PSCA | 9.08 | 11.7 | 4.51 | 1.01 | 5.39 |
| | 11.5 | 16 | 5.45 | 3.44 | 6.26 |

TABLE 14
IL-2 release from canonical CARs co-cultured with 22RV1 cells.

| DONER #1: IL-2 | 4211 | 4212 | 4215 | 1550 | UTD |
|---|---|---|---|---|---|
| 22RV1-PSMA KO | 3.99 | 0.455 | 2.22 | 0.529 | 2.62 |
| | 0.822 | 25.6 | 0 | 0.549 | 0.099 |
| 22RV1 | 12.6 | 7.18 | 6.53 | 6.18 | 2.06 |
| | 14.3 | 6.11 | 8.97 | 4.7 | 0.993 |
| 22RV1-PSCA | 13.8 | 7.14 | 8.69 | 7.98 | 10.2 |
| | 13.5 | 8.96 | 7.01 | 7.33 | 1.09 |
| 22RV1-PSMA KO- | 2.27 | 0.087 | 0.96 | 0.123 | 0.404 |
| PSCA | 1.95 | 0.35 | 0.567 | 0.621 | 0.576 |

TABLE 15
IFNγ release from canonical CARs co-cultured with K562 cells.

| DONER #1: IFNγ | 4211 | 4212 | 4215 | 1550 | UTD |
|---|---|---|---|---|---|
| K562 | 3.35 | 4.2 | 3.12 | 1.28 | 2.15 |
| | 3.15 | 4.78 | 2.29 | 2.55 | 2.6 |
| K562-PSCA | 2.39 | 5.13 | 0.832 | 1.24 | 1.3 |
| | 3.19 | 4.86 | 1.4 | 0.047 | 0.536 |
| K562-PSMA | 21043 | 13396 | 12236 | 13666 | 1.16 |
| | 19426 | 18943 | 10892 | 15000 | 1.61 |
| K562-PSCA-PSMA | 22344 | 16388 | 15661 | 16464 | 5.85 |
| | 28231 | 25309 | 16407 | 22367 | 3.19 |

TABLE 16
IL-2 release from canonical CARs co-cultured with K562 cells.

| DONER #1: IL-2 | 4211 | 4212 | 4215 | 1550 | UTD |
|---|---|---|---|---|---|
| K562 | 1.63 | 0.531 | 1.36 | 0.555 | 0.691 |
| | 0 | 0.448 | 7.33 | 0.58 | 0.644 |
| K562-PSCA | 0.822 | 0.384 | 1.42 | 0 | 0.273 |
| | 0 | 0 | 0 | 0.062 | 0 |
| K562-PSMA | 16661 | 17413 | 25446 | 21569 | 0 |
| | 20680 | 21031 | 25759 | 23822 | 0 |
| K562-PSCA-PSMA | 23093 | 20216 | 29370 | 28190 | 0.063 |
| | 24756 | 22018 | 33154 | 25095 | 0.028 |

TABLE 17
IFNγ release from synNotch T cells co-cultured with 22RV1 cells.

| DONER #1: IFNγ | 1912/4204 | 1912/4206 | 1912/4207 | 1912/4209 | UTD |
|---|---|---|---|---|---|
| 22RV1-PSMA KO | 1.74 | 6.31 | 2.89 | 2.47 | 5.69 |
| | 2.97 | 6.13 | 2.61 | 3.16 | 6.48 |
| 22RV1 | 31.6 | 48.7 | 21.4 | 18.3 | 6.28 |
| | 13.2 | 84.9 | 21.1 | 18.8 | 5.65 |
| 22RV1-PSCA | 13081 | 16970 | 11493 | 11273 | 7.28 |
| | 14236 | 12507 | 11823 | 8820 | 6.6 |
| 22RV1-PSMA KO-PSCA | 11 | 37.8 | 7.31 | 5.11 | 5.39 |
| | 11.4 | 30.5 | 6.57 | 4.78 | 6.26 |

TABLE 18
IL-2 release from synNotch T cells co-cultured with 22RV1 cells.

| DONER #1: IL-2 | 1912/4204 | 1912/4206 | 1912/4207 | 1912/4209 | UTD |
|---|---|---|---|---|---|
| 22RV1-PSMA KO | 5.08 | 0.638 | 3.53 | 0 | 2.62 |
| | 1.86 | 1.51 | 1.11 | 0 | 0.099 |
| 22RV1 | 27.4 | 32.7 | 31.4 | 26.3 | 2.06 |
| | 20 | 27.2 | 27.3 | 32.8 | 0.993 |
| 22RV1-PSCA | 2204 | 1022 | 937 | 715 | 10.2 |
| | 2544 | 966 | 835 | 829 | 1.09 |
| 22RV1-PSMA KO-PSCA | 24.7 | 9.39 | 4.94 | 10.7 | 0.404 |
| | 23.7 | 2.64 | 2.33 | 10.3 | 0.576 |

TABLE 19
IFNγ release from synNotch T cells co-cultured with K562 cells.

| DONER | | | | | |
|---|---|---|---|---|---|
| #1: IFNγ | 1912/4204 | 1912/4206 | 1912/4207 | 1912/4209 | UTD |
| K562 | 1.93 | 2.4 | 1.99 | 0.317 | 2.15 |
| | 1.03 | 5.36 | 1.35 | 2.66 | 2.6 |
| K562-PSCA | 7.83 | 41.1 | 4.31 | 0 | 1.3 |
| | 8.63 | 45.3 | 3.15 | 4.16 | 0.536 |
| K562-PSMA | 31.2 | 17.5 | 18.2 | 2.56 | 1.16 |
| | 28.9 | 32.4 | 15.6 | 10 | 1.61 |
| K562-PSCA- | 39889 | 37494 | 38511 | 29710 | 5.85 |
| PSMA | 40127 | 47355 | 42126 | 35489 | 3.19 |

TABLE 20
IL-2 release from synNotch T cells co-cultured with K562 cells.

| DONER | | | | | |
|---|---|---|---|---|---|
| #1: IL-2 | 1912/4204 | 1912/4206 | 1912/4207 | 1912/4209 | UTD |
| K562 | 1.59 | 1.54 | 2.16 | 0 | 0.691 |
| | 1.16 | 0.655 | 0.355 | 0.615 | 0.644 |
| K562-PSCA | 3.22 | 0.819 | 3.02 | 2.37 | 0.273 |
| | 5.65 | 0.187 | 0.45 | 2.23 | 0 |
| K562-PSMA | 9.42 | 8.49 | 9.77 | 2.5 | 0 |
| | 5.22 | 5.99 | 7.43 | 4.26 | 0 |
| K562-PSCA- | 44348 | 85959 | 29228 | 37515 | 0.063 |
| PSMA | 35870 | 55799 | 25689 | 25276 | 0.028 |

TABLE 21
IFNγ release from canonical CARs co-cultured with 22RV1 cells.

| DONER #2: IFNγ | 4211 | 4212 | 4215 | 1550 | UTD |
|---|---|---|---|---|---|
| 22RV1-PSMA KO | 16 | 15.3 | 5.81 | 2.19 | 0.787 |
| | 14.5 | 19.5 | 4.77 | 2.21 | 4.4 |
| 22RV1 | 4537 | 4056 | 3063 | 1891 | 2.19 |
| | 4417 | 3047 | 2301 | 1408 | 1.38 |
| 22RV1-PSCA | 4928 | 4636 | 2657 | 2080 | 1.44 |
| | 5795 | 3771 | 3106 | 1993 | 0.591 |
| 22RV1-PSMA KO-PSCA | 13.2 | 15.8 | 5.5 | 0.645 | 1.3 |
| | 11.9 | 9.08 | 4.42 | 0.933 | 1.53 |

TABLE 22
IL-2 release from canonical CARs co-cultured with 22RV1 cells.

| DONER | | | | | |
|---|---|---|---|---|---|
| #2: IL-2 | 4211 | 4212 | 4215 | 1550 | UTD |
| 22RV1-PSMA | 3.07 | 1.79 | 2.32 | 0 | 0.655 |
| KO | 0.192 | 0 | 1.78 | 3.1 | 1.6 |
| 22RV1 | 9.61 | 4.28 | 6.86 | 7.59 | 0.956 |
| | 7.49 | 3.96 | 3.97 | 0.849 | 0.704 |
| 22RV1-PSCA | 8.1 | 3.93 | 6.32 | 5 | 0.486 |
| | 8.81 | 3.15 | 3.92 | 4.08 | 0.501 |
| 22RV1-PSMA | 0.143 | 1.4 | 2.57 | 0 | 0.481 |
| KO-PSCA | 0.427 | 0 | 1.55 | 0 | 0 |

TABLE 23
IFNγ release from Canonical CARs co-cultured with K562 cells.

| DONER #2: IFNγ | 4211 | 4212 | 4215 | 1550 | UTD |
|---|---|---|---|---|---|
| K562 | 11 | 10.2 | 3.68 | 4.93 | 1.06 |
| | 11.1 | 7.76 | 5.76 | 4.7 | 2.95 |
| K562-PSCA | 12.8 | 11.4 | 7.68 | 4.95 | 2.74 |
| | 11.3 | 8.26 | 3.69 | 2.04 | 2.33 |
| K562-PSMA | 29454 | 32708 | 29759 | 25791 | 2.63 |
| | 27437 | 26993 | 29358 | 25893 | 1.85 |
| K562-PSCA- | 38792 | 37657 | 25578 | 36122 | 3.21 |
| PSMA | 33466 | 28020 | 34273 | 33250 | 1.63 |

TABLE 24
IL-2 release from Canonical CARs co-cultured with K562 cells.

| DONER #2: IL-2 | 4211 | 4212 | 4215 | 1550 | UTD |
|---|---|---|---|---|---|
| K562 | 0 | 0.342 | 0.416 | 1.06 | 0.113 |
| | 0 | 0 | 0.146 | 0 | 0 |
| K562-PSCA | 0.108 | 0 | 0.498 | 0.162 | 0 |
| | 0.041 | 0 | 0 | 0.578 | 0 |
| K562-PSMA | 23311 | 17945 | 29384 | 32289 | 0.336 |
| | 23012 | 18898 | 32330 | 35611 | 0 |
| K562-PSCA- | 30215 | 29211 | 40977 | 43314 | 0 |
| PSMA | 26986 | 24065 | 43911 | 47153 | 0.016 |

TABLE 25
IFNγ release from synNotch T cells co-cultured with 22RV1 cells.

| DONER | | | | | |
|---|---|---|---|---|---|
| #2: IFNγ | 1912/4204 | 1912/4206 | 1912/4207 | 1912/4209 | UTD |
| 22RV1-PSMA | 1.56 | 13.6 | 2.94 | 2.22 | 0.787 |
| KO | 2.75 | 16.2 | 3.96 | 1.62 | 4.4 |
| 22RV1 | 68.9 | 101 | 42.7 | 21.4 | 2.19 |
| | 50.4 | 66.6 | 37.5 | 22.4 | 1.38 |
| 22RV1-PSCA | 16212 | 15199 | 12526 | 8831 | 1.44 |
| | 15016 | 17913 | 13830 | 9328 | 0.591 |
| 22RV1-PSMA | 27.4 | 84.1 | 16 | 4.5 | 1.3 |
| KO-PSCA | 24.6 | 117 | 14.7 | 6.81 | 1.53 |

TABLE 26
IL-2 release from synNotch T cells co-cultured with 22RV1 cells.

| DONER | | | | | |
|---|---|---|---|---|---|
| #2: IL-2 | 1912/4204 | 1912/4206 | 1912/4207 | 1912/4209 | UTD |
| 22RV1-PSMA | 0.368 | 0 | 1.92 | 0.506 | 0.655 |
| KO | 0.121 | 0.748 | 0.134 | 0.709 | 1.6 |
| 22RV1 | 34.8 | 20 | 26.3 | 17 | 0.956 |
| | 20.4 | 9.11 | 16.9 | 12 | 0.704 |
| 22RV1-PSCA | 1241 | 94.6 | 287 | 440 | 0.486 |
| | 1307 | 115 | 258 | 556 | 0.501 |
| 22RV1-PSMA | 24.6 | 3.84 | 2.11 | 3.46 | 0.481 |
| KO-PSCA | 12.3 | 2.01 | 3.11 | 3.57 | 0 |

TABLE 27
IFNγ release from synNotch T cells co-cultured with K562 cells.

| DONER | | | | | |
|---|---|---|---|---|---|
| #2: IFNγ | 1912/4204 | 1912/4206 | 1912/4207 | 1912/4209 | UTD |
| K562 | 1.93 | 11.9 | 3.58 | 2.4 | 1.06 |
| | 0.916 | 11.7 | 2.52 | 1.71 | 2.95 |
| K562-PSCA | 13.8 | 230 | 10.2 | 11.8 | 2.74 |
| | 7.68 | 213 | 8.92 | 9.95 | 2.33 |
| K562-PSMA | 37.7 | 63.4 | 92.1 | 11.6 | 2.63 |
| | 48.4 | 38.7 | 43 | 12.7 | 1.85 |
| K562-PSCA- | 36941 | 49398 | 49600 | 40575 | 3.21 |
| PSMA | 43708 | 43476 | 49110 | 36315 | 1.63 |

TABLE 28
IL-2 release from synNotch T cells co-cultured with K562 cells.

| DONER | | | | | |
|---|---|---|---|---|---|
| #2: IL-2 | 1912/4204 | 1912/4206 | 1912/4207 | 1912/4209 | UTD |
| K562 | 0 | 0 | 0.618 | 0 | 0.113 |
| | 0 | 2.03 | 0 | 0.104 | 0 |
| K562-PSCA | 2.28 | 0.907 | 1.19 | 4.97 | 0 |
| | 0 | 0.522 | 1.11 | 0 | 0 |
| K562-PSMA | 9.73 | 10.1 | 12.4 | 15.7 | 0.336 |
| | 9.4 | 5.04 | 6.94 | 6.17 | 0 |
| K562-PSCA- | 56844 | 53309 | 41059 | 33992 | 0 |
| PSMA | 56812 | 56739 | 28879 | 26926 | 0.016 |

TABLE 29
IFNγ release from Canonical CARs co-cultured with 22RV1 cells.

| DONER #3: IFNγ | 4211 | 4212 | 4215 | 1550 | UTD |
|---|---|---|---|---|---|
| 22RV1-PSMA KO | 15.7 | 31.8 | 2.89 | 0 | 2.91 |
| | 14.9 | 23.1 | 3.11 | 0 | 3.52 |
| 22RV1 | 963 | 1087 | 333 | 266 | 8.43 |
| | 1095 | 1233 | 406 | 248 | 3.77 |
| 22RV1-PSCA | 1453 | 1509 | 392 | 313 | 5.41 |
| | 1352 | 1270 | 431 | 299 | 2.42 |
| 22RV1-PSMA KO- | 14.4 | 42.4 | 1.19 | 0 | 2.15 |
| PSCA | 12.1 | 25.6 | 1.31 | 0 | 2.67 |

TABLE 30
IL-2 release Canonical CARs co-cultured with 22RV1 cells.

| DONER #3: IL-2 | 4211 | 4212 | 4215 | 1550 | UTD |
|---|---|---|---|---|---|
| 22RV1-PSMA | 1.93 | 3.46 | 0.137 | 0.13 | 0 |
| KO | 0 | 3.64 | 0 | 1.35 | 1.16 |
| 22RV1 | 7.01 | 6.67 | 4.83 | 6.16 | 21.4 |
| | 9.46 | 5.1 | 3.25 | 4.59 | 6.83 |
| 22RV1-PSCA | 6.85 | 2.77 | 3.55 | 4.78 | 8.2 |
| | 9.16 | 2.82 | 2.74 | 4.36 | 1.88 |
| 22RV1-PSMA | 0 | 0.907 | 2.11 | 0 | 0 |
| KO-PSCA | 0.022 | 0.465 | 0.147 | 0 | 0.948 |

TABLE 31
IFNγ release from Canonical CARs co-cultured with K562 cells.

| DONER #3: IFNγ | 4211 | 4212 | 4215 | 1550 | UTD |
|---|---|---|---|---|---|
| K562 | 3.95 | 12.1 | 2.44 | 0.122 | 1.74 |
| | 7.59 | 10.9 | 2.57 | 1.56 | 2.7 |
| K562-PSCA | 5.17 | 20 | 1.64 | 2.16 | 1.14 |
| | 4.76 | 24.4 | 0 | 0.684 | 1.43 |
| K562-PSMA | 20686 | 23162 | 10258 | 13894 | 37.9 |
| | 24434 | 22309 | 14384 | 19840 | 33.7 |
| K562-PSCA- | 26446 | 18179 | 18075 | 17588 | 50.9 |
| PSMA | 21796 | 27232 | 15897 | 17508 | 52.4 |

TABLE 32
IL-2 release from synNotch T cells co-cultured with K562 cells.

| DONER #3: IL-2 | 4211 | 4212 | 4215 | 1550 | UTD |
|---|---|---|---|---|---|
| K562 | 4.15 | 5.44 | 5.44 | 5.65 | 8.79 |
| | 3.01 | 5.5 | 4.1 | 5.66 | 4.97 |
| K562-PSCA | 3.84 | 6.64 | 3.18 | 6.29 | 5.75 |
| | 3.84 | 4.89 | 2.78 | 4.32 | 4.46 |
| K562-PSMA | 16547 | 11304 | 17363 | 14695 | 16.7 |
| | 15454 | 12690 | 17380 | 16003 | 27.4 |
| K562-PSCA-PSMA | 22756 | 17217 | 29446 | 21885 | 29.9 |
| | 19618 | 16775 | 22623 | 21235 | 32.5 |

TABLE 33
IFNγ release from synNotch T cells co-cultured with 22RV1 cells.

| DONER | | | | | |
|---|---|---|---|---|---|
| #3: IFNγ | 1912/4204 | 1912/4206 | 1912/4207 | 1912/4209 | UTD |
| 22RV1-PSMA | 1.85 | 2.4 | 0.691 | 2.57 | 2.91 |
| KO | 1.97 | 1.52 | 2.05 | 0.436 | 3.52 |
| 22RV1 | 33.2 | 58.6 | 26.4 | 12.8 | 8.43 |
| | 16.6 | 47.4 | 24 | 10.9 | 3.77 |
| 22RV1-PSCA | 6124 | 9040 | 8200 | 5535 | 5.41 |
| | 6378 | 9062 | 6616 | 5078 | 2.42 |
| 22RV1-PSMA | | 49.2 | 6.68 | 20.5 | 2.15 |
| KO-PSCA | 38.3 | 36.8 | 11.3 | 15.6 | 2.67 |

TABLE 34
IL-2 release from synNotch T cells co-cultured with 22RV1 cells.

| DONER | | | | | |
|---|---|---|---|---|---|
| #3: IL-2 | 1912/4204 | 1912/4206 | 1912/4207 | 1912/4209 | UTD |
| 22RV1-PSMA | 1.39 | 0 | 1.4 | 1.25 | 0 |
| KO | 0.439 | 0 | 0 | 0.338 | 1.16 |
| 22RV1 | 44.1 | 43.8 | 45.3 | 10.4 | 21.4 |
| | 24.8 | 40 | 39.2 | 17.6 | 6.83 |
| 22RV1-PSCA | 1031 | 1179 | 503 | 225 | 8.2 |
| | 1120 | 1243 | 609 | 323 | 1.88 |
| 22RV1-PSMA | 93 | 13.7 | 12.2 | 45.9 | 0 |
| KO-PSCA | 71.4 | 10.1 | 12.1 | 40.3 | 0.948 |

TABLE 35
IFNγ release from synNotch T cells co-cultured with K562 cells.

| DONER | | | | | |
|---|---|---|---|---|---|
| #3: IFNγ | 1912/4204 | 1912/4206 | 1912/4207 | 1912/4209 | UTD |
| K562 | 1.64 | 3.65 | 1.73 | 0.13 | 1.74 |
| | 1.44 | 3.53 | 0.594 | 0.654 | 2.7 |
| K562-PSCA | 16.9 | 59.4 | 10.7 | 3.56 | 1.14 |
| | 38.1 | 75.6 | 7.93 | 10.6 | 1.43 |
| K562-PSMA | 18.5 | 14.7 | 20.3 | 3.97 | 37.9 |
| | 34.2 | 10.5 | 23.1 | 2.94 | 33.7 |
| K562-PSCA- | 33444 | 46610 | 38118 | 25258 | 50.9 |
| PSMA | 31062 | 37241 | 30922 | 25004 | 52.4 |

TABLE 36
IL-2 release from synNotch T cells co-cultured with K562 cells.

| DONER | | | | | |
|---|---|---|---|---|---|
| #3: IL-2 | 1912/4204 | 1912/4206 | 1912/4207 | 1912/4209 | UTD |
| K562 | 5.42 | 4.53 | 4.4 | 4.98 | 8.79 |
| | 4.72 | 4.39 | 5.03 | 4.97 | 4.97 |
| K562-PSCA | 18.1 | 8.88 | 13.9 | 9.25 | 5.75 |
| | 21.1 | 6.29 | 11.7 | 12.9 | 4.46 |
| K562-PSMA | 12.3 | 17.5 | 17.5 | 13.3 | 16.7 |
| | 14.7 | 16.1 | 13.1 | 8.45 | 27.4 |
| K562-PSCA- | 35691 | 57642 | 28190 | 33381 | 29.9 |
| PSMA | 33075 | 44658 | 22130 | 26937 | 32.5 |

### Example 5

22RV1 or mitomycin C treated-K562 cells were co-cultured with Cell Trace Violet (CTV) labeled-T cells in a 96 well plate for 96 hrs. Following incubation. plates were analyzed via flow cytometry for dilution of CTV.

TABLE 37
% Divided canonical CAR T cells following K562 co-culture.

| | | | | | | K562- | T |
|---|---|---|---|---|---|---|---|
| Donor | | | | K562- | K562- | PSCA- | cells |
| ID | Construct | scFv | K562 | PSCA | PSMA | PSMA | only |
| #1 | 104 | Ab4 | 1.54 | 1.62 | 87.9 | 89.15 | 1.86 |
| | 101 | Ab1 | 2.73 | 3.41 | 93.85 | 94.5 | 4.26 |
| | 103 | Ab3 | 1.99 | 2.23 | 92.5 | 93.85 | 3.1 |
| | 105 | Ab5 | 4.34 | 5.12 | 88.95 | 87.1 | 6.19 |
| | UTD | | | | | | |
| #2 | 104 | Ab4 | 0.28 | 0.56 | 89.1 | 90.45 | 0.41 |
| | 101 | Ab1 | 0.58 | 0.47 | 91 | 93.3 | 0.54 |
| | 103 | Ab3 | 0.31 | 0.92 | 90.95 | 92.95 | 0.49 |
| | 105 | Ab5 | 0.17 | 1.3 | 92.65 | 93.55 | 0.15 |
| | UTD | | 5.83 | 3.01 | 7.35 | 5.91 | 8.31 |
| #3 | 104 | Ab4 | 0.5 | 0.77 | 93.4 | 94.8 | 2.21 |
| | 101 | Ab1 | 1.47 | 1.22 | 92.9 | 94 | 4.07 |
| | 103 | Ab3 | 0.96 | 1.36 | 95.6 | 96.6 | 3.11 |
| | 105 | Ab5 | 1.85 | 2.28 | 96.3 | 96.3 | 7.5 |
| | UTD | | 3.57 | 4.99 | 5.57 | 3.99 | 6.58 |

TABLE 38
% Divided canonical CAR T cells following 22RV1 co-culture.

| | | | 22RV1- | | | 22RV1- | T |
|---|---|---|---|---|---|---|---|
| Donor | | | PSMA | | 22RV1- | PSMA | cells |
| ID | Construct | scFv | KO | 22RV1 | PSCA | KO-PSCA | only |
| #1 | 104 | Ab4 | 0.89 | 89 | 89.3 | 0.67 | 1.86 |
| | 101 | Ab1 | 3.76 | 94.25 | 94.15 | 3.27 | 4.26 |
| | 103 | Ab3 | 1.03 | 87.7 | 89.75 | 0.88 | 3.1 |
| | 105 | Ab5 | 4.63 | 78.1 | 81.35 | 3.72 | 6.19 |
| | UTD | | | | | | |
| #2 | 104 | Ab4 | | 91.55 | 90.75 | 0.14 | 0.41 |
| | 101 | Ab1 | 0.61 | 91.6 | 92.3 | 0.73 | 0.54 |
| | 103 | Ab3 | 0.08 | 90.1 | 90.9 | 0.21 | 0.49 |
| | 105 | Ab5 | 0 | 90.7 | 91.55 | 0.05 | 0.15 |
| | UTD | | 2.98 | 3.08 | 2.95 | 2.33 | 8.31 |
| #3 | 104 | Ab4 | 0.16 | 87.9 | 90.3 | 0.2 | 2.21 |
| | 101 | Ab1 | 0.7 | 91 | 92.95 | 0.47 | 4.07 |
| | 103 | Ab3 | 0.41 | 93.75 | 94.65 | 0.33 | 3.11 |
| | 105 | Ab5 | 2.35 | 84.35 | 87.6 | 1.17 | 7.5 |
| | UTD | | 7.03 | 7.02 | 5.72 | 5.76 | 6.58 |

TABLE 39
% Divided synNotch T cells following K562 co-culture.

| | | | | | | K562- | T |
|---|---|---|---|---|---|---|---|
| Donor | | | | K562- | K562- | PSCA- | cells |
| ID | Construct | scFv | K562 | PSCA | PSMA | PSMA | only |
| #1 | Ab6 | Ab1 | 1.75 | 1.79 | 7.46 | 31.15 | 0.93 |
| | SynNotch/ | | | | | | |
| | 201 | | | | | | |
| | Ab6 | Ab2 | 25.8 | 33.35 | 35.8 | 68.65 | 31.25 |
| | SynNotch/ | | | | | | |
| | 202 | | | | | | |
| | Ab6 | Ab3 | 1.01 | 2.22 | 6.49 | 37.65 | 1.41 |
| | SynNotch/ | | | | | | |
| | 203 | | | | | | |
| | Ab6 | Ab5 | 7.14 | 6.3 | 11.26 | 43.05 | 8.09 |
| | SynNotch/ | | | | | | |
| | 205 | | | | | | |
| | UTD | | | | | | |
| #2 | Ab6 | Ab1 | 1.82 | 3.47 | 7.78 | 16.6 | 0.98 |
| | SynNotch/ | | | | | | |
| | 201 | | | | | | |
| | Ab6 | Ab2 | 29.6 | 31.4 | 40.55 | 59.5 | 35.9 |
| | SynNotch/ | | | | | | |
| | 202 | | | | | | |
| | Ab6 | Ab3 | 0.34 | 2.93 | 7.92 | 15.95 | |
| | SynNotch/ | | | | | | |
| | 203 | | | | | | |
| | Ab6 | Ab5 | 1.62 | 3.12 | 10.34 | 16.15 | 2.49 |
| | SynNotch/ | | | | | | |
| | 205 | | | | | | |
| | UTD | | 5.83 | 3.01 | 7.35 | 5.91 | 8.31 |
| #3 | Ab6 | Ab1 | 3.82 | 17.15 | 12.55 | 56.65 | 5.66 |
| | SynNotch/ | | | | | | |
| | 201 | | | | | | |
| | Ab6 | Ab2 | 6.32 | 26.6 | 17 | 61.35 | 7.45 |
| | SynNotch/ | | | | | | |
| | 202 | | | | | | |
| | Ab6 | Ab3 | 7.47 | 24.74 | 21.95 | 67.85 | 8.34 |
| | SynNotch/ | | | | | | |
| | 203 | | | | | | |
| | Ab6 | Ab5 | 7.67 | 35.9 | 18.45 | 75.65 | 12.3 |
| | SynNotch/ | | | | | | |
| | 205 | | | | | | |
| | UTD | | 3.57 | 4.99 | 5.57 | 3.99 | 6.58 |

TABLE 40
% Divided synNotch T cells following 22RV1 co-culture.

| Donor | | | 22RV1- | | 22RV1- | 22RV1-PSMA | T cells |
|---|---|---|---|---|---|---|---|
| ID | Construct | scFv | PSMA KO | 22RV1 | PSCA | KO-PSCA | only |
| #1 | Ab6 | Ab1 | 0.88 | 1.56 | 28.2 | 1.75 | 0.93 |
| | SynNotch/201 | | | | | | |
| | Ab6 | Ab2 | 29.45 | 31.45 | 77.75 | 23.9 | 31.25 |
| | SynNotch/202 | | | | | | |
| | Ab6 | Ab3 | 1.11 | 2.29 | 48.1 | 1.17 | 1.41 |
| | SynNotch/203 | | | | | | |
| | Ab6 | Ab5 | 2.68 | 3.25 | 52.1 | 2.52 | 8.09 |
| | SynNotch/205 | | | | | | |
| | UTD | | | | | | |
| #2 | Ab6 | Ab1 | | 0.69 | 15.85 | 0.52 | 0.98 |
| | SynNotch/201 | | | | | | |
| | Ab6 | Ab2 | | 18.65 | 47.95 | 16.55 | 35.9 |
| | SynNotch/202 | | | | | | |
| | Ab6 | Ab3 | | 0.42 | 16.8 | 0.59 | |
| | SynNotch/203 | | | | | | |
| | Ab6 | Ab5 | | 1.83 | 22.1 | 0.91 | 2.49 |
| | SynNotch/205 | | | | | | |
| | UTD | | 2.98 | 3.08 | 2.95 | 2.33 | 8.31 |
| #3 | Ab6 | Ab1 | 7.08 | 10.58 | 53.7 | 10.62 | 5.66 |
| | SynNotch/201 | | | | | | |
| | Ab6 | Ab2 | 9.39 | 13.55 | 62.25 | 18.5 | 7.45 |
| | SynNotch/202 | | | | | | |
| | Ab6 | Ab3 | 10.45 | 18.5 | 65.35 | 12.65 | 8.34 |
| | SynNotch/203 | | | | | | |
| | Ab6 | Ab5 | 11.6 | 14.75 | 75.95 | 14.6 | 12.3 |
| | SynNotch/205 | | | | | | |
| | UTD | | 7.03 | 7.02 | 5.72 | 5.76 | 6.58 |

### Example 6

Female NSG mice were implanted with 5×10⁵ K562-PSCA-PSMA subcutaneously in the dorsal flank in 50% Matrigel. On day 14, mice were dosed with either 2×10⁶ or 5×10⁶ CAR+ T cells. Tumors were measured via calipers.

TABLE 41

| Canonical | | | | | | | | |
|---|---|---|---|---|---|---|---|---|
| CAR | | | | | | | | |
| Days post | | | | | | | | |
| tumor | | | | | | | | |
| implantation | | | | | | | | |
| | Vehicle | | | | | | | |
| 14 | 108 | 108 | 108 | 126 | 126 | 126 | 126 | 144 |
| 17 | 365 | 365 | 288 | 405 | 405 | 405 | 405 | 405 |
| 21 | 1183 | 864 | 787 | 847 | 1183 | 1080 | 1008 | 1268 |
| 24 | 1666 | 1470 | 1437 | 1437 | 1666 | 1764 | 1764 | 1862 |
| 27 | 2560 | 2025 | 2138 | 2432 | 2560 | 2890 | 2560 | 2890 |
| | UT | | | | | | | |
| 14 | 108 | 108 | 108 | 126 | 126 | 126 | 126 | 144 |
| 17 | 365 | 196 | 405 | 320 | 405 | 405 | 405 | 405 |
| 21 | 726 | 666 | 864 | 908 | 1183 | 936 | 1099 | 1183 |
| 24 | 1470 | 1183 | 1913 | 1296 | 1913 | 1568 | 1913 | 1666 |
| 27 | | 2025 | 2746 | 2250 | 2560 | | 2746 | 2304 |
| | Ab1-17 canonical 2e6 | | | | | | | |
| 14 | 108 | 108 | 108 | 126 | 126 | 126 | 126 | 144 |
| 17 | 288 | 172 | 172 | 288 | 256 | 288 | 256 | 288 |
| 21 | 726 | 550 | 500 | 1008 | 936 | 847 | 726 | 1099 |
| 24 | 1099 | 936 | 666 | 1268 | 1268 | 1080 | 936 | 1568 |
| 27 | | 1080 | 726 | 1352 | 1521 | 1437 | 1008 | 2025 |
| 30 | | 1008 | 550 | 1352 | 1521 | 1862 | 1268 | |
| 34 | | 726 | 365 | 1080 | 1437 | 1437 | 1268 | |
| 37 | | 550 | 256 | 600 | 1080 | 847 | 936 | |
| 41 | | 500 | 196 | 405 | 726 | 650 | 550 | |
| 44 | | 500 | 196 | 320 | | 405 | 405 | |
| 48 | | 405 | | | | 288 | 365 | |
| 51 | | 288 | | | | 172 | 288 | |
| 55 | | 196 | | | | 108 | 256 | |
| | Ab1-17 canonical 5e6 | | | | | | | |
| 14 | 108 | 108 | 108 | 126 | 126 | 126 | 126 | 144 |
| 17 | 405 | 172 | 256 | 365 | 256 | 320 | 320 | 405 |
| 21 | 936 | 550 | 666 | 864 | 936 | 787 | 847 | 1268 |
| 24 | 1268 | 864 | 936 | 1008 | 1268 | 1080 | 1080 | 1764 |
| 27 | 726 | 550 | 405 | | 1470 | 1080 | 1152 | 1960 |
| 30 | 320 | 365 | 172 | | 1470 | 550 | 1080 | 1666 |
| 34 | 126 | 108 | 63 | | 500 | 365 | 486 | 908 |
| 37 | 32 | 63 | 14 | | 288 | 256 | 486 | 600 |
| 41 | 4 | 14 | 1 | | 288 | 172 | 320 | |
| 44 | 1 | 4 | | | 288 | 172 | | |
| 48 | | 1 | | | | 63 | | |
| 51 | | 1 | | | | 32 | | |
| 55 | | 1 | | | | 32 | | |
| | Ab4 canonical 2e6 | | | | | | | |
| 14 | 108 | 108 | 108 | 126 | 126 | 126 | 126 | 144 |
| 17 | 256 | 256 | 196 | 320 | 320 | 288 | 365 | 500 |
| 21 | 864 | 726 | 600 | 1008 | 908 | 847 | 1008 | 1470 |
| 24 | 1183 | 936 | 726 | 1666 | 1152 | 1080 | 1268 | 1764 |
| 27 | 1568 | 1008 | 936 | | 1224 | 1764 | 2025 | 2432 |
| 30 | 1352 | 1008 | 1183 | | 1606 | 1764 | | |
| 34 | 1666 | 1080 | 1568 | | 1606 | | | |
| 37 | 1666 | 1080 | 1568 | | 1960 | | | |
| 41 | 2025 | 1080 | 1568 | | 2250 | | | |
| 44 | | 1080 | 2025 | | | | | |
| | Ab4 canonical 5e6 | | | | | | | |
| 14 | 108 | 108 | 108 | 126 | 126 | 126 | 126 | 144 |
| 17 | 196 | 288 | 196 | 405 | 320 | 288 | 405 | 320 |
| 21 | 787 | 936 | 666 | 1008 | 936 | 1099 | 1268 | 1008 |
| 24 | 1008 | 1268 | 726 | 1352 | 1268 | 1764 | 1666 | 1352 |
| 27 | 1152 | 1568 | 864 | 1666 | 1268 | 1862 | 2025 | 1764 |
| 30 | 1152 | 1568 | 936 | 1666 | 1268 | 2025 | | 1764 |
| 34 | 1008 | 1183 | 726 | 936 | | | | 1183 |
| 37 | 288 | 446 | 288 | 405 | | | | 550 |
| 41 | 63 | 320 | 172 | 365 | | | | 320 |
| 44 | 4 | 288 | 108 | 288 | | | | |
| 48 | 1 | 172 | | | | | | |
| 51 | | 63 | | | | | | |
| | Ab3-21 canonical 2e6 | | | | | | | |
| 14 | 108 | 108 | 108 | 108 | 126 | 126 | 126 | 144 |
| 17 | 196 | 196 | 196 | 288 | 365 | 288 | 288 | 288 |
| 21 | 666 | 666 | 864 | 1099 | 864 | 1008 | 936 | 847 |
| 24 | 787 | 726 | 1268 | 1568 | 1183 | 1268 | 1470 | 1268 |
| 27 | 1008 | | 1666 | 2304 | 2176 | 2025 | 1913 | 1666 |
| 30 | 1080 | | 2138 | | | | 2304 | 2025 |
| 34 | 1080 | | | | | | | |
| 37 | 1268 | | | | | | | |
| | Ab3-21 canonical 5e6 | | | | | | | |
| 14 | 108 | 108 | 108 | 108 | 126 | 126 | 126 | 144 |
| 17 | 256 | 256 | 256 | 365 | 256 | 320 | 365 | 320 |
| 21 | 666 | 936 | 864 | 1183 | 847 | 1008 | 1099 | 1080 |
| 24 | 1008 | 1183 | 1008 | 1372 | 1268 | 1352 | 1470 | 1666 |
| 27 | 1008 | 1183 | 1183 | 1372 | 1268 | 1437 | 1688 | |
| 30 | 1080 | 1183 | 1470 | 1372 | | 1666 | 2048 | |
| 34 | 1080 | 864 | 1800 | 1099 | | 1352 | | |
| 37 | 847 | 666 | 1800 | | | 1352 | | |
| 41 | 847 | 666 | | | | | | |
| 44 | | 365 | | | | | | |
| 48 | | 288 | | | | | | |
| 51 | | 172 | | | | | | |
| | Ab5 canonical 2e6 | | | | | | | |
| 14 | 108 | 108 | 108 | 108 | 126 | 126 | 126 | 144 |
| 17 | 172 | 172 | 256 | 196 | 405 | 365 | 256 | 288 |
| 21 | 500 | 726 | 726 | 600 | 936 | 1099 | 787 | 1008 |
| 24 | 864 | 1152 | 1183 | 1099 | 1470 | | 1183 | 1470 |
| 27 | 864 | 1352 | 1666 | 1372 | | | 1800 | 1666 |
| 30 | 936 | 1568 | 2025 | 1800 | | | | 2025 |
| 34 | 1008 | 1470 | | 2176 | | | | |
| 37 | 1008 | 1470 | | | | | | |
| 41 | 1268 | 1183 | | | | | | |
| 44 | 2025 | 1183 | | | | | | |
| 48 | | 1183 | | | | | | |
| 51 | | 1183 | | | | | | |
| | Ab5 canonical 5e6 | | | | | | | |
| 14 | 108 | 108 | 108 | 108 | 126 | 126 | 126 | 144 |
| 17 | 196 | 288 | 196 | 256 | 256 | 365 | 405 | 320 |
| 21 | 550 | 726 | 600 | 600 | 864 | 847 | 1008 | 700 |
| 24 | 726 | 936 | 600 | 787 | | 1268 | 1568 | 847 |
| 27 | 500 | 936 | 365 | 550 | | 1352 | | 847 |
| 30 | 365 | 726 | 365 | 365 | | | | 847 |
| 34 | 172 | 320 | 172 | 256 | | | | 288 |
| 37 | 63 | 288 | 63 | 172 | | | | 172 |
| 41 | 63 | 256 | 32 | 172 | | | | 48 |
| 44 | 32 | 108 | 14 | 108 | | | | 32 |
| 48 | 4 | 63 | 4 | 63 | | | | |
| 51 | 4 | 32 | 4 | 14 | | | | |
| 55 | 4 | 4 | 1 | 4 | | | | |

TABLE 41

| synNotch | | | | | | | | |
|---|---|---|---|---|---|---|---|---|
| Days post | | | | | | | | |
| tumor | | | | | | | | |
| implantation | | | | | | | | |
| | Vehicle | | | | | | | |
| 14 | 108 | 108 | 108 | 126 | 126 | 126 | 126 | 144 |
| 17 | 365 | 365 | 288 | 405 | 405 | 405 | 405 | 405 |
| 21 | 1183 | 864 | 787 | 847 | 1183 | 1080 | 1008 | 1268 |
| 24 | 1666 | 1470 | 1437 | 1437 | 1666 | 1764 | 1764 | 1862 |
| 27 | 2560 | 2025 | 2138 | 2432 | 2560 | 2890 | 2560 | 2890 |
| | UT | | | | | | | |
| 14 | 108 | 108 | 108 | 126 | 126 | 126 | 126 | 144 |
| 17 | 365 | 196 | 405 | 320 | 405 | 405 | 405 | 405 |
| 21 | 726 | 666 | 864 | 908 | 1183 | 936 | 1099 | 1183 |
| 24 | 1470 | 1183 | 1913 | 1296 | 1913 | 1568 | 1913 | 1666 |
| 27 | | 2025 | 2746 | 2250 | 2560 | | 2746 | 2304 |
| | Ab1-17 synNotch 2e6 | | | | | | | |
| 14 | 108 | 108 | 108 | 126 | 126 | 126 | 126 | 144 |
| 17 | 172 | 288 | 245 | 256 | 320 | 405 | 256 | 446 |
| 21 | 666 | 726 | 700 | 787 | 650 | 1268 | 864 | 847 |
| 24 | 1008 | 1268 | 908 | 1183 | 847 | | 1183 | 1080 |
| 27 | 1080 | 2025 | 908 | 1268 | 1183 | | 1568 | |
| 30 | 1080 | | 908 | 1268 | 1470 | | 1568 | |
| 34 | 288 | | 908 | 1268 | 1470 | | 1268 | |
| 37 | 288 | | 600 | 1268 | 1268 | | 1268 | |
| 41 | 288 | | 600 | | 1268 | | 1268 | |
| 44 | 196 | | 320 | | 1352 | | 1470 | |
| 48 | 172 | | 126 | | 1437 | | 1688 | |
| 51 | | | 108 | | | | | |
| 55 | | | 108 | | | | | |
| | Ab1-17 synNotch 5e6 | | | | | | | |
| 14 | 108 | 108 | 108 | 126 | 126 | 126 | 126 | 144 |
| 17 | 256 | 256 | 196 | 365 | 256 | 446 | 365 | 405 |
| 21 | 726 | 936 | 600 | 936 | 666 | 1089 | 1089 | 864 |
| 24 | 936 | 936 | 787 | 1568 | 666 | 1368 | 1368 | 1183 |
| 27 | 1008 | 726 | 600 | 2025 | 666 | 1368 | 1368 | |
| 30 | 666 | 550 | 550 | | 405 | 1368 | 1080 | |
| 34 | 365 | 500 | 288 | | 172 | 567 | 550 | |
| 37 | 365 | 365 | 172 | | 108 | 416 | 256 | |
| 41 | 256 | 172 | 108 | | 32 | 245 | 196 | |
| 44 | 172 | 108 | 63 | | 32 | 144 | 172 | |
| 48 | 126 | 108 | 32 | | 32 | 88 | 108 | |
| 51 | 63 | 32 | 32 | | 32 | 88 | 32 | |
| 55 | 63 | 32 | 32 | | 32 | 88 | 32 | |
| | Ab2-7 synNotch 2e6 | | | | | | | |
| 14 | 108 | 108 | 108 | 126 | 126 | 126 | 126 | 144 |
| 17 | 256 | 288 | 196 | 320 | 365 | 365 | 405 | 320 |
| 21 | 666 | 787 | 666 | 936 | 864 | 1183 | 1080 | 1080 |
| 24 | 864 | 1080 | 726 | 1080 | 1268 | 1268 | 1352 | 1521 |
| 27 | 864 | | 864 | 1152 | 1352 | 1268 | 1521 | 1862 |
| 30 | 864 | | 864 | 1152 | 1666 | 1800 | 1521 | 2156 |
| 34 | 666 | | 726 | 847 | 1568 | 2176 | 1080 | |
| 37 | 288 | | 405 | 446 | 1268 | | 847 | |
| 41 | 172 | | 288 | 405 | 1268 | | 787 | |
| 44 | 172 | | 288 | 365 | 1268 | | 446 | |
| 48 | 108 | | 88 | 256 | 1268 | | 446 | |
| 51 | 108 | | 88 | 172 | 1268 | | 405 | |
| 55 | 108 | | 75 | 172 | 1268 | | | |
| | Ab2-7 synNotch 5e6 | | | | | | | |
| 14 | 108 | 108 | 108 | 126 | 126 | 126 | 126 | 144 |
| 17 | 172 | 172 | 256 | 245 | 365 | 288 | 320 | 288 |
| 21 | 600 | 550 | 936 | 800 | 1008 | 787 | 787 | 787 |
| 24 | 600 | 600 | 936 | 1029 | 1183 | 787 | 1008 | 847 |
| 27 | 365 | 365 | 726 | 968 | 1183 | 500 | 1008 | 527 |
| 30 | 256 | 256 | 365 | 650 | 550 | 365 | 650 | |
| 34 | 108 | 75 | 172 | 320 | 288 | 256 | 352 | |
| 37 | 63 | 32 | 63 | 221 | 172 | 172 | 320 | |
| 41 | 63 | 14 | 63 | | 108 | 108 | 221 | |
| 44 | 14 | 14 | 63 | | 108 | 108 | 221 | |
| 48 | 4 | 4 | 32 | | 63 | 63 | 126 | |
| 51 | | 4 | 32 | | 32 | 32 | 63 | |
| 55 | | 4 | 32 | | 32 | 32 | 32 | |
| | Ab3-21 synNotch 2e6 | | | | | | | |
| 14 | 108 | 108 | 108 | 108 | 126 | 126 | 126 | 144 |
| 17 | 288 | 365 | 256 | 196 | 365 | 256 | 405 | 320 |
| 21 | 936 | 936 | 666 | 600 | 1008 | 726 | 1268 | 1008 |
| 24 | 1470 | 1099 | 1099 | 936 | 1568 | 1268 | 1913 | |
| 27 | 1913 | 1372 | 1183 | 1183 | 2025 | 1666 | 2432 | |
| 30 | 2138 | 1800 | 1268 | | | 2138 | | |
| 34 | | 2025 | 2025 | | | | | |
| | Ab3-21 synNotch 5e6 | | | | | | | |
| 14 | 108 | 108 | 108 | 108 | 126 | 126 | 126 | 144 |
| 17 | 256 | 288 | 256 | 196 | 196 | 288 | 288 | 365 |
| 21 | 550 | 847 | 666 | 550 | 486 | 936 | 864 | 787 |
| 24 | 936 | 847 | 864 | 787 | 908 | | 1470 | 1080 |
| 27 | 1099 | 1008 | 726 | 1008 | 968 | | 1800 | 1960 |
| 30 | 1099 | 1268 | 726 | 1437 | 1029 | | 2025 | |
| 34 | 1688 | 1470 | 726 | | 1089 | | | |
| 37 | 1800 | | 726 | | 1440 | | | |
| 41 | 2176 | | 726 | | 2058 | | | |
| | Ab5 synNotch 2e6 | | | | | | | |
| 14 | 108 | 108 | 108 | 108 | 126 | 126 | 126 | 144 |
| 17 | 365 | 288 | 320 | 256 | 365 | 320 | 405 | 405 |
| 21 | 864 | 650 | 1183 | 550 | 936 | 726 | 787 | 1008 |
| 24 | 1372 | 1183 | 1913 | 1099 | 1568 | 1268 | 1183 | 1666 |
| 27 | 1800 | 1800 | 2890 | | 2304 | 2304 | 1568 | 2025 |
| 30 | 2025 | 2304 | | | | | 2025 | |
| | Ab5 synNotch 5e6 | | | | | | | |
| 14 | 108 | 108 | 108 | 108 | 126 | 126 | 126 | 144 |
| 17 | 288 | 196 | 221 | 288 | 365 | 196 | 365 | 288 |
| 21 | 726 | 500 | 650 | 936 | 1183 | 726 | 1099 | 1080 |
| 24 | 936 | 550 | 650 | 1183 | | 726 | 1372 | 1268 |
| 27 | 666 | 405 | 600 | 1183 | | 726 | 1470 | 1690 |
| 30 | 500 | 288 | 446 | 1372 | | 486 | | 1960 |
| 34 | 500 | 196 | 288 | 1470 | | 486 | | 2250 |
| 37 | 365 | 63 | 108 | 1470 | | 352 | | |
| 41 | | 32 | 75 | 1913 | | 352 | | |
| 44 | | 14 | 14 | 2025 | | 352 | | |
| 48 | | 14 | 14 | | | 352 | | |
| 51 | | 14 | 14 | | | 256 | | |
| 55 | | 14 | 14 | | | | | |

### Example 7

Female NSG mice were implanted with 5×10⁵ K562-PSCA-PSMA subcutaneously in the dorsal flank in 50% Matrigel. On day 13. mice were dosed with either 2×10⁶ or 5×10⁶ CAR+ T cells. Tumors were measured via calipers.

| Day post | | | | | | | | |
|---|---|---|---|---|---|---|---|---|
| inoculation | | | | | | | | |
| | Vehicle | | | | | | | |
| 13 | 108 | 108 | 108 | 126 | 126 | 172 | 172 | 172 |
| 16 | 172 | 172 | 256 | 365 | 221 | 288 | 256 | 256 |
| 20 | 550 | 500 | 726 | 787 | 650 | 600 | 726 | 936 |
| 23 | 1008 | 787 | 1268 | 1352 | 968 | 1080 | 1568 | 1568 |
| 27 | 2138 | 2048 | 2304 | | 1606 | 2025 | 2304 | 2890 |
| 30 | | | | | 2058 | | | |
| | Nontransduced | | | | | | | |
| 13 | 108 | 108 | 126 | 126 | 126 | 144 | 172 | 172 |
| 16 | 172 | 288 | 256 | 256 | 196 | 320 | 256 | 288 |
| 20 | 500 | 726 | 666 | 726 | 787 | 1008 | 650 | 864 |
| 23 | 1099 | 1183 | 1268 | 1268 | 1183 | 1764 | 1152 | 1470 |
| 27 | 2048 | 2890 | 2457 | 2601 | 2432 | 2890 | 2432 | 2916 |
| | synNotch/mAb1 anti-PSMA CAR (2e6) | | | | | | | |
| 13 | 108 | 108 | 126 | 126 | 126 | 144 | 172 | 172 |
| 16 | 172 | 172 | 256 | 365 | 196 | 365 | 405 | 256 |
| 20 | 446 | 365 | 650 | 864 | 446 | 787 | 1099 | 550 |
| 23 | 486 | 405 | 1080 | 1372 | 700 | 1080 | 1688 | 864 |
| 27 | 650 | 288 | 1437 | 1470 | 527 | 1470 | 2176 | 1099 |
| 30 | 700 | 196 | 1666 | 1470 | 486 | 1800 | | 787 |
| 34 | 700 | 172 | 2025 | 1268 | 245 | 1568 | | 726 |
| 38 | 486 | 108 | | 936 | 196 | 1268 | | 500 |
| 41 | 196 | 108 | | 787 | 126 | 787 | | 500 |
| 44 | 172 | 75 | | 486 | 108 | 550 | | 500 |
| 48 | 172 | 75 | | 550 | 32 | 550 | | 500 |
| | synNotch/mAb1 anti-PSMA CAR + DNR (2e6) | | | | | | | |
| 13 | 108 | 108 | 126 | 126 | 126 | 144 | 172 | 172 |
| 16 | 256 | 108 | 256 | 320 | 365 | 196 | 365 | 256 |
| 20 | 446 | 256 | 550 | 666 | 600 | 666 | 864 | 726 |
| 23 | 666 | 320 | 726 | 666 | 787 | 936 | 1183 | 1099 |
| 27 | 726 | 352 | 787 | 726 | 650 | 1470 | 1470 | 1183 |
| 30 | 666 | 245 | 726 | 666 | 446 | 1470 | 1470 | 1080 |
| 34 | 446 | 126 | 550 | 365 | 320 | 1568 | 1470 | 1080 |
| 38 | 320 | 126 | 405 | 288 | 196 | 1372 | 787 | 787 |
| 41 | 144 | 108 | 256 | 256 | 172 | 936 | 666 | 600 |
| 44 | 144 | 108 | 288 | 256 | 126 | 864 | 550 | 550 |
| 48 | 75 | 32 | 196 | 75 | 63 | 666 | 550 | 446 |
| | synNotch/mAb1 anti-PSMA CAR (5e6) | | | | | | | |
| 13 | 108 | 108 | 126 | 126 | 126 | 144 | 172 | 172 |
| 16 | 172 | 196 | 172 | 196 | 256 | 365 | 365 | 405 |
| 20 | 288 | 245 | 288 | 405 | 405 | 936 | 1099 | 936 |
| 23 | 288 | 352 | 288 | 405 | 405 | 1080 | 1470 | 1268 |
| 27 | 108 | 221 | 172 | 196 | 196 | 1152 | 1800 | 1183 |
| 30 | 63 | 172 | 144 | 172 | 126 | 787 | 1470 | 726 |
| 34 | 63 | 126 | 75 | 108 | 108 | 486 | 600 | 666 |
| 38 | 14 | 108 | 88 | 108 | 108 | 352 | 550 | 500 |
| 41 | 14 | 88 | 88 | 88 | 63 | 320 | 500 | 405 |
| 44 | 14 | 108 | 126 | 63 | 63 | 320 | 365 | 320 |
| 48 | 14 | 108 | 32 | 32 | 32 | 288 | 288 | 288 |
| | synNotch/mAb1 anti-PSMA CAR + DNR (5e6) | | | | | | | |
| 13 | 108 | 108 | 126 | 126 | 126 | 126 | 172 | 172 |
| 16 | 172 | 172 | 256 | 288 | 256 | 256 | 288 | 405 |
| 20 | 256 | 446 | 550 | 726 | 500 | 666 | 666 | 1008 |
| 23 | 196 | 787 | 666 | 864 | 550 | 600 | 726 | 1568 |
| 27 | 126 | 787 | 550 | 787 | 405 | 500 | 864 | 1666 |
| 30 | 108 | 405 | 288 | 550 | 256 | 288 | 726 | 1470 |
| 34 | 75 | 320 | 256 | 446 | 126 | 196 | 405 | 550 |
| 38 | 32 | 221 | 196 | 320 | 75 | 172 | 256 | 365 |
| 41 | 18 | 221 | 196 | 288 | 63 | 172 | 196 | 196 |
| 44 | 14 | 221 | 196 | 288 | | 196 | 256 | 196 |
| 48 | 0 | 172 | 126 | 256 | | 75 | | 172 |

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-16. (canceled)

17. A synthetic notch (synNotch) receptor polypeptide comprising from N to C terminus:
an extracellular anti-PSCA binding domain,
a Notch core domain comprising one or more proteolytic cleavage sites, and
an intracellular domain comprising a transcriptional activator comprising a DNA binding domain and a transactivation domain, wherein binding of the binding extracellular anti-PSCA binding domain to PSCA induces cleavage of the Notch core domain at the one or more proteolytic cleavage sites, thereby releasing the intracellular domain and the transcriptional regulator.

18. The synNotch receptor polypeptide of claim 17, wherein the anti-PSCA binding domain comprising a first domain comprising three heavy chain complementarity determining regions (HCDR1, HCDR2 and HCDR3) and a second domain comprising three light chain complementarity determining regions (LCDR1, LCDR2 and LCDR3), wherein
(i) the HCDR1 has a sequence according to any one of SEQ ID NOs: 152-154,
(ii) the HCDR2 has a sequence according to any one of SEQ ID NOs: 155-157;
(iii) the HCDR3 has a sequence according to any one of SEQ ID NOs: 158-160;
(iv) the LCDR1 has a sequence according to any one of SEQ ID NOs: 163-165;
(v) the LCDR2 has a sequence according to any one of SEQ ID NOs: 166-168; and
(vi) the LCDR3 has a sequence according to any one of SEQ ID NOs: 169-171.

19. The synNotch receptor polypeptide of claim 18, wherein the transcriptional regulator is a transcriptional activator.

20. The synNotch receptor polypeptide of claim 19, wherein the transcriptional activator comprises GAL4, HNF1 alpha or HNF1 beta.

21. The synNotch receptor polypeptide of claim 19, wherein the transcriptional activator comprises a transactivation domain selected from the group consisting of VP64, RelA (p65), YAP, WWTR1 (TAZ), CREB3 (LZIP), and MyoD.

22. A nucleic encoding the synNotch receptor polypeptide of claim 17.

23. A recombinant vector comprising the nucleic acid of claim 22.
